SEQUENCE LISTING (8 sequences)

SEQ ID NO: 1 - RNA, 19 nt, Artificial Sequence
  SETD2 inhibitor
uaaaggagguauaucgaau

SEQ ID NO: 2 - RNA, 19 nt, Artificial Sequence
  SETD2 inhibitor
gagagguacucgaucauaa

SEQ ID NO: 3 - RNA, 19 nt, Artificial Sequence
  SETD2 inhibitor
gcucagaguuaacguuuga

SEQ ID NO: 4 - RNA, 19 nt, Artificial Sequence
  SETD2 inhibitor
ccaaagauucagacauaua

SEQ ID NO: 5 - DNA, 20 bp, Artificial Sequence
  SETD2 inhibitor
cccattcactgtccacttga

SEQ ID NO: 6 - DNA, 20 bp, Artificial Sequence
  SETD2 inhibitor
ccctcaagtggacagtgaat

SEQ ID NO: 7 - DNA, 20 bp, Artificial Sequence
  sgRNA Target Sequence #1
agcaccagtaacagagccag

SEQ ID NO: 8 - DNA, 20 bp, Artificial Sequence
  sgRNA Target Sequence #2
gactgtgaacggacaactga

(19) United States
(12) Patent Application Publication
(10) Pub. No.: US 2023/0049113 A1
(43) Pub. Date: Feb. 16, 2023

(54) METHODS OF TREATING WHSC1-OVEREXPRESSING CANCERS BY INHIBITING SETD2

(71) Applicant: EPIZYME, INC., Cambridge, MA (US)

(72) Inventors: Michael THOMENIUS, Arlington, MA (US); Katherine Louise COSMOPOULOS, Medford, MA (US); Jennifer Anne TOTMAN, Berlin, MA (US)

(21) Appl. No.: 17/294,959

(22) PCT Filed: Nov. 26, 2019
(86) PCT No.: PCT/US19/63405

§ 371 Date: May 18, 2021

Related U.S. Application Data
(63) Continuation-in-part of application No. PCT/US2019/046569, filed on Aug. 14, 2019.
(60) Provisional application No. 62/773,770, filed on Nov. 30, 2018.
(60) Provisional application No. 62/857,120, filed on Jun. 4, 2019.
(60) Provisional application No. 62/886,880, filed on Aug. 14, 2019.

(51) Int. Cl. A61K 31/404; A61P 35/00; A61K 31/4985; A61K 31/496; A61K 31/438; A61K 31/541; A61K 31/4439; A61K 31/437; A61K 31/5377; A61K 31/407; A61K 31/422; A61K 31/519; A61K 31/4709; A61K 31/551; A61K 31/454; A61K 31/55; A61K 31/439; A61K 31/506; A61K 31/4245; A61K 31/433; A61K 31/4725; A61K 31/5386; A61K 31/427; A61K 31/415; A61K 31/4995; A61K 31/4196; A61K 31/4155; A61K 31/517; A61K 31/4178; A61K 31/537; A61K 31/499

(52) U.S. Cl. CPC A61K 31/404; A61K 31/499; A61K 31/4985; A61K 31/496; A61K 31/438; A61K 31/541; A61K 31/4439; A61K 31/437; A61K 31/5377; A61K 31/407; A61K 31/422; A61K 31/519; A61K 31/4709; A61K 31/551; A61K 31/454; A61K 31/55; A61K 31/439; A61K 31/506; A61K 31/4245; A61K 31/433; A61K 31/4725; A61K 31/5386; A61K 31/427; A61K 31/415; A61K 31/4995; A61K 31/4196; A61K 31/4155; A61K 31/517; A61K 31/4178; A61K 31/537; A61P 35/00

(57) ABSTRACT

The present disclosure provides methods and pharmaceutical compositions for treating or slowing the progression of cancers that overexpress the histone methyltransferase WHSC1, e.g., t(4; 14) multiple myeloma, by administering to a subject in need thereof a therapeutically effective amount of an inhibitor of the histone methyltransferase, SETD2.

### REFERENCE TO SEQUENCE LISTING SUBMITTED ELECTRONICALLY

The content of the electronically submitted sequence listing in ASCII text file (Name: 3562_018PC04_Seqlisting_ST25.txt; Size: 1,882 bytes; and Date of Creation: Nov. 26, 2019), filed with the application, is incorporated herein by reference in its entirety.

### FIELD OF DISCLOSURE

The disclosure relates generally to the field of epigenetic-based cancer therapy. More particularly, the present disclosure relates to methods and pharmaceutical compositions for treating cancers that overexpress the histone methyltransferase, WHSC1, by inhibiting the histone methyltransferase, SETD2.

### BACKGROUND

Histone lysine methylation is a principal chromatin-regulatory mechanism that influences fundamental nuclear processes. The selective addition of methyl groups to specific amino acid sites on histones is controlled by the action of a family of enzymes known as histone methyltransferases (HMTs). The level of expression of a particular gene is influenced by the presence or absence of one or more methyl groups at a relevant histone site. The specific effect of a methyl group at a particular histone site persists until the methyl group is removed by a histone demethylase, or until the modified histone is replaced through nucleosome turnover. In a like manner, other enzyme classes can decorate DNA and histones with other chemical species, and still other enzymes can remove these species to provide control of gene expression.

WHSC1 (also known as Wolf-Hirschhorn Syndrome Candidate Gene 1, MMSET, NSD2, REIIBP, TRX5, and WHS) is a HMT located at cytogenic band p16.3 of chromosome 4 (4p16.3). The principal chromatin-regulatory effect of WHSC1 is dimethylation of histone H3 at lysine 36 (H3K36me2), which activates transcription. Kuo, A. J. et al., Mol. Cell. 44:609-620 (2011). WHSC1 is overexpressed in numerous cancers compared to their normal counterparts, and is linked with tumor aggressiveness. Kassambara, A. et. al., Biochem. Biophys. Res. Comm. 379:840-845 (2009). In particular, WHSC1 has been shown to be highly overexpressed in t(4; 14) multiple myeloma (MM), which has been associated with a poor prognosis. Id.

SETD2 is another HMT that is located at cytogenic band p21.31 of chromosome 3 (3p21.31). The acronym “SETD2” stands for Suppressor of variegation, Enhancer of zeste, and Trithorax domain containing 2. The SETD2 protein comprises three conserved functional domains: (1) the triplicate AWS-SET-PostSET domain; (2) a WW domain; and (3) a Set2-Rbp1 interacting (“SRI”) domain. These three functional domains define the biological function of SETD2. See, Li, J. et al., Oncotarget 7:50719-50734 (2016). SETD2 is believed to be the single human gene responsible for the trimethylation of lysine 36 (Lys-36) of histone H3 (H3K36me3) using dimethylated Lys-36 (H3K36me2) as a substrate. Edmunds, J. W. et al., The EMBO Journal 27:406-420 (2008).

Human SETD2 is also a putative tumor suppressor. Li, J. et al., Oncotarget 7:50719-50734 (2016). For example, inactivation of human SETD2 has been reported in renal cell carcinoma (RCC). Larkin, J., et al., Nature Reviews 9:147-155 (2012). Also, expression levels of SETD2 in breast cancer samples have been reported as significantly lower than in adjacent non-cancerous tissue (ANCT) samples. Newbold, R. F. and Mokbel, K., Anticancer Research 30: 3309-3311 (2010). Additionally, biallelic mutations and loss-of-function point mutations in SETD2 were reported in patients with acute leukemia. Zhu, X. et al., Nature Genetics 46: 287-293 (2014). Mutations in SETD2 have also been reported in pediatric high-grade gliomas. Fontebasso, A. M. et al., Acta Neuropathol. 125: 659-669 (2013).

Despite more than a century of dedicated scientific and clinical research, curing cancer remains one of the biggest medical challenges to date. Cancer treatments have mainly relied on the combination of surgery, radiotherapy, and/or cytotoxic chemotherapies. And while effective cancer therapies exist, suboptimal response, relapsed-refractory disease, and/or resistance to one or more therapeutic agents have remained a challenge, especially for certain subtypes of multiple myeloma (i.e., t(4; 14) multiple myeloma). Accordingly, there is a medical need for more effective, safe, and durable therapies for the treatment of all types of cancer.

### SUMMARY

The present disclosure relates to epigenetic-based cancer therapy, and the unexpected discovery that inhibiting SETD2, despite its functionality as a tumor suppressor, can be used to treat cancer. Additionally, the present disclosure relates to the unexpected discovery that inhibiting SETD2 can be used to treat cancers that overexpress WHSC1.

In one aspect, the present disclosure is directed to a method of treating cancer in a subject in need thereof, the method comprising administering to the subject a therapeutically effective amount of a SETD2 inhibitor, wherein the cancer overexpresses WHSC1.

In certain embodiments, overexpression of WHSC1 by said cancer is determined prior to administering said SETD2 inhibitor.

In certain embodiments, the SETD2 inhibitor is a “Substituted Indole Compound” as defined in the “Definitions” section of the DETAILED DESCRIPTION.

In certain embodiments, the SETD2 inhibitor is a compound of Table 1, or a pharmaceutically acceptable salt thereof.

In certain embodiments, the SETD2 inhibitor is not a Substituted Indole Compound.

In certain embodiments, the cancer that overexpresses WHSC1 is a hematologic cancer.

In certain embodiments, the hematologic cancer is selected from the group consisting of acute lymphocytic leukemia (ALL), acute myeloid leukemia (AML), chronic lymphocytic leukemia (CLL), small lymphocytic lymphoma (SLL), multiple myeloma (MM), Hodgkin's lymphoma (HL), non-Hodgkin's lymphoma (NHL), mantle cell lymphoma (MCL), marginal zone B-cell lymphoma, splenic marginal zone lymphoma, follicular lymphoma (FL), Waldenstrom's macroglobulinemia (WM), diffuse large B-cell lymphoma (DLBCL), marginal zone lymphoma (MZL), hairy cell leukemia (HCL), Burkitt's lymphoma (BL), Richter's transformation, acute eosinophilic leukemia, acute erythroid leukemia, acute lymphoblastic leukemia, acute megakaryoblastic leukemia, acute monocytic leukemia, acute promyelocytic leukemia, acute myelogenous leukemia, B-cell prolymphocytic leukemia, B-cell lymphoma, MALT lymphoma, precursor T-lymphoblastic lymphoma, T-cell lymphoma, mast cell leukemia, adult T cell leukemia/lymphoma, aggressive NK-cell leukemia, and angioimmunoblastic T-cell lymphoma.

In certain embodiments, the hematologic cancer is multiple myeloma.

In certain embodiments, the multiple myeloma contains a chromosomal translocation or a chromosomal deletion.

In certain embodiments, the chromosomal translocation involves chromosome 14. In certain embodiments, the chromosomal translocation is a t (4; 14) translocation. In certain embodiments, the chromosomal translocation is a non-t(4;14) translocation. In certain embodiments, the non-t(4;14) translocation is selected from the group consisting of a t(14;16); t(11; 14); t(14;20), t(8; 14), and t(6; 14) translocation.

In certain embodiments, the multiple myeloma contains a deletion. In certain embodiments, the deletion is selected from the group consisting of del(17p) and del(13).

In certain embodiments, the cancer that overexpresses WHSC1 is a solid tumor.

In certain embodiments, the solid tumor is selected from the group consisting of esophageal cancer, kidney cancer, stomach cancer, hepatocellular carcinoma, glioblastoma, central nervous system (CNS) cancer, soft tissue cancer, lung cancer, breast cancer, bladder/urinary tract cancer, head and neck cancer, melanoma, prostate cancer, testicular cancer, pancreatic cancer, skin cancer, endometrial cancer, ovarian cancer, colon cancer, and colorectal cancer.

In certain embodiments, the subject is a mammal. In certain embodiments, the subject is a human.

In certain embodiments, the SETD2 inhibitor is formulated for systemic or local administration. In certain embodiments, the SETD2 inhibitor is formulated for oral, nasal, intra-peritoneal, or intra-tumoral administration. In certain embodiments, the SETD2 inhibitor is formulated for intravenous administration, intramuscular administration, or subcutaneous administration.

In one aspect, the present disclosure is directed to a method of inhibiting the trimethylation of lysine 36 on histone H3 (H3K36me3) in a cell, the method comprising contacting said cell with a SETD2 inhibitor, wherein the cell overexpresses WHSC1.

In certain embodiments, the SETD2 inhibitor is a “Substituted Indole Compound” as defined in the “Definitions” section of the DETAILED DESCRIPTION.

In certain embodiments, the SETD2 inhibitor is a compound of Table 1, or a pharmaceutically acceptable salt thereof.

In certain embodiments, the SETD2 inhibitor is not a Substituted Indole Compound.

In certain embodiments, inhibiting trimethylation of lysine 36 on histone H3 in a cell occurs in vitro. In certain embodiments, inhibiting trimethylation of lysine 36 on histone H3 in a cell occurs in vivo.

In certain embodiments, the cell is derived from a hematologic cancer. In certain embodiments, the hematologic cancer is selected from the group consisting of acute lymphocytic leukemia (ALL), acute myeloid leukemia (AML), chronic lymphocytic leukemia (CLL), small lymphocytic lymphoma (SLL), multiple myeloma (MM), Hodgkin's lymphoma (HL), non-Hodgkin's lymphoma (NHL), mantle cell lymphoma (MCL), marginal zone B-cell lymphoma, splenic marginal zone lymphoma, follicular lymphoma (FL), Waldenstrom's macroglobulinemia (WM), diffuse large B-cell lymphoma (DLBCL), marginal zone lymphoma (MZL), hairy cell leukemia (HCL), Burkitt's lymphoma (BL), Richter's transformation, acute eosinophilic leukemia, acute erythroid leukemia, acute lymphoblastic leukemia, acute megakaryoblastic leukemia, acute monocytic leukemia, acute promyelocytic leukemia, acute myelogenous leukemia, B-cell prolymphocytic leukemia, B-cell lymphoma, MALT lymphoma, precursor T-lymphoblastic lymphoma, T-cell lymphoma, mast cell leukemia, adult T cell leukemia/lymphoma, aggressive NK-cell leukemia, and angioimmunoblastic T-cell lymphoma.

In certain embodiments, the hematologic cancer is multiple myeloma.

In certain embodiments, the multiple myeloma contains a chromosomal translocation or a chromosomal deletion.

In certain embodiments, the chromosomal translocation involves chromosome 14. In certain embodiments, the chromosomal translocation is a t (4; 14) translocation. In certain embodiments, the chromosomal translocation is a non-t(4;14) translocation. In certain embodiments, the non-t(4;14) translocation is selected from the group consisting of a t(14;16); t(11; 14); t(14;20), t(8; 14), and t(6; 14) translocation.

In certain embodiments, the multiple myeloma contains a deletion. In certain embodiments, the deletion is selected from the group consisting of del(17p) and del(13).

In certain embodiments, the cell is derived from a solid tumor.

In certain embodiments, the solid tumor is selected from the group consisting of esophageal cancer, kidney cancer, stomach cancer, hepatocellular carcinoma, glioblastoma, central nervous system (CNS) cancer, soft tissue cancer, lung cancer, breast cancer, bladder/urinary tract cancer, head and neck cancer, melanoma, prostate cancer, testicular cancer, pancreatic cancer, skin cancer, endometrial cancer, ovarian cancer, colon cancer, and colorectal cancer.

In certain embodiments, the in vivo cell is in a mammal. In certain embodiments, the in vivo cell is in a human.

### BRIEF DESCRIPTION OF THE DRAWINGS

FIG. 1A-FIG. 1B show the anti-proliferative effects of Cpd. No. 15 (N-((1R,3S)-3-(4-acetylpiperazin-1-yl)cyclohexyl)-4-fluoro-7-methyl-1H-indole-2-carboxamide), see Table 1, on a panel of multiple myeloma cell lines. FIG. 1A is a table of multiple myeloma (MM) cell lines that were tested in a Long-Term Proliferation (LTP) 14-day assay, with translocation status, isotype, and 14-day proliferation half maximal inhibitory concentration IC₅₀. FIG. 1B is a graph depicting 14-day proliferation IC₅₀ for the MM cell lines tested. Each point represents a different cell line. Cell lines are grouped by t(4; 14) status. 14-day proliferation IC₅₀ is shown on the y-axis.

FIG. 2A-FIG. 2D show that Cpd. No. 15 mediated inhibition of the t(4; 14) multiple myeloma cell line KMS-34 is due to SETD2 inhibition. FIG. 2A is a graph showing that KMS-34 has a cytotoxic response to Cpd. No. 15 with an 80 nM proliferation IC₅₀ in a 14-day LTP assay. Each point represents the mean for each concentration (n=3). FIG. 2B is a developed Western blot showing that KMS-34 shows a dose-dependent decrease in H3K36me3 with Cpd. No. 15, while H3K36me2 is unaffected after 14 days. FIG. 2C is a table showing that the activity of Cpd. No. 15 is stereo-specific, as the most biochemically active enantiomer. FIG. 2D is a graph showing the structure-activity relationship (SAR) observed with SETD2 inhibitors, when comparing H3K36me3 inhibition potency and anti-proliferative activity. Each point represents a SETD2 inhibitor run in an A549 H3K36me3 assay and a KMS-34 long-term proliferation assay.

FIG. 3A-FIG. 3C show that sensitivity to Cpd. No. 15, in some embodiments, is associated with WHSC1 overexpression status. Two isogenic variants of KMS11 cells, the TKOs and NTKOs, have been characterized previously (Kuo A. J. et al., Mol. Cell. 44 609-620 (2011)). Non-translocation knock-out (NTKO) cells express only the translocated WHSC1. Translocation knock-out (TKO) cells express only the non-translocated allele of WHSC1. FIG. 3A is a graph showing the results of a 14-day proliferation assay with Cpd. No. 15. FIG. 3B and FIG. 3C depict H3K36me2 and H3K36me3 western blots (FIG. 3B) from KMS11 parental, TKO, and NTKO cell lines treated with Cpd. No. 15; FIG. 3C is a bar graph depicting the proliferation IC₅₀ and H3K36me3 levels for each cell line found in FIG. 3A and FIG. 3B, respectively.

FIG. 4A-FIG. 4B show that, in some embodiments, KMS34 cells require WHSC1 for proliferation, but KMS-28-BM cells do not. FIG. 4A are graphs depicting Long-Term Proliferation (LTP) assay results in WHSC1-CRISPR targeted cell lines. FIG. 4B are bar graphs depicting WHSC1 genotype over time after CRISPR targeting.

FIG. 5A-FIG. 5C depict drug exposure and body weights for a 7-day dose range finding study in mice, and shows that Cpd. No. 15 is tolerated and can be maintained at concentrations that are effective in vitro. FIG. 5A is a graph showing that levels of Cpd. No. 15 are maintained that exceed 10× the KMS11 proliferation IC₅₀ in mice with either twice a day (BID) or once a day (QD) dosing. FIG. 5B is a graph showing that no body weight loss is observed at 62.5 and 125 mg/kg BID dosing, when compared with vehicle control. FIG. 5C depicts a Western blot and a graphical presentation of histone H3 probed with H3K36me3 or total H3 antibodies.

FIG. 6A-FIG. 6C show that Cpd. No. 15 demonstrates strong anti-tumor activity in KMS11, a t(4; 14) multiple myeloma xenograft model. FIG. 6A is a graph showing KMS11 xenograft tumor growth regression with BID dosing of Cpd. No. 15 for 28 days.

FIG. 6B is a graph showing minimal body weight loss at 31.25 and 62.5 mg/kg (28 days BID) when compared to vehicle control. FIG. 6C shows fluorescent-based ELISA of KMS11 tumor samples.

FIG. 7A-FIG. 7C show that Cpd. No. 15 demonstrates anti-tumor activity in MM.1S, a non-t(4;14) multiple myeloma xenograft model. FIG. 7A is a graph showing MM.1S xenograft tumor growth inhibition with BID dosing of Cpd. No. 15 for 23 days. FIG. 7B is a graph showing minimal body weight loss at all doses when compared to vehicle control. FIG. 7C shows fluorescent-based ELISA data depicting the reduction of H3K36me3 with Cpd. No. 15 treatment in MM.1S tumor samples.

### DETAILED DESCRIPTION

### Definitions

To facilitate an understanding of the present invention, a number of terms and phrases are defined below.

Open terms such as “include,” “including,” “contain,” “containing” and the like mean “comprising.” These open-ended transitional phrases are used to introduce an open ended list of elements, method steps, or the like that does not exclude additional, unrecited elements or method steps. Wherever aspects are described herein with the language “comprising,” otherwise analogous aspects described in terms of “consisting of” and/or “consisting essentially of” are also provided.

As used in the present disclosure and claims, the singular forms “a,” “an,” and “the” include plural forms unless the context clearly dictates otherwise. For example, “a cell” includes a single cell as well as a plurality of cells, including mixtures thereof.

The term “Substituted Indole Compound” as used herein refers to a compound disclosed in International Application No. PCT/US2019/046569, filed Aug. 14, 2019, and the pharmaceutically acceptable salts and solvates thereof. Thus, in one embodiment, a Substituted Indole Compound is a compound having Formula I:

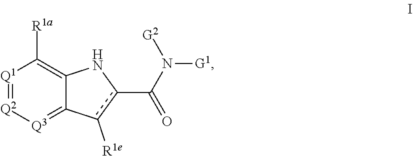

wherein:

R^{1a} is selected from the group consisting of halogen, alkyl, alkoxy, cycloalkyl, (hydroxy)alkyl, and (cycloalkyl)alkyl;

Q¹ is selected from the group consisting of —C(R^{1b})═ and —N═;

Q² is selected from the group consisting of —C(R^{1c})═ and —N═;

Q³ is selected from the group consisting of —C(R^{1d})═ and —N═;

provided that at least one of Q¹, Q², or Q³ is —C(R^{1b})═, —C(R^{1c})═, or —C(R^{1d})═, respectively;

R^{1b}, R^{1c}, and R^{1d} are each independently selected from the group consisting of hydrogen, halogen, alkyl, alkenyl, (hydroxy)alkyl, and alkoxy;

R^{1e} is selected from the group consisting of hydrogen, halogen, alkyl, cycloalkyl, (hydroxy)alkyl, and (cycloalkyl)alkyl;

[symbol] is a single or double bond;

G¹ is selected from the group consisting of optionally substituted aryl, optionally substituted heteroaryl, optionally substituted heterocyclo, optionally substituted cycloalkyl, (aryl)alkyl, (heteroaryl)alkyl, (heterocyclo)alkyl, (amino)(aryl)alkyl, (heteroaryl)(aryl)alkyl, (heteroaryl)(heterocyclo)alkyl, (heteroaryl)(carboxamido)alkyl, (heteroaryl)(cycloalkyl)alkyl, (aryl)(alkoxycarbonyl)alkyl, (cycloalkyl)alkyl, (heteroaryl)(amino)alkyl, (cycloalkyl)(alkoxycarbonyl)alkyl, (heteroaryl)(alkoxycarbonyl)alkyl, (heterocyclo)(cycloalkyl)alkyl, (aryl)(cycloalkyl)alkyl, (aryl)(hydroxy)alkyl, (cycloalkyl)(hydroxy)alkyl, (hydroxy)alkyl, optionally substituted alkyl, (aryl)(haloalkyl)alkyl, (cycloalkyl)(haloalkyl)alkyl, (hydroxy)(haloalkyl)alkyl, and (alkoxycarbonyl)(haloalkyl)alkyl; and

G² is selected from the group consisting of hydrogen and alkyl; or

G¹ and G² taken together with the nitrogen atom to which they are attached form an optionally substituted heterocyclo, or a pharmaceutically acceptable salt or solvate thereof.

In another embodiment, a Substituted Indole Compound is a compound having Formula I, wherein:

R^{1a} is selected from the group consisting of halogen, C₁-C₆ alkyl, C₁-C₆ alkoxy, C₃-C₈ cycloalkyl, (hydroxy)C₁₋₆ alkyl, and (C₃-C₆ cycloalkyl)C₁₋₆ alkyl;

R^{1b}, R^{1c}, and R^{1d} are each independently selected from the group consisting of hydrogen, halogen, C₁-C₆ alkyl, C₂-C₆ alkenyl, (hydroxy)C₁-C₆ alkyl, and C₁-C₆ alkoxy;

R^{1e} is selected from the group consisting of hydrogen and C₁-C₆ alkyl;

G¹ is selected from the group consisting of optionally substituted C₆-C₁₀ aryl, optionally substituted 5- to 10-membered heteroaryl, optionally substituted 3- to 10-membered heterocyclo, optionally substituted C₃-C₈ cycloalkyl, (C₆-C₁₀ aryl)C₁-C₆ alkyl, (5- to 10-membered heteroaryl)C₁-C₆ alkyl, (3- to 10-membered heterocyclo)C₁-C₆ alkyl, (amino)(C₆-C₁₀ aryl)C₁-C₆ alkyl, (5- to 14-membered heteroaryl)(C₆-C₁₀ aryl)C₁-C₆ alkyl, (5- to 10-membered heteroaryl)(3- to 10-membered heterocyclo)C₁-C₆ alkyl, (5- to 10-membered heteroaryl)(carboxamido)C₁-C₆ alkyl, (5- to 10-membered heteroaryl)(C₃-C₆ cycloalkyl)C₁-C₆ alkyl, (C₆-C₁₀ aryl)(alkoxycarbonyl)C₁-C₆ alkyl, (C₃-C₆ cycloalkyl)C₁-C₆ alkyl, (5- to 10-membered heteroaryl)(amino)C₁-C₆ alkyl, (C₃-C₆ cycloalkyl)(alkoxycarbonyl)C₁-C₆ alkyl, (5- to 14-membered heteroaryl)(alkoxycarbonyl)C₁-C₆ alkyl, (3- to 14-membered heterocyclo)(C₃-C₈ cycloalkyl)C₁-C₆ alkyl, (C₆₋₁₀ aryl)(C₃-C₈ cycloalkyl)C₁-C₆ alkyl, (C₆-C₁₀ aryl)(hydroxy)C₁-C₆ alkyl, (C₃-C₆ cycloalkyl)(hydroxy)C₁-C₆ alkyl, (hydroxy)C₁-C₆ alkyl, optionally substituted C₁-C₆ alkyl, (C₆-C₁₀ aryl)(C₁-C₆haloalkyl)C₁-C₆ alkyl, (C₃-C₆ cycloalkyl)(C₁-C₆haloalkyl)C₁-C₆ alkyl, (hydroxy)(C₁-C₆haloalkyl)C₁-C₆ alkyl; and (alkoxycarbonyl)(C₁-C₆haloalkyl)C₁-C₆ alkyl; and

G² is selected from the group consisting of hydrogen and C₁-C₆ alkyl; or

G¹ and G² taken together with the nitrogen atom to which they are attached form a 5- to 10-membered optionally substituted heterocyclo, or a pharmaceutically acceptable salt or solvate thereof.

In another embodiment, a Substituted Indole Compound is a compound having Formula I, wherein:

R^{1a} is selected from the group consisting of halogen, C₁-C₃ alkyl, C₁-C₃ alkoxy, C₃-C₆ cycloalkyl, (hydroxy)C₁₋₄ alkyl, and (C₃-C₆ cycloalkyl)C₁₋₄ alkyl;

R^{1b}, R^{1c}, and R^{1d} are each independently selected from the group consisting of hydrogen, halogen, C₁-C₃ alkyl, C₂-C₄ alkenyl, (hydroxy)C₁-C₄ alkyl, and C₁-C₃ alkoxy;

R^{1e} is selected from the group consisting of hydrogen and C₁-C₃ alkyl;

G¹ is selected from the group consisting of optionally substituted C₆-C₁₀ aryl, optionally substituted 5- to 10-membered heteroaryl, optionally substituted 3- to 10-membered heterocyclo, optionally substituted C₃-C₈ cycloalkyl, (C₆-C₁₀ aryl)C₁-C₄ alkyl, (5- to 10-membered heteroaryl)C₁-C₆ alkyl, (3- to 10-membered heterocyclo)C₁-C₄ alkyl, (amino)(C₆-C₁₀ aryl)C₁-C₆ alkyl, (5- to 14-membered heteroaryl)(C₆-C₁₀ aryl)C₁-C₄ alkyl, (5- to 10-membered heteroaryl)(3- to 10-membered heterocyclo)C₁-C₄ alkyl, (5- to 10-membered heteroaryl)(carboxamido)C₁-C₄ alkyl, (5- to 10-membered heteroaryl)(C₃-C₆ cycloalkyl)C₁-C₄ alkyl, (C₆-C₁₀ aryl)(alkoxycarbonyl)C₁-C₄ alkyl, (C₃-C₆ cycloalkyl)C₁-C₄ alkyl, (5- to 10-membered heteroaryl)(amino)C₁-C₄ alkyl, (C₃-C₆ cycloalkyl)(alkoxycarbonyl)C₁-C₄ alkyl, (5- to 14-membered heteroaryl)(alkoxycarbonyl)C₁-C₄ alkyl, (3- to 14-membered heterocyclo)(C₃-C₆ cycloalkyl)C₁-C₄ alkyl, (C₆₋₁₀ aryl)(C₃-C₆ cycloalkyl)C₁-C₄ alkyl, (C₆-C₁₀ aryl)(hydroxy)C₁-C₄ alkyl, (C₃-C₆ cycloalkyl)(hydroxy)C₁-C₄ alkyl, (hydroxy)C₁-C₄ alkyl, optionally substituted C₁-C₄ alkyl, (C₆-C₁₀ aryl)(C₁-C₄haloalkyl)C₁-C₄ alkyl, (C₃-C₆ cycloalkyl)(C₁-C₄haloalkyl)C₁-C₄ alkyl, (hydroxy)(C₁-C₄haloalkyl)C₁-C₄ alkyl, and (alkoxycarbonyl)(C₁-C₄haloalkyl)C₁-C₄ alkyl; and

G² is selected from the group consisting of hydrogen and C₁-C₄ alkyl; or

G¹ and G² taken together with the nitrogen atom to which they are attached form a 5- to 10-membered optionally substituted heterocyclo, or a pharmaceutically acceptable salt or solvate thereof.

In another embodiment, a Substituted Indole Compound is a compound having Formula I, wherein [symbol] is a double bond, or a pharmaceutically acceptable salt or solvate thereof.

In another embodiment, a Substituted Indole Compound is a compound having Formula I, wherein Q¹ and Q² are —C(H)═, or a pharmaceutically acceptable salt or solvate thereof.

In another embodiment, a Substituted Indole Compound is a compound having Formula I, wherein Q³ is —C(R^{1d})═; and R^{1d} is selected from the group consisting of hydrogen and halo, or a pharmaceutically acceptable salt or solvate thereof.

In another embodiment, a Substituted Indole Compound is a compound having Formula I, wherein R^{1e} is hydrogen, or a pharmaceutically acceptable salt or solvate thereof.

In another embodiment, a Substituted Indole Compound is a compound having Formula I, wherein R^{1a} is C₁-C₃ alkyl, or a pharmaceutically acceptable salt or solvate thereof.

In another embodiment, a Substituted Indole Compound is a compound having Formula I, wherein G² is hydrogen, or a pharmaceutically acceptable salt or solvate thereof.

In another embodiment, a Substituted Indole Compound is a compound having Formula II:

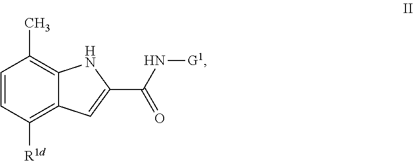

or a pharmaceutically acceptable salt or solvate thereof, wherein R^{1d} and G¹ are as defined in connection with Formula I.

In another embodiment, a Substituted Indole Compound is a compound having Formulae I or II, wherein R^{1d} is selected from the group consisting of hydrogen and fluoro, or a pharmaceutically acceptable salt or solvate thereof.

In another embodiment, a Substituted Indole Compound is a compound having Formula II-A:

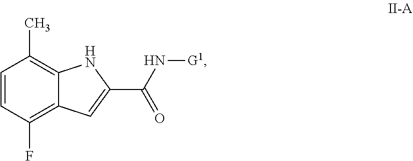

or a pharmaceutically acceptable salt or solvate thereof, wherein G¹ is as defined in connection with Formula II.

In another embodiment, a Substituted Indole Compound is a compound having Formulae I, II, or II-A, wherein G¹ is selected from the group consisting of optionally substituted C₆-C₁₀ aryl, optionally substituted 5- to 9-membered heteroaryl, optionally substituted 3- to 10-membered heterocyclo, optionally substituted C₆-C₈ cycloalkyl, (5- to 9-membered heteroaryl)C₁-C₆ alkyl, (5- to 9-membered heteroaryl)(C₆₋₁₀ aryl)C₁-C₄ alkyl, (5- to 9-membered heteroaryl heteroaryl)(C₃-C₆ cycloalkyl)C₁-C₄ alkyl, and (C₃-C₆ cycloalkyl)C₁-C₄ alkyl, or a pharmaceutically acceptable salt or solvate thereof.

In another embodiment, a Substituted Indole Compound is a compound having Formula III:

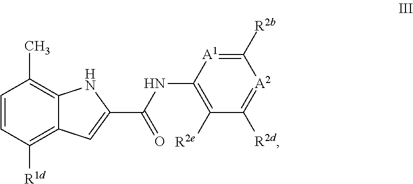

wherein:

A¹ is selected from the group consisting of —N═ and —C(R^{2a})═;

R^{2a} is selected from the group consisting of hydrogen, alkyl, halogen, and haloalkyl;

R^{2b} is selected from the group consisting of optionally substituted alkyl, optionally substituted heterocyclo, optionally substituted cycloalkyl, optionally substituted heteroaryl, optionally substituted aryl, (carboxamido)alkyl, —OR^{10c}, amino, (heterocyclo)alkyl, (amino)alkyl, (hydroxy)alkyl, carboxamido, (heteroaryl)alkyl, —S(═O)R^{9b}, —S(═O)₂R^{9b}, and —C(═O)R^{9c};

A² is selected from the group consisting of —N═ and —C(R^{2c})═;

R^{2c} is selected from the group consisting of hydrogen, alkyl, halogen, and haloalkyl;

R^{2d} is selected from the group consisting of hydrogen, alkyl, halogen, cyano, and haloalkyl;

R^{2e} is selected from the group consisting of hydrogen, alkyl, halogen, and haloalkyl;

R^{9b} is selected from the group consisting of amino, alkyl, optionally substituted cycloalkyl, optionally substituted aryl, optionally substituted heterocyclo, and optionally substituted heteroaryl;

R^{9c} is selected from the group consisting of amino, alkyl, optionally substituted cycloalkyl, optionally substituted aryl, optionally substituted heterocyclo, and optionally substituted heteroaryl; and

R^{10c} is selected from the group consisting of alkyl, (hydroxy)alkyl, and (amino)alkyl; and

R^{1d} is as defined in connection with Formula I, or a pharmaceutically acceptable salt or solvate thereof.

In another embodiment, a Substituted Indole Compound is a compound having Formula III-A:

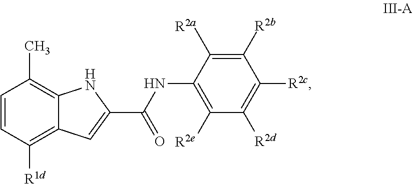

wherein R^{1d}, R^{2a}, R^{2b}, R^{2c}, R^{2d}, and R^{2e} are as defined in connection with Formula III, or a pharmaceutically acceptable salt or solvate thereof.

In another embodiment, a Substituted Indole Compound is a compound having Formula III or Formula III-A, wherein:

R^{2a} is selected from the group consisting of hydrogen, C₁-C₄ alkyl, halogen, and C₁-C₄ haloalkyl;

R^{2b} is selected from the group consisting of:

(A) unsubstituted 4- to 10-membered heterocyclo;

(B) substituted 4- to 10-membered heterocyclo having one, two, three, or four substituents independently selected from the group consisting of (i) —N(R^{3a})C(═O)R^{4a}; (ii) —NR^{5a}R^{5b}; (iii) unsubstituted 4- to 10-membered heterocyclo; (iv) substituted 4- to 10-membered heterocyclo having one, two, or three substituents independently selected from the group consisting of hydroxy, —NR^{5c}R^{5d}, C₁-C₄ alkyl, C₁-C₆ alkoxy, —C(R^{6a})(R^{6b})C(═O)NR^{5e}R^{5f}, —C(═O)R^{4b}, (hydroxy)C₁-C₄ alkyl, and halo; (v) unsubstituted C₃-C₆ cycloalkyl; (vi) (hydroxy)C₁-C₄ alkyl; (vii) C₁-C₆ alkyl; (viii) —C(═O)NR^{5g}R^{5h}; (ix) halo; (x) —C(═O)R^{4c}; (xi) C₁-C₆ haloalkyl; (xii) hydroxy; (xiii) (amino)C₁-C₄ alkyl; (xiv) (C₁-C₄ alkoxy)C₁-C₄ alkyl; (xv) —S(═O)₂R^{9a}; (xvi) (3- to 8-membered heterocyclo)C₁-C₄ alkyl; (xvii) C₁-C₆ alkoxy; (xviii) (C₃-C₆ cycloalkyl)C₁₋₄ alkyl; (xix) (C₆₋₁₀ aryl)C₁-C₄ alkyl; and (xxii) —OR^{10b};

(C) unsubstituted C₃-C₈ cycloalkyl;

(D) substituted C₃-C₈ cycloalkyl having one, two, three, or four substituents independently selected from the group consisting of (i) unsubstituted 4- to 10-membered heterocyclo; (ii) substituted 4- to 10-membered heterocyclo having one or two substituents, independently selected from the group consisting of amino and C₁-C₄ alkyl; (iii) unsubstituted 5- or 6-membered heteroaryl; (iv) substituted 5- or 6-membered heteroaryl having one, two, or three substituents independently selected from the group consisting of halo, C₁-C₄ alkyl, (3- to 8-membered heterocyclo)alkyl, hydroxy, and amino; (v) —NR⁵ⁱR^{5j}; (vi) cyano; (vii) —N(R^{3d})C(═O)R^{4f}; (viii) hydroxy; and (ix) C₁-C₄ alkyl;

(E) unsubstituted 5- to 10-membered heteroaryl;

(F) substituted 5- to 10-membered heteroaryl having one, two, three, or four substituents independently selected from the group consisting of (i) halo; (ii) C₁-C₄ alkyl; (C₁-C₄ alkoxy)C₁-C₄ alkyl; (hydroxy)C₁-C₄ alkyl; C₃-C₆ cycloalkyl; (amino)C₁-C₄ alkyl; unsubstituted C₃-C₆ cycloalkyl; substituted C₃-C₆ cycloalkyl having one, two, three, or four substituents independently selected from the group consisting of —NR^{5g}R^{5h}; unsubstituted 4- to 14-membered heterocyclo; substituted 4- to 14-membered heterocyclo having one or two substituents independently selected from the group consisting of hydroxy, amino, and C₁-C₄ alkyl; —NR^{5q}R^{5r}; and (ix) (3- to 8-membered heterocyclo)C₁-C₄ alkyl;

(G) unsubstituted C₆-C₁₀ aryl;

(H) substituted C₆-C₁₀ aryl, having one, two, three, or four substituents independently selected from the group consisting of (i) halo; (ii) C₁-C₄ alkyl; (iii) —CH-₂N(H)S(═O)₂R⁸; (iv) (5- to 9-membered heteroaryl)C₁-C₄ alkyl; (v) —OR^{10a}; (vi) —N(R^{3b})C(═O)R^{4b}; (vii) (amino)C₁-C₄ alkyl; and (viii) (hydroxy)C₁-C₄ alkyl;

(I) (carboxamido)C₁-C₄ alkyl;

(J) —OR^{10c};

(K) —NR^{5o}R^{5p};

(L) (3- to 8-membered heterocyclo)C₁-C₄ alkyl;

(M) (amino)C₁-C₄ alkyl;

(N) (hydroxy)C₁-C₄ alkyl;

(O) —C(═O)NR^{5s}R^{5t};

(P) (5- to 9-membered heteroaryl)C₁-C₄ alkyl; and

(Q) —S(═O)₂R^{9b};

R^{2c} is selected from the group consisting of hydrogen, C₁-C₄ alkyl, halogen, and C₁-C₄ haloalkyl;

R^{2d} is selected from the group consisting of hydrogen, C₁-C₄ alkyl, halogen, cyano, and C₁-C₄ haloalkyl;

R^{2e} is selected from the group consisting of hydrogen, C₁-C₄ alkyl, halogen, and C₁-C₄ haloalkyl;

R^{3a}, R^{3b}, R^{3c}, and R^{3d} are each independently selected from the group consisting of hydrogen, C₁-C₄ alkyl, optionally substituted C₃-C₆ cycloalkyl, and optionally substituted 4- to 14-membered heterocyclo;

R^{4a}, R^{4b}, R^{4c}, R^{4d}, R^{4e}, and R^{4f} are each independently selected from the group consisting of C₁-C₆ alkyl; C₁-C₆ haloalkyl; C₃-C₆ cycloalkyl; C₁-C₆ alkoxy; (C₁-C₄ alkoxy)C₁-C₄ alkyl; (C₆₋₁₀ aryl)C₁-C₄ alkyl; (5- to 9-membered heteroaryl)C₁-C₄ alkyl; (amino)C₁-C₄ alkyl; (hydroxy)C₁-C₄ alkyl; (cyano)C₁-C₄ alkyl; unsubstituted 4- to 14-membered heterocyclo; substituted 4- to 14-membered heterocyclo having one or two substituents independently selected from the group consisting of halo and C₁-C₄ alkyl; unsubstituted C₆-C₁₀ aryl; substituted C₆-C₁₀ aryl, having one, two, three, or four substituents independently selected from the group consisting of halo and C₁-C₄ alkyl; unsubstituted 5- or 6-membered heteroaryl; and substituted 5- or 6-membered heteroaryl having one, two, three, or four substituents independently selected from the group consisting of halo and C₁-C₄ alkyl;

R^{5a} and R^{5b} are independently selected from the group consisting of hydrogen; C₁-C₄ alkyl; C₁-C₄ haloalkyl; (hydroxy)C₁-C₄ alkyl; (amino)C₁-C₄ alkyl; (C₁-C₄ alkoxy)C₁-C₄ alkyl; (5- to 9-membered heteroaryl)C₁-C₄ alkyl; unsubstituted 5- or 6-membered heteroaryl; substituted 5- or 6-membered heteroaryl having one or two substituents independently selected from the group consisting of halo and C₁-C₄ alkyl; unsubstituted 4- to 14-membered heterocyclo; and substituted 4- to 14-membered heterocyclo having one or two substituents independently selected from the group consisting of hydroxy, amino, and C₁-C₄ alkyl;

R^{5c} and R^{5d} are independently selected from the group consisting of hydrogen; C₁-C₄ alkyl; C₁-C₄ haloalkyl; (hydroxy)C₁-C₄ alkyl; (amino)C₁-C₄ alkyl; (C₁-C₄ alkoxy)C₁-C₄ alkyl; (5- to 9-membered heteroaryl)C₁-C₄ alkyl; unsubstituted 5- or 6-membered heteroaryl; substituted 5- or 6-membered heteroaryl having one or two substituents independently selected from the group consisting of halo and C₁-C₄ alkyl; unsubstituted 4- to 14-membered heterocyclo; and substituted 4- to 14-membered heterocyclo having one or two substituents independently selected from the group consisting of hydroxy, amino, and C₁-C₄ alkyl; or

R^{5c} and R^{5d} taken together with the nitrogen atom to which they are attached form an optionally substituted 4- to 14-membered heterocyclo;

R^{5e} and R^{5f} are independently selected from the group consisting of hydrogen; C₁-C₄ alkyl; C₁-C₄ haloalkyl; (hydroxy)C₁-C₄ alkyl; (amino)C₁-C₄ alkyl; (C₁-C₄ alkoxy)C₁-C₄ alkyl; (5- to 9-membered heteroaryl)C₁-C₄ alkyl; unsubstituted 5- or 6-membered heteroaryl; substituted 5- or 6-membered heteroaryl having one or two substituents independently selected from the group consisting of halo and C₁-C₄ alkyl; unsubstituted 4- to 14-membered heterocyclo; and substituted 4- to 14-membered heterocyclo having one or two substituents independently selected from the group consisting of hydroxy, amino, and C₁-C₄ alkyl; or

R^{5e} and R^{5f} taken together with the nitrogen atom to which they are attached form an optionally substituted 4- to 14-membered heterocyclo;

R^{5g} and R^{5h} are independently selected from the group consisting of hydrogen; C₁-C₄ alkyl; C₁-C₄ haloalkyl; (hydroxy)C₁-C₄ alkyl; (amino)C₁-C₄ alkyl; (C₁-C₄ alkoxy)C₁-C₄ alkyl; (5- to 9-membered heteroaryl)C₁-C₄ alkyl; unsubstituted 5- or 6-membered heteroaryl; substituted 5- or 6-membered heteroaryl having one or two substituents independently selected from the group consisting of halo and C₁-C₄ alkyl; unsubstituted 4- to 14-membered heterocyclo; and substituted 4- to 14-membered heterocyclo having one or two substituents independently selected from the group consisting of hydroxy, amino, and C₁-C₄ alkyl; or

R^{5g} and R^{5h} taken together with the nitrogen atom to which they are attached form an optionally substituted 4- to 14-membered heterocyclo;

R⁵ⁱ and R^{5j} are independently selected from the group consisting of hydrogen; C₁-C₄ alkyl; C₁-C₄ haloalkyl; (hydroxy)C₁-C₄ alkyl; (amino)C₁-C₄ alkyl; (C₁-C₄ alkoxy)C₁-C₄ alkyl; (5- to 9-membered heteroaryl)C₁-C₄ alkyl; unsubstituted 5- or 6-membered heteroaryl; substituted 5- or 6-membered heteroaryl having one or two substituents independently selected from the group consisting of halo and C₁-C₄ alkyl; unsubstituted 4- to 14-membered heterocyclo; and substituted 4- to 14-membered heterocyclo having one or two substituents independently selected from the group consisting of hydroxy, amino, and C₁-C₄ alkyl; or

R⁵ⁱ and R^{5j} taken together with the nitrogen atom to which they are attached form an optionally substituted 4- to 14-membered heterocyclo;

R^{5k} and R^{5l} are independently selected from the group consisting of hydrogen; C₁-C₄ alkyl; C₁-C₄ haloalkyl; (hydroxy)C₁-C₄ alkyl; (amino)C₁-C₄ alkyl; (C₁-C₄ alkoxy)C₁-C₄ alkyl; (5- to 9-membered heteroaryl)C₁-C₄ alkyl; unsubstituted 5- or 6-membered heteroaryl; substituted 5- or 6-membered heteroaryl having one or two substituents independently selected from the group consisting of halo and C₁-C₄ alkyl; unsubstituted 4- to 14-membered heterocyclo; and substituted 4- to 14-membered heterocyclo having one or two substituents independently selected from the group consisting of hydroxy, amino, and C₁-C₄ alkyl; or

R^{5k} and R^{5l} taken together with the nitrogen atom to which they are attached form an optionally substituted 4- to 14-membered heterocyclo;

R^{5m} and R⁵ⁿ are independently selected from the group consisting of hydrogen; C₁-C₄ alkyl; C₁-C₄ haloalkyl; (hydroxy)C₁-C₄ alkyl; (amino)C₁-C₄ alkyl; (C₁-C₄ alkoxy)C₁-C₄ alkyl; (5- to 9-membered heteroaryl)C₁-C₄ alkyl; unsubstituted 5- or 6-membered heteroaryl; substituted 5- or 6-membered heteroaryl having one or two substituents independently selected from the group consisting of halo and C₁-C₄ alkyl; unsubstituted 4- to 14-membered heterocyclo; and substituted 4- to 14-membered heterocyclo having one or two substituents independently selected from the group consisting of hydroxy, amino, and C₁-C₄ alkyl; or

R^{5m} and R⁵ⁿ taken together with the nitrogen atom to which they are attached form an optionally substituted 4- to 14-membered heterocyclo;

R⁵⁰ and R^{5p} are independently selected from the group consisting of hydrogen; C₁-C₄ alkyl; C₁-C₄ haloalkyl; (hydroxy)C₁-C₄ alkyl; (amino)C₁-C₄ alkyl; (C₁-C₄ alkoxy)C₁-C₄ alkyl; (5- to 9-membered heteroaryl)C₁-C₄ alkyl; unsubstituted 5- or 6-membered heteroaryl; substituted 5- or 6-membered heteroaryl having one or two substituents independently selected from the group consisting of halo and C₁-C₄ alkyl; unsubstituted 4- to 14-membered heterocyclo; and substituted 4- to 14-membered heterocyclo having one or two substituents independently selected from the group consisting of hydroxy, amino, and C₁-C₄ alkyl; or

R⁵⁰ and R^{5p} taken together with the nitrogen atom to which they are attached form an optionally substituted 4- to 14-membered heterocyclo;

R^{5q} and R^{5r} are independently selected from the group consisting of hydrogen; C₁-C₄ alkyl; C₁-C₄ haloalkyl; (hydroxy)C₁-C₄ alkyl; (amino)C₁-C₄ alkyl; (C₁-C₄ alkoxy)C₁-C₄ alkyl; (5- to 9-membered heteroaryl)C₁-C₄ alkyl; unsubstituted 5- or 6-membered heteroaryl; substituted 5- or 6-membered heteroaryl having one or two substituents independently selected from the group consisting of halo and C₁-C₄ alkyl; unsubstituted 4- to 14-membered heterocyclo; and substituted 4- to 14-membered heterocyclo having one or two substituents independently selected from the group consisting of hydroxy, amino, and C₁-C₄ alkyl;

R^{5s} and R^{5t} are independently selected from the group consisting of hydrogen; C₁-C₄ alkyl; C₁-C₄ haloalkyl; (hydroxy)C₁-C₄ alkyl; (amino)C₁-C₄ alkyl; (C₁-C₄ alkoxy)C₁-C₄ alkyl; (5- to 9-membered heteroaryl)C₁-C₄ alkyl; unsubstituted 5- or 6-membered heteroaryl; substituted 5- or 6-membered heteroaryl having one or two substituents independently selected from the group consisting of halo and C₁-C₄ alkyl; unsubstituted 4- to 14-membered heterocyclo; and substituted 4- to 14-membered heterocyclo having one or two substituents independently selected from the group consisting of hydroxy, amino, and C₁-C₄ alkyl;

R^{6a}, R^{6b}, R^{6c}, and R^{6d} are independently selected from the group consisting of hydrogen and C₁-C₄ alkyl;

R⁸ is C₁-C₆ alkyl;

R^{9a} is selected from the group consisting of C₁-C₆ alkyl; unsubstituted C₃-C₈ cycloalkyl; and substituted C₃-C₈ cycloalkyl having one or two substituents independently selected from the group consisting of halo, C₁-C₄ alkyl, amino, and (amino)C₁-C₄ alkyl;

R^{9b} is selected from the group consisting of C₁-C₆ alkyl and amino;

R^{10a} is selected from the group consisting of alkyl, (hydroxy)C₁-C₄ alkyl, and (amino)C₁-C₄ alkyl;

R^{10b} is (amino)C₁-C₄ alkyl; and

R^{10c} is (amino)C₁-C₄ alkyl, or a pharmaceutically acceptable salt or solvate thereof.

In another embodiment, a Substituted Indole Compound is a compound having Formula III or Formula III-A, wherein R^{2b} is an optionally substituted 3- to 10-membered heterocycle linked to the rest of the molecule through a nitrogen atom, e.g., R^{2b} is:

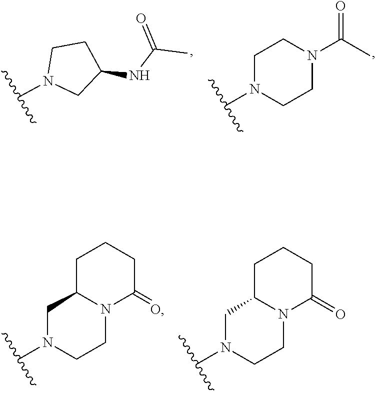

and the like.

In another embodiment, a Substituted Indole Compound is a compound having Formula III or Formula III-A, wherein:

R^{2b} is selected from the group consisting of:

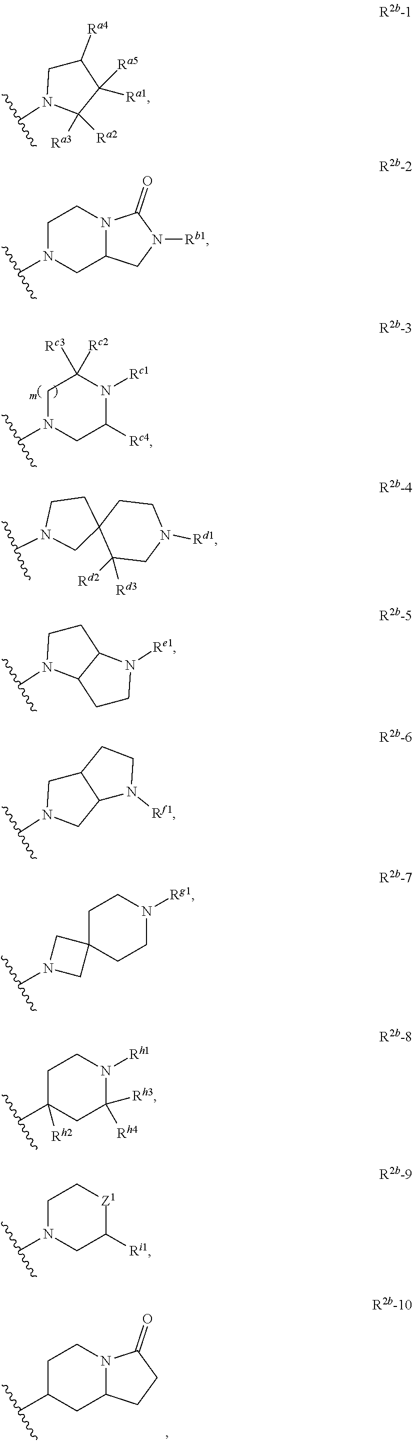

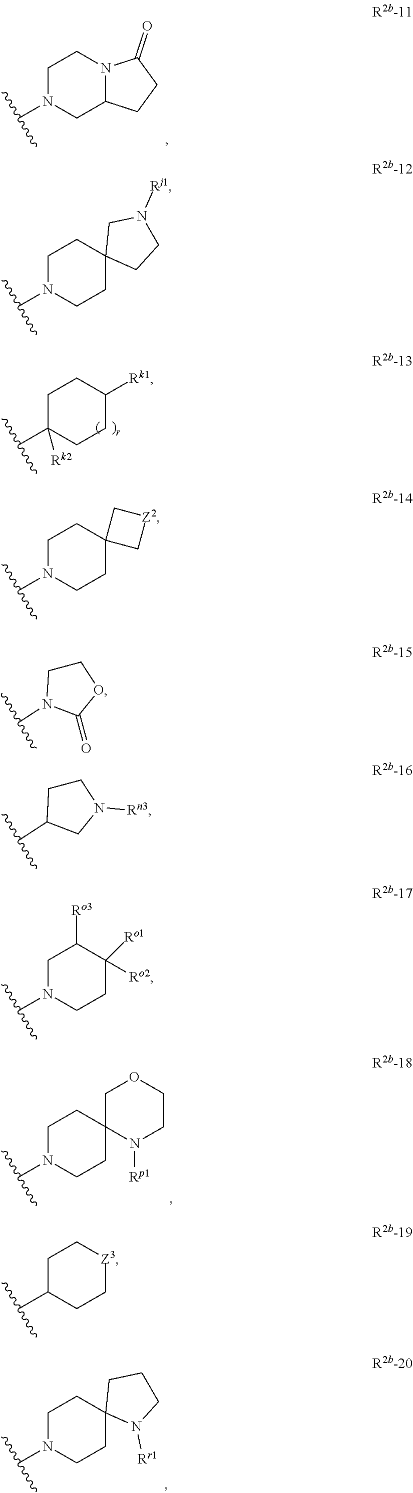

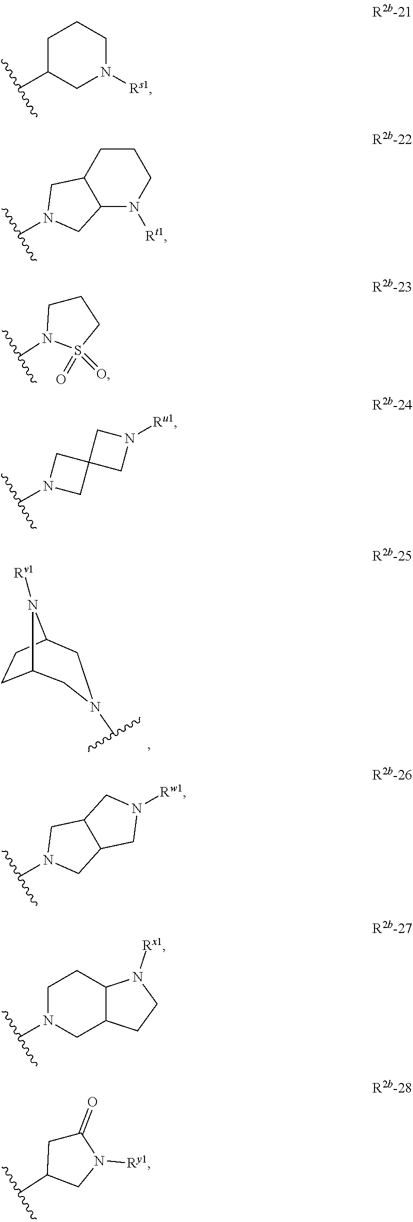

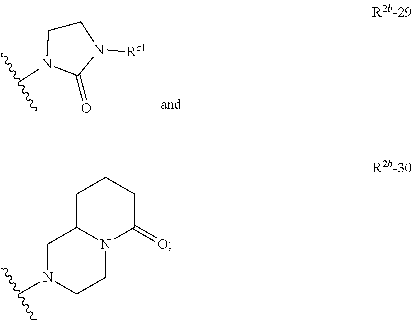

R^{a1} is selected from the group consisting of —N(R^{3a})C(═O)R^{4a}; —NR^{5a}R^{5b}; unsubstituted 4- to 10-membered heterocyclo; substituted 4- to 10-membered heterocyclo having one, two, or three substituents independently selected from the group consisting of hydroxy, —NR^{5c}R^{5d}, C₁-C₄ alkyl, C₁-C₆ alkoxy, —C(R^{6a})(R^{6b})C(═O)NR^{5e}R^{5f}, —C(═O)R^{4b}, (hydroxy)C₁-C₄ alkyl, and halo;

R^{a2} and R^{a3} are each hydrogen; or

R^{a2} and R^{a3} taken together with the carbon atom to which they are attached form a C(═O) group;

R^{a4} is selected from the group consisting of hydrogen, halo, and hydroxy;

R^{a5} is selected from the group consisting of hydrogen, C₁-C₄ alkyl, and C₃-C₆ cycloalkyl;

R^{b1} is selected from the group consisting of hydrogen, C₁-C₄ alkyl, and C₃-C₆ cycloalkyl;

R^{c1} is selected from the group consisting of hydrogen, C₁-C₄ alkyl, C₃-C₆ cycloalkyl, and —C(═O)R^{4c};

R^{c2} and R^{c3} are each independently selected from the group consisting of hydrogen, C₁-C₄ alkyl, and C₁-C₄ haloalkyl; or

R^{c2} and R^{c3} taken together with the carbon atom to which they are attached form a C(═O) group;

R^{c4} is selected from the group consisting of hydrogen and C₁-C₄ alkyl;

m is 1 or 2;

R^{d1} is selected from the group consisting of hydrogen, C₁-C₄ alkyl, and —C(═O)R^{4c},

R^{d2} and R^{d3} are each independently selected from the group consisting of hydrogen and fluoro;

R^{e1} is selected from the group consisting of hydrogen, C₁-C₄ alkyl, C₃-C₆ cycloalkyl, and —C(═O)R^{4c};

R^{f1} is selected from the group consisting of hydrogen, C₁-C₄ alkyl, C₃-C₆ cycloalkyl, and —C(═O)R^{4c},

R^{g1} is selected from the group consisting of hydrogen, C₁-C₄ alkyl, —C(═O)R^{4c}, C₁-C₄ haloalkyl, (C₁-C₄ alkoxy)C₁-C₄ alkyl

R^{h1} is selected from the group consisting of hydrogen, C₁-C₄ alkyl, C₃-C₆ cycloalkyl, and —C(═O)R^{4c};

R^{h2} is selected from the group consisting of hydrogen and C₁-C₄ alkyl;

R^{h3} and R^{h4} are each independently selected from the group consisting of hydrogen and C₁-C₄ alkyl; or

R^{h3} and R^{h4} taken together with the carbon atom to which they are attached form a C(═O) group;

Rⁱ¹ is selected from the group consisting of hydrogen, C₁-C₄ alkyl, C₃-C₆ cycloalkyl, (hydroxy)C₁-C₄ alkyl, —N(R^{3a})C(═O)R^{4a}, and (amino)C₁-C₄ alkyl;

Z¹ is selected from the group consisting of —CH₂— and —O—;

R^{j1} is selected from the group consisting of hydrogen, C₁-C₄ alkyl, C₃-C₆ cycloalkyl, and —C(═O)R^{4c};

R^{k1} is selected from the group consisting of C₁-C₄ alkyl, unsubstituted 4- to 14-membered heterocyclo and —NR^{5a}R^{5b};

R^{k2} is selected from the group consisting of hydrogen, hydroxy, and C₁-C₄ alkyl;

r is 0, 1, or 2;

Z² is selected from the group consisting of —O— and —N(R^{m3})—;

R^{m3} is selected from the group consisting of hydrogen, C₁-C₄ alkyl, and C₁-C₄ haloalkyl;

Rⁿ³ is selected from the group consisting of hydrogen, C₁-C₄ alkyl, and —C(═O)R^{4c};

R^{o1} is selected from the group consisting of hydroxy, (hydroxy)C₁-C₄ alkyl, (amino)C₁-C₄ alkyl, (C₁-C₄ alkoxy)C₁-C₄ alkyl, C₁-C₄ alkoxy, —NR^{5a}R^{5b}, unsubstituted 4-to 14-membered heterocyclo, substituted 4- to 14-membered heterocyclo having one, two, or three substituents independently selected from the group consisting of halo, C₁-C₄ alkyl and C₁-C₄ alkoxy;

R^{o2} is selected from the group consisting of hydrogen, C₁-C₄ alkyl, and (C₁-C₄ alkoxy)C₁-C₄ alkyl

R^{o3} is selected from the group consisting of hydrogen, fluoro, and C₁-C₄ alkyl;

R^{p1} is selected from the group consisting of hydrogen, C₁-C₄ alkyl, and —C(═O)R^{4c};

Z³ is selected from the group consisting of —O— and —N(R^{q1})—;

R^{q1} is selected from the group consisting of hydrogen and C₁-C₄ alkyl;

R^{r1} is selected from the group consisting of hydrogen, C₁-C₄ alkyl, and —C(═O)R^{4c};

R^{s1} is selected from the group consisting of hydrogen, C₁-C₄ alkyl, and —C(═O)R^{4c};

R^{t1} is selected from the group consisting of hydrogen, C₁-C₄ alkyl, and —C(═O)R^{4c};

R^{u1} is selected from the group consisting of hydrogen, C₁-C₄ alkyl, and —C(═O)R^{4c};

R^{v1} is selected from the group consisting of hydrogen, C₁-C₄ alkyl, and —C(═O)R^{4c};

R^{w1} is selected from the group consisting of hydrogen, C₁-C₄ alkyl, and —C(═O)R^{4c};

R^{x1} is selected from the group consisting of hydrogen, C₁-C₄ alkyl, and —C(═O)R^{4c};

R^{y1} is selected from the group consisting of hydrogen and C₁-C₄ alkyl; and

R^{z1} is selected from the group consisting of hydrogen and C₁-C₄ alkyl, or a pharmaceutically acceptable salt or solvate thereof.

In another embodiment, a Substituted Indole Compound is a compound having Formula III or Formula III-A, wherein:

R^{2b} is selected from the group consisting of:

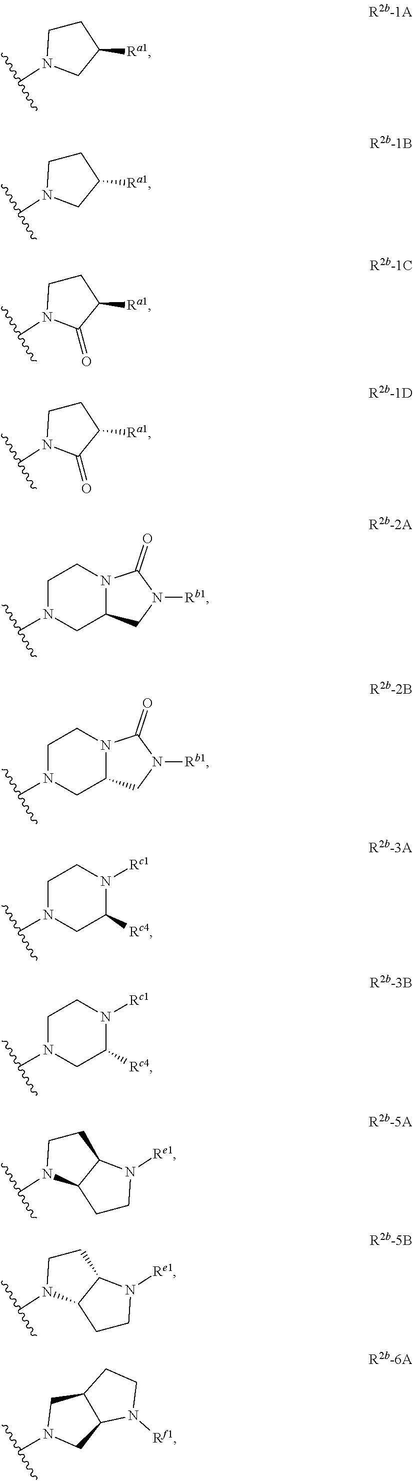

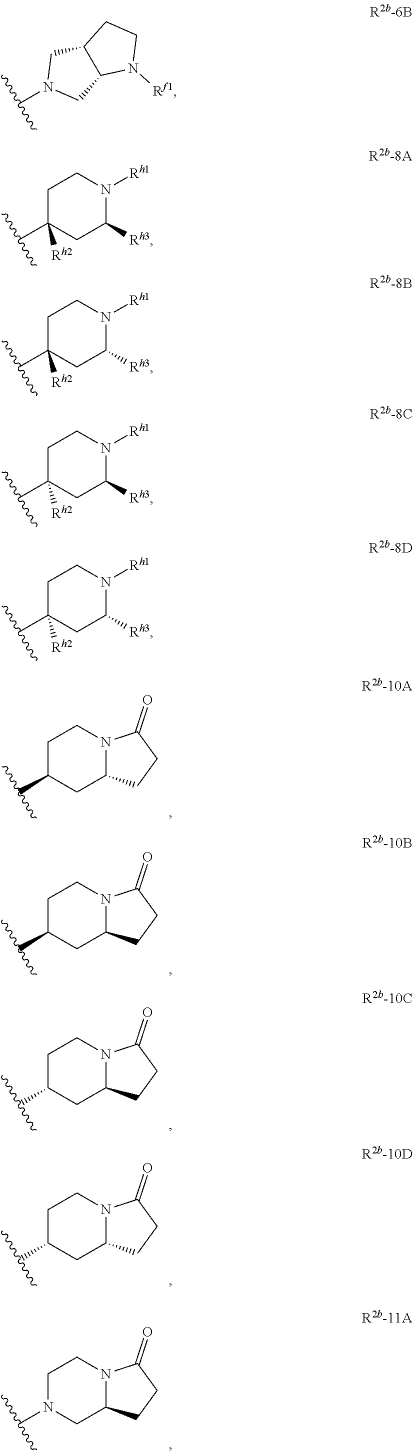

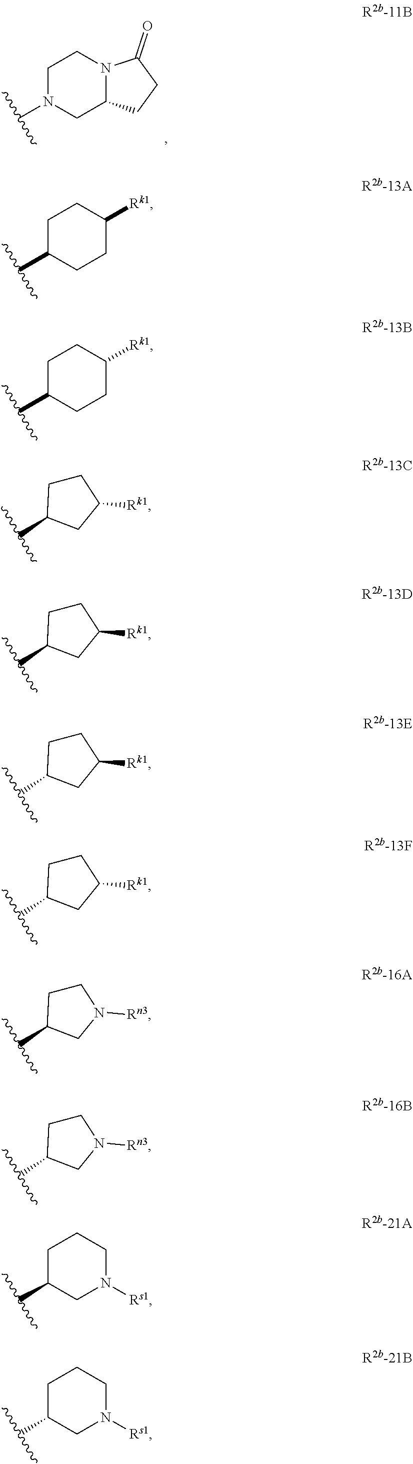

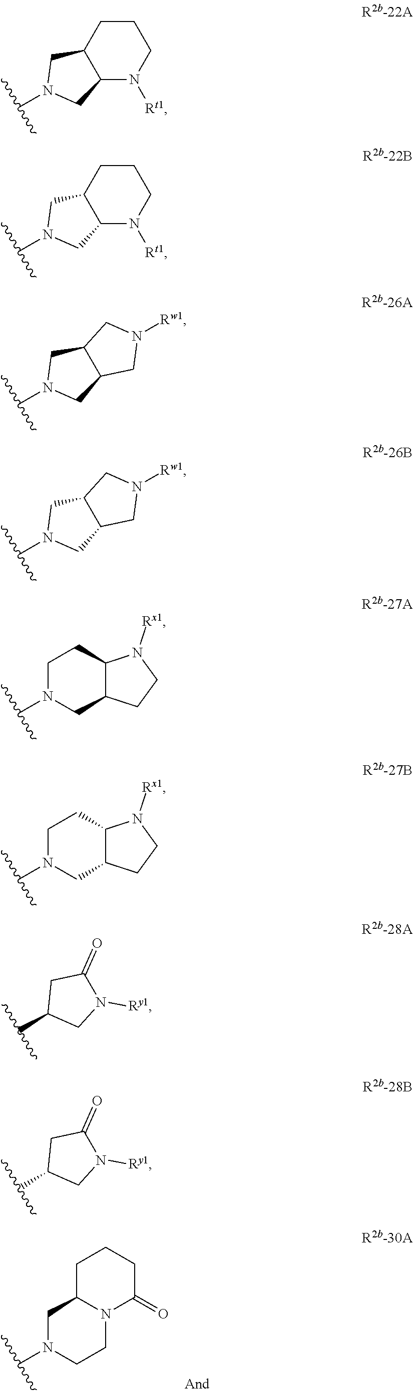

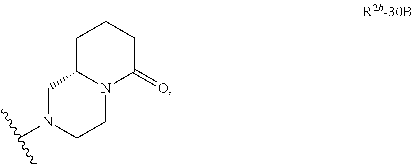 [SMILES: *N1CCN2C(=O)CCC[C@H]2C1]

or a pharmaceutically acceptable salt or solvate thereof.

In another embodiment, a Substituted Indole Compound is a compound having Formula III or Formula III-A, wherein R^{2b} is R^{2b}-1, R^{2b}-1A, R^{2b}-1B, R^{2b}-1C, or R^{2b}-1D, or a pharmaceutically acceptable salt or solvate thereof. In another embodiment, R^{a1} is —N(R^{3a})C(═O)R^{4a}. In another embodiment, R^{a1} is —NR^{5a}R^{5b}. In another embodiment, R^{a1} is —NR^{5a}R^{5b} and R^{5a} and R^{5b} are independently selected from the group consisting of hydrogen and C₁-C₄ alkyl. In another embodiment, R^{a1} is optionally substituted 4- to 10-membered heterocyclo.

In another embodiment, a Substituted Indole Compound is a compound having Formula III or Formula III-A, wherein R^{2b} is R^{2b}-2, R^{2b}-2A, or R^{2b}-2b, or a pharmaceutically acceptable salt or solvate thereof. In another embodiment, R^{b1} is C₁-C₄ alkyl.

In another embodiment, a Substituted Indole Compound is a compound having Formula III or Formula III-A, wherein R^{2b} is R^{2b}-3, R^{2b}-3A, or R^{2b}-3B, or a pharmaceutically acceptable salt or solvate thereof. In another embodiment, R^{c1} is selected from the group consisting of C₁-C₄ alkyl, C₃-C₆ cycloalkyl, and —C(═O)R^{4c}. In another embodiment, R^{c2} and R^{c3} are each hydrogen. In another embodiment, R^{c2} and R^{c3} taken together with the carbon atom to which they are attached form a C(═O) group. In another embodiment, R^{c4} is hydrogen. In another embodiment, m is 1.

In another embodiment, a Substituted Indole Compound is a compound having Formula III or Formula III-A, wherein R^{2b} is R^{2b}-4, or a pharmaceutically acceptable salt or solvate thereof. In another embodiment, R^{d1} is C(═O)R^{4c}. In another embodiment, R^{d2} and R^{d3} are each hydrogen or fluoro.

In another embodiment, a Substituted Indole Compound is a compound having Formula III or Formula III-A, wherein R^{2b} is R^{2b}-5, R^{2b}-5A, or R^{2b}-5B, or a pharmaceutically acceptable salt or solvate thereof. In another embodiment, R^{e1} is —C(═O)R^{4c}.

In another embodiment, a Substituted Indole Compound is a compound having Formula III or Formula III-A, wherein R^{2b} is R^{2b}-6, R^{2b}-6A, or R^{2b}-6B, or a pharmaceutically acceptable salt or solvate thereof. In another embodiment, R^{f1} is C(═O)R^{4c}.

In another embodiment, a Substituted Indole Compound is a compound having Formula III or Formula III-A, wherein R^{2b} is R^{2b}-7, or a pharmaceutically acceptable salt or solvate thereof. In another embodiment, R^{g1} is C(═O)R^{4c}.

In another embodiment, a Substituted Indole Compound is a compound having Formula III or Formula III-A, wherein R^{2b} is R^{2b}-8, R^{2b}-8A, R^{2b}-8B, R^{2b}-8C, or R^{2b}-8D, or a pharmaceutically acceptable salt or solvate thereof. In another embodiment, R^{h1} is —C(═O)R^{4c}. In another embodiment, R^{h2} is selected from the group consisting of hydrogen and C₁-C₃ alkyl. In another embodiment, R^{h3} is hydrogen.

In another embodiment, a Substituted Indole Compound is a compound having Formula III or Formula III-A, wherein R^{2b} is R^{2b}-9, or a pharmaceutically acceptable salt or solvate thereof.

In another embodiment, a Substituted Indole Compound is a compound having Formula III or Formula III-A, wherein R^{2b} is selected from the group consisting of R^{2b}-10, R^{2b}-10A, R^{2b}-10B, R^{2b}-10C, and R^{2b}-10d, or a pharmaceutically acceptable salt or solvate thereof.

In another embodiment, a Substituted Indole Compound is a compound having Formula III or Formula III-A, wherein R^{2b} is selected from the group consisting of R^{2b}-11, R^{2b}-11A and R^{2b}-11B, or a pharmaceutically acceptable salt or solvate thereof.

In another embodiment, a Substituted Indole Compound is a compound having Formula III or Formula III-A, wherein R^{2b} is R^{2b}-12, or a pharmaceutically acceptable salt or solvate thereof. In another embodiment, R^{j1} is —C(═O)R^{4c}.

In another embodiment, a Substituted Indole Compound is a compound having Formula III or Formula III-A, wherein R^{2b} is selected from the group consisting of R^{2b}-13, R^{2b}-13A, R^{2b}-13B, R^{2b}-13C, R^{2b}-13D, R^{2b}-13E, and R^{2b}-13F, or a pharmaceutically acceptable salt or solvate thereof.

In another embodiment, a Substituted Indole Compound is a compound having Formula III or Formula III-A, wherein R^{2b} is R^{2b}-14, or a pharmaceutically acceptable salt or solvate thereof.

In another embodiment, a Substituted Indole Compound is a compound having Formula III or Formula III-A, wherein R^{2b} is R^{2b}-15, or a pharmaceutically acceptable salt or solvate thereof.

In another embodiment, a Substituted Indole Compound is a compound having Formula III or Formula III-A, wherein R^{2b} is selected from the group consisting of R^{2b}-16, R^{2b}-16A and R^{2b}-16B, or a pharmaceutically acceptable salt or solvate thereof. In another embodiment, Rⁿ³ is —C(═O)R^{4c}.

In another embodiment, a Substituted Indole Compound is a compound having Formula III or Formula III-A, wherein R^{2b} is R^{2b}-17, or a pharmaceutically acceptable salt or solvate thereof.

In another embodiment, a Substituted Indole Compound is a compound having Formula III or Formula III-A, wherein R^{2b} is R^{2b}-18, or a pharmaceutically acceptable salt or solvate thereof.

In another embodiment, a Substituted Indole Compound is a compound having Formula III or Formula III-A, wherein R^{2b} is R^{2b}-19, or a pharmaceutically acceptable salt or solvate thereof.

In another embodiment, a Substituted Indole Compound is a compound having Formula III or Formula III-A, wherein R^{2b} is R^{2b}-20, or a pharmaceutically acceptable salt or solvate thereof.

In another embodiment, a Substituted Indole Compound is a compound having Formula III or Formula III-A, wherein R^{2b} is selected from the group consisting of R^{2b}-21, R^{2b}-21A and R^{2b}-21B, or a pharmaceutically acceptable salt or solvate thereof.

In another embodiment, a Substituted Indole Compound is a compound having Formula III, wherein R^{2b} is selected from the group consisting of R^{2b}-22, R^{2b}-22A and R^{2b}-22B, or a pharmaceutically acceptable salt or solvate thereof.

In another embodiment, a Substituted Indole Compound is a compound having Formula III or Formula III-A, wherein R^{2b} is R^{2b}-23, or a pharmaceutically acceptable salt or solvate thereof.

In another embodiment, a Substituted Indole Compound is a compound having Formula III or Formula III-A, wherein R^{2b} is R^{2b}-24, or a pharmaceutically acceptable salt or solvate thereof.

In another embodiment, a Substituted Indole Compound is a compound having Formula III or Formula III-A, wherein R^{2b} is R^{2b}-25, or a pharmaceutically acceptable salt or solvate thereof.

In another embodiment, a Substituted Indole Compound is a compound having Formula III or Formula III-A, wherein R^{2b} is selected from the group consisting of R^{2b}-26, R^{2b}-26A and R^{2b}-26B, or a pharmaceutically acceptable salt or solvate thereof.

In another embodiment, a Substituted Indole Compound is a compound having Formula III or Formula III-A, wherein R^{2b} is selected from the group consisting of R^{2b}-27, R^{2b}-27A and R^{2b}-27B, or a pharmaceutically acceptable salt or solvate thereof.

In another embodiment, a Substituted Indole Compound is a compound having Formula III or Formula III-A, wherein R^{2b} is selected from the group consisting of R^{2b}-28, R^{2b}-28A and R^{2b}-28B, or a pharmaceutically acceptable salt or solvate thereof.

In another embodiment, a Substituted Indole Compound is a compound having Formula III or Formula III-A, wherein R^{2b} is R^{2b}-29, or a pharmaceutically acceptable salt or solvate thereof.

In another embodiment, a Substituted Indole Compound is a compound having Formula III or Formula III-A, wherein R^{2b} is R^{2b}-30, R^{2b}-30A, or R^{2b}-30B, or a pharmaceutically acceptable salt or solvate thereof.

In another embodiment, a Substituted Indole Compound is a compound having Formula III or Formula III-A, wherein R^{2b} is any one or more of the R^{11a} groups provided in connection with Formula IV, see below, or a pharmaceutically acceptable salt or solvate thereof.

In another embodiment, a Substituted Indole Compound is a compound having Formula III or Formula III-A, wherein R^{4c} is C₁-C₄ alkyl, or a pharmaceutically acceptable salt or solvate thereof.

In another embodiment, a Substituted Indole Compound is a compound having Formula III or Formula III-A, wherein R^{2d} is selected from the group consisting of hydrogen, fluoro, and chloro, or a pharmaceutically acceptable salt or solvate thereof.

In another embodiment, a Substituted Indole Compound is a compound having Formula III or Formula III-A, wherein R^{2d} is hydrogen, or a pharmaceutically acceptable salt or solvate thereof.

In another embodiment, a Substituted Indole Compound is a compound having Formula III in any of the above described embodiments, wherein A¹ and A² are —C(H)═; R^{2e} is hydrogen; and R^{2d} is selected from the group consisting of hydrogen and halogen, or a pharmaceutically acceptable salt or solvate thereof.

In another embodiment, a Substituted Indole Compound is a compound having Formula III or Formula III-A, wherein R^{2d} is fluoro, or a pharmaceutically acceptable salt or solvate thereof.

In another embodiment, a Substituted Indole Compound is a compound having Formula IV:

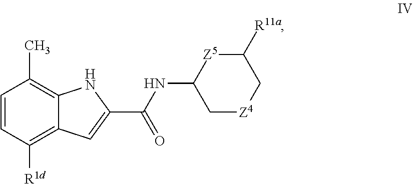

wherein:

Z⁴ is selected from the group consisting of —O—, —C(R^{28a})(R^{28b})—, and —N(R²³)—; or Z⁴ is absent;

Z⁵ is selected from the group consisting of —CH₂— and —CH₂CH₂—;

R^{11a} is selected from the group consisting of optionally substituted alkyl, optionally substituted heterocyclo, optionally substituted heteroaryl, and —N(R^{12b})C(═O)R^{13c};

R^{12b} is selected from the group consisting of hydrogen, alkyl, cycloalkyl, and heterocyclo;

R^{13c} is selected from the group consisting of alkyl, haloalkyl, alkoxy, (alkoxy)alkyl, (hydroxy)alkyl, (cyano)alkyl, optionally substituted aryl, optionally substituted heteroaryl, optionally substituted cycloalkyl, and optionally substituted heterocycle, amino, (amino)alkyl, (C₃-C₆ cycloalkyl)oxy, and (4- to 8-membered heterocyclo)oxy;

R²³ is selected from the group consisting of hydrogen and C₁-C₄ alkyl;

R^{28a} and R^{28b} are independently selected from the group consisting of hydrogen, alkyl, and halo; and

R^{1d} is as defined in connection with Formula I, or a pharmaceutically acceptable salt or solvate thereof.

In another embodiment, a Substituted Indole Compound is a compound having Formula IV, wherein Z⁴ is selected from the group consisting of —O— and —CH₂—; or Z⁴ is absent, or a pharmaceutically acceptable salt or solvate thereof.

In another embodiment, a Substituted Indole Compound is a compound having Formula IV, wherein:

Z⁴ is selected from the group consisting of —O— and —CH₂—; or Z⁴ is absent;

Z⁵ is selected from the group consisting of —CH₂— and —CH₂CH₂—;

R^{13c} is selected from the group consisting of alkyl, haloalkyl, alkoxy, (alkoxy)alkyl, (hydroxy)alkyl, (cyano)alkyl, optionally substituted aryl, optionally substituted heteroaryl, optionally substituted cycloalkyl, and optionally substituted heterocycle, and

R^{1d} is as defined in connection with Formula I, or a pharmaceutically acceptable salt or solvate thereof.

In another embodiment, a Substituted Indole Compound is a compound having Formula IV-A:

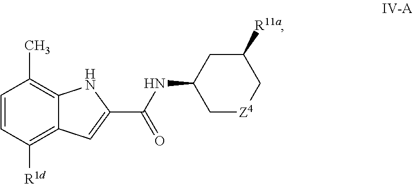

or a pharmaceutically acceptable salt or solvate thereof, wherein R^{1d}, R^{11a}, and Z⁴ are as defined in connection with Formula IV.

In another embodiment, a Substituted Indole Compound is a compound having Formula IV-B:

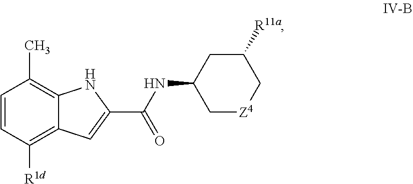

or a pharmaceutically acceptable salt or solvate thereof, wherein R^{1d}, R^{11a}, and Z⁴ are as defined in connection with Formula IV.

In another embodiment, a Substituted Indole Compound is a compound having Formula IV-C:

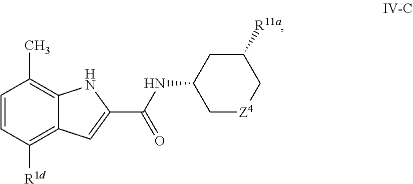

or a pharmaceutically acceptable salt or solvate thereof, wherein R^{1d}, R^{11a}, and Z⁴ are as defined in connection with Formula IV.

In another embodiment, a Substituted Indole Compound is a compound having Formula IV-D:

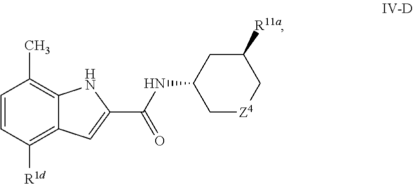

or a pharmaceutically acceptable salt or solvate thereof, wherein R^{1d}, R^{11a}, and Z⁴ are as defined in connection with Formula IV.

In another embodiment, a Substituted Indole Compound is a compound having any one of Formulae IV, IV-A, IV-B, IV-C, or IV-D, wherein:

R^{11a} is selected from the group consisting of: (A) unsubstituted 4- to 14-membered heterocyclo; (B) substituted 4- to 14-membered heterocyclo having one, two or three substituents independently selected from the group consisting of —N(R^{12a})C(═O)R^{13a}; —C(═O)R^{13b}; C₁-C₄ alkyl; (C₁-C₄ alkoxy)C₁-C₄ alkyl; (hydroxy)C₁-C₄ alkyl; C₁-C₄ haloalkyl; amino; hydroxy; —N(R^{12a})S(═O)₂R²⁴; —S(═O)₂R²⁴; unsubstituted C₃-C₆ cycloalkyl; substituted C₃-C₆ cycloalkyl having one or two substituents independently selected from the group consisting of halo, hydroxy, C₁-C₄ alkyl, amino, and (amino)C₁-C₄ alkyl; unsubstituted 4- to 14-membered heterocyclo; and substituted 4-to 14-membered heterocyclo having one or two substituents independently selected from the group consisting of amino, hydroxy, and C₁-C₄ alkyl; (C) unsubstituted 5- to 10-membered heteroaryl; (D) substituted 5- or 6-membered heteroaryl having one, two, three, or four substituents independently selected from the group consisting of halo, C₁-C₄ alkyl, and (amino)alkyl; (E) C₁-C₆ alkyl; and (F) —N(R^{12b})C(═O)R^{13c};

R^{12a} and R^{12b} are each independently selected from the group consisting of hydrogen, C₁-C₄ alkyl, (C₁-C₄ alkoxy)C₁-C₄ alkyl, and (hydroxy)C₁-C₄ alkyl;

R^{13a}, R^{13b}, and R^{13c} are each independently selected from the group consisting of C₁-C₆ alkyl; C₁-C₆ haloalkyl; unsubstituted C₃-C₆ cycloalkyl; C₁-C₆ alkoxy; (C₁-C₄ alkoxy)C₁-C₄ alkyl; (hydroxy)C₁-C₄ alkyl; (cyano)alkyl; unsubstituted C₆-C₁₀ aryl; substituted C₆-C₁₀ aryl, having one, two, three, or four substituents independently selected from the group consisting of halo, amino, hydroxy, and C₁-C₄ alkyl; unsubstituted 5- or 6-membered heteroaryl; substituted 5- or 6-membered heteroaryl having one, two, three, or four substituents independently selected from the group consisting of halo, amino, hydroxy, and C₁-C₄ alkyl; unsubstituted 4- to 14-membered heterocyclo; substituted 4- to 14-membered heterocyclo having one or two substituents independently selected from the group consisting of amino, hydroxy, and C₁-C₄ alkyl; amino; (amino)alkyl; (C₃-C₆ cycloalkyl)oxy; and (4- to 8-membered heterocyclo)oxy; and

R²⁴ is selected from the group consisting of C₁-C₄ alkyl and (hydroxy)C₁-C₄ alkyl.

In another embodiment, a Substituted Indole Compound is a compound having Formula IV, IV-A, IV-B, IV-C, or IV-D, wherein Z⁴ is —C(R^{28a})(R^{28b})—; and R^{28a} and R^{28b} are independently selected from the group consisting of hydrogen, C₁-C₄ alkyl, and fluoro, or a pharmaceutically acceptable salt or solvate thereof.

In another embodiment, a Substituted Indole Compound is a compound having Formula IV, IV-A, IV-B, IV-C, or IV-D, wherein Z⁴ is —C(R^{28a})(R^{28b})—; R^{28a} is hydrogen; and R^{28b} is selected from the group consisting of C₁-C₄ alkyl and fluoro, or a pharmaceutically acceptable salt or solvate thereof.

In another embodiment, a Substituted Indole Compound is a compound having Formula IV, IV-A, IV-B, IV-C, or IV-D, wherein Z⁴ is —C(R^{28a})(R^{28b})—; and R^{28a} and R^{28b} are independently C₁-C₄ alkyl, or a pharmaceutically acceptable salt or solvate thereof.

In another embodiment, a Substituted Indole Compound is a compound having Formula IV, IV-A, IV-B, IV-C, or IV-D, wherein Z⁴ is selected from the group consisting of —O—, —CH₂—, and —N(R²³), or Z⁴ is absent, or a pharmaceutically acceptable salt or solvate thereof.

In another embodiment, a Substituted Indole Compound is a compound having any one of Formulae IV, IV-A, IV-B, IV-C, or IV-D, wherein Z⁴ is —CH₂—, or a pharmaceutically acceptable salt or solvate thereof.

In another embodiment, a Substituted Indole Compound is a compound having any one of Formulae IV, IV-A, IV-B, IV-C, or IV-D, wherein R^{11a} is an optionally substituted 3- to 10-membered heterocycle linked to the rest of the molecule through a nitrogen atom, e.g., R^{11a} is

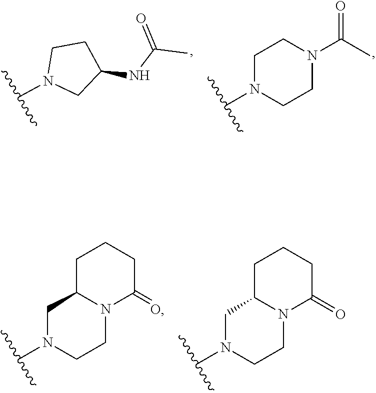

and the like.

In another embodiment, a Substituted Indole Compound is a compound having any one of Formulae IV, IV-A, IV-B, IV-C, or IV-D, wherein R^{11a} is a substituted 4- to 14-membered heterocyclo selected from the group consisting of:

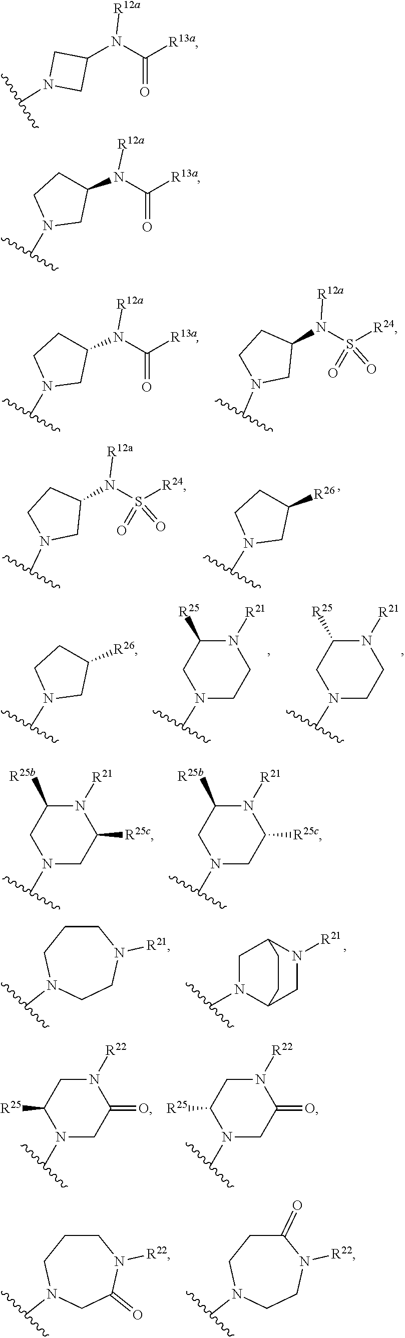

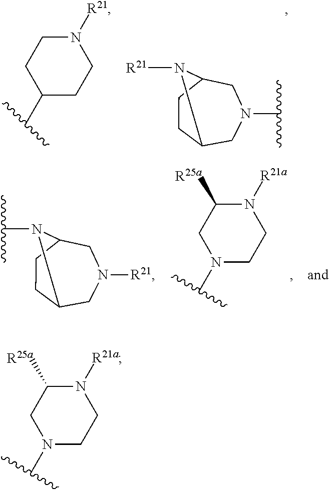

R^{12a} is selected from the group consisting of hydrogen, C₁-C₃ alkyl, (C₁-C₄ alkoxy)C₁-C₄ alkyl; and (hydroxy)C₁-C₄ alkyl;

R^{13a} is selected from the group consisting of C₁-C₄ alkyl; amino; unsubstituted C₃-C₆ cycloalkyl; substituted C₃-C₆ cycloalkyl having one or two substituents independently selected from the group consisting of halo, hydroxy, C₁-C₄ alkyl, amino, and (amino)C₁-C₄ alkyl; (C₁-C₄ alkoxy)C₁-C₄ alkyl; (hydroxy)C₁-C₄ alkyl; unsubstituted 4- to 14-membered heterocyclo; and substituted 4- to 14-membered heterocyclo having one or two substituents independently selected from the group consisting of amino, hydroxy, and C₁-C₄ alkyl;

R^{13b} is selected from the group consisting of C₁-C₄ alkyl; amino; C₁-C₄ haloalkyl; C₁-C₄ alkoxy; (hydroxy)C₁-C₄ alkyl; (C₁-C₄ alkoxy)C₁-C₄ alkyl; (amino)alkyl; unsubstituted C₃-C₆ cycloalkyl; substituted C₃-C₆ cycloalkyl having one or two substituents independently selected from the group consisting of halo, hydroxy, C₁-C₄ alkyl, amino, and (amino)C₁-C₄ alkyl; unsubstituted 4- to 14-membered heterocyclo; and substituted 4- to 14-membered heterocyclo having one or two substituents independently selected from the group consisting of amino, hydroxy, and C₁-C₄ alkyl; (C₃-C₆ cycloalkyl)oxy; and (4- to 8-membered heterocyclo)oxy;

R²¹ is selected from the group consisting of hydrogen, —C(═O)R^{13b}, C₁-C₄ alkyl, C₁-C₄ haloalkyl, unsubstituted 4- to 14-membered heterocyclo, and —S(═O)₂R²⁴;

R²² is C₁-C₄ alkyl; unsubstituted C₃-C₆ cycloalkyl; substituted C₃-C₆ cycloalkyl having one or two substituents independently selected from the group consisting of halo, hydroxy, C₁-C₄ alkyl, amino, and (amino)C₁-C₄ alkyl; unsubstituted 4- to 14-membered heterocyclo; and substituted 4- to 14-membered heterocyclo having one or two substituents independently selected from the group consisting of amino, hydroxy, and C₁-C₄ alkyl;

R²⁴ is selected from the group consisting of C₁-C₄ alkyl and (hydroxy)C₁-C₄ alkyl;

R²⁵ is selected from the group consisting of hydrogen, C₁-C₄ alkyl, and C₁-C₄ haloalkyl;

R^{25b} and R^{25c} are independently selected from the group consisting of C₁-C₄ alkyl and C₁-C₄ haloalkyl;

R²⁶ is selected from the group consisting of unsubstituted 4- to 14-membered heterocyclo; and substituted 4- to 14-membered heterocyclo having one or two substituents independently selected from the group consisting of amino, hydroxy, and C₁-C₄ alkyl; and

R^{21a} and R^{25a} taken together with the atoms to which they are attached form an optionally substituted 4- to 8-membered heterocyclo, or a pharmaceutically acceptable salt or solvate thereof.

In another embodiment, a Substituted Indole Compound is a compound having any one of Formulae IV, IV-A, IV-B, IV-C, or IV-D, wherein R^{11a} is selected from the group consisting of:

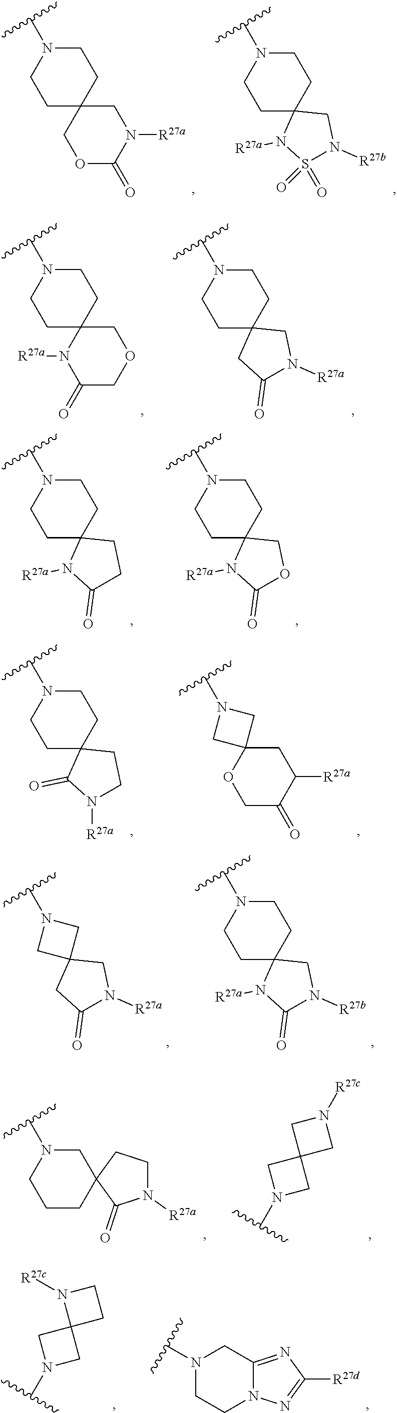

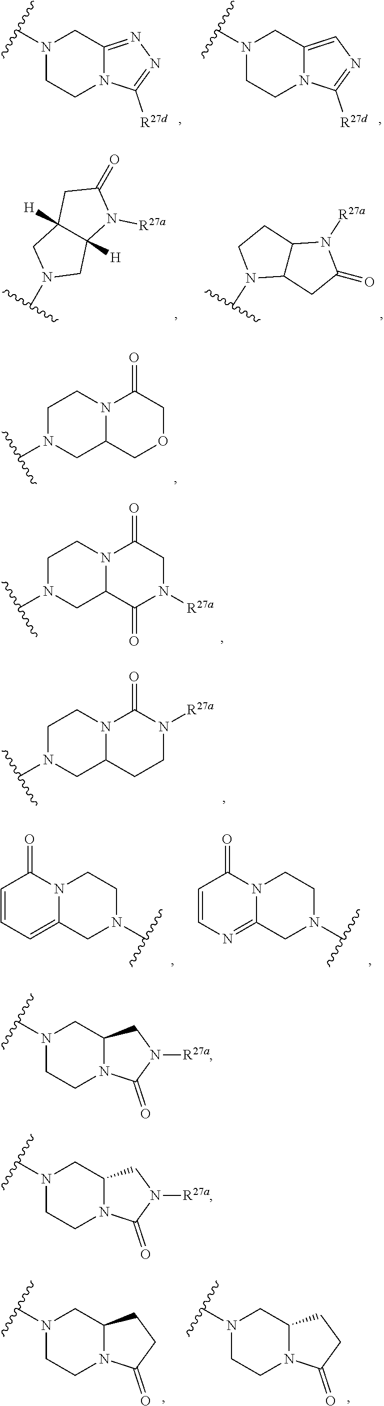

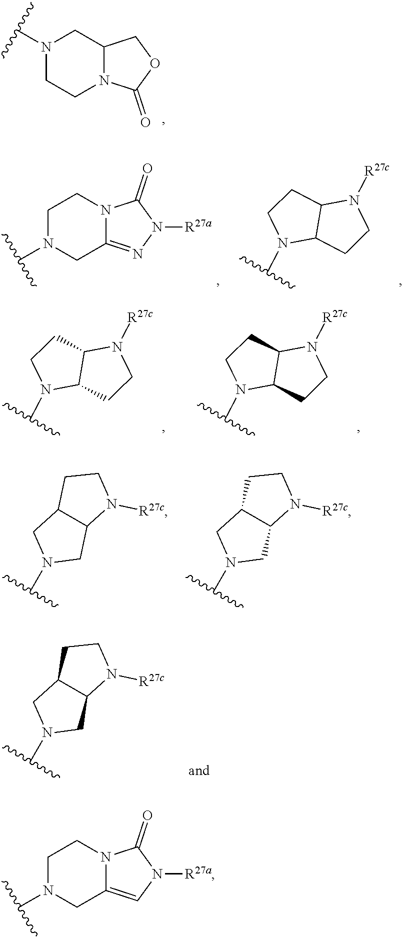

wherein:

R^{27a} and R^{27b} are each independently selected from the group consisting of hydrogen, C₁-C₄ alkyl, C₁-C₄ haloalkyl, (C₁-C₄ alkoxy)C₁-C₄ alkyl; and (hydroxy)C₁-C₄ alkyl;

R^{27C} is selected from the group consisting of hydrogen; —C(═O)R^{13b}; C₁-C₄ alkyl; C₁-C₄ haloalkyl; unsubstituted 4- to 14-membered heterocyclo; and substituted 4- to 14-membered heterocyclo having one or two substituents independently selected from the group consisting of amino, hydroxy, and C₁-C₄ alkyl; and —S(═O)₂R²⁴;

R^{27d} is selected from the group consisting of hydrogen; C₁-C₄ alkyl; and C₁-C₄ haloalkyl;

R^{13b} is selected from the group consisting of C₁-C₄ alkyl; aminoC₁-C₄ haloalkyl; C₁-C₄ alkoxy; (hydroxy)C₁-C₄ alkyl; (C₁-C₄ alkoxy)C₁-C₄ alkyl; (amino)alkyl; unsubstituted C₃-C₆ cycloalkyl; substituted C₃-C₆ cycloalkyl having one or two substituents independently selected from the group consisting of halo, hydroxy, C₁-C₄ alkyl, amino, and (amino)C₁-C₄ alkyl; unsubstituted 4- to 14-membered heterocyclo; substituted 4- to 14-membered heterocyclo having one or two substituents independently selected from the group consisting of amino, hydroxy, and C₁-C₄ alkyl; (C₃-C₆ cycloalkyl)oxy; and (4- to 8-membered heterocyclo)oxy; and

R²⁴ is selected from the group consisting of C₁-C₄ alkyl and (hydroxy)C₁-C₄ alkyl, or a pharmaceutically acceptable salt or solvate thereof.

In another embodiment, a Substituted Indole Compound is a compound having any one of Formulae IV, IV-A, IV-B, IV-C, or IV-D, wherein R^{11a} is selected from the group consisting of

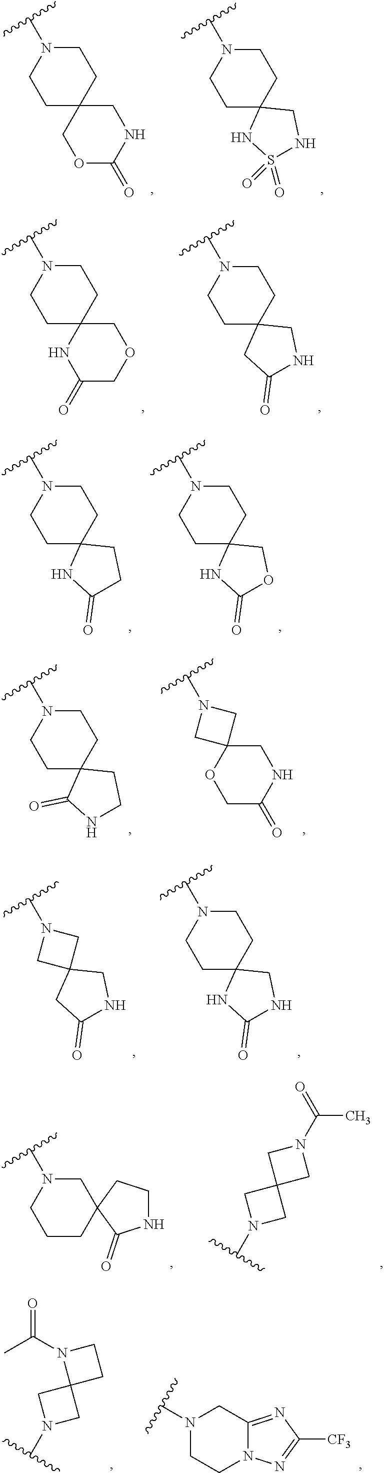

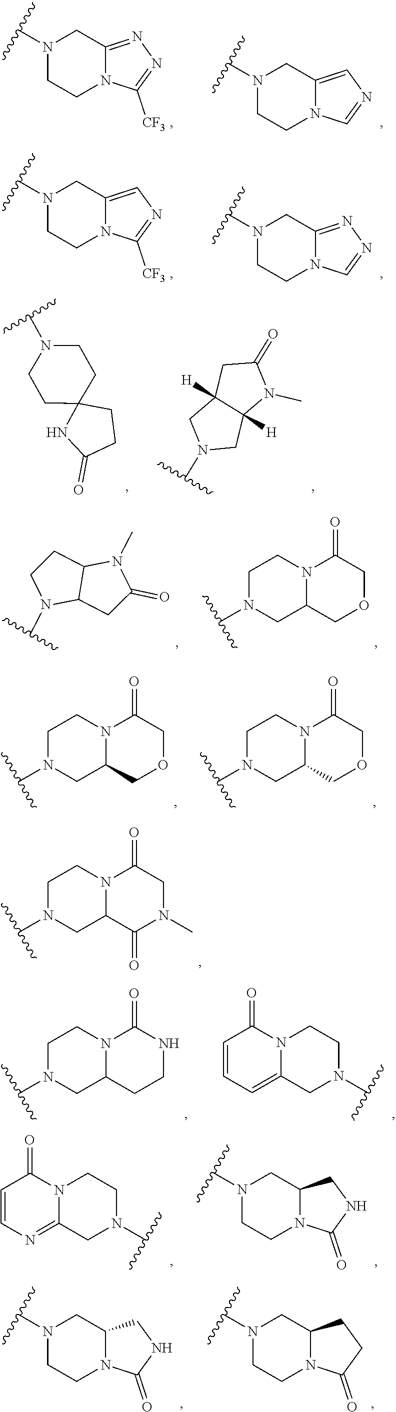

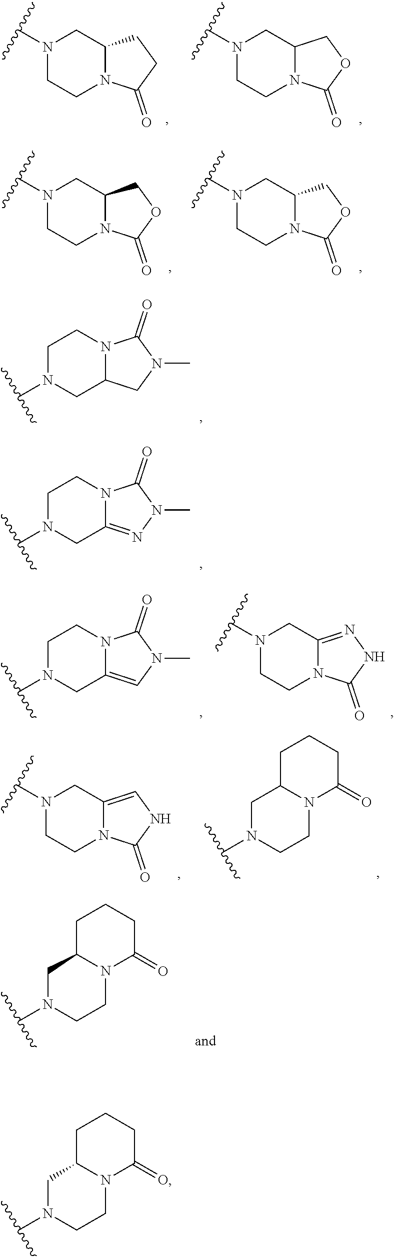

or a pharmaceutically acceptable salt or solvate thereof.

In another embodiment, a Substituted Indole Compound is a compound having any one of Formulae IV, IV-A, IV-B, IV-C, or IV-D, wherein R^{11a} is selected from the group consisting of unsubstituted 4- to 14-membered heterocyclo; substituted 4- to 14-membered heterocyclo having one or two substituents independently selected from the group consisting of —N(R^{12a})C(═O)R^{13a}, —C(═O)R^{13b}, and C₁-C₄ alkyl; unsubstituted 5- to 10-membered heteroaryl; and substituted 5- or 6-membered heteroaryl having one or two substituents independently selected from the group consisting of halo and C₁-C₄ alkyl, or a pharmaceutically acceptable salt or solvate thereof.

In another embodiment, a Substituted Indole Compound is a compound having any one of Formulae IV, IV-A, IV-B, IV-C, or IV-D, wherein R^{11a} is a substituted 4- to 14-membered heterocyclo is selected from the group consisting of:

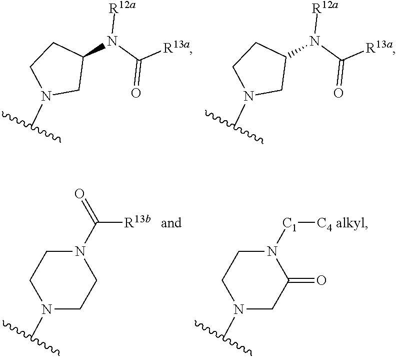

or a pharmaceutically acceptable salt or solvate thereof.

In another embodiment, R^{12a} is selected from the group consisting of hydrogen and C₁-C₃ alkyl; R^{13a} is C₁-C₄ alkyl; and R^{13b} is C₁-C₄ alkyl, or a pharmaceutically acceptable salt or solvate thereof. In another embodiment, R^{12a} is selected from the group consisting of hydrogen and methyl; R^{13a} is methyl; and R^{13b} is methyl, or a pharmaceutically acceptable salt or solvate thereof.

In another embodiment, a Substituted Indole Compound is a compound having any one of Formulae IV, IV-A, IV-B, IV-C, or IV-D, wherein R^{11a} is any one or more of the R^{2b} groups provided in connection with Formula III, see above, or a pharmaceutically acceptable salt or solvate thereof.

In another embodiment, a Substituted Indole Compound is a compound having any one of Formulae IV, IV-A, IV-B, IV-C, or IV-D, wherein:

R^{11a} is selected from the group consisting of:

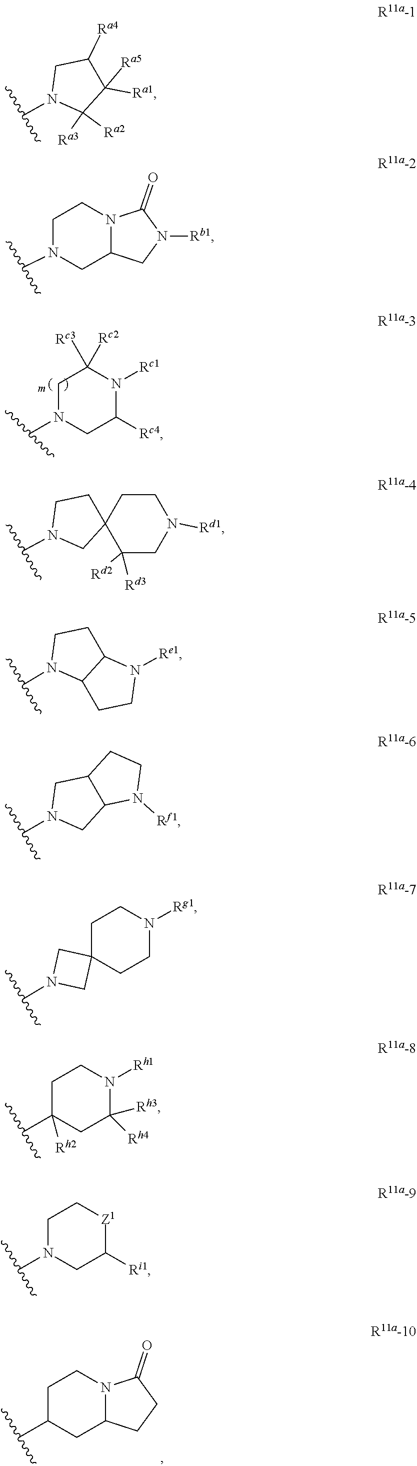

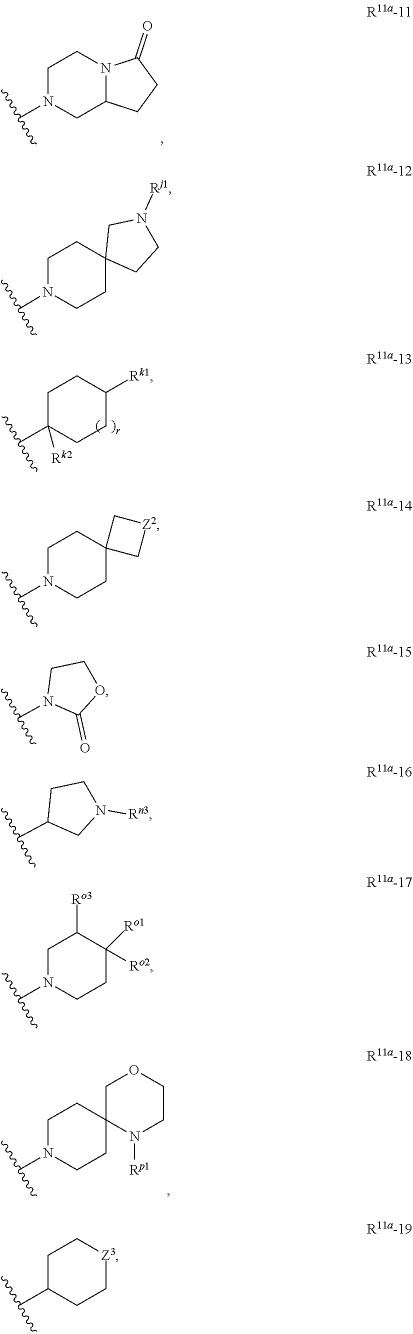

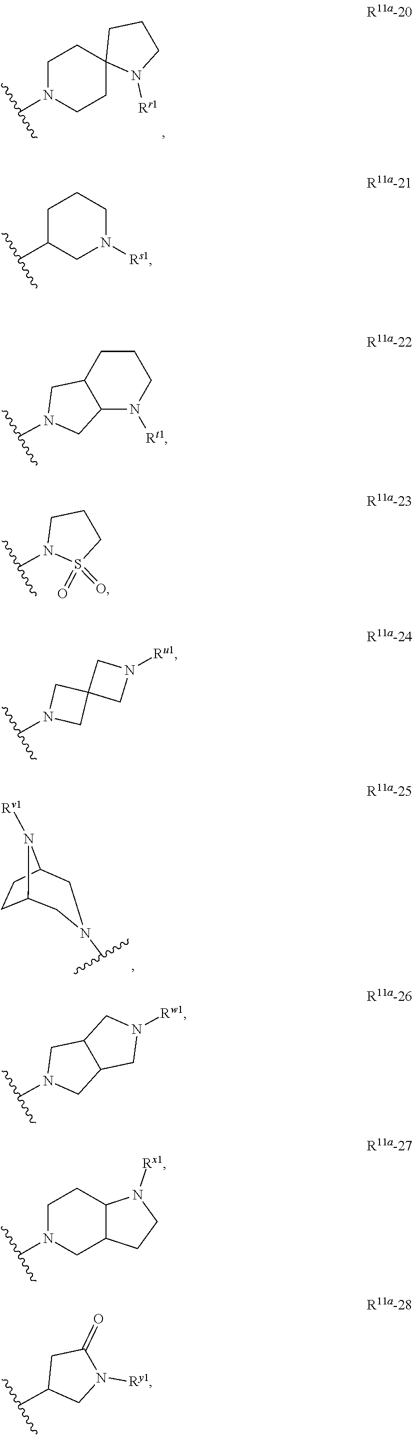

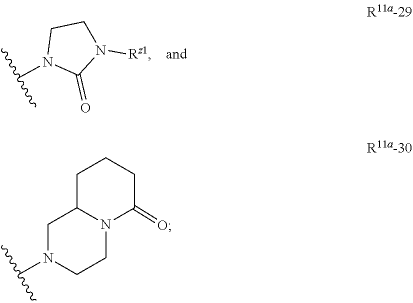

and R^{a1}, R^{a2}, R^{a3}, R^{a4}, R^{a5}, R^{b1}, R^{c1}, R^{c2}, R^{c3}, R^{c4}, m, R^{d1}, R^{d2}, R^{d3}, R^{e1}, R^{f1}, R^{y1}, R^{h1}, R^{h2}, R^{h3}, R^{h4}, Rⁱ¹, Z¹, R^{j1}, R^{k1}, R^{k2}, r, Z², Rⁿ³, R^{o1}, R^{o2}, R^{o3}, R^{p1}, Z³, R^{r1}, R^{s1}, R^{t1}, R^{u1}, R^{v1}, R^{w1}, R^{x1}, R^{y1}, and R^{z1} are as defined in connection with Formula III; or a pharmaceutically acceptable salt or solvate thereof.

In another embodiment, a Substituted Indole Compound is a compound having any one of Formulae IV, IV-A, IV-B, IV-C, or IV-D, wherein:

R^{11a} is selected from the group consisting of:

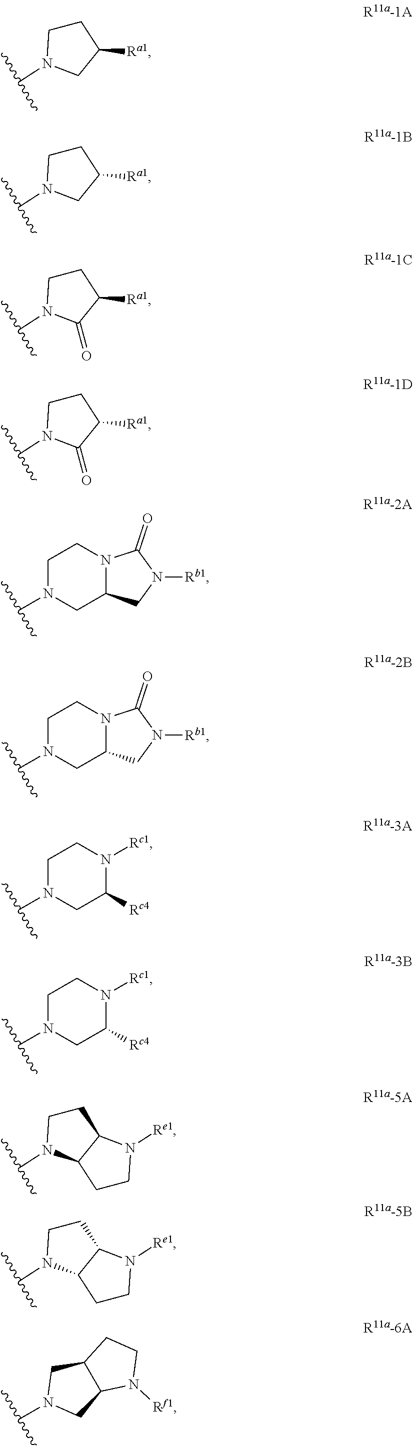

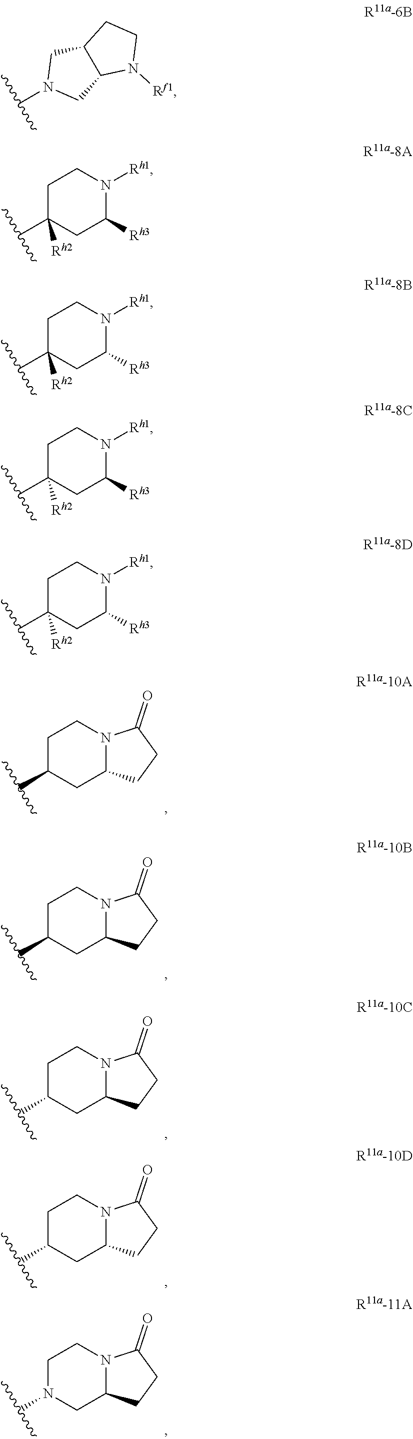

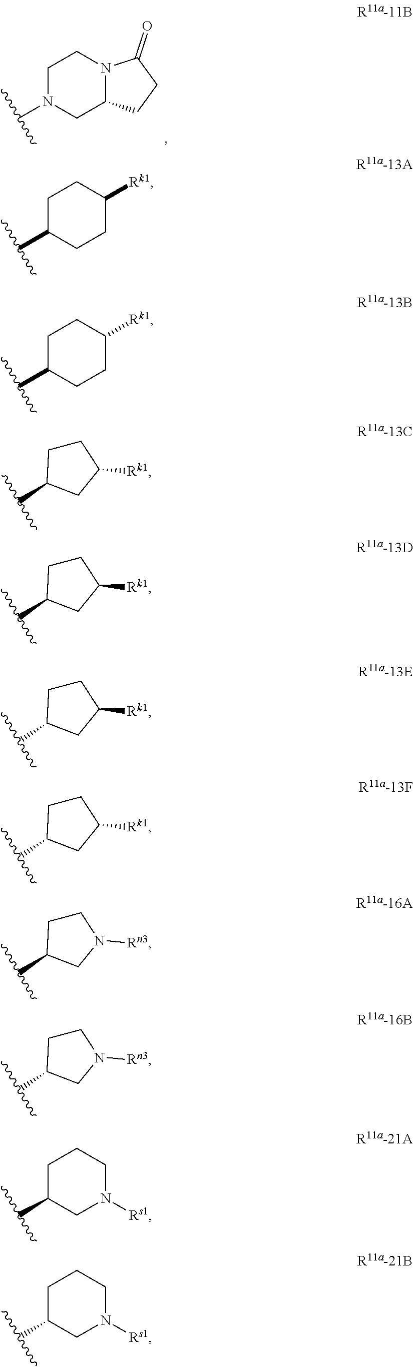

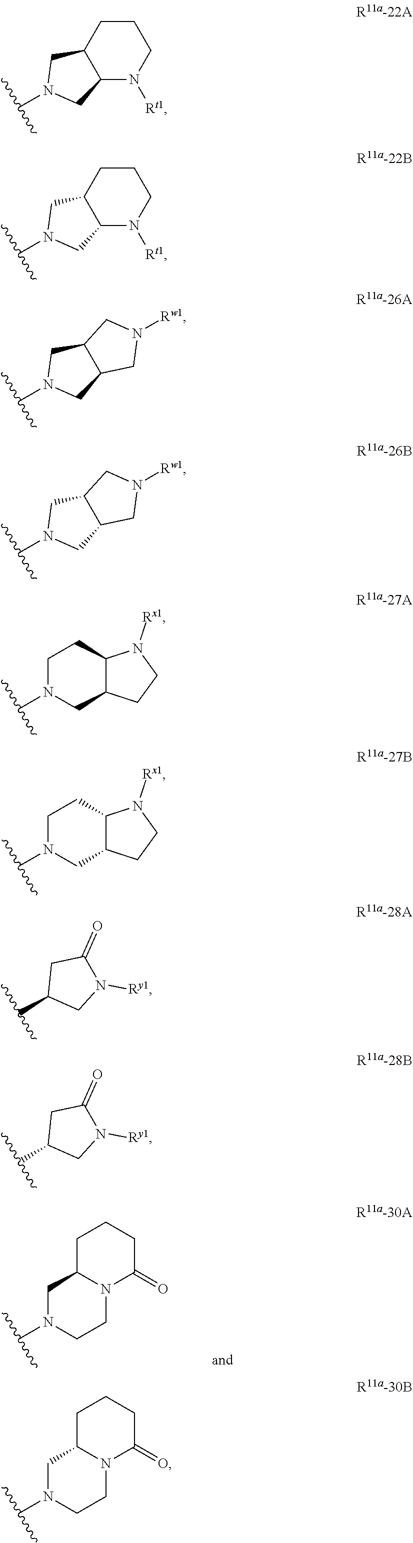

and R^{a1}, R^{a5}, R^{b1}, R^{e1}, R^{f1}, R^{h1}, R^{m}, R^{h3}, R^{k1}, Rⁿ³, R^{s1}, R^{t1}, R^{w1}, R^{x1}, and R^{y1} are as defined in connection with Formula III; or a pharmaceutically acceptable salt or solvate thereof.

In another embodiment, a Substituted Indole Compound is a compound having any one of Formulae IV, IV-A, IV-B, IV-C, or IV-D, wherein R^{11a} is R^{11a}-1, R^{11a}-1A, R^{11a}-1B, R^{11a}-1C, or R^{11a}-1D, or a pharmaceutically acceptable salt or solvate thereof. In another embodiment, R^{a1} is —N(R^{3a})C(═O)R^{4a}. In another embodiment, R^{a1} is —NR^{5a}R^{5b}. In another embodiment, R^{a1} is —NR^{5a}R^{5b} and R^{5a} and R^{5b} are independently selected from the group consisting of hydrogen and C₁-C₄ alkyl. In another embodiment, R^{a1} is optionally substituted 4- to 10-membered heterocyclo.

In another embodiment, a Substituted Indole Compound is a compound having any one of Formulae IV, IV-A, IV-B, IV-C, or IV-D, wherein R^{11a} is R^{11a}-2, R^{11a}-2A, or R^{11a}-2b, or a pharmaceutically acceptable salt or solvate thereof. In another embodiment, R^{b1} is C₁-C₄ alkyl.

In another embodiment, a Substituted Indole Compound is a compound having any one of Formulae IV, IV-A, IV-B, IV-C, or IV-D, wherein R^{11a} is R^{11a}-3, R^{11a}-3A, or R^{11a}-3B, or a pharmaceutically acceptable salt or solvate thereof. In another embodiment, R^{c1} is selected from the group consisting of C₁-C₄ alkyl, C₃-C₆ cycloalkyl, and —C(═O)R^{4c}. In another embodiment, R^{c2} and R^{c3} are each hydrogen. In another embodiment, R^{c2} and R^{c3} taken together with the carbon atom to which they are attached form a C(═O) group. In another embodiment, R^{c4} is hydrogen. In another embodiment, m is 1.

In another embodiment, a Substituted Indole Compound is a compound having any one of Formulae IV, IV-A, IV-B, IV-C, or IV-D, wherein R^{11a} is R^{11a}-4, or a pharmaceutically acceptable salt or solvate thereof. In another embodiment, R^{d1} is C(═O)R^{4c}. In another embodiment, R^{d2} and R^{d3} are each hydrogen or fluoro.

In another embodiment, a Substituted Indole Compound is a compound having any one of Formulae IV, IV-A, IV-B, IV-C, or IV-D, wherein R^{11a} is R^{11a}-5, R^{11a}-5A, or R^{11a}-5B, or a pharmaceutically acceptable salt or solvate thereof. In another embodiment, R^{e1} is —C(═O)R^{4c}.

In another embodiment, a Substituted Indole Compound is a compound having any one of Formulae IV, IV-A, IV-B, IV-C, or IV-D, wherein R^{11a} is R^{11a}-6, R^{11a}-6A, or R^{11a}-6B, or a pharmaceutically acceptable salt or solvate thereof. In another embodiment, R^{f1} is C(═O)R^{4c}.

In another embodiment, a Substituted Indole Compound is a compound having any one of Formulae IV, IV-A, IV-B, IV-C, or IV-D, wherein R^{11a} is R^{11a}-7, or a pharmaceutically acceptable salt or solvate thereof. In another embodiment, R^{g1} is C(═O)R^{4c}.

In another embodiment, a Substituted Indole Compound is a compound having any one of Formulae IV, IV-A, IV-B, IV-C, or IV-D, wherein R^{11a} is R^{11a}-8, R^{11a}-8A, R^{11a}-8B, R^{11a}-8C, or R^{11a}-8D, or a pharmaceutically acceptable salt or solvate thereof. In another embodiment, R^{h1} is —C(═O)R^{4c}. In another embodiment, R^{h2} is selected from the group consisting of hydrogen and C₁-C₃ alkyl. In another embodiment, R^{h3} is hydrogen.

In another embodiment, a Substituted Indole Compound is a compound having any one of Formulae IV, IV-A, IV-B, IV-C, or IV-D, wherein R^{11a} is R^{11a}-9, or a pharmaceutically acceptable salt or solvate thereof.

In another embodiment, a Substituted Indole Compound is a compound having any one of Formulae IV, IV-A, IV-B, IV-C, or IV-D, wherein R^{11a} is selected from the group consisting of R^{11a}-10, R^{11a}-10A, R^{11a}-10B, R^{11a}-10C, and R^{11a}-10d, or a pharmaceutically acceptable salt or solvate thereof.

In another embodiment, a Substituted Indole Compound is a compound having any one of Formulae IV, IV-A, IV-B, IV-C, or IV-D, wherein R^{11a} is selected from the group consisting of R^{11a}-11, R^{11a}-11A and R^{11a}-11B, or a pharmaceutically acceptable salt or solvate thereof.

In another embodiment, a Substituted Indole Compound is a compound having any one of Formulae IV, IV-A, IV-B, IV-C, or IV-D, wherein R^{11a} is R^{11a}-12, or a pharmaceutically acceptable salt or solvate thereof. In another embodiment, R^{j1} is —C(═O)R^{4c}.

In another embodiment, a Substituted Indole Compound is a compound having any one of Formulae IV, IV-A, IV-B, IV-C, or IV-D, wherein R^{11a} is selected from the group consisting of R^{11a}-13, R^{11a}-13A, R^{11a}-13B, R^{11a}-13C, R^{11a}-13D, R^{11a}-13E, and R^{11a}-13F, or a pharmaceutically acceptable salt or solvate thereof.

In another embodiment, a Substituted Indole Compound is a compound having any one of Formulae IV, IV-A, IV-B, IV-C, or IV-D, wherein R^{11a} is R^{11a}-14, or a pharmaceutically acceptable salt or solvate thereof.

In another embodiment, a Substituted Indole Compound is a compound having any one of Formulae IV, IV-A, IV-B, IV-C, or IV-D, wherein R^{11a} is R^{11a}-15, or a pharmaceutically acceptable salt or solvate thereof.

In another embodiment, a Substituted Indole Compound is a compound having any one of Formulae IV, IV-A, IV-B, IV-C, or IV-D, wherein R^{11a} is selected from the group consisting of R^{11a}-16, R^{11a}-16A and R^{11a}-16B, or a pharmaceutically acceptable salt or solvate thereof. In another embodiment, Rⁿ³ is —C(═O)R^{4c}.

In another embodiment, a Substituted Indole Compound is a compound having any one of Formulae IV, IV-A, IV-B, IV-C, or IV-D, wherein R^{11a} is R^{11a}-17, or a pharmaceutically acceptable salt or solvate thereof.

In another embodiment, a Substituted Indole Compound is a compound having any one of Formulae IV, IV-A, IV-B, IV-C, or IV-D, wherein R^{11a} is R^{11a}-18, or a pharmaceutically acceptable salt or solvate thereof.

In another embodiment, a Substituted Indole Compound is a compound having any one of Formulae IV, IV-A, IV-B, IV-C, or IV-D, wherein R^{11a} is R^{11a}-19, or a pharmaceutically acceptable salt or solvate thereof.

In another embodiment, a Substituted Indole Compound is a compound having any one of Formulae IV, IV-A, IV-B, IV-C, or IV-D, wherein R^{11a} is R^{11a}-20, or a pharmaceutically acceptable salt or solvate thereof.

In another embodiment, a Substituted Indole Compound is a compound having any one of Formulae IV, IV-A, IV-B, IV-C, or IV-D, wherein R^{11a} is selected from the group consisting of R^{11a}-21, R^{11a}-21A and R^{11a}-21B, or a pharmaceutically acceptable salt or solvate thereof.

In another embodiment, a Substituted Indole Compound is a compound having any one of Formulae IV, IV-A, IV-B, IV-C, or IV-D, wherein R^{11a} is selected from the group consisting of R^{11a}-22, R^{11a}-22A and R^{11a}-22B, or a pharmaceutically acceptable salt or solvate thereof.

In another embodiment, a Substituted Indole Compound is a compound having any one of Formulae IV, IV-A, IV-B, IV-C, or IV-D, wherein R^{11a} is R^{11a}-23, or a pharmaceutically acceptable salt or solvate thereof.

In another embodiment, a Substituted Indole Compound is a compound having any one of Formulae IV, IV-A, IV-B, IV-C, or IV-D, wherein R^{11a} is R^{11a}-24, or a pharmaceutically acceptable salt or solvate thereof.

In another embodiment, a Substituted Indole Compound is a compound having any one of Formulae IV, IV-A, IV-B, IV-C, or IV-D, wherein R^{11a} is R^{11a}-25, or a pharmaceutically acceptable salt or solvate thereof.

In another embodiment, a Substituted Indole Compound is a compound having any one of Formulae IV, IV-A, IV-B, IV-C, or IV-D, wherein R^{11a} is selected from the group consisting of R^{11a}-26, R^{11a}-26A and R^{11a}-26B, or a pharmaceutically acceptable salt or solvate thereof.

In another embodiment, a Substituted Indole Compound is a compound having any one of Formulae IV, IV-A, IV-B, IV-C, or IV-D, wherein R^{11a} is selected from the group consisting of R^{11a}-27, R^{11a}-27A and R^{11a}-27B, or a pharmaceutically acceptable salt or solvate thereof.

In another embodiment, a Substituted Indole Compound is a compound having any one of Formulae IV, IV-A, IV-B, IV-C, or IV-D, wherein R^{11a} is selected from the group consisting of R^{11a}-28, R^{11a}-28A and R^{11a}-28B, or a pharmaceutically acceptable salt or solvate thereof.

In another embodiment, a Substituted Indole Compound is a compound having any one of Formulae IV, IV-A, IV-B, IV-C, or IV-D, wherein R^{11a} is R^{11a}-29, or a pharmaceutically acceptable salt or solvate thereof.

In another embodiment, a Substituted Indole Compound is a compound having any one of Formulae IV, IV-A, IV-B, IV-C, or IV-D, wherein R^{11a} is R^{11a}-30, R^{11a}-30A, or R^{11a}-30B, or a pharmaceutically acceptable salt or solvate thereof.

In another embodiment, a Substituted Indole Compound is a compound having any one of Formulae IV-A, IV-B, IV-C, or IV-D, wherein:

Z⁴ is —CH₂—;

R^{11a} is selected from the group consisting of:

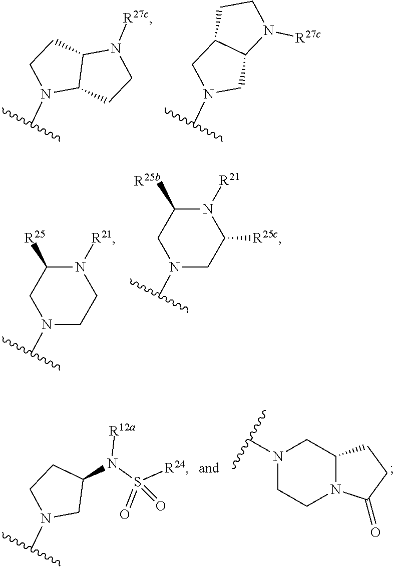

R^{12a} is selected from the group consisting of hydrogen and C₁-C₃ alkyl;

R²¹ is —C(═O)R^{13b};

R^{27C} is —C(═O)R^{13b};

R^{13b} is selected from the group consisting of C₁-C₄ alkyl and (hydroxy)C₁-C₄ alkyl;

R²⁴ is C₁-C₄ alkyl;

R²⁵ is selected from the group consisting of hydrogen, C₁-C₄ alkyl, and C₁-C₄ haloalkyl; and

R^{25b} and R^{25c} are independently selected from the group consisting of C₁-C₄ alkyl and C₁-C₄ haloalkyl, or a pharmaceutically acceptable salt or solvate thereof.

In another embodiment, a Substituted Indole Compound is a compound having any one of Formulae IV-A, IV-B, IV-C, or IV-D, wherein:

Z⁴ is —CH₂—;

R^{11a} is selected from the group consisting of:

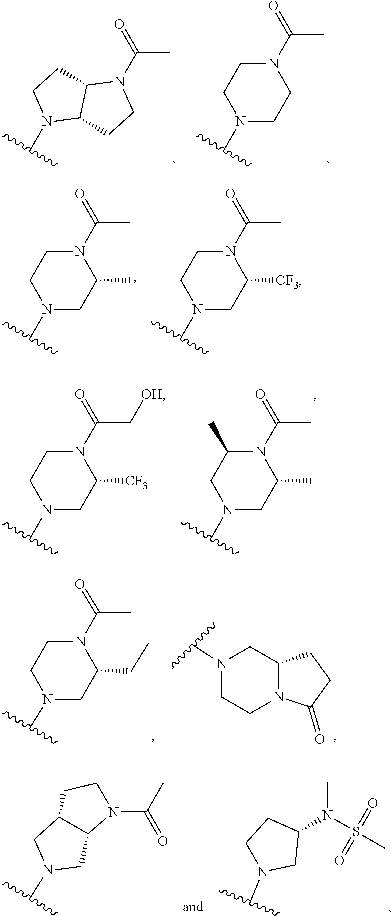

or a pharmaceutically acceptable salt or solvate thereof.

In another embodiment, a Substituted Indole Compound is a compound having Formula V:

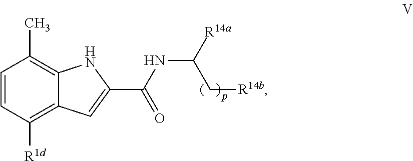

wherein:

R^{14a} is selected from the group consisting of optionally substituted alkyl and optionally substituted heteroaryl;

R^{14b} is selected from the group consisting of optionally substituted alkyl, optionally substituted heteroaryl, optionally substituted aryl, optionally substituted heterocyclo, optionally substituted cycloalkyl, and carboxamido; and

p is 0, 1, 2, or 3; or a pharmaceutically acceptable salt or solvate thereof.

In another embodiment, a Substituted Indole Compound is a compound having Formula V-A:

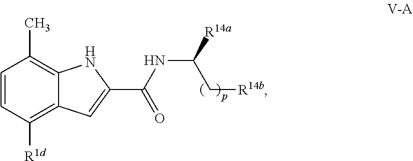

wherein R^{1d}, R^{14a}, R^{14d}, and p are as defined in connection with Formula V, or a pharmaceutically acceptable salt or solvate thereof.

In another embodiment, a Substituted Indole Compound is a compound having Formula V-B:

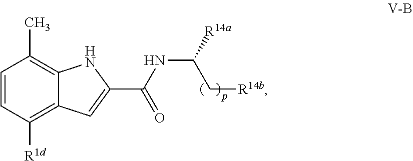

wherein R^{1d}, R^{14a}, R^{14d}, and p are as defined in connection with Formula V, or a pharmaceutically acceptable salt or solvate thereof.

In another embodiment, a Substituted Indole Compound is a compound having any one of Formulae V, V-A, or V-B, wherein:

R^{14a} is selected from the group consisting of (A) unsubstituted 5- to 10-membered heteroaryl; (B) substituted 5- or 10-membered heteroaryl having one, two, three, or four substituents independently selected from the group consisting of (i) halo; (ii) C₁-C₄ alkyl; (iii) C₁-C₄ alkoxy; (iv) (3- to 8-membered heterocyclo)C₁-C₄ alkyl; (v) (5- to 9-membered heteroaryl)C₁-C₄ alkyl; (vi) —C(═O)NR^{15a}R^{15b}; (vii) unsubstituted 5- to 10-membered heteroaryl; (viii) substituted 5- or 10-membered heteroaryl having one, two, or three substituents independently selected from the group consisting of halo, C₁-C₄ alkyl, (3- to 8-membered heterocyclo)C₁-C₄ alkyl, 5- to 9-membered heteroaryl, and —NR^{15e}R^{15f}; (ix) —OR¹⁶ (x) unsubstituted C₃-C₆ cycloalkyl; (xi) substituted C₃-C₆ cycloalkyl having one, two, three, or four substituents independently selected from the group consisting of C₁-C₄ alkyl and —N(R^{17a})C(═O)R^{18a}; (xii) cyano; (xiii) unsubstituted 4- to 14-membered heterocyclo; (xiv) substituted 4- to 14-membered heterocyclo having one or two substituents independently selected from the group consisting of C₁-C₄ alkyl, (5- to 9-membered heteroaryl)C₁-C₄ alkyl; (xv) (carboxy)C₁-C₄ alkyl; (xvi) (carboxamido)C₁-C₄ alkyl; and (xvii) carboxy; and (C) C₁-C₆ alkyl;

R^{14b} is selected from the group consisting of: (A) unsubstituted 5- to 10-membered heteroaryl; (B) substituted 5- or 10-membered heteroaryl having one, two, three, or four substituents independently selected from the group consisting of halo, C₁-C₄ alkyl, and (C₃-C₆ cycloalkyl)C₁-C₄ alkyl; (C) unsubstituted C₆-C₁₀ aryl; (D) substituted C₆-C₁₀ aryl, having one, two, three, or four substituents independently selected from the group consisting of halo, C₁-C₄ alkyl, and (3- to 8-membered heterocyclo)C₁-C₄ alkyl; (E) unsubstituted 4- to 14-membered heterocyclo; (F) substituted 4- to 14-membered heterocyclo having one, two, three, or four substituents independently selected from the group consisting of hydroxy, amino, and C₁-C₄ alkyl; (G) —C(═O)NR^{15c}R^{15d}; (H) unsubstituted C₃-C₆ cycloalkyl; and (I) C₁-C₆ alkyl;

p is 0, 1, 2, or 3;

R^{15a} and R^{15b} are independently selected from the group consisting of: (A) hydrogen; (B) C₁-C₆ alkyl; (C) C₁-C₆ haloalkyl; (D) (C₁-C₄ alkoxy)C₁-C₄ alkyl; (E) (hydroxy)C₁-C₄ alkyl; (F) (cyano)alkyl; (G) unsubstituted C₆-C₁₀ aryl; (H) substituted C₆-C₁₀ aryl, having one, two, three, or four substituents independently selected from the group consisting of halo, amino, hydroxy, and C₁-C₄ alkyl; (I) unsubstituted 5- or 6-membered heteroaryl; (J) substituted 5- or 6-membered heteroaryl having one, two, three, or four substituents independently selected from the group consisting of halo, amino, hydroxy, and C₁-C₄ alkyl; (K) unsubstituted 4- to 14-membered heterocyclo; (L) substituted 4- to 14-membered heterocyclo having one or two substituents independently selected from the group consisting of amino, hydroxy, and C₁-C₄ alkyl; (M) unsubstituted C₃-C₈ cycloalkyl; and (N) substituted C₃-C₈ cycloalkyl having one, two, three, or four substituents independently selected from the group consisting of C₁-C₆ alkyl and —NR^{15g}R^{15h}; or

R^{15a} and R^{15b} taken together with the nitrogen atom to which they are attached form an optionally substituted 4- to 14-membered heterocyclo;

R^{15C} and R^{15d} are independently selected from the group consisting of: (A) hydrogen; (B) C₁-C₆ alkyl; (C) C₁-C₆ haloalkyl; (D) (C₁-C₄ alkoxy)C₁-C₄ alkyl; (E) (hydroxy)C₁-C₄ alkyl; (F) (cyano)alkyl; (G) unsubstituted C₆-C₁₀ aryl; (H) substituted C₆-C₁₀ aryl, having one, two, three, or four substituents independently selected from the group consisting of halo, amino, hydroxy, and C₁-C₄ alkyl; (I) unsubstituted 5- or 6-membered heteroaryl; (J) substituted 5- or 6-membered heteroaryl having one, two, three, or four substituents independently selected from the group consisting of halo, amino, hydroxy, and C₁-C₄ alkyl; (K) unsubstituted 4- to 14-membered heterocyclo; (L) substituted 4- to 14-membered heterocyclo having one or two substituents independently selected from the group consisting of amino, hydroxy, and C₁-C₄ alkyl; (M) unsubstituted C₃-C₈ cycloalkyl; and (N) substituted C₃-C₈ cycloalkyl having one, two, three, or four substituents independently selected from the group consisting of C₁-C₆ alkyl and —NR^{15g}R^{15h}; or

R^{15C} and R^{15d} taken together with the nitrogen atom to which they are attached form an optionally substituted 4- to 14-membered heterocyclo;

R^{15e} and R^{15f} are independently selected from the group consisting of: (A) hydrogen; (B) C₁-C₆ alkyl; (C) C₁-C₆ haloalkyl; (D) (C₁-C₄ alkoxy)C₁-C₄ alkyl; (E) (hydroxy)C₁-C₄ alkyl; (F) (cyano)alkyl; (G) unsubstituted C₆-C₁₀ aryl; (H) substituted C₆-C₁₀ aryl, having one, two, three, or four substituents independently selected from the group consisting of halo, amino, hydroxy, and C₁-C₄ alkyl; (I) unsubstituted 5- or 6-membered heteroaryl; (J) substituted 5- or 6-membered heteroaryl having one, two, three, or four substituents independently selected from the group consisting of halo, amino, hydroxy, and C₁-C₄ alkyl; (K) unsubstituted 4- to 14-membered heterocyclo; (L) substituted 4- to 14-membered heterocyclo having one or two substituents independently selected from the group consisting of amino, hydroxy, and C₁-C₄ alkyl; (M) unsubstituted C₃-C₈ cycloalkyl; and (N) substituted C₃-C₈ cycloalkyl having one, two, three, or four substituents independently selected from the group consisting of C₁-C₆ alkyl and —NR^{15g}R^{15h}; or

R^{15e} and R^{15f} taken together with the nitrogen atom to which they are attached form an optionally substituted 4- to 14-membered heterocyclo;

R^{15g} and R^{15h} are independently selected from the group consisting of: (A) hydrogen; (B) C₁-C₆ alkyl; (C) C₁-C₆ haloalkyl; (D) C₁-C₆ alkoxy; (E) (C₁-C₄ alkoxy)C₁-C₄ alkyl; (F) (hydroxy)C₁-C₄ alkyl; (G) (cyano)alkyl; (H) unsubstituted C₆-C₁₀ aryl; (I) substituted C₆-C₁₀ aryl, having one, two, three, or four substituents independently selected from the group consisting of halo, amino, hydroxy, and C₁-C₄ alkyl; (J) unsubstituted 5- or 6-membered heteroaryl; (K) substituted 5- or 6-membered heteroaryl having one, two, three, or four substituents independently selected from the group consisting of halo, amino, hydroxy, and C₁-C₄ alkyl; (L) unsubstituted 4- to 14-membered heterocyclo; (M) substituted 4- to 14-membered heterocyclo having one or two substituents independently selected from the group consisting of amino, hydroxy, and C₁-C₄ alkyl; (N) unsubstituted C₃-C₈ cycloalkyl; and (O) substituted C₃-C₈ cycloalkyl having one, two, three, or four substituents independently selected from the group consisting of C₁-C₆ alkyl and —NR^{15g}R^{15h}; or

R^{15g} and R^{15g} taken together with the nitrogen atom to which they are attached form an optionally substituted 4- to 14-membered heterocyclo;

R¹⁶ is (amino)(hydroxy)C₁-C₄ alkyl;

R^{17a} is selected from the group consisting of hydrogen and C₁-C₄ alkyl;

R^{18a} is selected from the group consisting of: (A) C₁-C₆ alkyl; (B) C₁-C₆ haloalkyl; (C) C₁-C₆ alkoxy; (D) (C₁-C₄ alkoxy)C₁-C₄ alkyl; (E) (hydroxy)C₁-C₄ alkyl; (F) (cyano)alkyl; (G) unsubstituted C₆-C₁₀ aryl; (H) substituted C₆-C₁₀ aryl, having one, two, three, or four substituents independently selected from the group consisting of halo, amino, hydroxy, and C₁-C₄ alkyl; (I) unsubstituted 5- or 6-membered heteroaryl; (J) substituted 5- or 6-membered heteroaryl having one, two, three, or four substituents independently selected from the group consisting of halo, amino, hydroxy, and C₁-C₄ alkyl; (K) unsubstituted 4- to 14-membered heterocyclo; (L) substituted 4- to 14-membered heterocyclo having one or two substituents independently selected from the group consisting of amino, hydroxy, and C₁-C₄ alkyl; (M) unsubstituted C₃-C₈ cycloalkyl; and (N) substituted C₃-C₈ cycloalkyl having one, two, three, or four substituents independently selected from the group consisting of amino, hydroxy, and C₁-C₄ alkyl, or a pharmaceutically acceptable salt or solvate thereof.

In another embodiment, a Substituted Indole Compound is a compound having any one of Formulae V, V-A, or V-B, wherein R^{14a} is selected from the group consisting of unsubstituted 5- to 10-membered heteroaryl; and substituted 5- or 10-membered heteroaryl having one, two, or three substituents independently selected from the group consisting of C₁-C₄ alkyl; C₁-C₄ alkoxy; (3- to 8-membered heterocyclo)C₁-C₄ alkyl; (5-to 9-membered heteroaryl)C₁-C₄ alkyl; —C(═O)NR^{15a}R^{15b}; unsubstituted 5- to 10-membered heteroaryl; substituted 5- or 10-membered heteroaryl having one, two, or three substituents independently selected from the group consisting of halo, C₁-C₄ alkyl, (3- to 8-membered heterocyclo)C₁-C₄ alkyl, 5- to 9-membered heteroaryl, and —NR^{15e}R^{15f}; unsubstituted C₃-C₆ cycloalkyl; and substituted C₃-C₆ cycloalkyl having one, two, or three substituents independently selected from the group consisting of C₁-C₄ alkyl and —N(R^{17a})C(═O)R^{18a}, or a pharmaceutically acceptable salt or solvate thereof.

In another embodiment, a Substituted Indole Compound is a compound having any one of Formulae V, V-A, or V-B, wherein R^{14a} is a substituted pyridyl having one, two, or three substituents independently selected from the group consisting of C₁-C₄ alkyl; C₁-C₄ alkoxy; (3- to 8-membered heterocyclo)C₁-C₄ alkyl; (5- to 9-membered heteroaryl)C₁-C₄ alkyl; —C(═O)NR^{15a}R^{15b}; unsubstituted 5- to 10-membered heteroaryl; substituted 5- to 10-membered heteroaryl having one, two, or three substituents independently selected from the group consisting of halo, C₁-C₄ alkyl, (3- to 8-membered heterocyclo)C₁-C₄ alkyl, 5- to 9-membered heteroaryl, and —NR^{15e}R^{15f}; unsubstituted C₃-C₆ cycloalkyl; and substituted C₃-C₆ cycloalkyl having one, two, or three substituents independently selected from the group consisting of C₁-C₄ alkyl and —N(R^{17a})C(═O)R^{18a}, or a pharmaceutically acceptable salt or solvate thereof.

In another embodiment, a Substituted Indole Compound is a compound having any one of Formulae V, V-A, or V-B, wherein R^{14b} is selected from the group consisting of unsubstituted 5- to 10-membered heteroaryl; substituted 5- to 10-membered heteroaryl having one or two substituents independently selected from the group consisting of C₁-C₄ alkyl and (C₃-C₆ cycloalkyl)C₁-C₄ alkyl; unsubstituted C₆-C₁₀ aryl; substituted C₆-C₁₀ aryl, having one or two substituents independently selected from the group consisting of C₁-C₄ alkyl and (3- to 8-membered heterocyclo)C₁-C₄ alkyl; unsubstituted 4- to 14-membered heterocyclo; substituted 4- to 14-membered heterocyclo having one or two substituents independently selected from the group consisting of hydroxy, amino, and C₁-C₄ alkyl; and unsubstituted C₃-C₆ cycloalkyl, or a pharmaceutically acceptable salt or solvate thereof.

In another embodiment, a Substituted Indole Compound is a compound having any one of Formulae V, V-A, or V-B, wherein R^{14b} is selected from the group consisting of unsubstituted 5- or 6-membered heteroaryl; substituted 5- or 6-membered heteroaryl having one or two substituents independently selected from the group consisting of C₁-C₄ alkyl and (C₃-C₆ cycloalkyl)C₁-C₄ alkyl; unsubstituted phenyl; substituted phenyl, having one or two substituents independently selected from the group consisting of C₁-C₄ alkyl and (3- to 8-membered heterocyclo)C₁-C₄ alkyl; and unsubstituted C₃-C₆ cycloalkyl, or a pharmaceutically acceptable salt or solvate thereof.

In another embodiment, a Substituted Indole Compound is a compound having any one of Formulae V, V-A, or V-B, wherein p is 0, or a pharmaceutically acceptable salt or solvate thereof.

In another embodiment, a Substituted Indole Compound is a compound having any one of Formulae V, V-A, or V-B, wherein p is 1, or a pharmaceutically acceptable salt or solvate thereof.

In another embodiment, a Substituted Indole Compound is a compound having Formula VI:

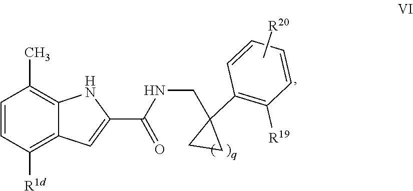

wherein:

R¹⁹ is selected from the group consisting of unsubstituted 4- to 14-membered heterocyclo; and substituted 4- to 14-membered heterocyclo having one or two substituents independently selected from the group consisting of amino, hydroxy, and C₁-C₄ alkyl;

R²⁰ is selected from the group consisting of hydrogen, halo, and C₁-C₄ alkyl; and

q is 1, 2, or 3, or a pharmaceutically acceptable salt or solvate thereof.

In another embodiment, a Substituted Indole Compound is a compound having Formula VI, wherein q is 1.

In another embodiment, a Substituted Indole Compound is a compound having Formula VII:

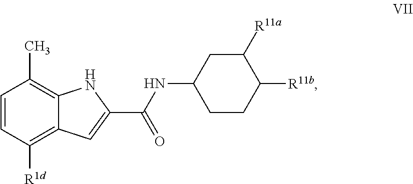

wherein:

R^{11b} is selected from the group consisting of C₁-C₄ alkyl, halo, and C₁-C₄ haloalkyl; and

R^{1d} and R^{11a} are as defined in connection with Formula IV, or a pharmaceutically acceptable salt or solvate thereof.

In another embodiment, a Substituted Indole Compound is a compound having Formula VII-A:

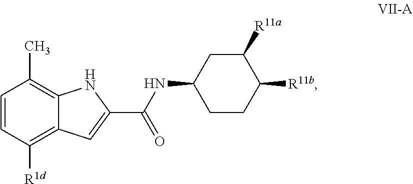

wherein R^{1d}, R^{11a}, and R^{11b} are as defined in connection with Formula VII, or a pharmaceutically acceptable salt or solvate thereof.

In another embodiment, a Substituted Indole Compound is a compound having Formula VII-B:

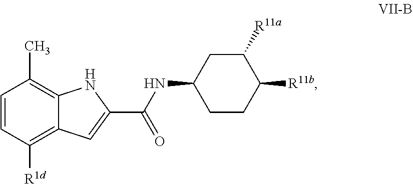

wherein R^{1d}, R^{11a}, and R^{11b} are as defined in connection with Formula VII, or a pharmaceutically acceptable salt or solvate thereof.

In another embodiment, a Substituted Indole Compound is a compound having Formula VII-C:

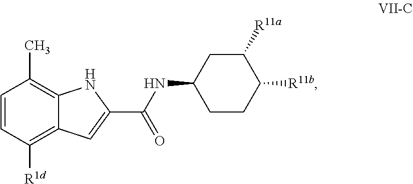

wherein R^{1d}, R^{11a}, and R^{11b} are as defined in connection with Formula VII, or a pharmaceutically acceptable salt or solvate thereof.

In another embodiment, a Substituted Indole Compound is a compound having Formula VII-D:

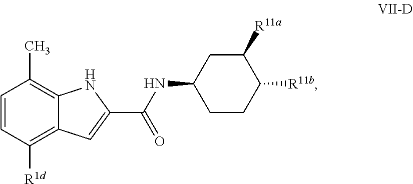

wherein R^{1d}, R^{11a}, and R^{11b} are as defined in connection with Formula VII, or a pharmaceutically acceptable salt or solvate thereof.

In another embodiment, a Substituted Indole Compound is a compound having Formula VII-E:

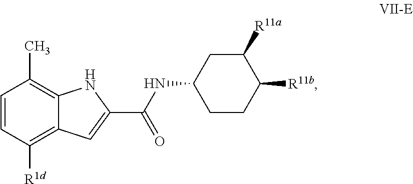

wherein R^{1d}, R^{11a}, and R^{11b} are as defined in connection with Formula VII, or a pharmaceutically acceptable salt or solvate thereof.

In another embodiment, a Substituted Indole Compound is a compound having Formula VII-F:

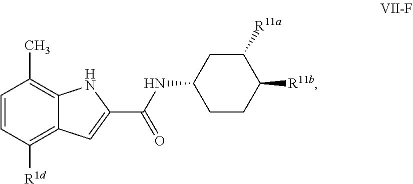

wherein R^{1d}, R^{11a}, and R^{11b} are as defined in connection with Formula VII, or a pharmaceutically acceptable salt or solvate thereof.

In another embodiment, a Substituted Indole Compound is a compound having Formula VII-G:

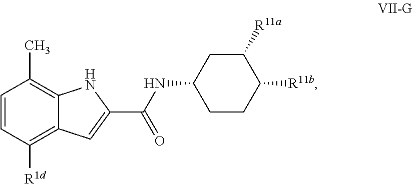

wherein R^{1d}, R^{11a}, and R^{11b} are as defined in connection with Formula VII, or a pharmaceutically acceptable salt or solvate thereof.

In another embodiment, a Substituted Indole Compound is a compound having Formula VII-II:

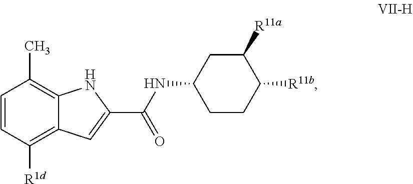

wherein R^{1d}, R^{11a}, and R^{11b} are as defined in connection with Formula VII, or a pharmaceutically acceptable salt or solvate thereof.

In another embodiment, a Substituted Indole Compound is a compound having Formula VIII:

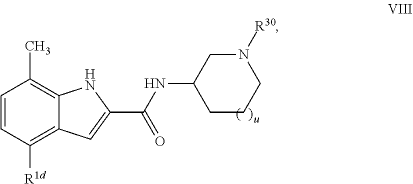

wherein:

R³⁰ is selected from the group consisting of hydrogen; C₁-C₆ alkyl; unsubstituted C₃-C₆ cycloalkyl; substituted C₃-C₆ cycloalkyl having one or two substituents independently selected from the group consisting of halo, hydroxy, C₁-C₄ alkyl, amino, and (amino)C₁-C₄ alkyl; unsubstituted 4- to 14-membered heterocyclo; and substituted 4-to 14-membered heterocyclo having one or two substituents independently selected from the group consisting of amino, hydroxy, and C₁-C₄ alkyl; —C(═O)R^{13b}, and —S(═O)₂R²⁴;

R^{13b} is selected from the group consisting of C₁-C₄ alkyl; amino; C₁-C₄ haloalkyl; C₁-C₄ alkoxy; (hydroxy)C₁-C₄ alkyl; (C₁-C₄ alkoxy)C₁-C₄ alkyl; (amino)alkyl; unsubstituted C₃-C₆ cycloalkyl; substituted C₃-C₆ cycloalkyl having one or two substituents independently selected from the group consisting of halo, hydroxy, C₁-C₄ alkyl, amino, and (amino)C₁-C₄ alkyl; unsubstituted 4- to 14-membered heterocyclo; substituted 4- to 14-membered heterocyclo having one or two substituents independently selected from the group consisting of amino, hydroxy, and C₁-C₄ alkyl; (C₃-C₆ cycloalkyl)oxy; and (4- to 8-membered heterocyclo)oxy;

R²⁴ is selected from the group consisting of C₁-C₄ alkyl and (hydroxy)C₁-C₄ alkyl;

u is 0, 1, 2, or 3; and

R^{1d} is as defined in connection with Formula I, or a pharmaceutically acceptable salt or solvate thereof.

In another embodiment, a Substituted Indole Compound is a compound having Formula VIII-A:

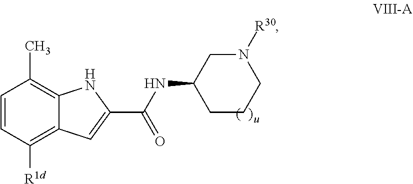

wherein R^{1d}, R³⁰, and u are as defined in connection with Formula VIII, or a pharmaceutically acceptable salt or solvate thereof.

In another embodiment, a Substituted Indole Compound is a compound having Formula VIII-B:

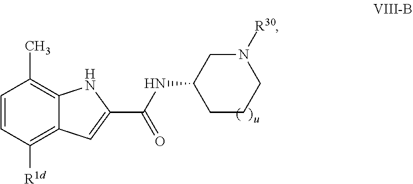

wherein R^{1d}, R³⁰, and u are as defined in connection with Formula VIII, or a pharmaceutically acceptable salt or solvate thereof.

In another embodiment, a Substituted Indole Compound is a compound according to Embodiments 1-73 as follows:

Embodiment 1. A compound of Formula I:

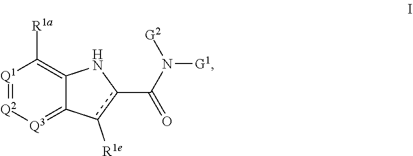

wherein:

R^{1a} is selected from the group consisting of halogen, alkyl, alkoxy, cycloalkyl, (hydroxy)alkyl, and (cycloalkyl)alkyl;

Q¹ is selected from the group consisting of —C(R^{1b})═ and —N═;

Q² is selected from the group consisting of —C(R^{1c})═ and —N═;

Q³ is selected from the group consisting of —C(R^{1d})═ and —N═;

provided that at least one of Q¹, Q², or Q³ is —C(R^{1b})═, —C(R^{1c})═, or —C(R^{1d})═, respectively;

R^{1b}, R^{1c}, and R^{1d} are each independently selected from the group consisting of hydrogen, halogen, alkyl, alkenyl, (hydroxy)alkyl, and alkoxy;

R^{1e} is selected from the group consisting of hydrogen, halogen, alkyl, cycloalkyl, (hydroxy)alkyl, and (cycloalkyl)alkyl;

[symbol] is a single or double bond;

G¹ is selected from the group consisting of: optionally substituted aryl;

optionally substituted heteroaryl; optionally substituted heterocyclo; optionally substituted cycloalkyl; (aryl)alkyl; (heteroaryl)alkyl; (heterocyclo)alkyl; (amino)(aryl)alkyl; (heteroaryl)(aryl)alkyl; (heteroaryl)(heterocyclo)alkyl; (heteroaryl)(carboxamido)alkyl; (heteroaryl)(cycloalkyl)alkyl; (aryl)(alkoxycarbonyl)alkyl; (cycloalkyl)alkyl; (heteroaryl)(amino)alkyl; (cycloalkyl)(alkoxycarbonyl)alkyl; (heteroaryl)(alkoxycarbonyl)alkyl; (heterocyclo)(cycloalkyl)alkyl; (aryl)(cycloalkyl)alkyl; (aryl)(hydroxy)alkyl; (cycloalkyl)(hydroxy)alkyl; (hydroxy)alkyl; optionally substituted alkyl; (aryl)(haloalkyl)alkyl; (cycloalkyl)(haloalkyl)alkyl; (hydroxy)(haloalkyl)alkyl; and (alkoxycarbonyl)(haloalkyl)alkyl; and

G² is selected from the group consisting of hydrogen and alkyl; or

G¹ and G² taken together with the nitrogen atom to which they are attached form an optionally substituted heterocyclo.

Embodiment 2. The compound of Embodiment 1, wherein:

R^{1a} is selected from the group consisting of halogen, C₁-C₆ alkyl, C₁-C₆ alkoxy, C₃-C₈ cycloalkyl, (hydroxy)C₁₋₆ alkyl, and (C₃-C₆ cycloalkyl)C₁₋₆ alkyl;

R^{1b}, R^{1c}, and R^{1d} are each independently selected from the group consisting of hydrogen, halogen, C₁-C₆ alkyl, C₂-C₆ alkenyl, (hydroxy)C₁-C₆ alkyl, and C₁-C₆ alkoxy;

R^{1e} is selected from the group consisting of hydrogen and C₁-C₆ alkyl;

G¹ is selected from the group consisting of:

optionally substituted C₆-C₁₀ aryl; optionally substituted 5- to 10-membered heteroaryl; optionally substituted 3- to 10-membered heterocyclo; optionally substituted C₃-C₈ cycloalkyl; (C₆-C₁₀ aryl)C₁-C₆ alkyl; (5- to 10-membered heteroaryl)C₁-C₆ alkyl; (3- to 10-membered heterocyclo)C₁-C₆ alkyl; (amino)(C₆-C₁₀ aryl)C₁-C₆ alkyl; (5- to 14-membered heteroaryl)(C₆-C₁₀ aryl)C₁-C₆ alkyl; (5- to 10-membered heteroaryl)(3- to 10-membered heterocyclo)C₁-C₆ alkyl; (5- to 10-membered heteroaryl)(carboxamido)C₁-C₆ alkyl; (5- to 10-membered heteroaryl)(C₃-C₆ cycloalkyl)C₁-C₆ alkyl; (C₆-C₁₀ aryl)(alkoxycarbonyl)C₁-C₆ alkyl; (C₃-C₆ cycloalkyl)C₁-C₆ alkyl; (5- to 10-membered heteroaryl)(amino)C₁-C₆ alkyl; (C₃-C₆ cycloalkyl)(alkoxycarbonyl)C₁-C₆ alkyl; (5- to 14-membered heteroaryl)(alkoxycarbonyl)C₁-C₆ alkyl; (3- to 14-membered heterocyclo)(C₃-C₈ cycloalkyl)C₁-C₆ alkyl; (C₆₋₁₀ aryl)(C₃-C₈ cycloalkyl)C₁-C₆ alkyl; (C₆-C₁₀ aryl)(hydroxy)C₁-C₆ alkyl; (C₃-C₆ cycloalkyl)(hydroxy)C₁-C₆ alkyl; (hydroxy)C₁-C₆ alkyl; optionally substituted C₁-C₆ alkyl; (C₆-C₁₀ aryl)(C₁-C₆ haloalkyl)C₁-C₆ alkyl; (C₃-C₆ cycloalkyl)(C₁-C₆haloalkyl)C₁-C₆ alkyl; (hydroxy)(C₁-C₆haloalkyl)C₁-C₆ alkyl; and (alkoxycarbonyl)(C₁-C₆haloalkyl)C₁-C₆ alkyl; and

G² is selected from the group consisting of hydrogen and C₁-C₆ alkyl; or

G¹ and G² taken together with the nitrogen atom to which they are attached form a 5- to 10-membered optionally substituted heterocyclo, or a pharmaceutically acceptable salt or solvate thereof.

Embodiment 3. The compound of Embodiment 2, wherein:

R^{1a} is selected from the group consisting of halogen, C₁-C₃ alkyl, C₁-C₃ alkoxy, C₃-C₆ cycloalkyl, (hydroxy)C₁₋₄ alkyl, and (C₃-C₆ cycloalkyl)C₁₋₄ alkyl;

R^{1b}, R^{1c}, and R^{1d} are each independently selected from the group consisting of hydrogen, halogen, C₁-C₃ alkyl, C₂-C₄ alkenyl, (hydroxy)C₁-C₄ alkyl, and C₁-C₃ alkoxy;

R^{1e} is selected from the group consisting of hydrogen and C₁-C₃ alkyl;

G¹ is selected from the group consisting of optionally substituted C₆-C₁₀ aryl; optionally substituted 5- to 10-membered heteroaryl; optionally substituted 3- to 10-membered heterocyclo; optionally substituted C₃-C₈ cycloalkyl; (C₆-C₁₀ aryl)C₁-C₄ alkyl; (5- to 10-membered heteroaryl)C₁-C₆ alkyl; (3- to 10-membered heterocyclo)C₁-C₄ alkyl; (amino)(C₆-C₁₀ aryl)C₁-C₆ alkyl; (5- to 14-membered heteroaryl)(C₆-C₁₀ aryl)C₁-C₄ alkyl; (5- to 10-membered heteroaryl)(3- to 10-membered heterocyclo)C₁-C₄ alkyl; (5- to 10-membered heteroaryl)(carboxamido)C₁-C₄ alkyl; (5- to 10-membered heteroaryl)(C₃-C₆ cycloalkyl)C₁-C₄ alkyl; (C₆-C₁₀ aryl)(alkoxycarbonyl)C₁-C₄ alkyl; (C₃-C₆ cycloalkyl)C₁-C₄ alkyl; (5- to 10-membered heteroaryl)(amino)C₁-C₄ alkyl; (C₃-C₆ cycloalkyl)(alkoxycarbonyl)C₁-C₄ alkyl; (5- to 14-membered heteroaryl)(alkoxycarbonyl)C₁-C₄ alkyl; (3- to 14-membered heterocyclo)(C₃-C₆ cycloalkyl)C₁-C₄ alkyl; (C₆₋₁₀ aryl)(C₃-C₆ cycloalkyl)C₁-C₄ alkyl; (C₆-C₁₀ aryl)(hydroxy)C₁-C₄ alkyl; (C₃-C₆ cycloalkyl)(hydroxy)C₁-C₄ alkyl; (hydroxy)C₁-C₄ alkyl; optionally substituted C₁-C₄ alkyl; (C₆-C₁₀ aryl)(C₁-C₄haloalkyl)C₁-C₄ alkyl; (C₃-C₆ cycloalkyl)(C₁-C₄haloalkyl)C₁-C₄ alkyl; (hydroxy)(C₁-C₄haloalkyl)C₁-C₄ alkyl; and (alkoxycarbonyl)(C₁-C₄haloalkyl)C₁-C₄ alkyl; and

G² is selected from the group consisting of hydrogen and C₁-C₄ alkyl; or

G¹ and G² taken together with the nitrogen atom to which they are attached form a 5- to 10-membered optionally substituted heterocyclo, or a pharmaceutically acceptable salt or solvate thereof.

Embodiment 4. The compound of any one of Embodiments 1-3, wherein is a double bond, or a pharmaceutically acceptable salt or solvate thereof.

Embodiment 5. The compound of any one of Embodiments 1-4, wherein Q¹ and Q² are —C(H)═, or a pharmaceutically acceptable salt or solvate thereof.

Embodiment 6. The compound of any one of Embodiments 1-4, wherein Q³ is —C(R^{1d})═; and R^{1d} is selected from the group consisting of hydrogen and halo, or a pharmaceutically acceptable salt or solvate thereof.

Embodiment 7. The compound of any one of Embodiments 1-6, wherein R^{1e} is hydrogen, or a pharmaceutically acceptable salt or solvate thereof.

Embodiment 8. The compound of any one of Embodiments 1-7, wherein R^{1a} is C₁-C₃ alkyl, or a pharmaceutically acceptable salt or solvate thereof.

Embodiment 9. The compound of any one of Embodiments 1-8, wherein G² is hydrogen, or a pharmaceutically acceptable salt or solvate thereof.

Embodiment 10. The compound of any one of Embodiments 1-9 of Formula II:

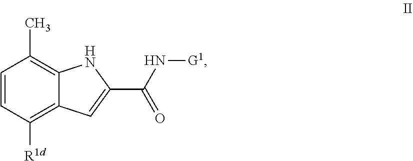

or a pharmaceutically acceptable salt or solvate thereof.

Embodiment 11. The compound of Embodiment 10, wherein R^{1d} is selected from the group consisting of hydrogen and fluoro, or a pharmaceutically acceptable salt or solvate thereof.

Embodiment 12. The compound of Embodiment 11 of Formula II-A:

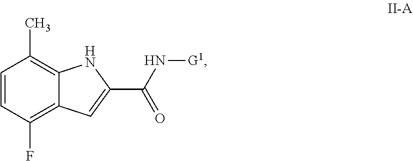

or a pharmaceutically acceptable salt or solvate thereof.

Embodiment 13. The compound any one of Embodiments 10-12, wherein G¹ is selected from the group consisting of: optionally substituted C₆-C₁₀ aryl; optionally substituted 5- to 9-membered heteroaryl; optionally substituted 3- to 10-membered heterocyclo; optionally substituted C₆-C₈ cycloalkyl; (5- to 9-membered heteroaryl)C₁-C₆ alkyl; (5- to 9-membered heteroaryl)(C₆₋₁₀ aryl)C₁-C₄ alkyl; (5- to 9-membered heteroaryl heteroaryl)(C₃-C₆ cycloalkyl)C₁-C₄ alkyl; and (C₃-C₆ cycloalkyl)C₁-C₄ alkyl, or a pharmaceutically acceptable salt or solvate thereof.

Embodiment 14. The compound of Embodiment 13 of Formula III:

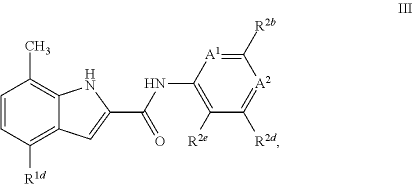

wherein:

A¹ is selected from the group consisting of —N═ and —C(R^{2a})═;

R^{2a} is selected from the group consisting of hydrogen, alkyl, halogen, and haloalkyl;

R^{2b} is selected from the group consisting of optionally substituted alkyl, optionally substituted heterocyclo, optionally substituted cycloalkyl, optionally substituted heteroaryl, optionally substituted aryl, (carboxamido)alkyl, —OR^{10c}, amino, (heterocyclo)alkyl, (amino)alkyl, (hydroxy)alkyl, carboxamido, (heteroaryl)alkyl, —S(═O)R^{9b}, —S(═O)₂R^{9b}, and —C(═O)R^{9c};

A² is selected from the group consisting of —N═ and —C(R^{2c})═;

R^{2c} is selected from the group consisting of hydrogen, alkyl, halogen, and haloalkyl;

R^{2d} is selected from the group consisting of hydrogen, alkyl, halogen, cyano, and haloalkyl;

R^{2e} is selected from the group consisting of hydrogen, alkyl, halogen, and haloalkyl;

R^{9b} is selected from the group consisting of amino, alkyl, optionally substituted cycloalkyl, optionally substituted aryl, optionally substituted heterocyclo, and optionally substituted heteroaryl;

R^{9c} is selected from the group consisting of amino, alkyl, optionally substituted cycloalkyl, optionally substituted aryl, optionally substituted heterocyclo, and optionally substituted heteroaryl; and

R^{10c} is selected from the group consisting of alkyl, (hydroxy)alkyl, and (amino)alkyl, or a pharmaceutically acceptable salt or solvate thereof.

Embodiment 15 The compound of Embodiment 14 having Formula III-A:

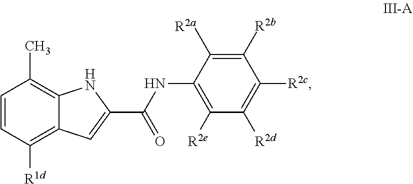

or a pharmaceutically acceptable salt or solvate thereof.

Embodiment 16. The compound of Embodiments 14 or 15, wherein:

R^{2a} is selected from the group consisting of hydrogen, C₁-C₄ alkyl, halogen, and C₁-C₄ haloalkyl;

R^{2b} is selected from the group consisting of:

(A) unsubstituted 4- to 10-membered heterocyclo;

(B) substituted 4- to 10-membered heterocyclo having one, two, three, or four substituents independently selected from the group consisting of (i) —N(R^{3a})C(═O)R^{4a}; (ii) —NR^{5a}R^{5b}; (iii) unsubstituted 4- to 10-membered heterocyclo; (iv) substituted 4- to 10-membered heterocyclo having one, two, or three substituents independently selected from the group consisting of hydroxy, —NR^{5c}R^{5d}, C₁-C₄ alkyl, C₁-C₆ alkoxy, —C(R^{6a})(R^{6b})C(═O)NR^{5e}R^{5f}, —C(═O)R^{4b}, (hydroxy)C₁-C₄ alkyl, and halo; (v) unsubstituted C₃-C₆ cycloalkyl; (vi) (hydroxy)C₁-C₄ alkyl; (vii) C₁-C₆ alkyl; (viii) —C(═O)NR^{5g}R^{5h}; (ix) halo; (x) —C(═O)R^{4c}; (xi) C₁-C₆ haloalkyl; (xii) hydroxy; (xiii) (amino)C₁-C₄ alkyl; (xiv)(C₁-C₄ alkoxy)C₁-C₄ alkyl; (xv) —S(═O)₂R^{9a}; (xvi) (3- to 8-membered heterocyclo)C₁-C₄ alkyl; (xvii) C₁-C₆ alkoxy; (xviii) (C₃-C₆ cycloalkyl)C₁₋₄ alkyl; (xix) (C₆₋₁₀ aryl)C₁-C₄ alkyl; and (xxii) —OR^{10b};

(C) unsubstituted C₃-C₈ cycloalkyl;

(D) substituted C₃-C₈ cycloalkyl having one, two, three, or four substituents independently selected from the group consisting of: (i) unsubstituted 4- to 10-membered heterocyclo; (ii) substituted 4- to 10-membered heterocyclo having one or two substituents, independently selected from the group consisting of amino and C₁-C₄ alkyl;

(iii) unsubstituted 5- or 6-membered heteroaryl; (iv) substituted 5- or 6-membered heteroaryl having one, two, or three substituents independently selected from the group consisting of halo, C₁-C₄ alkyl, (3- to 8-membered heterocyclo)alkyl, hydroxy, and amino; (v) —NR⁵ⁱR^{5j}; (vi) cyano; (vii) —N(R^{3d})C(═O)R^{4f}; (viii) hydroxy; and (ix) C₁-C₄ alkyl;

(E) unsubstituted 5-to 10-membered heteroaryl;

(F) substituted 5- to 10-membered heteroaryl having one, two, three, or four substituents independently selected from the group consisting of: (i) halo; (ii) C₁-C₄ alkyl; (iii) (C₁-C₄ alkoxy)C₁-C₄ alkyl; (iv) (hydroxy)C₁-C₄ alkyl; (v) C₃-C₆ cycloalkyl; (vi) (amino)C₁-C₄ alkyl; (vii) unsubstituted C₃-C₆ cycloalkyl; (viii) substituted C₃-C₆ cycloalkyl having one, two, three, or four substituents independently selected from the group consisting of —NR^{5g}R^{5h}; (xi) unsubstituted 4- to 14-membered heterocyclo; (xii) substituted 4- to 14-membered heterocyclo having one or two substituents independently selected from the group consisting of hydroxy, amino, and C₁-C₄ alkyl; (xiii) —NR^{5q}R^{5r}; and (ix) (3- to 8-membered heterocyclo)C₁-C₄ alkyl;

(G) unsubstituted C₆-C₁₀ aryl;

(H) substituted C₆-C₁₀ aryl, having one, two, three, or four substituents independently selected from the group consisting of: (i) halo; (ii) C₁-C₄ alkyl; (iii) —CH-₂N(H)S(═O)₂R⁸; (iv) (5- to 9-membered heteroaryl)C₁-C₄ alkyl; (v) —OR^{10a}; (vi) —N(R^{3b})C(═O)R^{4b}; (vii) (amino)C₁-C₄ alkyl; and (viii) (hydroxy)C₁-C₄ alkyl;

(I) (carboxamido)C₁-C₄ alkyl;

(J) —OR^{10c};

(K) —NR^{5o}R^{5p};

(L) (3- to 8-membered heterocyclo)C₁-C₄ alkyl;

(M) (amino)C₁-C₄ alkyl;

(N) (hydroxy)C₁-C₄ alkyl;

(O) —C(═O)NR^{5s}R^{5t};

(P) (5- to 9-membered heteroaryl)C₁-C₄ alkyl; and

(Q) —S(═O)₂R^{9b};

R^{2c} is selected from the group consisting of hydrogen, C₁-C₄ alkyl, halogen, and C₁-C₄ haloalkyl;

R^{2d} is selected from the group consisting of hydrogen, C₁-C₄ alkyl, halogen, cyano, and C₁-C₄ haloalkyl;

R^{2e} is selected from the group consisting of hydrogen, C₁-C₄ alkyl, halogen, and C₁-C₄ haloalkyl;

R^{3a}, R^{3b}, R^{3c}, and R^{3d} are each independently selected from the group consisting of hydrogen, C₁-C₄ alkyl, optionally substituted C₃-C₆ cycloalkyl, and optionally substituted 4- to 14-membered heterocyclo;

R^{4a}, R^{4b}, R^{4c}, R^{4d}, R^{4e}, and R^{4f} are each independently selected from the group consisting of C₁-C₆ alkyl; C₁-C₆ haloalkyl; C₃-C₆ cycloalkyl; C₁-C₆ alkoxy; (C₁-C₄ alkoxy)C₁-C₄ alkyl; (C₆₋₁₀ aryl)C₁-C₄ alkyl; (5- to 9-membered heteroaryl)C₁-C₄ alkyl; (amino)C₁-C₄ alkyl; (hydroxy)C₁-C₄ alkyl; (cyano)C₁-C₄ alkyl; unsubstituted 4- to 14-membered heterocyclo; substituted 4- to 14-membered heterocyclo having one or two substituents independently selected from the group consisting of halo and C₁-C₄ alkyl; unsubstituted C₆-C₁₀ aryl; substituted C₆-C₁₀ aryl, having one, two, three, or four substituents independently selected from the group consisting of halo and C₁-C₄ alkyl; unsubstituted 5- or 6-membered heteroaryl; and substituted 5- or 6-membered heteroaryl having one, two, three, or four substituents independently selected from the group consisting of halo and C₁-C₄ alkyl;

R^{5a} and R^{5b} are independently selected from the group consisting of hydrogen; C₁-C₄ alkyl; C₁-C₄ haloalkyl; (hydroxy)C₁-C₄ alkyl; (amino)C₁-C₄ alkyl; (C₁-C₄ alkoxy)C₁-C₄ alkyl; (5- to 9-membered heteroaryl)C₁-C₄ alkyl; unsubstituted 5- or 6-membered heteroaryl; substituted 5- or 6-membered heteroaryl having one or two substituents independently selected from the group consisting of halo and C₁-C₄ alkyl; unsubstituted 4- to 14-membered heterocyclo; and substituted 4- to 14-membered heterocyclo having one or two substituents independently selected from the group consisting of hydroxy, amino, and C₁-C₄ alkyl;

R^{5c} and R^{5d} are independently selected from the group consisting of hydrogen; C₁-C₄ alkyl; C₁-C₄ haloalkyl; (hydroxy)C₁-C₄ alkyl; (amino)C₁-C₄ alkyl; (C₁-C₄ alkoxy)C₁-C₄ alkyl; (5- to 9-membered heteroaryl)C₁-C₄ alkyl; unsubstituted 5- or 6-membered heteroaryl; substituted 5- or 6-membered heteroaryl having one or two substituents independently selected from the group consisting of halo and C₁-C₄ alkyl; unsubstituted 4- to 14-membered heterocyclo; and substituted 4- to 14-membered heterocyclo having one or two substituents independently selected from the group consisting of hydroxy, amino, and C₁-C₄ alkyl; or

R^{5c} and R^{5d} taken together with the nitrogen atom to which they are attached form an optionally substituted 4- to 14-membered heterocyclo;

R^{5e} and R^{5f} are independently selected from the group consisting of hydrogen; C₁-C₄ alkyl; C₁-C₄ haloalkyl; (hydroxy)C₁-C₄ alkyl; (amino)C₁-C₄ alkyl; (C₁-C₄ alkoxy)C₁-C₄ alkyl; (5- to 9-membered heteroaryl)C₁-C₄ alkyl; unsubstituted 5- or 6-membered heteroaryl; substituted 5- or 6-membered heteroaryl having one or two substituents independently selected from the group consisting of halo and C₁-C₄ alkyl; unsubstituted 4- to 14-membered heterocyclo; and substituted 4- to 14-membered heterocyclo having one or two substituents independently selected from the group consisting of hydroxy, amino, and C₁-C₄ alkyl; or

R^{5e} and R^{5f} taken together with the nitrogen atom to which they are attached form an optionally substituted 4- to 14-membered heterocyclo;

R^{5g} and R^{5h} are independently selected from the group consisting of hydrogen; C₁-C₄ alkyl; C₁-C₄ haloalkyl; (hydroxy)C₁-C₄ alkyl; (amino)C₁-C₄ alkyl; (C₁-C₄ alkoxy)C₁-C₄ alkyl; (5- to 9-membered heteroaryl)C₁-C₄ alkyl; unsubstituted 5- or 6-membered heteroaryl; substituted 5- or 6-membered heteroaryl having one or two substituents independently selected from the group consisting of halo and C₁-C₄ alkyl; unsubstituted 4- to 14-membered heterocyclo; and substituted 4- to 14-membered heterocyclo having one or two substituents independently selected from the group consisting of hydroxy, amino, and C₁-C₄ alkyl; or

R^{5g} and R^{5h} taken together with the nitrogen atom to which they are attached form an optionally substituted 4- to 14-membered heterocyclo;

R⁵ⁱ and R^{5j} are independently selected from the group consisting of hydrogen; C₁-C₄ alkyl; C₁-C₄ haloalkyl; (hydroxy)C₁-C₄ alkyl; (amino)C₁-C₄ alkyl; (C₁-C₄ alkoxy)C₁-C₄ alkyl; (5- to 9-membered heteroaryl)C₁-C₄ alkyl; unsubstituted 5- or 6-membered heteroaryl; substituted 5- or 6-membered heteroaryl having one or two substituents independently selected from the group consisting of halo and C₁-C₄ alkyl; unsubstituted 4- to 14-membered heterocyclo; and substituted 4- to 14-membered heterocyclo having one or two substituents independently selected from the group consisting of hydroxy, amino, and C₁-C₄ alkyl; or

R⁵ⁱ and R^{5j} taken together with the nitrogen atom to which they are attached form an optionally substituted 4- to 14-membered heterocyclo;

R^{5k} and R^{5l} are independently selected from the group consisting of hydrogen; C₁-C₄ alkyl; C₁-C₄ haloalkyl; (hydroxy)C₁-C₄ alkyl; (amino)C₁-C₄ alkyl; (C₁-C₄ alkoxy)C₁-C₄ alkyl; (5- to 9-membered heteroaryl)C₁-C₄ alkyl; unsubstituted 5- or 6-membered heteroaryl; substituted 5- or 6-membered heteroaryl having one or two substituents independently selected from the group consisting of halo and C₁-C₄ alkyl; unsubstituted 4- to 14-membered heterocyclo; and substituted 4- to 14-membered heterocyclo having one or two substituents independently selected from the group consisting of hydroxy, amino, and C₁-C₄ alkyl; or

R^{5k} and R^{5l} taken together with the nitrogen atom to which they are attached form an optionally substituted 4- to 14-membered heterocyclo;

R^{5m} and R⁵ⁿ are independently selected from the group consisting of hydrogen; C₁-C₄ alkyl; C₁-C₄ haloalkyl; (hydroxy)C₁-C₄ alkyl; (amino)C₁-C₄ alkyl; (C₁-C₄ alkoxy)C₁-C₄ alkyl; (5- to 9-membered heteroaryl)C₁-C₄ alkyl; unsubstituted 5- or 6-membered heteroaryl; substituted 5- or 6-membered heteroaryl having one or two substituents independently selected from the group consisting of halo and C₁-C₄ alkyl; unsubstituted 4- to 14-membered heterocyclo; and substituted 4- to 14-membered heterocyclo having one or two substituents independently selected from the group consisting of hydroxy, amino, and C₁-C₄ alkyl; or

R^{5m} and R⁵ⁿ taken together with the nitrogen atom to which they are attached form an optionally substituted 4- to 14-membered heterocyclo;

R⁵⁰ and R^{5p} are independently selected from the group consisting of hydrogen; C₁-C₄ alkyl; C₁-C₄ haloalkyl; (hydroxy)C₁-C₄ alkyl; (amino)C₁-C₄ alkyl; (C₁-C₄ alkoxy)C₁-C₄ alkyl; (5- to 9-membered heteroaryl)C₁-C₄ alkyl; unsubstituted 5- or 6-membered heteroaryl; substituted 5- or 6-membered heteroaryl having one or two substituents independently selected from the group consisting of halo and C₁-C₄ alkyl; unsubstituted 4- to 14-membered heterocyclo; and substituted 4- to 14-membered heterocyclo having one or two substituents independently selected from the group consisting of hydroxy, amino, and C₁-C₄ alkyl; or

R⁵⁰ and R^{5p} taken together with the nitrogen atom to which they are attached form an optionally substituted 4- to 14-membered heterocyclo;

R^{5q} and R^{5r} are independently selected from the group consisting of hydrogen; C₁-C₄ alkyl; C₁-C₄ haloalkyl; (hydroxy)C₁-C₄ alkyl; (amino)C₁-C₄ alkyl; (C₁-C₄ alkoxy)C₁-C₄ alkyl; (5- to 9-membered heteroaryl)C₁-C₄ alkyl; unsubstituted 5- or 6-membered heteroaryl; substituted 5- or 6-membered heteroaryl having one or two substituents independently selected from the group consisting of halo and C₁-C₄ alkyl; unsubstituted 4- to 14-membered heterocyclo; and substituted 4- to 14-membered heterocyclo having one or two substituents independently selected from the group consisting of hydroxy, amino, and C₁-C₄ alkyl;

R^{5s} and R^{5t} are independently selected from the group consisting of hydrogen; C₁-C₄ alkyl; C₁-C₄ haloalkyl; (hydroxy)C₁-C₄ alkyl; (amino)C₁-C₄ alkyl; (C₁-C₄ alkoxy)C₁-C₄ alkyl; (5- to 9-membered heteroaryl)C₁-C₄ alkyl; unsubstituted 5- or 6-membered heteroaryl; substituted 5- or 6-membered heteroaryl having one or two substituents independently selected from the group consisting of halo and C₁-C₄ alkyl; unsubstituted 4- to 14-membered heterocyclo; and substituted 4- to 14-membered heterocyclo having one or two substituents independently selected from the group consisting of hydroxy, amino, and C₁-C₄ alkyl;

R^{6a}, R^{6b}, R^{6c}, and R^{6d} are each independently selected from the group consisting of hydrogen and C₁-C₄ alkyl;

R⁸ is C₁-C₆ alkyl;

R^{9a} is selected from the group consisting of C₁-C₆ alkyl; unsubstituted C₃-C₈ cycloalkyl; and substituted C₃-C₈ cycloalkyl having one or two substituents independently selected from the group consisting of halo, C₁-C₄ alkyl, amino, and (amino)C₁-C₄ alkyl;

R^{9b} is selected from the group consisting of C₁-C₆ alkyl and amino;

R^{10a} is selected from the group consisting of alkyl, (hydroxy)C₁-C₄ alkyl, and (amino)C₁-C₄ alkyl;

R^{10b} is (amino)C₁-C₄ alkyl; and

R^{10c} is (amino)C₁-C₄ alkyl, or a pharmaceutically acceptable salt or solvate thereof.

Embodiment 17. The compound any one of Embodiments 14-16, wherein: A¹ and A² are —C(H)═; R^{2e} is hydrogen; and R^{2d} is selected from the group consisting of hydrogen and halogen, or a pharmaceutically acceptable salt or solvate thereof.

Embodiment 18. The compound of any one of Embodiments 14-17, wherein:

R^{2b} is selected from the group consisting of:

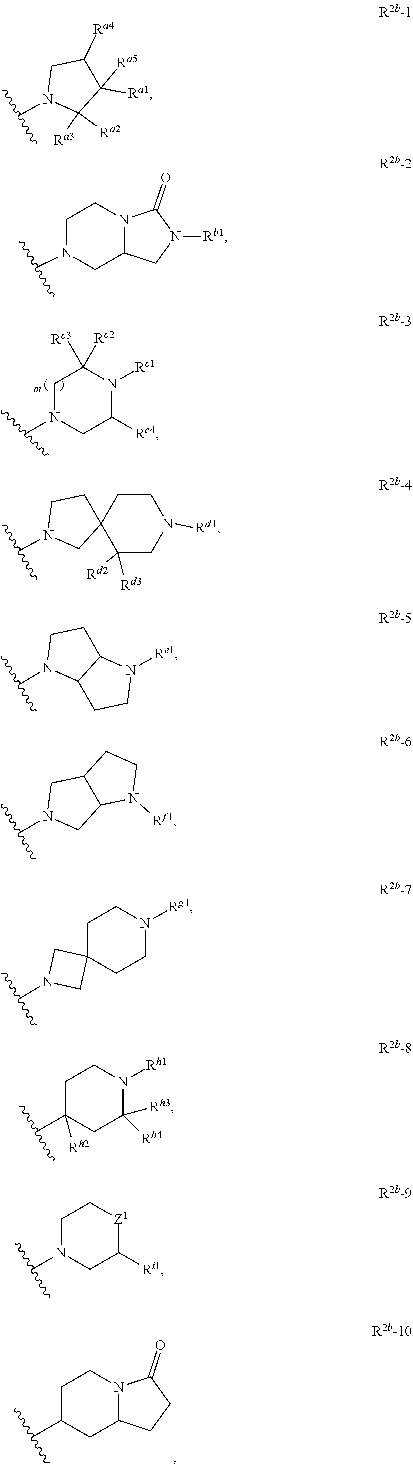

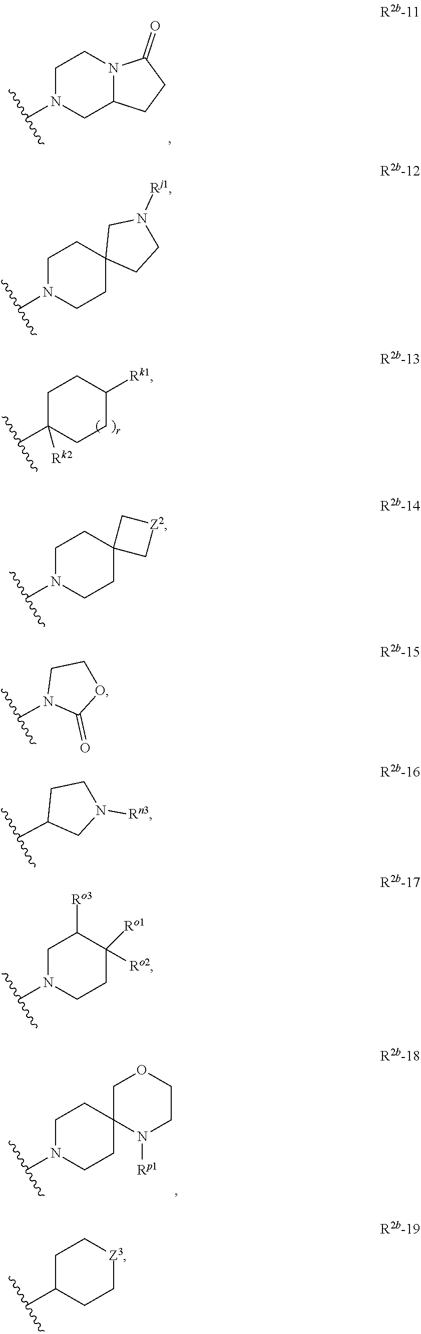

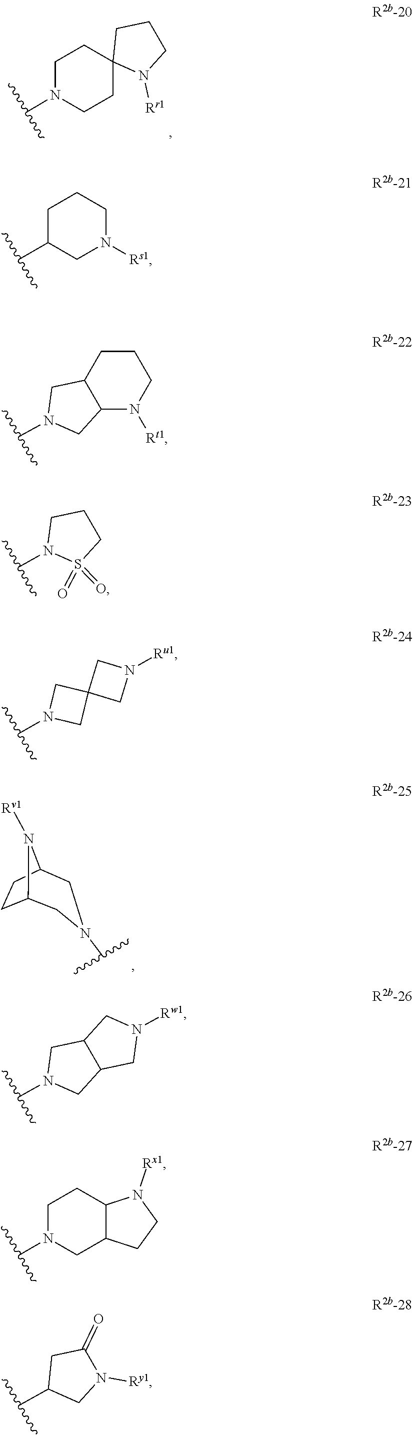

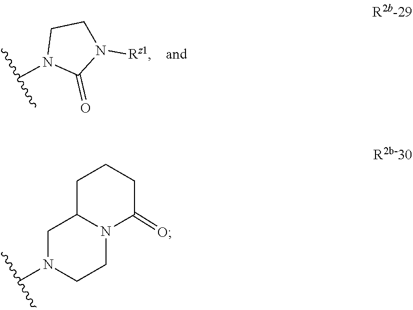

R^{a1} is selected from the group consisting of —N(R^{3a})C(═O)R^{4a}; —NR^{5a}R^{5b}; unsubstituted 4- to 10-membered heterocyclo; substituted 4- to 10-membered heterocyclo having one, two, or three substituents independently selected from the group consisting of hydroxy, —NR^{5c}R^{5d}, C₁-C₄ alkyl, C₁-C₆ alkoxy, —C(R^{6a})(R^{6b})C(═O)NR^{5e}R^{5f}, —C(═O)R^{4b}, (hydroxy)C₁-C₄ alkyl, and halo;

R^{a2} and R^{a3} are each hydrogen; or

R^{a2} and R^{a3} taken together with the carbon atom to which they are attached form a C(═O) group;

R^{a4} is selected from the group consisting of hydrogen, halo, and hydroxy;

R^{a5} is selected from the group consisting of hydrogen, C₁-C₄ alkyl, and C₃-C₆ cycloalkyl;

R^{b1} is selected from the group consisting of hydrogen, C₁-C₄ alkyl, and C₃-C₆ cycloalkyl;

R^{c1} is selected from the group consisting of hydrogen, C₁-C₄ alkyl, C₃-C₆ cycloalkyl, and —C(═O)R^{4c};

R^{c2} and R^{c3} are each independently selected from the group consisting of hydrogen, C₁-C₄ alkyl, and C₁-C₄ haloalkyl; or

R^{c2} and R^{c3} taken together with the carbon atom to which they are attached form a C(═O) group;

R^{c4} is selected from the group consisting of hydrogen and C₁-C₄ alkyl;

m is 1 or 2;

R^{d1} is selected from the group consisting of hydrogen, C₁-C₄ alkyl, and —C(═O)R^{4c},

R^{d2} and R^{d3} are each independently selected from the group consisting of hydrogen and fluoro;

R^{e1} is selected from the group consisting of hydrogen, C₁-C₄ alkyl, C₃-C₆ cycloalkyl, and —C(═O)R^{4c};

R^{f1} is selected from the group consisting of hydrogen, C₁-C₄ alkyl, C₃-C₆ cycloalkyl, and —C(═O)R^{4c},

R^{g1} is selected from the group consisting of hydrogen, C₁-C₄ alkyl, —C(═O)R^{4c}, C₁-C₄ haloalkyl, (C₁-C₄ alkoxy)C₁-C₄ alkyl

R^{h1} is selected from the group consisting of hydrogen, C₁-C₄ alkyl, C₃-C₆ cycloalkyl, and —C(═O)R^{4c};

R^{h2} is selected from the group consisting of hydrogen and C₁-C₄ alkyl;

R^{h3} and R^{h4} are each independently selected from the group consisting of hydrogen and C₁-C₄ alkyl; or

R^{h3} and R^{h4} taken together with the carbon atom to which they are attached form a C(═O) group;

Rⁱ¹ is selected from the group consisting of hydrogen, C₁-C₄ alkyl, C₃-C₆ cycloalkyl, (hydroxy)C₁-C₄ alkyl, —N(R^{3a})C(═O)R^{4a}, and (amino)C₁-C₄ alkyl;

Z¹ is selected from the group consisting of —CH₂— and —O—;

R^{j1} is selected from the group consisting of hydrogen, C₁-C₄ alkyl, C₃-C₆ cycloalkyl, and —C(═O)R^{4c};

R^{k1} is selected from the group consisting of C₁-C₄ alkyl, unsubstituted 4- to 14-membered heterocyclo and —NR^{5a}R^{5b};

R^{k2} is selected from the group consisting of hydrogen, hydroxy, and C₁-C₄ alkyl;

r is 0, 1, or 2;

Z² is selected from the group consisting of —O— and —N(R^{m3})—;

R^{m3} is selected from the group consisting of hydrogen, C₁-C₄ alkyl, and C₁-C₄ haloalkyl;

Rⁿ¹ is selected from the group consisting of hydrogen, C₁-C₄ alkyl, and —C(═O)R^{4c};

R^{o1} is selected from the group consisting of hydroxy, (hydroxy)C₁-C₄ alkyl, (amino)C₁-C₄ alkyl, (C₁-C₄ alkoxy)C₁-C₄ alkyl, C₁-C₄ alkoxy, —NR^{5a}R^{5b}, unsubstituted 4-to 14-membered heterocyclo, substituted 4- to 14-membered heterocyclo having one, two, or three substituents independently selected from the group consisting of halo, C₁-C₄ alkyl and C₁-C₄ alkoxy;

R^{o2} is selected from the group consisting of hydrogen, C₁-C₄ alkyl, and (C₁-C₄ alkoxy)C₁-C₄ alkyl

R^{o3} is selected from the group consisting of hydrogen, fluoro, and C₁-C₄ alkyl;

R^{p1} is selected from the group consisting of hydrogen, C₁-C₄ alkyl, and —C(═O)R^{4c};

Z³ is selected from the group consisting of —O— and —N(R^{q1})—;

R^{q1} is selected from the group consisting of hydrogen, C₁-C₄ alkyl, and —C(═O)R^{4c};

R^{r1} is selected from the group consisting of hydrogen, C₁-C₄ alkyl, and —C(═O)R^{4c};

R^{s1} is selected from the group consisting of hydrogen, C₁-C₄ alkyl, and —C(═O)R^{4c};

R^{t1} is selected from the group consisting of hydrogen, C₁-C₄ alkyl, and —C(═O)R^{4c};

R^{u1} is selected from the group consisting of hydrogen, C₁-C₄ alkyl, and —C(═O)R^{4c};

R^{v1} is selected from the group consisting of hydrogen, C₁-C₄ alkyl, and —C(═O)R^{4c};

R^{w1} is selected from the group consisting of hydrogen, C₁-C₄ alkyl, and —C(═O)R^{4c};

R^{x1} is selected from the group consisting of hydrogen, C₁-C₄ alkyl, and —C(═O)R^{4c};

R^{y1} is selected from the group consisting of hydrogen and C₁-C₄ alkyl; and

R^{z1} is selected from the group consisting of hydrogen and C₁-C₄ alkyl, or a pharmaceutically acceptable salt or solvate thereof.

Embodiment 19. The compound Embodiment 18, wherein R^{2b} is selected from the group consisting of:

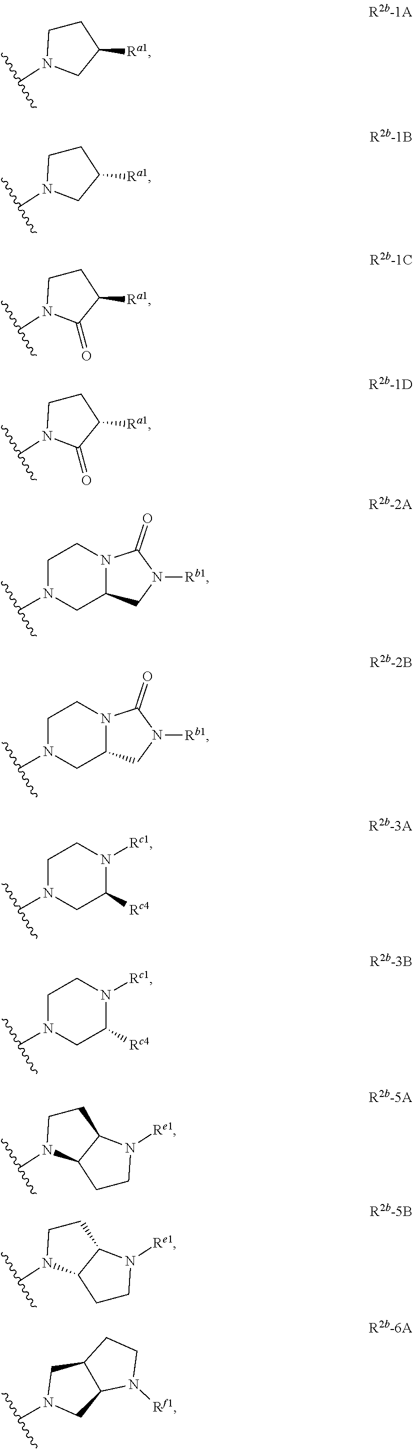

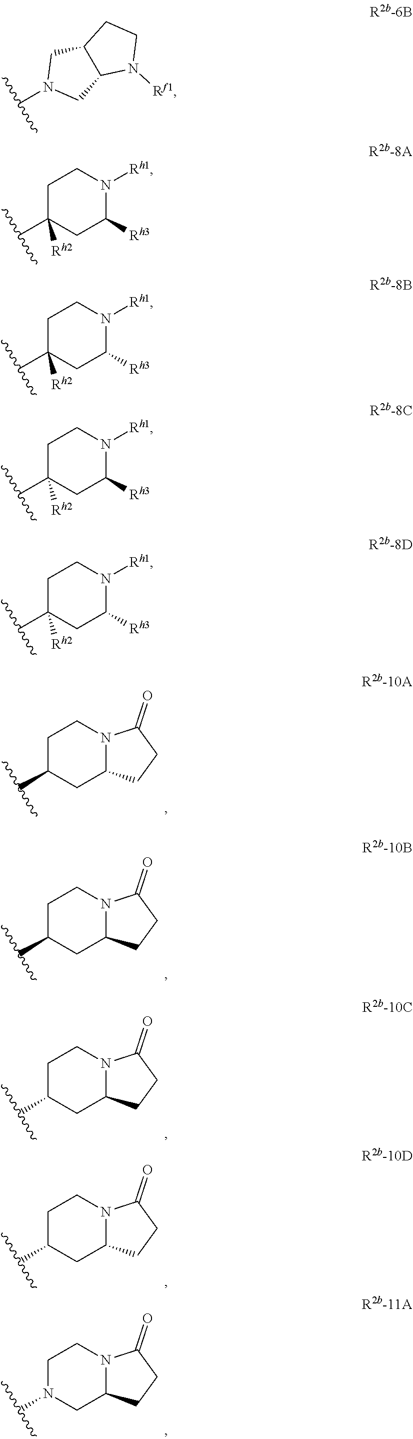

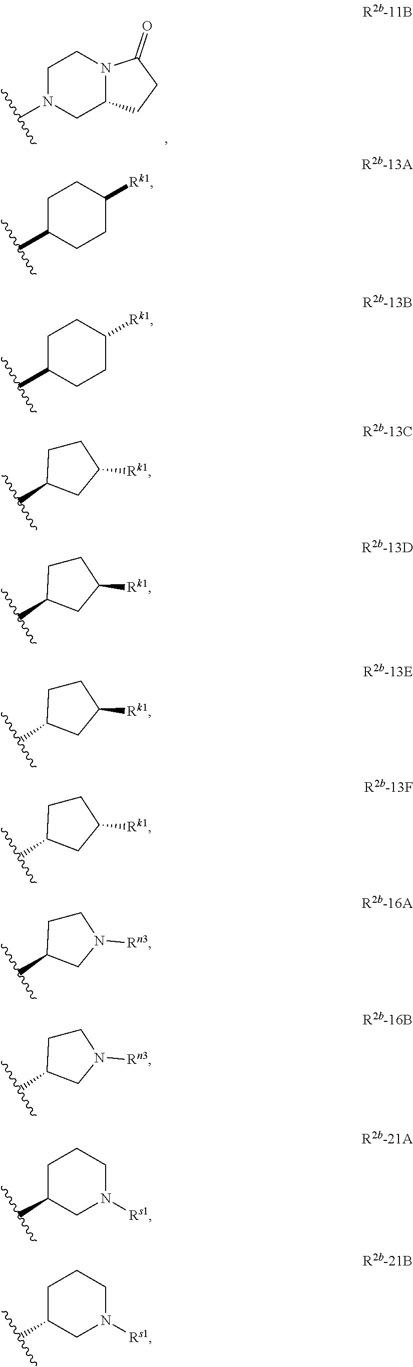

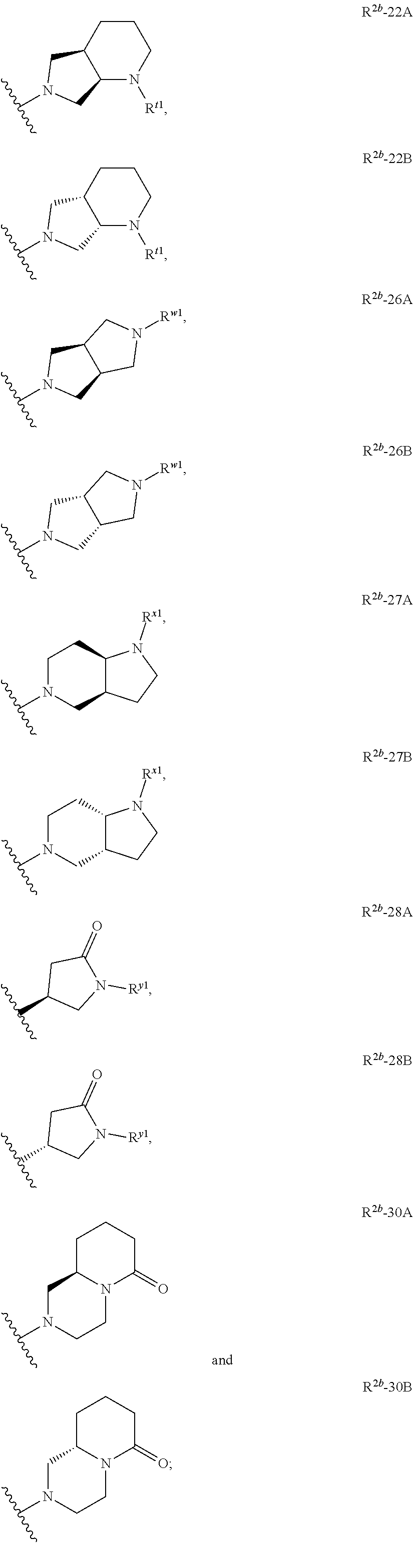

or a pharmaceutically acceptable salt or solvate thereof.

Embodiment 20. The compound of Embodiment 19, wherein R^{2b} is selected from the group consisting of R^{2b}-1A, R^{2b}-1B, R^{2b}-1C, and R^{2b}-1D, or a pharmaceutically acceptable salt or solvate thereof.

Embodiment 21. The compound of Embodiment 19, wherein: R^{2b} is selected from the group consisting of R^{2b}-2A and R^{2b}-2B; and R^{b1} is C₁-C₄ alkyl, or a pharmaceutically acceptable salt or solvate thereof.

Embodiment 22. The compound of Embodiment 19, wherein: R^{2b} is selected from the group consisting of R^{2b}-5A and R^{2b}-5B; and R^{e1} is —C(═O)R^{4c}, or a pharmaceutically acceptable salt or solvate thereof.

Embodiment 23. The compound of Embodiment 19, wherein: R^{2b} is selected from the group consisting of R^{2b}-6A and R^{2b}-6B; and R^{f1} is —C(═O)R^{4c}, or a pharmaceutically acceptable salt or solvate thereof.

Embodiment 24. The compound of Embodiment 19, wherein: R^{2b} is selected from the group consisting of R^{2b}-10A, R^{2b}-10B, R^{2b}-10C, and R^{2b}-10d, or a pharmaceutically acceptable salt or solvate thereof.

Embodiment 25. The compound of Embodiment 19, wherein: R^{2b} is selected from the group consisting of R^{2b}-11A and R^{2b}-11B, or a pharmaceutically acceptable salt or solvate thereof.

Embodiment 26. The compound of Embodiment 18, wherein: R^{2b} is R^{2b}-4; R^{d1} is —C(═O)R^{4c}; and R^{d2} and R^{d3} are each hydrogen or fluoro, or a pharmaceutically acceptable salt or solvate thereof.

Embodiment 27. The compound of Embodiment 18, wherein:

R^{2b} is R^{2b}-3;

R^{c1} is selected from the group consisting of C₁-C₄ alkyl, C₃-C₆ cycloalkyl, and —C(═O)R^{4c};

R^{c2} and R^{c3} are each hydrogen; or

R^{c2} and R^{c3} taken together with the carbon atom to which they are attached form a C(═O) group;

R^{c4} is hydrogen; and

m is 1;

or a pharmaceutically acceptable salt or solvate thereof.

Embodiment 28. The compound of Embodiment 18, wherein: R^{2b} is R^{2b}-8; R^{h1} is —C(═O)R^{4c}; and R^{h2} is selected from the group consisting of hydrogen and C₁-C₃ alkyl, or a pharmaceutically acceptable salt or solvate thereof.

Embodiment 29. The compound of Embodiment 18, wherein: R^{2b} is R^{2b}-12; and R^{j1} is —C(═O)R^{4c}, or a pharmaceutically acceptable salt or solvate thereof.

Embodiment 30. The compound of any one of Embodiments 18, 19, 22, 23, or 26-29, wherein R^{4c} is C₁-C₄ alkyl, or a pharmaceutically acceptable salt or solvate thereof.

Embodiment 31. The compound of any one of Embodiments 14-30, wherein R^{2d} is selected from the group consisting of hydrogen, fluoro, and chloro, or a pharmaceutically acceptable salt or solvate thereof.

Embodiment 32. The compound of Embodiment 13 of Formula IV:

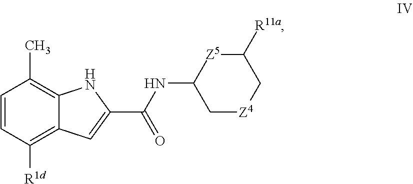

wherein:

Z⁴ is selected from the group consisting of —O—, —C(R^{28a})(R^{28b})—, and —N(R²³)—; or Z⁴ is absent;

Z⁵ is selected from the group consisting of —CH₂— and —CH₂CH₂—;

R^{11a} is selected from the group consisting of optionally substituted alkyl, optionally substituted heterocyclo, optionally substituted heteroaryl, and —N(R^{12b})C(═O)R^{13c};

R^{12b} is selected from the group consisting of hydrogen, alkyl, cycloalkyl, and heterocyclo, (C₁-C₄ alkoxy)C₁-C₄ alkyl, and (hydroxy)C₁-C₄ alkyl; and

R^{13c} is selected from the group consisting of alkyl, haloalkyl, alkoxy, (alkoxy)alkyl, (hydroxy)alkyl, (cyano)alkyl, optionally substituted aryl, optionally substituted heteroaryl, optionally substituted cycloalkyl, and optionally substituted heterocycle, amino, (amino)alkyl, (C₃-C₆ cycloalkyl)oxy, and (4- to 8-membered heterocyclo)oxy;

R²³ is selected from the group consisting of hydrogen and C₁-C₄ alkyl; and

R^{28a} and R^{28b} are independently selected from the group consisting of hydrogen, alkyl, and halo, or a pharmaceutically acceptable salt or solvate thereof.

Embodiment 33. The compound of Embodiment 32 of Formula IV-A:

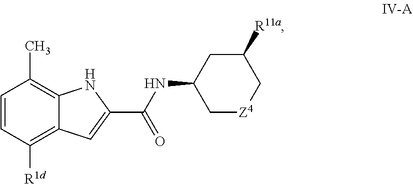

or a pharmaceutically acceptable salt or solvate thereof.

Embodiment 34. The compound of Embodiment 32 of Formula IV-B:

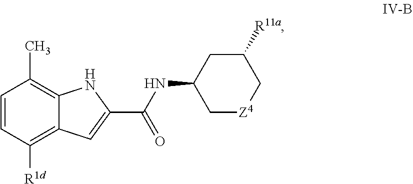

or a pharmaceutically acceptable salt or solvate thereof.

Embodiment 35. The compound of Embodiment 32 of Formula IV-C:

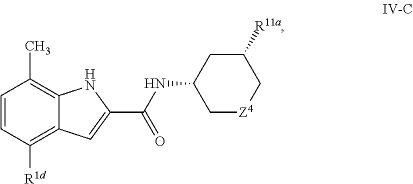

or a pharmaceutically acceptable salt or solvate thereof.

Embodiment 36. The compound of Embodiment 32 of Formula IV-D:

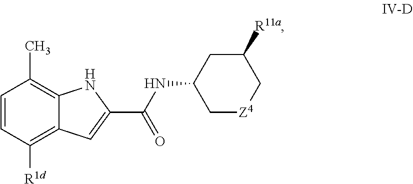

or a pharmaceutically acceptable salt or solvate thereof.

Embodiment 37. The compound of any one of Embodiments 32-36, wherein:

R^{11a} is selected from the group consisting of:

(A) unsubstituted 4- to 14-membered heterocyclo;

(B) substituted 4- to 14-membered heterocyclo having one, two or three substituents independently selected from the group consisting of:

(i) —N(R^{12a})C(═O)R^{13a}; (ii) —C(═O)R^{13b}; (iii) C₁-C₄ alkyl; (iv) (C₁-C₄ alkoxy)C₁-C₄ alkyl; (v) (hydroxy)C₁-C₄ alkyl; (vi) C₁-C₄ haloalkyl; (vii) amino; (vii) hydroxy; (viii) —N(R^{12a})S(═O)₂R²⁴; (ix) —S(═O)₂R²⁴; (x) unsubstituted C₃-C₆ cycloalkyl; (xi) substituted C₃-C₆ cycloalkyl having one or two substituents independently selected from the group consisting of halo, hydroxy, C₁-C₄ alkyl, amino, and (amino)C₁-C₄ alkyl; (xii) unsubstituted 4- to 14-membered heterocyclo; and substituted 4- to 14-membered heterocyclo having one or two substituents independently selected from the group consisting of amino, hydroxy, and C₁-C₄ alkyl; (xiii) —C(═N—R⁶⁰)R⁶¹; and (xiv) —C(═C—NO₂)R⁶⁴;

(C) unsubstituted 5- to 10-membered heteroaryl;

(D) substituted 5- or 6-membered heteroaryl having one, two, three, or four substituents independently selected from the group consisting of halo and C₁-C₄ alkyl;

(E) C₁-C₆ alkyl; and

(F) —N(R^{12b})C(═O)R^{13c};

R^{12a} and R^{12b} are each independently selected from the group consisting of hydrogen, C₁-C₄ alkyl, (C₁-C₄ alkoxy)C₁-C₄ alkyl, and (hydroxy)C₁-C₄ alkyl;

R^{13a}, R^{13b}, and R^{13c} are each independently selected from the group consisting of (A) C₁-C₆ alkyl; (B) C₁-C₆ haloalkyl; (C) unsubstituted C₃-C₆ cycloalkyl; (D) C₁-C₆ alkoxy; (E) (C₁-C₄ alkoxy)C₁-C₄ alkyl; (F) (hydroxy)C₁-C₄ alkyl; (G) (cyano)alkyl; (H) unsubstituted C₆-C₁₀ aryl; (I) substituted C₆-C₁₀ aryl, having one, two, three, or four substituents independently selected from the group consisting of halo, amino, hydroxy, and C₁-C₄ alkyl; (J) unsubstituted 5- or 6-membered heteroaryl; (K) substituted 5- or 6-membered heteroaryl having one, two, three, or four substituents independently selected from the group consisting of halo, amino, hydroxy, and C₁-C₄ alkyl; (L) unsubstituted 4-to 14-membered heterocyclo; (M) substituted 4- to 14-membered heterocyclo having one or two substituents independently selected from the group consisting of amino, hydroxy, and C₁-C₄ alkyl; (N) amino; (O) (amino)alkyl; (P) (C₃-C₆ cycloalkyl)oxy; and (Q) (4- to 8-membered heterocyclo)oxy; and

R²⁴ is selected from the group consisting of C₁-C₄ alkyl and (hydroxy)C₁-C₄ alkyl;

R⁶⁰ is selected from the group consisting of cyano, nitro, hydroxy, C₁-C₆ alkoxy, —C(═O)R⁶², and —S(═O)₂R⁶²;

R⁶¹ is selected from the group consisting of C₁-C₆ alkyl, C₃-C₆ cycloalkyl, and —NR^{63a}R^{63b};

R⁶² is selected from the group consisting of C₁-C₆ alkyl, C₃-C₆ cycloalkyl, and —NR^{63a}R^{63b};

R^{63a} is selected from the group consisting of hydrogen, C₁-C₆ alkyl, and C₃-C₆ cycloalkyl;

R^{63b} is selected from the group consisting of hydrogen, C₁-C₆ alkyl, and C₃-C₆ cycloalkyl; or

R^{63a} and R^{63b} taken together with the nitrogen atom to which they are attached form a 4- to 6-membered optionally substituted heterocyclo;

R⁶⁴ is selected from the group consisting of C₁-C₆ alkyl, C₃-C₆ cycloalkyl, and —NR^{63c}R^{63d};

R^{63C} is selected from the group consisting of hydrogen, C₁-C₆ alkyl, and C₃-C₆ cycloalkyl;

R^{63d} is selected from the group consisting of hydrogen, C₁-C₆ alkyl, and C₃-C₆ cycloalkyl; or

R^{63C} and R^{63d} taken together with the nitrogen atom to which they are attached form a 4- to 6-membered optionally substituted heterocyclo, or a pharmaceutically acceptable salt or solvate thereof.

Embodiment 38. The compound of Embodiment 37, wherein R^{11a} is a substituted 4- to 14-membered heterocyclo selected from the group consisting of:

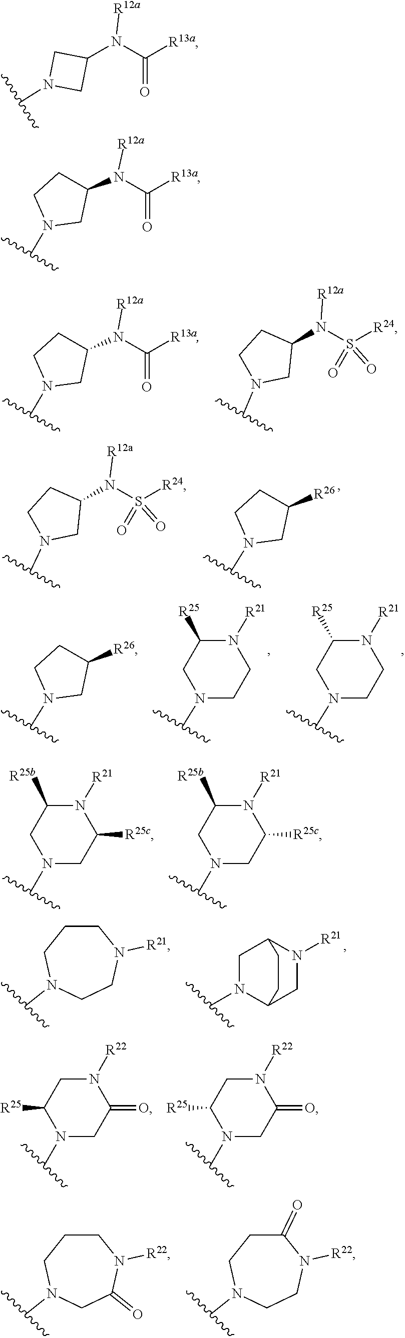

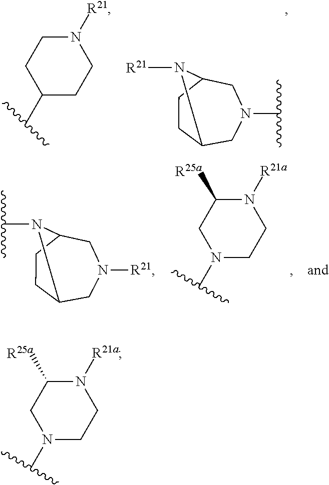

R^{12a} is selected from the group consisting of hydrogen, C₁-C₃ alkyl, (C₁-C₄ alkoxy)C₁-C₄ alkyl; and (hydroxy)C₁-C₄ alkyl;

R^{13a} is selected from the group consisting of C₁-C₄ alkyl; amino; unsubstituted C₃-C₆ cycloalkyl; substituted C₃-C₆ cycloalkyl having one or two substituents independently selected from the group consisting of halo, hydroxy, C₁-C₄ alkyl, amino, and (amino)C₁-C₄ alkyl; (C₁-C₄ alkoxy)C₁-C₄ alkyl; (hydroxy)C₁-C₄ alkyl; unsubstituted 4- to 14-membered heterocyclo; and substituted 4- to 14-membered heterocyclo having one or two substituents independently selected from the group consisting of amino, hydroxy, and C₁-C₄ alkyl;

R^{13b} is selected from the group consisting of C₁-C₄ alkyl; amino; C₁-C₄ haloalkyl; C₁-C₄ alkoxy; (hydroxy)C₁-C₄ alkyl; (C₁-C₄ alkoxy)C₁-C₄ alkyl; (amino)alkyl; unsubstituted C₃-C₆ cycloalkyl; substituted C₃-C₆ cycloalkyl having one or two substituents independently selected from the group consisting of halo, hydroxy, C₁-C₄ alkyl, amino, and (amino)C₁-C₄ alkyl; unsubstituted 4- to 14-membered heterocyclo; and substituted 4- to 14-membered heterocyclo having one or two substituents independently selected from the group consisting of amino, hydroxy, and C₁-C₄ alkyl; (C₃-C₆ cycloalkyl)oxy; and (4- to 8-membered heterocyclo)oxy;

R²¹ is selected from the group consisting of hydrogen, —C(═O)R^{13b}, C₁-C₄ alkyl, C₁-C₄ haloalkyl, unsubstituted 4- to 14-membered heterocyclo, and —S(═O)₂R²⁴;

R²² is C₁-C₄ alkyl; unsubstituted C₃-C₆ cycloalkyl; substituted C₃-C₆ cycloalkyl having one or two substituents independently selected from the group consisting of halo, hydroxy, C₁-C₄ alkyl, amino, and (amino)C₁-C₄ alkyl; unsubstituted 4- to 14-membered heterocyclo; and substituted 4- to 14-membered heterocyclo having one or two substituents independently selected from the group consisting of amino, hydroxy, and C₁-C₄ alkyl;

R²⁴ is selected from the group consisting of C₁-C₄ alkyl and (hydroxy)C₁-C₄ alkyl;

R²⁵ is selected from the group consisting of hydrogen, C₁-C₄ alkyl, and C₁-C₄ haloalkyl;

R^{25b} and R^{25C} are independently selected from the group consisting of C₁-C₄ alkyl and C₁-C₄ haloalkyl;

R²⁶ is selected from the group consisting of unsubstituted 4- to 14-membered heterocyclo; and substituted 4- to 14-membered heterocyclo having one or two substituents independently selected from the group consisting of amino, hydroxy, and C₁-C₄ alkyl; and

R^{21a} and R^{25a} taken together with the atoms to which they are attached form an optionally substituted 4- to 8-membered heterocyclo, or a pharmaceutically acceptable salt or solvate thereof.

Embodiment 39. The compound of Embodiment 37, wherein R^{11a} is a substituted 4- to 14-membered heterocyclo selected from the group consisting of:

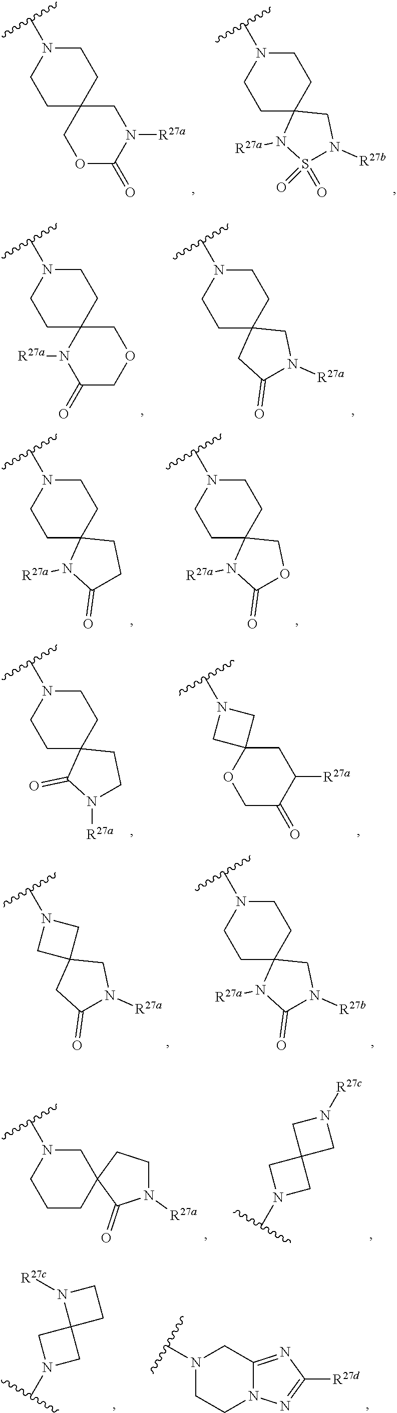

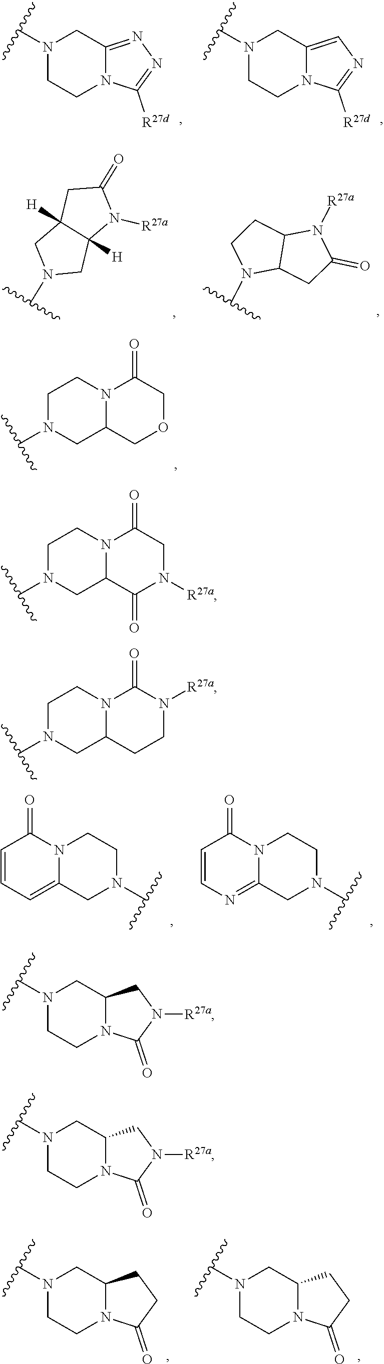

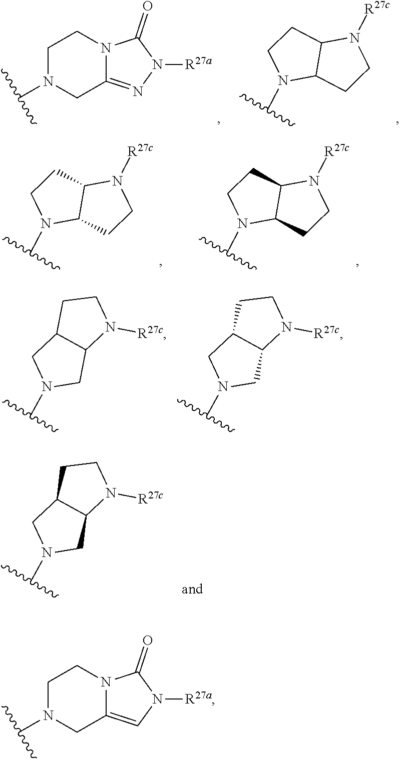

R^{27a} and R^{27b} are each independently selected from the group consisting of hydrogen, C₁-C₄ alkyl, C₁-C₄ haloalkyl, (C₁-C₄ alkoxy)C₁-C₄ alkyl; and (hydroxy)C₁-C₄ alkyl;

R^{27C} is selected from the group consisting of hydrogen; —C(═O)R^{13b}; C₁-C₄ alkyl; C₁-C₄ haloalkyl; unsubstituted 4- to 14-membered heterocyclo; and substituted 4- to 14-membered heterocyclo having one or two substituents independently selected from the group consisting of amino, hydroxy, and C₁-C₄ alkyl; and —S(═O)₂R²⁴;

R^{27d} is selected from the group consisting of hydrogen; C₁-C₄ alkyl; and C₁-C₄ haloalkyl;

R^{13b} is selected from the group consisting of C₁-C₄ alkyl; aminoC₁-C₄ haloalkyl; C₁-C₄ alkoxy; (hydroxy)C₁-C₄ alkyl; (C₁-C₄ alkoxy)C₁-C₄ alkyl; (amino)alkyl; unsubstituted C₃-C₆ cycloalkyl; substituted C₃-C₆ cycloalkyl having one or two substituents independently selected from the group consisting of halo, hydroxy, C₁-C₄ alkyl, amino, and (amino)C₁-C₄ alkyl; unsubstituted 4- to 14-membered heterocyclo; substituted 4- to 14-membered heterocyclo having one or two substituents independently selected from the group consisting of amino, hydroxy, and C₁-C₄ alkyl; (C₃-C₆ cycloalkyl)oxy; and (4- to 8-membered heterocyclo)oxy; and

R²⁴ is selected from the group consisting of C₁-C₄ alkyl and (hydroxy)C₁-C₄ alkyl, or a pharmaceutically acceptable salt or solvate thereof.

Embodiment 40. The compound of Embodiment 39, wherein R^{11a} is a substituted 4- to 14-membered heterocyclo selected from the group consisting of:

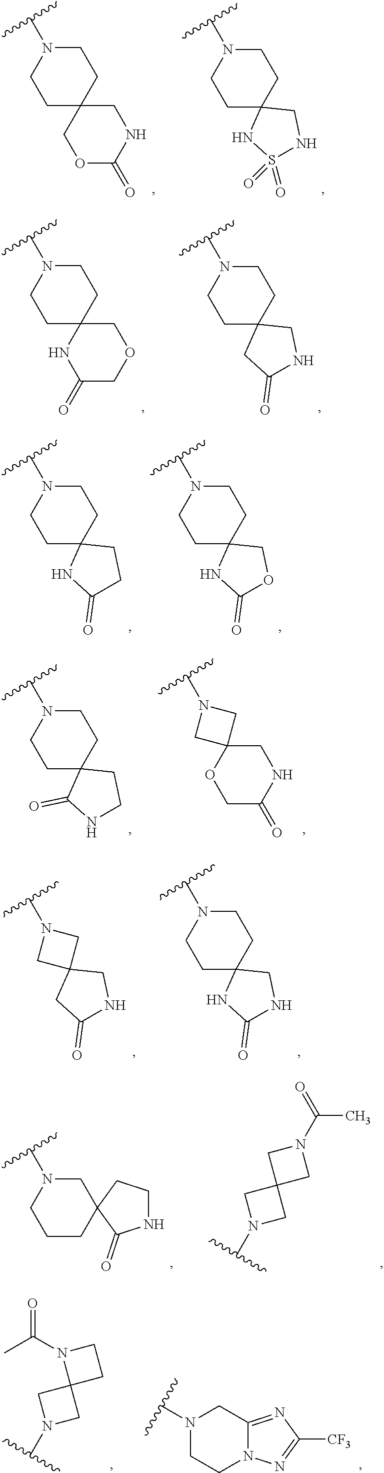

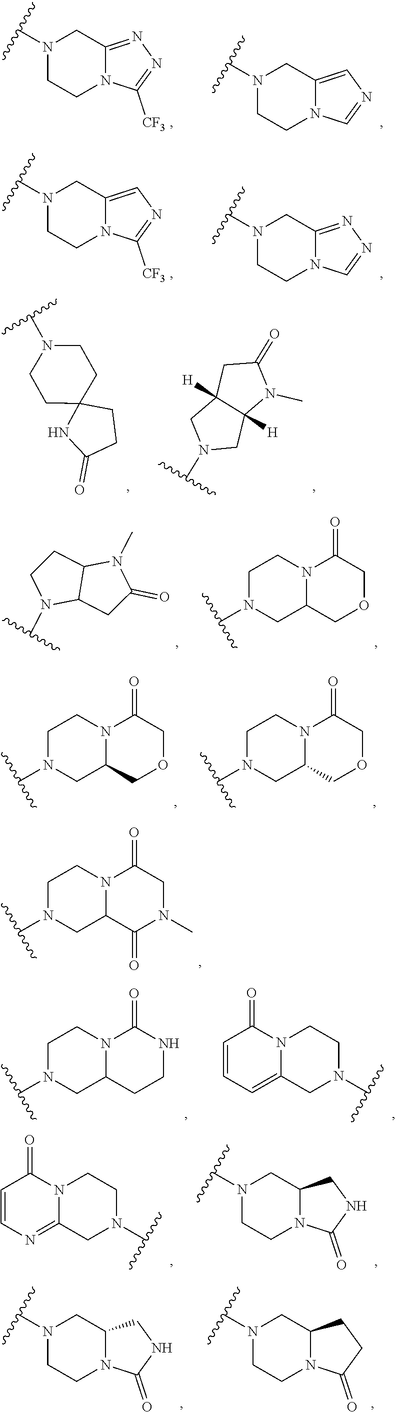

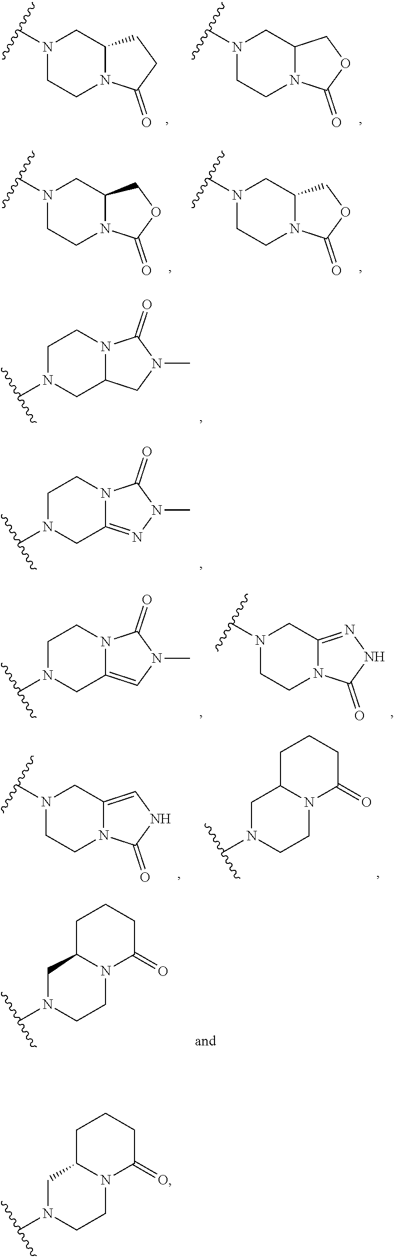

or a pharmaceutically acceptable salt or solvate thereof.

Embodiment 41. The compound of Embodiment 37, wherein R^{11a} is a substituted 4- to 14-membered heterocyclo selected from the group consisting of:

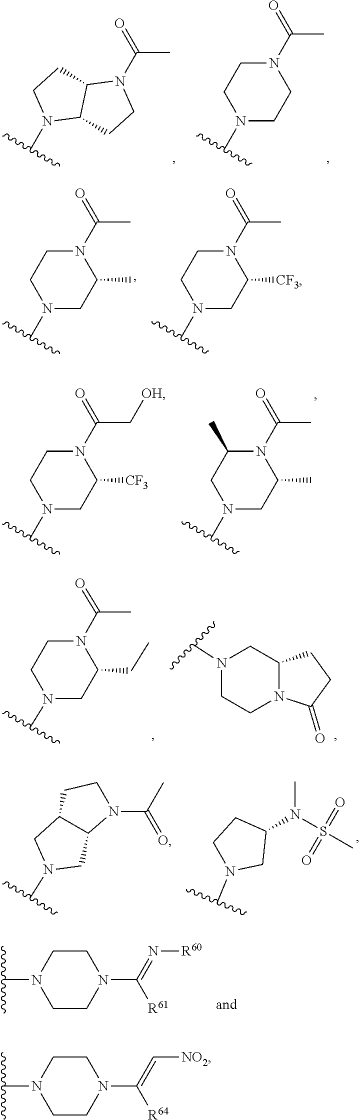

or a pharmaceutically acceptable salt or solvate thereof.

Embodiment 42. The compound of any one of Embodiments 32-41, wherein Z⁴ is —CH₂—, or a pharmaceutically acceptable salt or solvate thereof.

Embodiment 43. The compound of any one of Embodiments 32-38 or 42, wherein R^{11a} is selected from the group consisting of unsubstituted 4- to 14-membered heterocyclo; substituted 4- to 14-membered heterocyclo having one or two substituents independently selected from the group consisting of —N(R^{12a})C(═O)R^{13a}, —C(═O)R^{13b}, and C₁-C₄ alkyl; unsubstituted 5- to 10-membered heteroaryl; and substituted 5- or 6-membered heteroaryl having one or two substituents independently selected from the group consisting of halo and C₁-C₄ alkyl, or a pharmaceutically acceptable salt or solvate thereof.

Embodiment 44. The compound of any one of Embodiments 32-38, 42, or 43, wherein R^{11a} is a substituted 4- to 14-membered heterocyclo is selected from the group consisting of

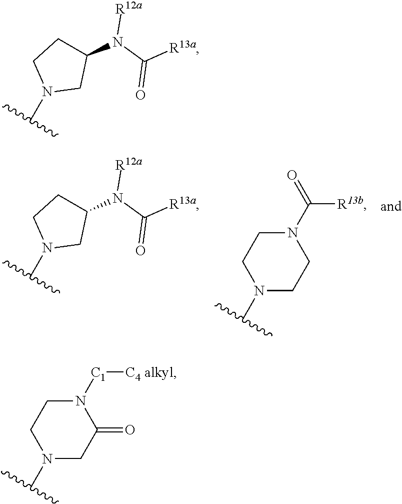

or a pharmaceutically acceptable salt or solvate thereof.

Embodiment 45. The compound of Embodiment 44, wherein: R^{12a} is selected from the group consisting of hydrogen and C₁-C₃ alkyl; R^{13a} is C₁-C₄ alkyl; and R^{13b} is C₁-C₄ alkyl, or a pharmaceutically acceptable salt or solvate thereof.

Embodiment 46. The compound of Embodiment 45, wherein: R^{12a} is selected from the group consisting of hydrogen and methyl; R^{13a} is methyl; and R^{13b} is methyl, or a pharmaceutically acceptable salt or solvate thereof.

Embodiment 47. The compound Embodiment 13 of Formula V:

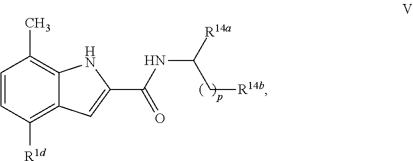

wherein:

R^{14a} is selected from the group consisting of optionally substituted alkyl and optionally substituted heteroaryl;

R^{14b} is selected from the group consisting of optionally substituted alkyl, optionally substituted heteroaryl, optionally substituted aryl, optionally substituted heterocyclo, optionally substituted cycloalkyl, and carboxamido; and

p is 0, 1, 2, or 3; or a pharmaceutically acceptable salt or solvate thereof.

Embodiment 48. The compound of Embodiment 47 of Formula V-A:

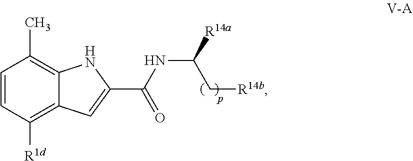

or a pharmaceutically acceptable salt or solvate thereof.

Embodiment 49. The compound of Embodiment 47 of Formula V-B:

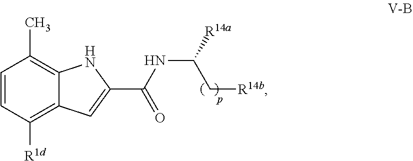

or a pharmaceutically acceptable salt or solvate thereof.

Embodiment 50. The compound of any one of Embodiments 47-49, wherein:

R^{14a} is selected from the group consisting of:

(A) unsubstituted 5- to 10-membered heteroaryl;

(B) substituted 5- to 10-membered heteroaryl having one, two, three, or four substituents independently selected from the group consisting of: (i) halo; (ii) C₁-C₄ alkyl; (iii) C₁-C₄ alkoxy; (iv) (3- to 8-membered heterocyclo)C₁-C₄ alkyl; (v) (5- to 9-membered heteroaryl)C₁-C₄ alkyl; (vi) —C(═O)NR^{15a}R^{15b}; (vii) unsubstituted 5- to 10-membered heteroaryl; (viii) substituted 5- to 10-membered heteroaryl having one, two, or three substituents independently selected from the group consisting of halo, C₁-C₄ alkyl, (3- to 8-membered heterocyclo)C₁-C₄ alkyl, 5- to 9-membered heteroaryl, and —NR^{15e}R^{15f}; (ix) —OR¹⁶; (x) unsubstituted C₃-C₆ cycloalkyl; (xi) substituted C₃-C₆ cycloalkyl having one, two, three, or four substituents independently selected from the group consisting of C₁-C₄ alkyl and —N(R^{17a})C(═O)R^{18a}; (xii) cyano; (xiii) unsubstituted 4- to 14-membered heterocyclo; (xiv) substituted 4- to 14-membered heterocyclo having one or two substituents independently selected from the group consisting of C₁-C₄ alkyl, (5- to 9-membered heteroaryl)C₁-C₄ alkyl; (xv) (carboxy)C₁-C₄ alkyl; (xvi) (carboxamido)C₁-C₄ alkyl; and (xvii) carboxy; and

(C) C₁-C₆ alkyl;

R^{14b} is selected from the group consisting of:

(A) unsubstituted 5-to 10-membered heteroaryl;

(B) substituted 5- or 10-membered heteroaryl having one, two, three, or four substituents independently selected from the group consisting of halo, C₁-C₄ alkyl, and (C₃-C₆ cycloalkyl)C₁-C₄ alkyl;

(C) unsubstituted C₆-C₁₀ aryl;

(D) substituted C₆-C₁₀ aryl, having one, two, three, or four substituents independently selected from the group consisting of halo, C₁-C₄ alkyl, and (3- to 8-membered heterocyclo)C₁-C₄ alkyl;

(E) unsubstituted 4- to 14-membered heterocyclo;

(F) substituted 4- to 14-membered heterocyclo having one, two, three, or four substituents independently selected from the group consisting of hydroxy, amino, and C₁-C₄ alkyl;

(G) —C(═O)NR^{15c}R^{15d};

(H) unsubstituted C₃-C₆ cycloalkyl; and

(I) C₁-C₆ alkyl;

p is 0, 1, 2, or 3;

R^{15a} and R^{15b} are independently selected from the group consisting of: (A) hydrogen; (B) C₁-C₆ alkyl; (C) C₁-C₆ haloalkyl; (D) (C₁-C₄ alkoxy)C₁-C₄ alkyl; (E) (hydroxy)C₁-C₄ alkyl; (F) (cyano)alkyl; (G) unsubstituted C₆-C₁₀ aryl; (H) substituted C₆-C₁₀ aryl, having one, two, three, or four substituents independently selected from the group consisting of halo, amino, hydroxy, and C₁-C₄ alkyl; (I) unsubstituted 5- or 6-membered heteroaryl; (J) substituted 5- or 6-membered heteroaryl having one, two, three, or four substituents independently selected from the group consisting of halo, amino, hydroxy, and C₁-C₄ alkyl; (K) unsubstituted 4- to 14-membered heterocyclo; (L) substituted 4- to 14-membered heterocyclo having one or two substituents independently selected from the group consisting of amino, hydroxy, and C₁-C₄ alkyl; (M) unsubstituted C₃-C₈ cycloalkyl; and (N) substituted C₃-C₈ cycloalkyl having one, two, three, or four substituents independently selected from the group consisting of C₁-C₆ alkyl and —NR^{15g}R^{15h}; or

R^{15a} and R^{15b} taken together with the nitrogen atom to which they are attached form an optionally substituted 4- to 14-membered heterocyclo;

R^{15C} and R^{15d} are independently selected from the group consisting of: (A) hydrogen; (B) C₁-C₆ alkyl; (C) C₁-C₆ haloalkyl; (D) (C₁-C₄ alkoxy)C₁-C₄ alkyl; (E) (hydroxy)C₁-C₄ alkyl; (F) (cyano)alkyl; (G) unsubstituted C₆-C₁₀ aryl; (H) substituted C₆-C₁₀ aryl, having one, two, three, or four substituents independently selected from the group consisting of halo, amino, hydroxy, and C₁-C₄ alkyl; (I) unsubstituted 5- or 6-membered heteroaryl; (J) substituted 5- or 6-membered heteroaryl having one, two, three, or four substituents independently selected from the group consisting of halo, amino, hydroxy, and C₁-C₄ alkyl; (K) unsubstituted 4- to 14-membered heterocyclo; (L) substituted 4- to 14-membered heterocyclo having one or two substituents independently selected from the group consisting of amino, hydroxy, and C₁-C₄ alkyl; (M) unsubstituted C₃-C₈ cycloalkyl; and (N) substituted C₃-C₈ cycloalkyl having one, two, three, or four substituents independently selected from the group consisting of C₁-C₆ alkyl and —NR^{15g}R^{15h}; or

R^{15C} and R^{15d} taken together with the nitrogen atom to which they are attached form an optionally substituted 4- to 14-membered heterocyclo;

R^{15e} and R^{15f} are independently selected from the group consisting of: (A) hydrogen (B) C₁-C₆ alkyl; (C) C₁-C₆ haloalkyl; (D) (C₁-C₄ alkoxy)C₁-C₄ alkyl; (E) (hydroxy)C₁-C₄ alkyl; (F) (cyano)alkyl; (G) unsubstituted C₆-C₁₀ aryl, (G¹) substituted C₆-C₁₀ aryl, having one, two, three, or four substituents independently selected from the group consisting of halo, amino, hydroxy, and C₁-C₄ alkyl; (I) unsubstituted 5- or 6-membered heteroaryl; (J) substituted 5- or 6-membered heteroaryl having one, two, three, or four substituents independently selected from the group consisting of halo, amino, hydroxy, and C₁-C₄ alkyl; (K) unsubstituted 4- to 14-membered heterocyclo; (L) substituted 4- to 14-membered heterocyclo having one or two substituents independently selected from the group consisting of amino, hydroxy, and C₁-C₄ alkyl; (M) unsubstituted C₃-C₈ cycloalkyl; and (N) substituted C₃-C₈ cycloalkyl having one, two, three, or four substituents independently selected from the group consisting of C₁-C₆ alkyl and —NR^{15g}R^{15h}; or

R^{15e} and R^{15f} taken together with the nitrogen atom to which they are attached form an optionally substituted 4- to 14-membered heterocyclo;

R^{15g} and R^{15h} are independently selected from the group consisting of: (A) hydrogen; (B) C₁-C₆ alkyl; (C) C₁-C₆ haloalkyl; (D) C₁-C₆ alkoxy; (E) (C₁-C₄ alkoxy)C₁-C₄ alkyl; (F) (hydroxy)C₁-C₄ alkyl; (G) (cyano)alkyl; (H) unsubstituted C₆-C₁₀ aryl; (I) substituted C₆-C₁₀ aryl, having one, two, three, or four substituents independently selected from the group consisting of halo, amino, hydroxy, and C₁-C₄ alkyl; (J) unsubstituted 5- or 6-membered heteroaryl; (K) substituted 5- or 6-membered heteroaryl having one, two, three, or four substituents independently selected from the group consisting of halo, amino, hydroxy, and C₁-C₄ alkyl; (L) unsubstituted 4- to 14-membered heterocyclo; (M) substituted 4- to 14-membered heterocyclo having one or two substituents independently selected from the group consisting of amino, hydroxy, and C₁-C₄ alkyl; (N) unsubstituted C₃-C₈ cycloalkyl; and (O) substituted C₃-C₈ cycloalkyl having one, two, three, or four substituents independently selected from the group consisting of C₁-C₆ alkyl and —NR^{15g}R^{15h}; or

R^{15g} and R^{15g} taken together with the nitrogen atom to which they are attached form an optionally substituted 4- to 14-membered heterocyclo;

R¹⁶ is (amino)(hydroxy)C₁-C₄ alkyl;

R^{17a} is selected from the group consisting of hydrogen and C₁-C₄ alkyl;

R^{18a} is selected from the group consisting of: (A) C₁-C₆ alkyl; (B) C₁-C₆ haloalkyl; (C) C₁-C₆ alkoxy; (D) (C₁-C₄ alkoxy)C₁-C₄ alkyl; (E) (hydroxy)C₁-C₄ alkyl; (F) (cyano)alkyl; (G) unsubstituted C₆-C₁₀ aryl; (H) substituted C₆-C₁₀ aryl, having one, two, three, or four substituents independently selected from the group consisting of halo, amino, hydroxy, and C₁-C₄ alkyl; (I) unsubstituted 5- or 6-membered heteroaryl; (J) substituted 5- or 6-membered heteroaryl having one, two, three, or four substituents independently selected from the group consisting of halo, amino, hydroxy, and C₁-C₄ alkyl; (K) unsubstituted 4- to 14-membered heterocyclo; (L) substituted 4- to 14-membered heterocyclo having one or two substituents independently selected from the group consisting of amino, hydroxy, and C₁-C₄ alkyl; (M) unsubstituted C₃-C₈ cycloalkyl; and (N) substituted C₃-C₈ cycloalkyl having one, two, three, or four substituents independently selected from the group consisting of amino, hydroxy, and C₁-C₄ alkyl, or a pharmaceutically acceptable salt or solvate thereof.

Embodiment 51. The compound of any one of Embodiments 47-50, wherein R^{14a} is selected from the group consisting of unsubstituted 5- to 10-membered heteroaryl; and substituted 5- to 10-membered heteroaryl having one, two, or three substituents independently selected from the group consisting of C₁-C₄ alkyl; C₁-C₄ alkoxy; (3- to 8-membered heterocyclo)C₁-C₄ alkyl; (5- to 9-membered heteroaryl)C₁-C₄ alkyl; —C(═O)NR^{15a}R^{15b}; unsubstituted 5- to 10-membered heteroaryl; substituted 5- to 10-membered heteroaryl having one, two, or three substituents independently selected from the group consisting of halo, C₁-C₄ alkyl, (3- to 8-membered heterocyclo)C₁-C₄ alkyl, 5- to 9-membered heteroaryl, and —NR^{15e}R^{15f}; unsubstituted C₃-C₆ cycloalkyl; and substituted C₃-C₆ cycloalkyl having one, two, or three substituents independently selected from the group consisting of C₁-C₄ alkyl and —N(R^{17a})C(═O)R^{18a}, or a pharmaceutically acceptable salt or solvate thereof.

Embodiment 52. The compound of any one of Embodiments 47-51, wherein R^{14a} is a substituted pyridyl having one, two, or three substituents independently selected from the group consisting of C₁-C₄ alkyl; C₁-C₄ alkoxy; (3- to 8-membered heterocyclo)C₁-C₄ alkyl; (5- to 9-membered heteroaryl)C₁-C₄ alkyl; —C(═O)NR^{15a}R^{15b}; unsubstituted 5- to 10-membered heteroaryl; substituted 5- to 10-membered heteroaryl having one, two, or three substituents independently selected from the group consisting of halo, C₁-C₄ alkyl, (3- to 8-membered heterocyclo)C₁-C₄ alkyl, 5- to 9-membered heteroaryl, and —NR^{15e}R^{15f}; unsubstituted C₃-C₆ cycloalkyl; and substituted C₃-C₆ cycloalkyl having one, two, or three substituents independently selected from the group consisting of C₁-C₄ alkyl and —N(R^{17a})C(═O)R^{18a}, or a pharmaceutically acceptable salt or solvate thereof.

Embodiment 53. The compound of any one of Embodiments 47-52, wherein R^{14b} is selected from the group consisting of unsubstituted 5- to 10-membered heteroaryl; substituted 5- to 10-membered heteroaryl having one or two substituents independently selected from the group consisting of C₁-C₄ alkyl and (C₃-C₆ cycloalkyl)C₁-C₄ alkyl; unsubstituted C₆-C₁₀ aryl; substituted C₆-C₁₀ aryl, having one or two substituents independently selected from the group consisting of C₁-C₄ alkyl and (3- to 8-membered heterocyclo)C₁-C₄ alkyl; unsubstituted 4- to 14-membered heterocyclo; substituted 4- to 14-membered heterocyclo having one or two substituents independently selected from the group consisting of hydroxy, amino, and C₁-C₄ alkyl; and unsubstituted C₃-C₆ cycloalkyl, or a pharmaceutically acceptable salt or solvate thereof.

Embodiment 54. The compound of any one of Embodiments 47-53, wherein R^{14b} is selected from the group consisting of unsubstituted 5- or 6-membered heteroaryl; substituted 5- or 6-membered heteroaryl having one or two substituents independently selected from the group consisting of C₁-C₄ alkyl and (C₃-C₆ cycloalkyl)C₁-C₄ alkyl; unsubstituted phenyl; substituted phenyl, having one or two substituents independently selected from the group consisting of C₁-C₄ alkyl and (3- to 8-membered heterocyclo)C₁-C₄ alkyl; and unsubstituted C₃-C₆ cycloalkyl, or a pharmaceutically acceptable salt or solvate thereof.

Embodiment 55. The compound of any one of Embodiments 47-54, wherein p is 0, or a pharmaceutically acceptable salt or solvate thereof.

Embodiment 56. The compound of any one of Embodiments 47-54, wherein p is 1, or a pharmaceutically acceptable salt or solvate thereof.

Embodiment 57. The compound of Embodiment 13 of Formula VI:

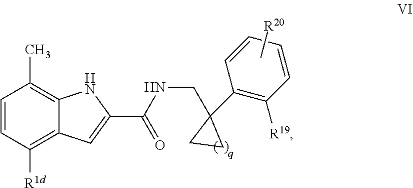

wherein:

R¹⁹ is selected from the group consisting of:

unsubstituted 4- to 14-membered heterocyclo; and

substituted 4- to 14-membered heterocyclo having one or two substituents independently selected from the group consisting of amino, hydroxy, and C₁-C₄ alkyl;

R²⁰ is selected from the group consisting of hydrogen, halo, and C₁-C₄ alkyl; and

q is 1, 2, or 3,

or a pharmaceutically acceptable salt or solvate thereof.

Embodiment 58. The compound of Embodiment 13 of Formula VII:

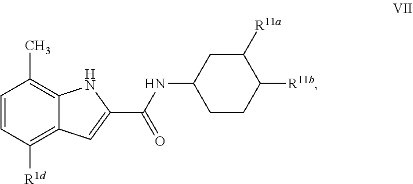

wherein:

R^{11a} is selected from the group consisting of optionally substituted alkyl, optionally substituted heterocyclo, optionally substituted heteroaryl, and —N(R^{12b})C(═O)R^{13c};

R^{12b} is selected from the group consisting of hydrogen, alkyl, cycloalkyl, and heterocyclo, (C₁-C₄ alkoxy)C₁-C₄ alkyl, and (hydroxy)C₁-C₄ alkyl;

R^{13c} is selected from the group consisting of alkyl, haloalkyl, alkoxy, (alkoxy)alkyl, (hydroxy)alkyl, (cyano)alkyl, optionally substituted aryl, optionally substituted heteroaryl, optionally substituted cycloalkyl, and optionally substituted heterocycle, amino, (amino)alkyl, (C₃-C₆ cycloalkyl)oxy, and (4- to 8-membered heterocyclo)oxy; and

R^{11b} is selected from the group consisting of C₁-C₄ alkyl, halo, and C₁-C₄ haloalkyl, or a pharmaceutically acceptable salt or solvate thereof.

Embodiment 59. The compound of Embodiment 58 of Formula VII-A:

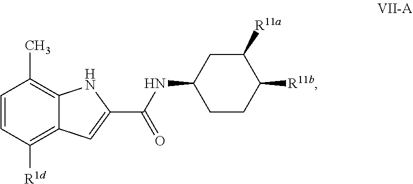

or a pharmaceutically acceptable salt or solvate thereof.

Embodiment 60. The compound of Embodiment 58 of Formula VII-B:

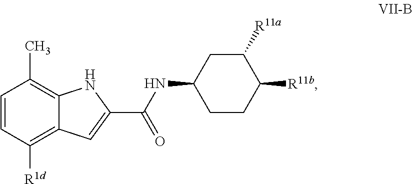

or a pharmaceutically acceptable salt or solvate thereof.

Embodiment 61. The compound of Embodiment 58 of Formula VII-C:

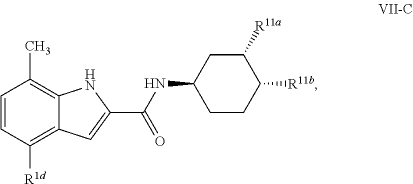

or a pharmaceutically acceptable salt or solvate thereof.

Embodiment 62. The compound of Embodiment 58 of Formula VII-D:

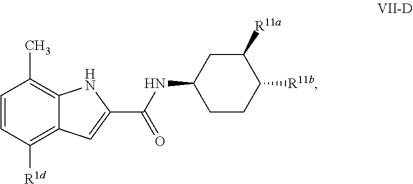

or a pharmaceutically acceptable salt or solvate thereof.

Embodiment 63. The compound of Embodiment 58 of Formula VII-E:

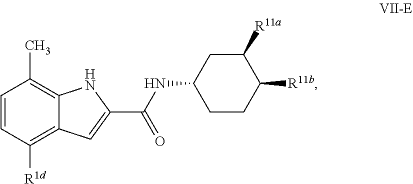

or a pharmaceutically acceptable salt or solvate thereof.

Embodiment 64. The compound of Embodiment 58 of Formula VII-F:

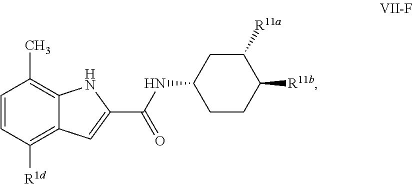

or a pharmaceutically acceptable salt or solvate thereof.

Embodiment 65. The compound of Embodiment 58 of Formula VII-G:

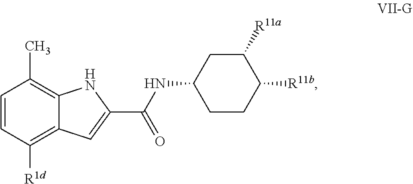

or a pharmaceutically acceptable salt or solvate thereof.

Embodiment 66. The compound of Embodiment 58 of Formula VII-H:

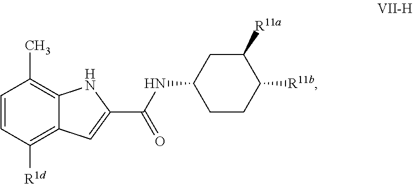

or a pharmaceutically acceptable salt or solvate thereof.

Embodiment 67. The compound of Embodiment 13 of Formula VIII:

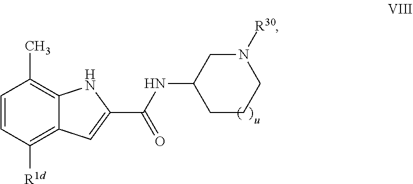

wherein:

R³⁰ is selected from the group consisting of hydrogen; C₁-C₆ alkyl; unsubstituted C₃-C₆ cycloalkyl; substituted C₃-C₆ cycloalkyl having one or two substituents independently selected from the group consisting of halo, hydroxy, C₁-C₄ alkyl, amino, and (amino)C₁-C₄ alkyl; unsubstituted 4- to 14-membered heterocyclo; and substituted 4-to 14-membered heterocyclo having one or two substituents independently selected from the group consisting of amino, hydroxy, and C₁-C₄ alkyl; —C(═O)R^{13b}, and —S(═O)₂R²⁴;

R^{13b} is selected from the group consisting of C₁-C₄ alkyl; amino; C₁-C₄ haloalkyl; C₁-C₄ alkoxy; (hydroxy)C₁-C₄ alkyl; (C₁-C₄ alkoxy)C₁-C₄ alkyl; (amino)alkyl; unsubstituted C₃-C₆ cycloalkyl; substituted C₃-C₆ cycloalkyl having one or two substituents independently selected from the group consisting of halo, hydroxy, C₁-C₄ alkyl, amino, and (amino)C₁-C₄ alkyl; unsubstituted 4- to 14-membered heterocyclo; substituted 4- to 14-membered heterocyclo having one or two substituents independently selected from the group consisting of amino, hydroxy, and C₁-C₄ alkyl; (C₃-C₆ cycloalkyl)oxy; and (4- to 8-membered heterocyclo)oxy;

R²⁴ is selected from the group consisting of C₁-C₄ alkyl and (hydroxy)C₁-C₄ alkyl; and

u is 0, 1, 2, or 3, or a pharmaceutically acceptable salt or solvate thereof.

Embodiment 68. The compound of Embodiment 67 of Formula VIII-A:

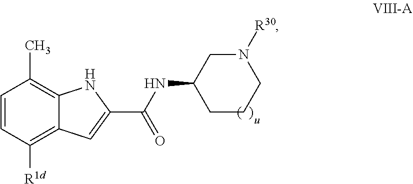

or a pharmaceutically acceptable salt or solvate thereof.

Embodiment 69. The compound of Embodiment 67 of Formula VIII-B:

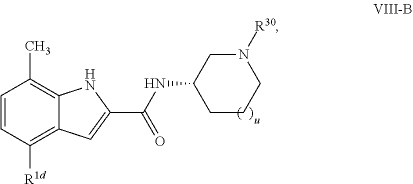

or a pharmaceutically acceptable salt or solvate thereof.

Embodiment 70. The compound of any one of Embodiments 1-11 or 13-69, wherein R^{1d} is fluoro, or a pharmaceutically acceptable salt or solvate thereof.

Embodiment 71. The compound of Embodiment 1 selected from any one or more of the compounds of Table 1:

TABLE 1

| Cpd. | |
|---|---|
| No. | Chemical Structure |
| 1 | 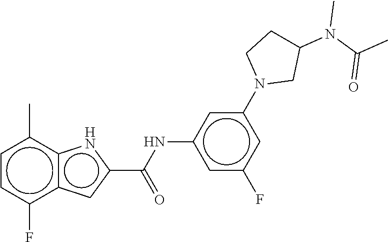 |
| 2 | 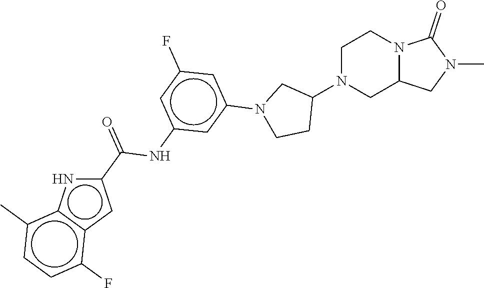 |
| 3 | 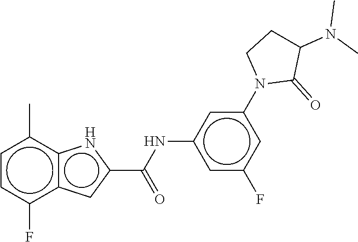 |
| 4 | 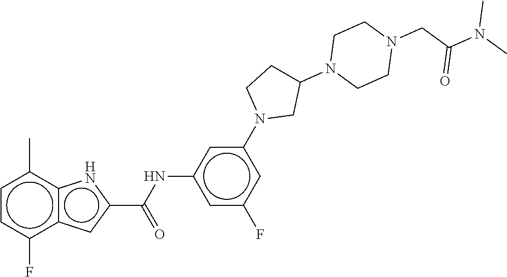 |
| 5 | 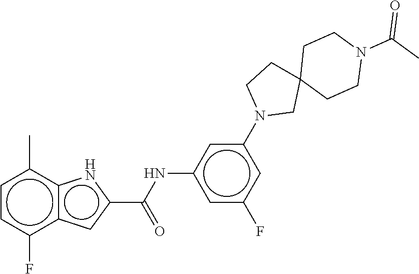 |
| 6 | 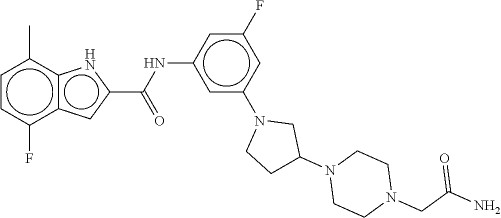 |
| 7 | 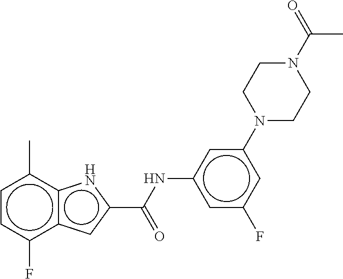 |
| 8 | 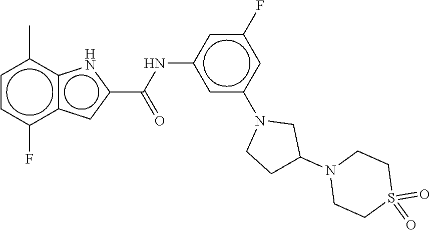 |
| 9 | 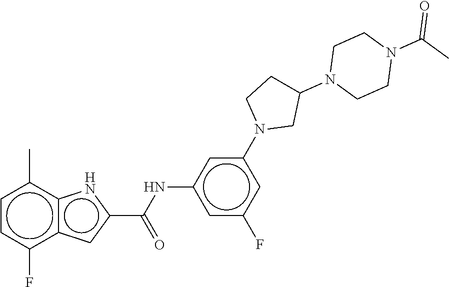 |
| 10 | 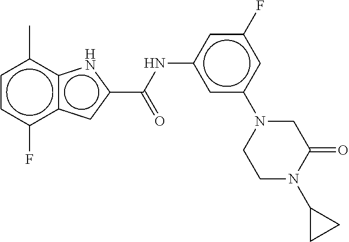 |
| 11 | 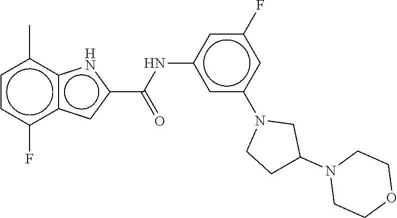 |
| 12 | 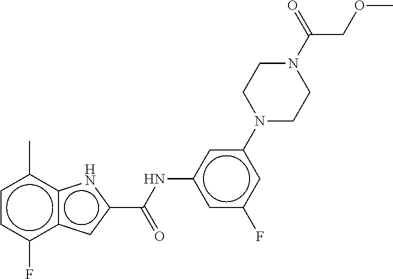 |
| 13 | 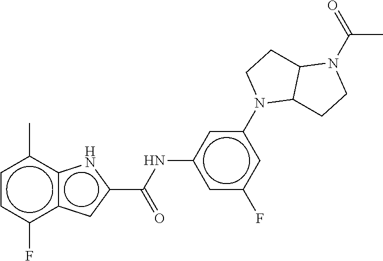 |
| 14 | 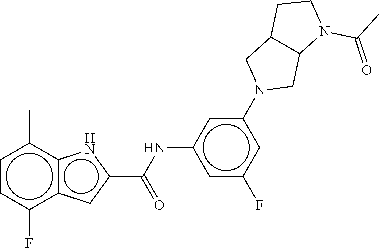 |
| 15 | 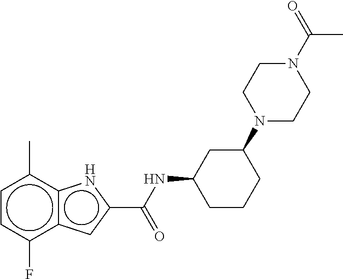 |
| 16 | 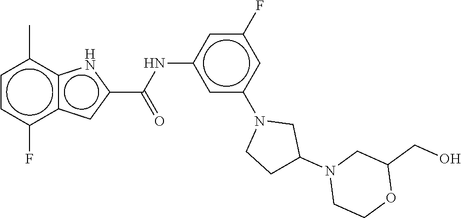 |
| 17 | 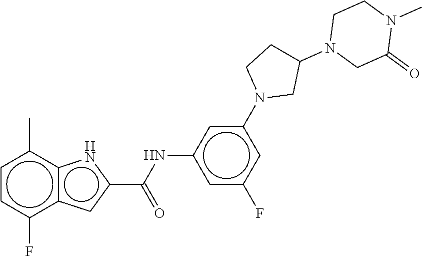 |
| 18 | 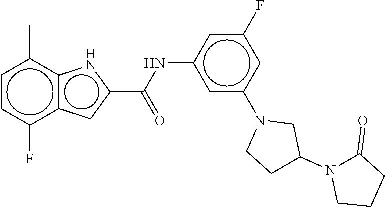 |
| 19 | 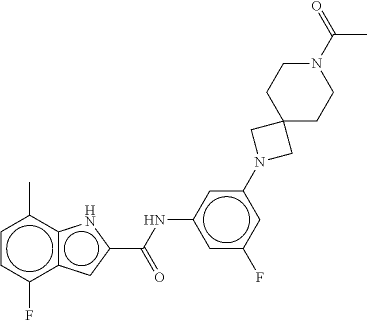 |
| 20 | 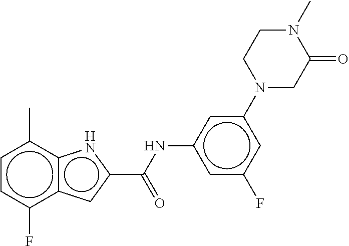 |
| 21 | 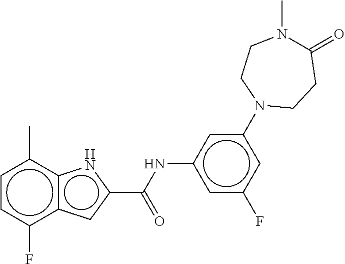 |
| 22 | 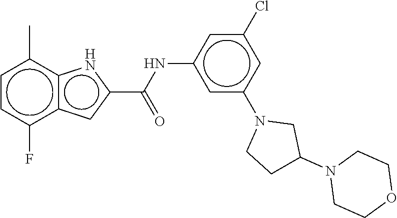 |
| 23 | 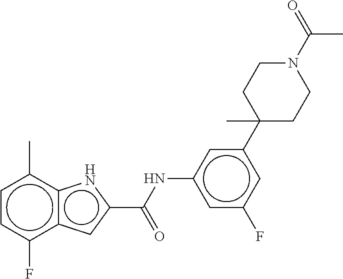 |
| 24 | 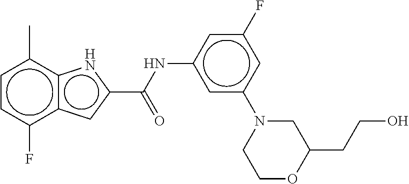 |
| 25 | 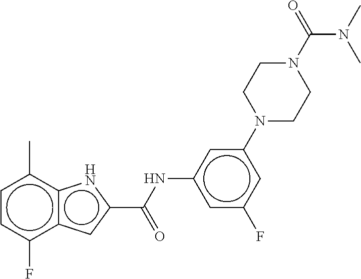 |
| 26 | 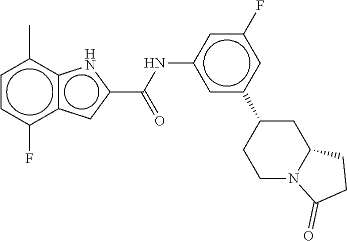 |
| 27 | 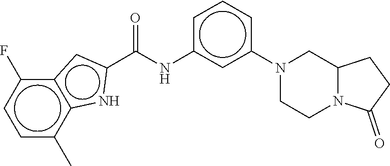 |
| 28 | 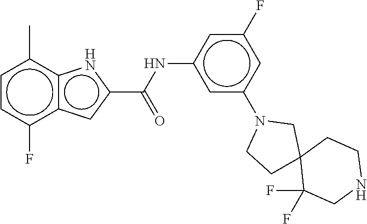 |
| 29 | 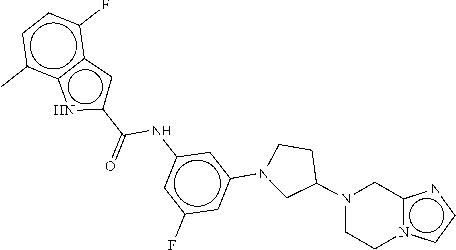 |
| 30 | 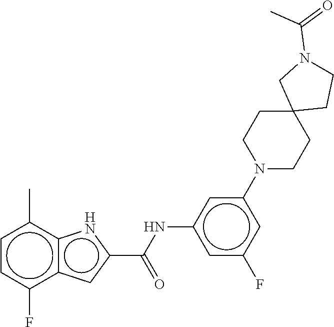 |
| 31 | 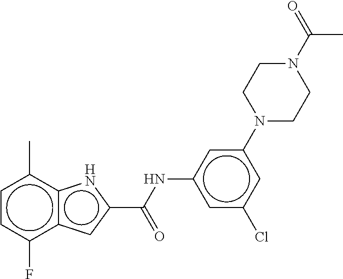 |
| 32 | 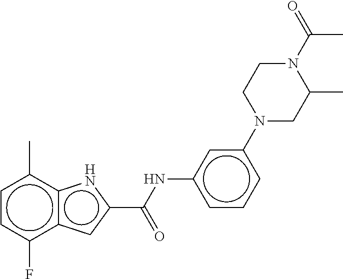 |
| 34 | 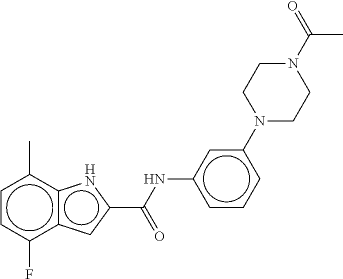 |
| 35 | 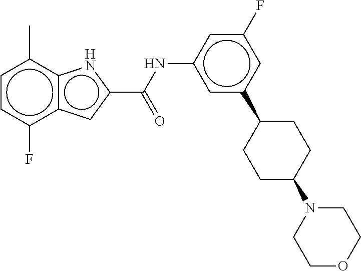 |
| 36 | 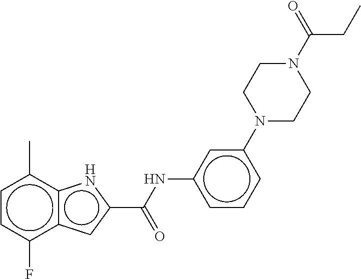 |
| 37 | 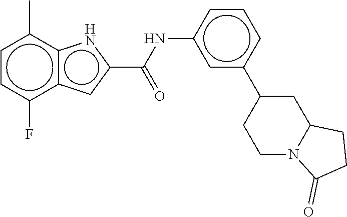 |
| 38 | 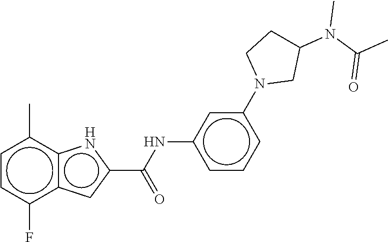 |
| 39 | 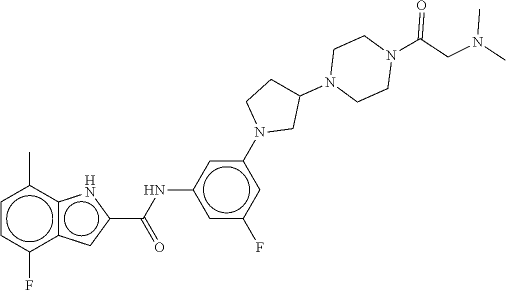 |
| 40 | 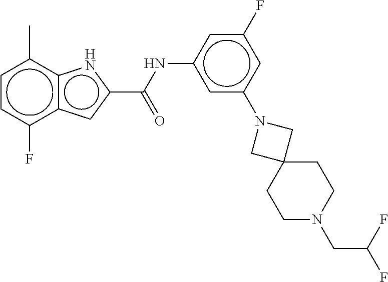 |
| 41 | 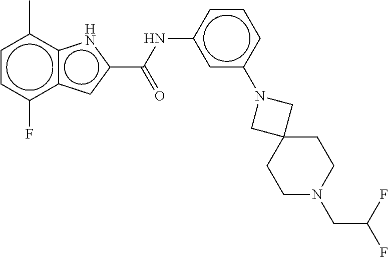 |
| 42 | 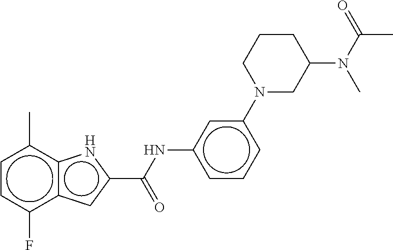 |
| 43 | 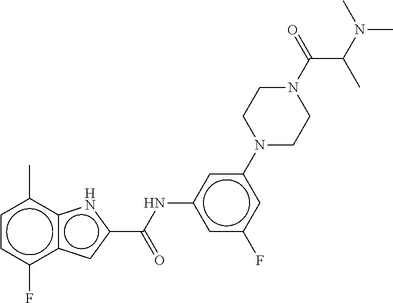 |
| 44 | 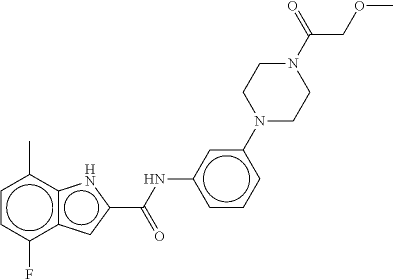 |
| 45 | 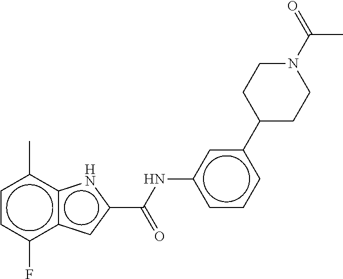 |
| 46 | 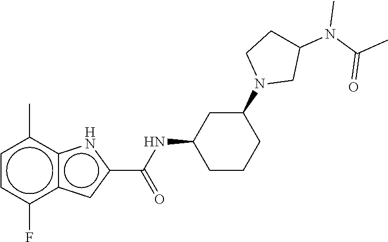 |
| 47 | 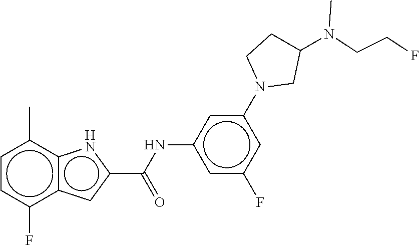 |
| 48 | 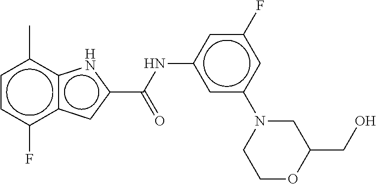 |
| 49 | 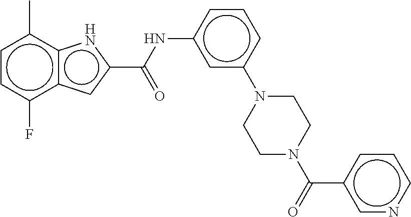 |
| 50 | 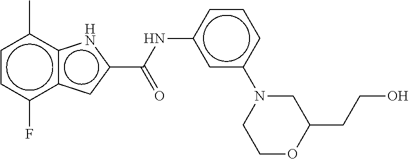 |
| 51 | 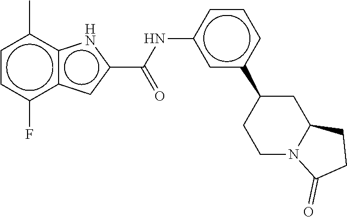 |
| 52 | 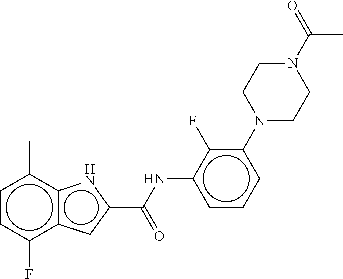 |
| 53 | 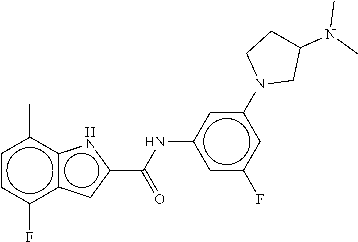 |
| 54 | 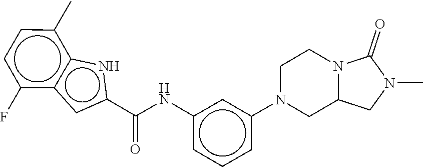 |
| 55 | 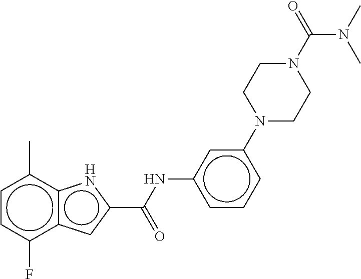 |
| 56 | 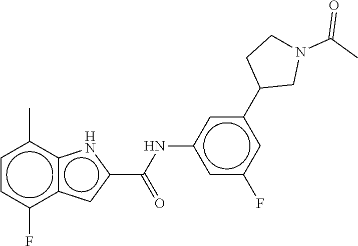 |
| 57 | 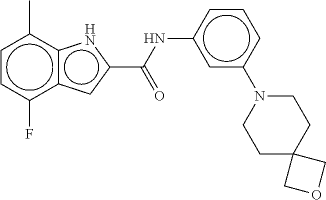 |
| 58 | 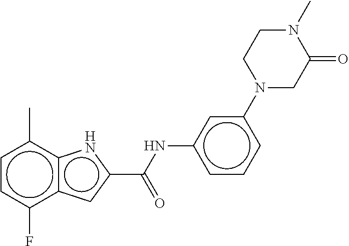 |
| 59 | 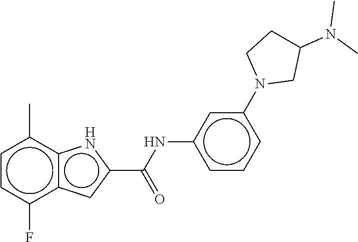 |
| 60 | 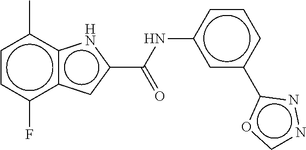 |
| 61 | 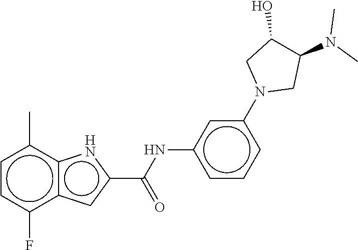 |
| 62 | 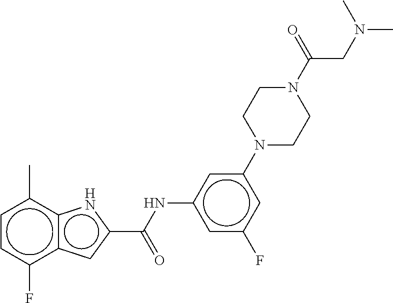 |
| 63 | 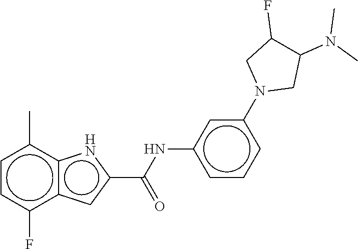 |
| 64 | 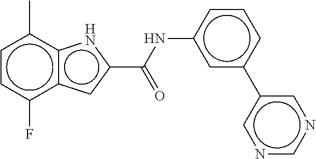 |
| 65 | 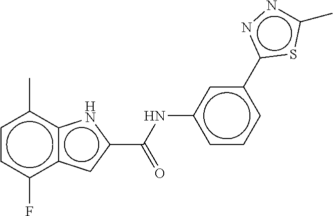 |
| 66 | 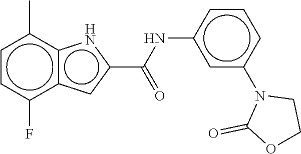 |
| 68 | 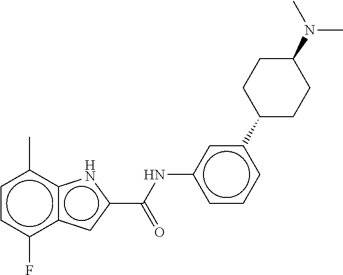 |
| 69 | 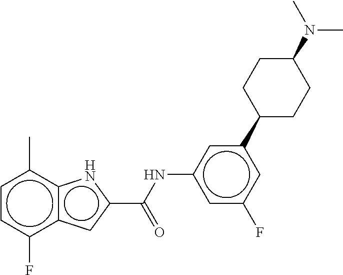 |
| 70 | 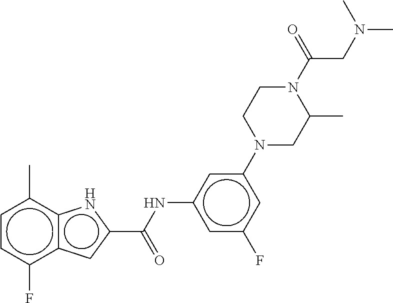 |
| 71 | 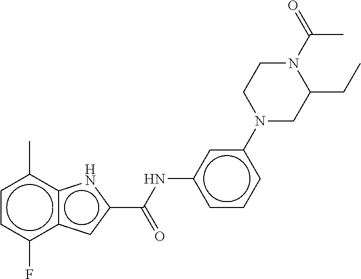 |
| 72 | 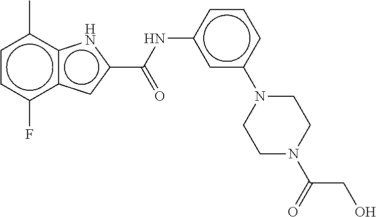 |
| 73 | 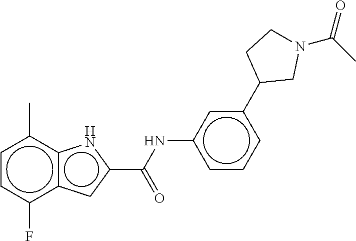 |
| 74 | 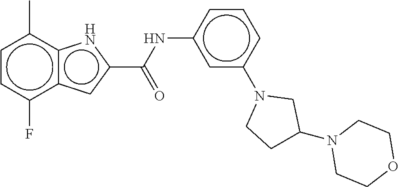 |
| 75 | 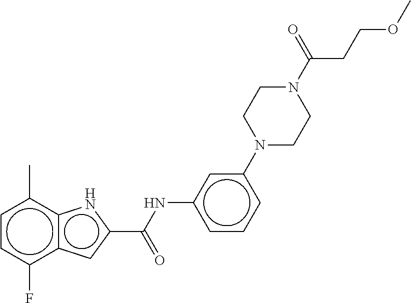 |
| 76 | 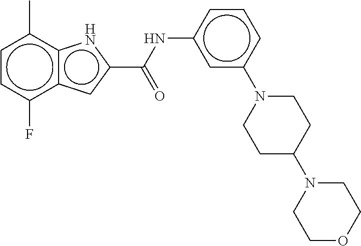 |
| 77 | 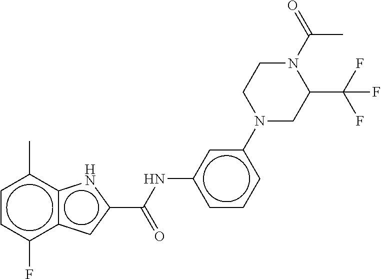 |
| 78 | 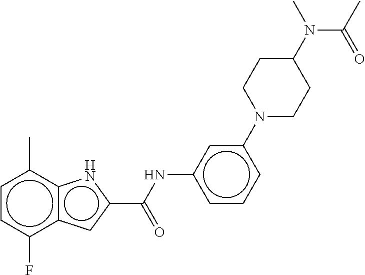 |
| 79 | 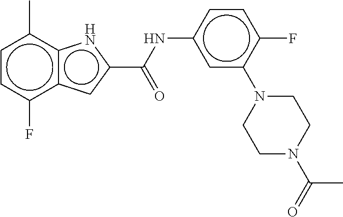 |
| 80 | 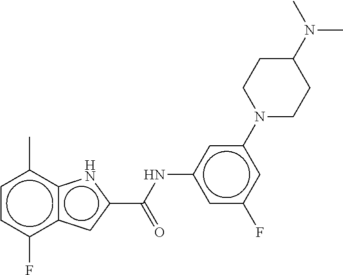 |
| 81 | 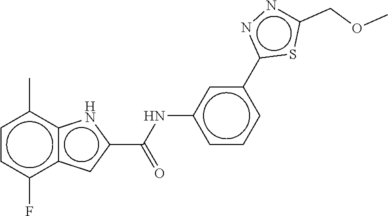 |
| 82 | 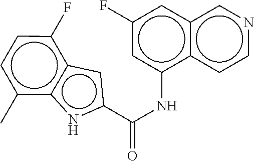 |
| 83 | 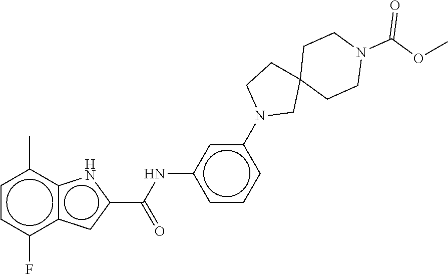 |
| 84 | 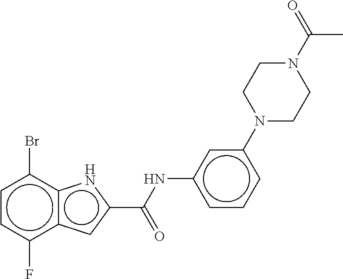 |
| 85 | 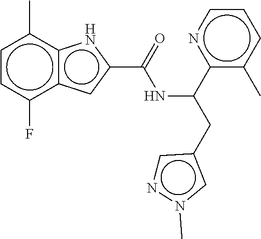 |
| 86 | 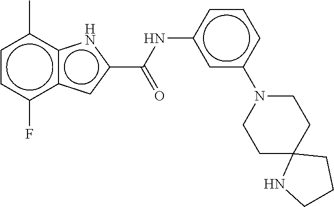 |
| 87 | 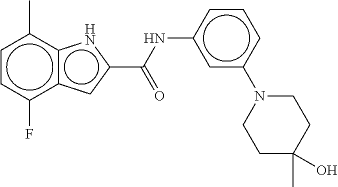 |
| 88 | 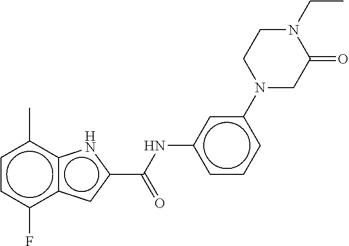 |
| 89 | 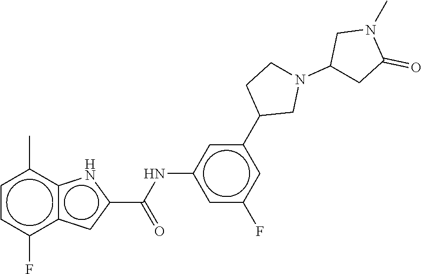 |
| 90 | 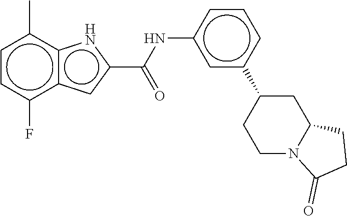 |
| 91 | 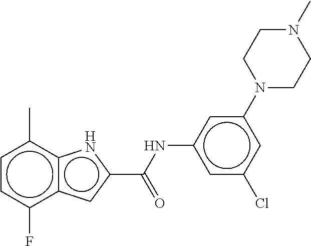 |
| 92 | 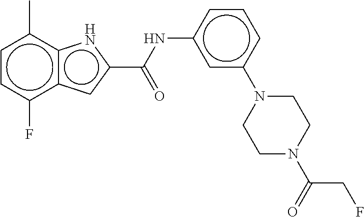 |
| 93 | 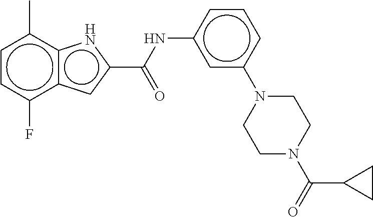 |
| 94 | 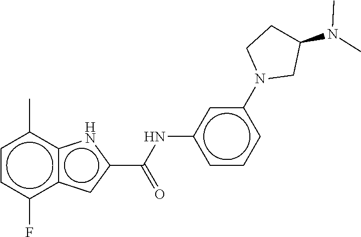 |
| 95 | 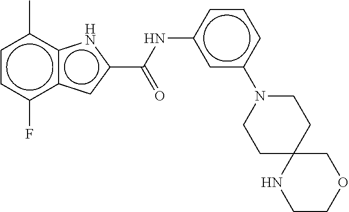 |
| 96 | 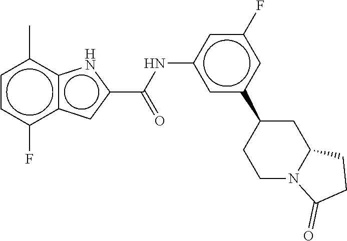 |
| 97 | 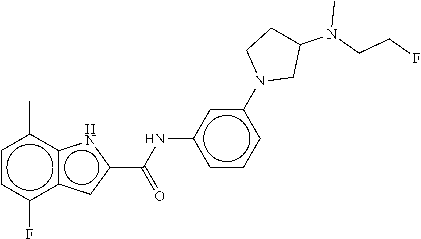 |
| 98 | 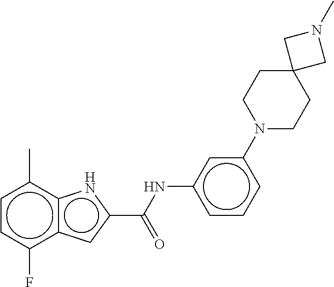 |
| 99 | 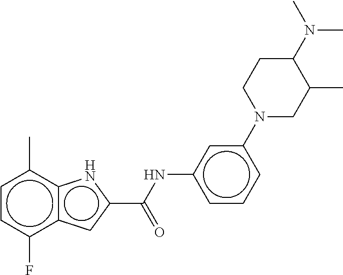 |
| 100 | 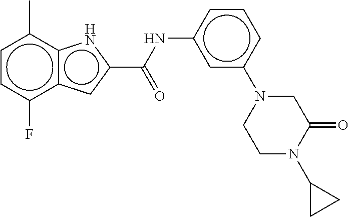 |
| 101 | 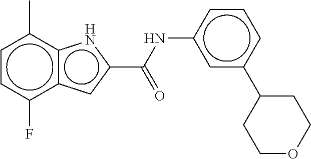 |
| 102 | 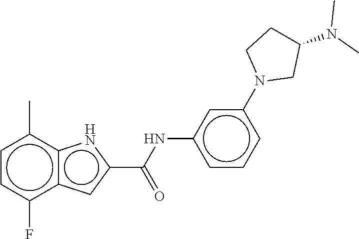 |
| 103 | 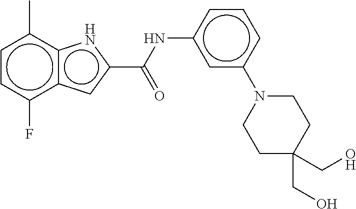 |
| 104 | 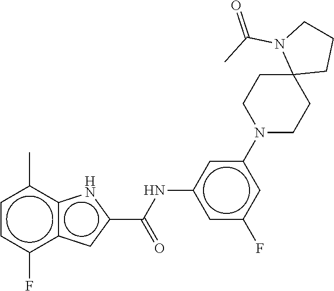 |
| 105 | 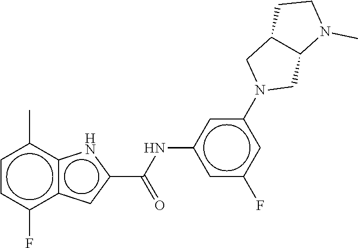 |
| 106 | 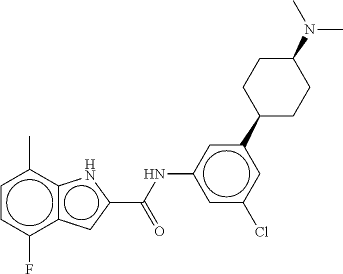 |
| 107 | 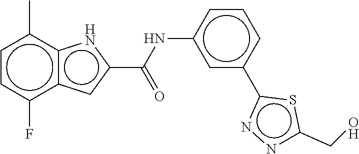 |
| 108 | 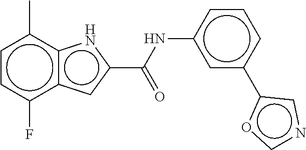 |
| 109 | 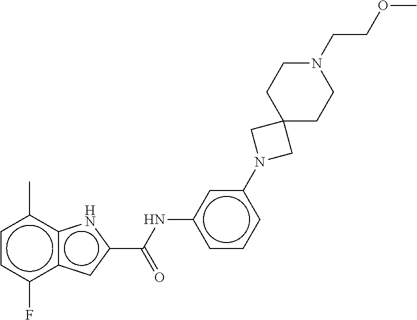 |
| 110 | 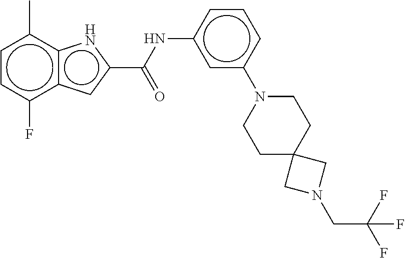 |
| 111 | 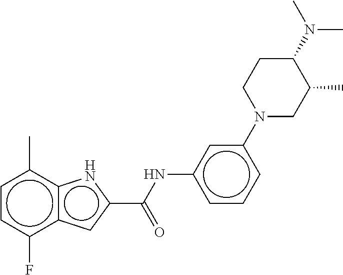 |
| 112 | 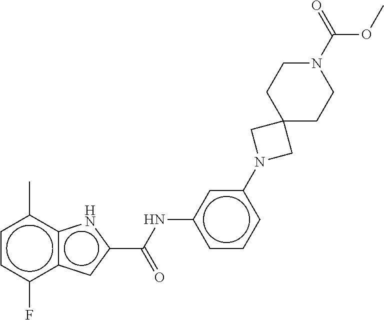 |
| 113 | 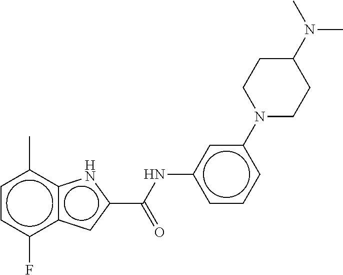 |
| 114 | 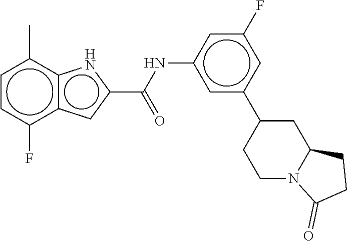 |
| 115 | 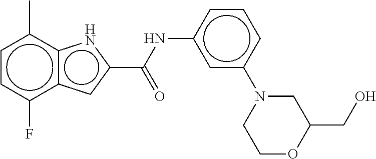 |
| 116 | 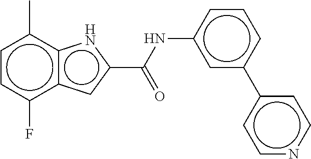 |
| 117 | 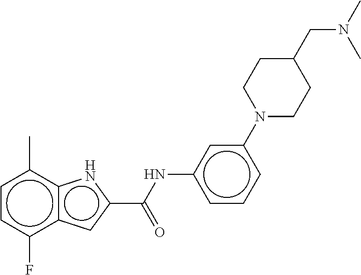 |
| 118 | 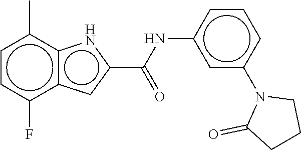 |
| 119 | 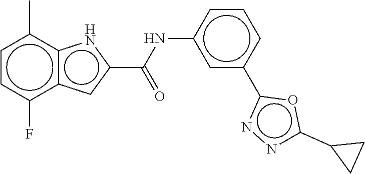 |
| 120 | 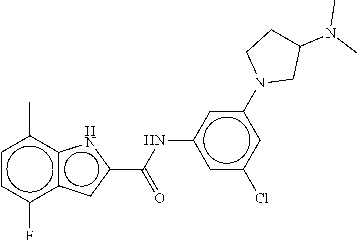 |
| 121 | 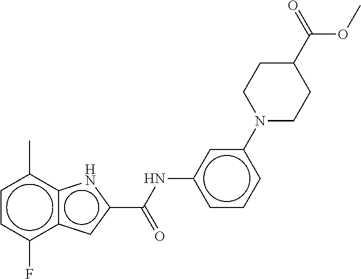 |
| 122 | 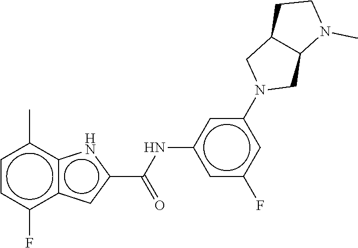 |
| 123 | 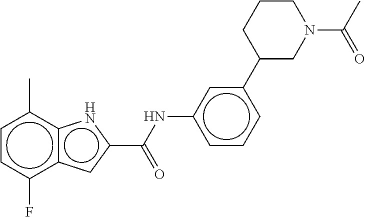 |
| 124 | 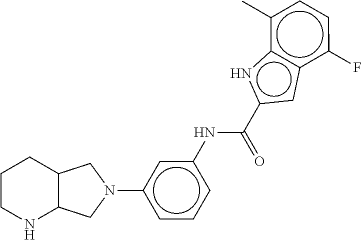 |
| 125 | 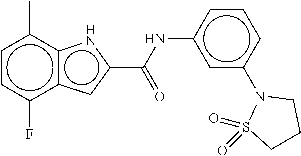 |
| 126 | 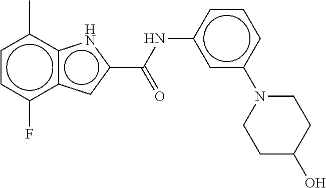 |
| 127 | 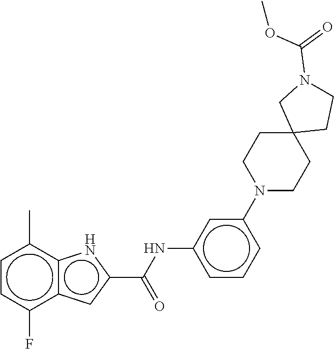 |
| 128 | 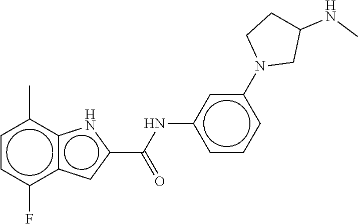 |
| 129 | 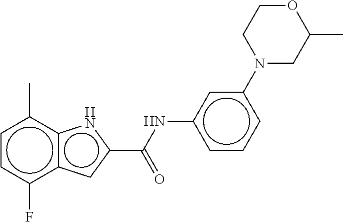 |
| 130 | 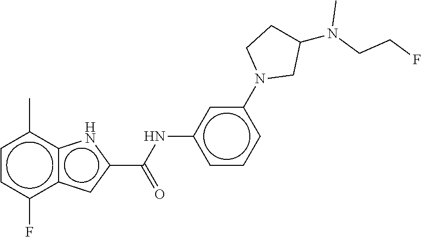 |
| 131 | 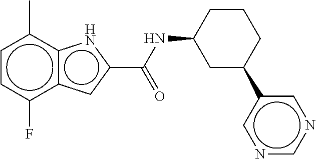 |
| 132 | 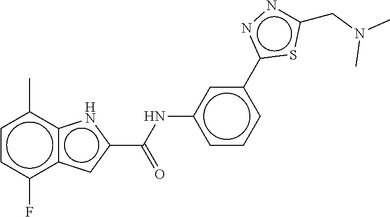 |
| 133 | 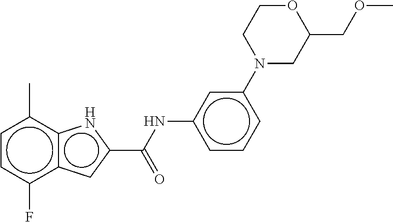 |
| 134 | 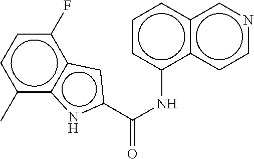 |
| 135 | 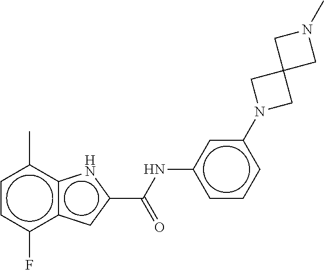 |
| 136 | 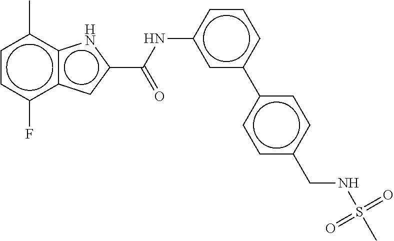 |
| 137 | 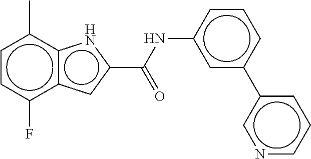 |
| 138 | 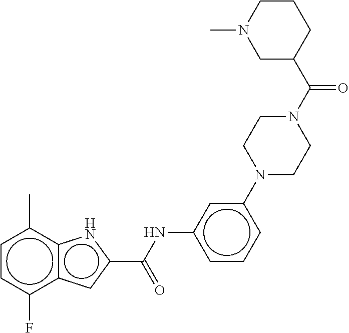 |
| 139 | 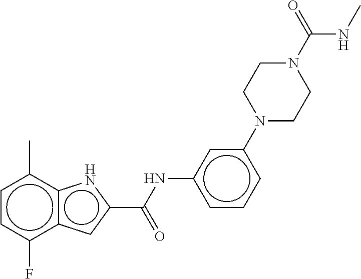 |
| 140 | 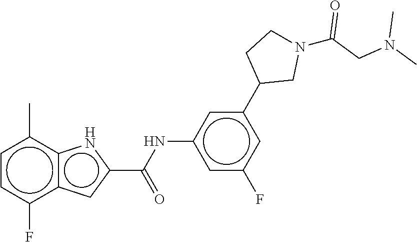 |
| 141 | 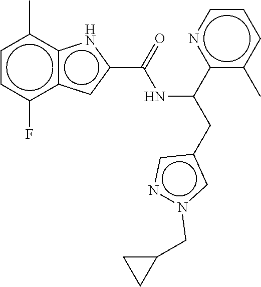 |
| 142 | 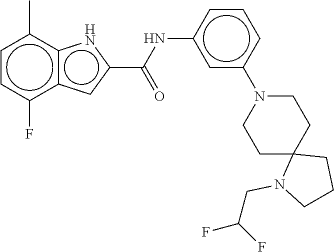 |
| 143 | 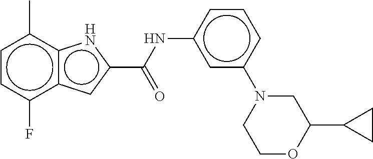 |
| 144 | 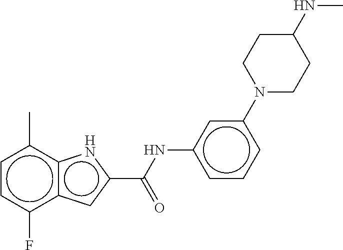 |
| 145 | 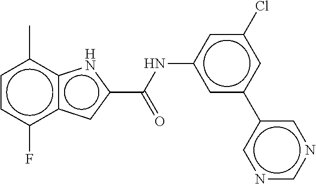 |
| 146 | 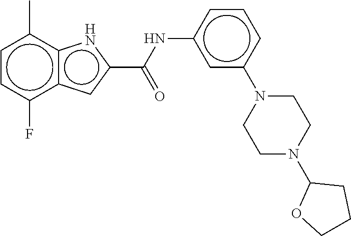 |
| 147 | 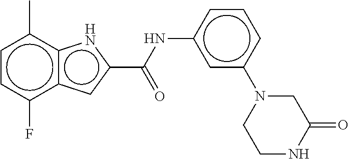 |
| 148 | 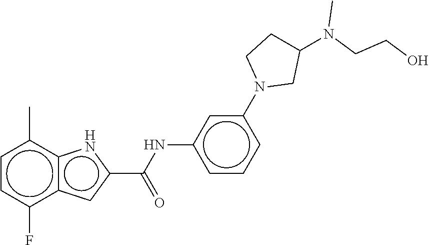 |
| 149 | 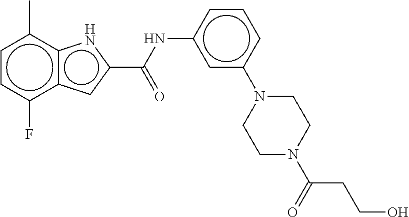 |
| 150 | 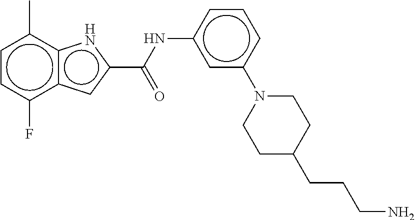 |
| 151 | 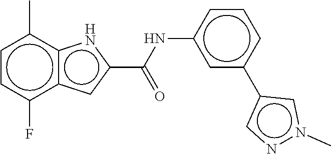 |
| 152 | 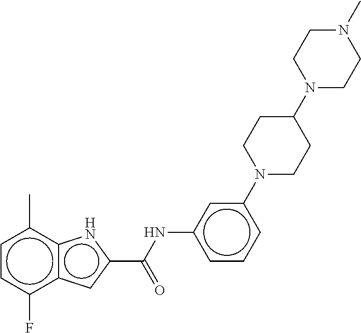 |
| 153 | 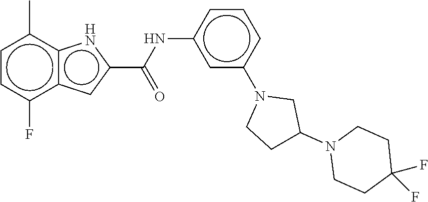 |
| 154 | 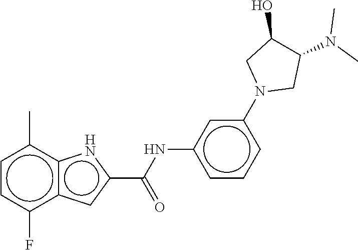 |
| 155 | 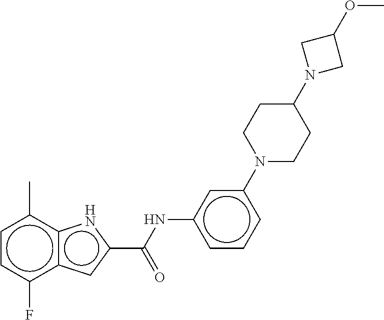 |
| 156 | 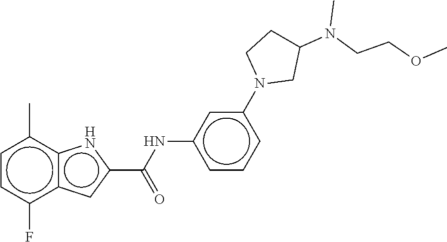 |
| 157 | 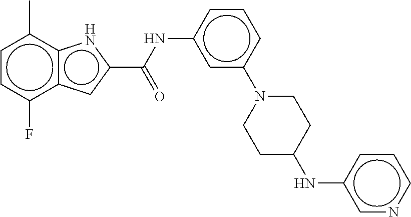 |
| 159 | 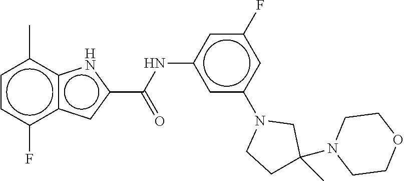 |
| 160 | 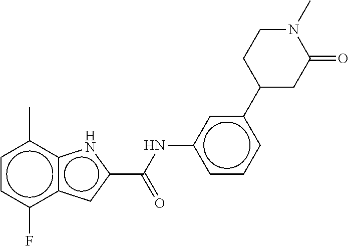 |
| 161 | 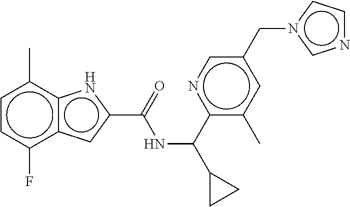 |
| 162 | 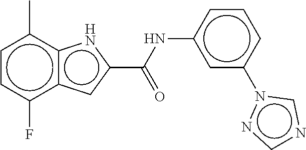 |
| 163 | 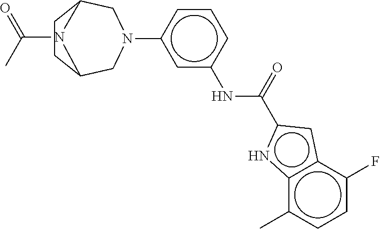 |
| 164 | 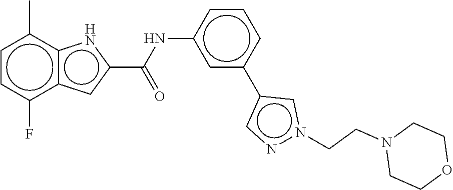 |
| 165 | 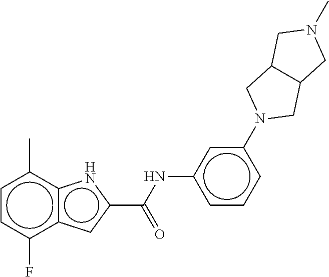 |
| 166 | 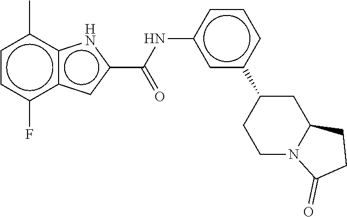 |
| 167 | 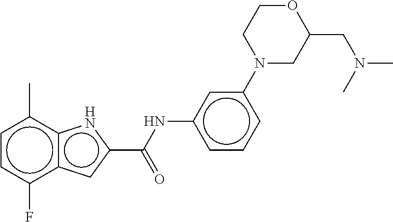 |
| 168 | 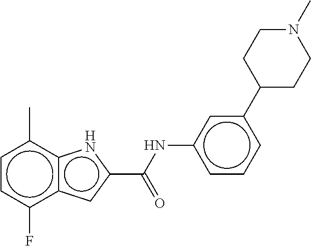 |
| 169 | 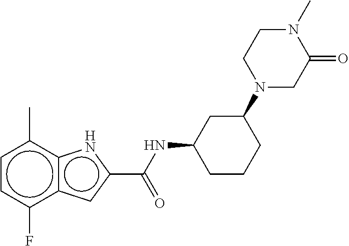 |
| 170 | 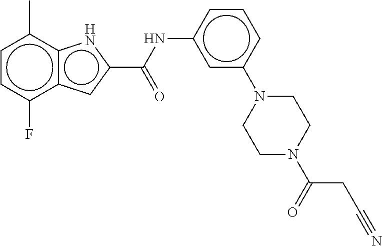 |
| 171 | 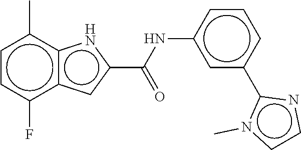 |
| 172 | 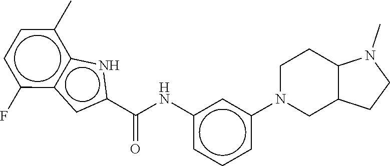 |
| 173 | 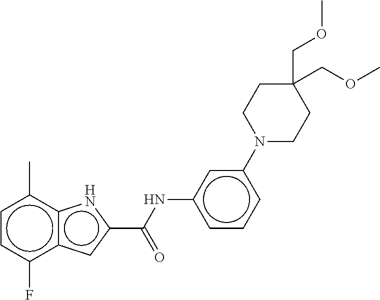 |
| 174 | 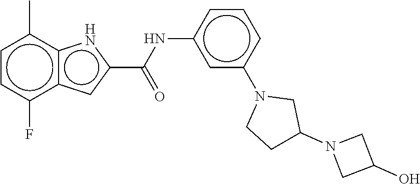 |
| 175 | 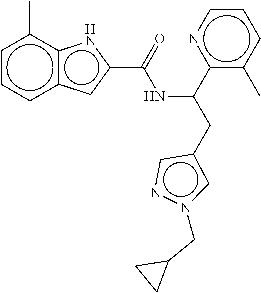 |
| 176 | 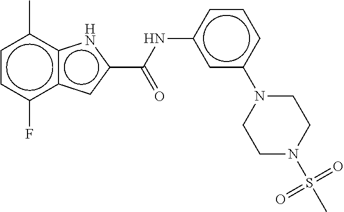 |
| 177 | 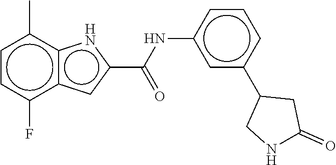 |
| 178 | 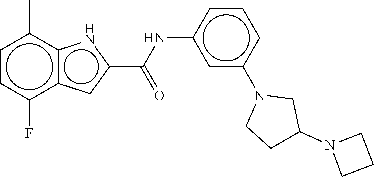 |
| 179 | 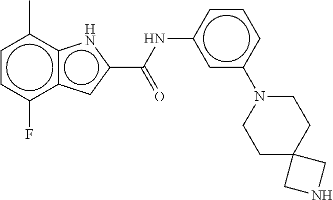 |
| 180 | 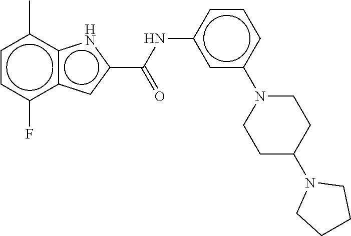 |
| 181 | 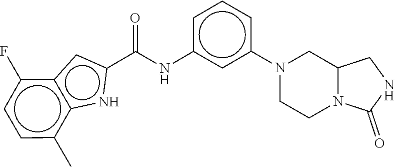 |
| 182 | 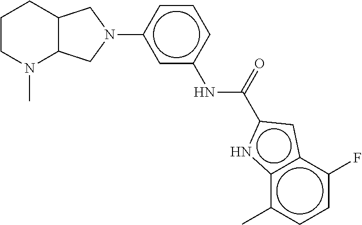 |
| 183 | 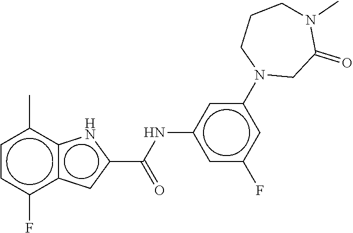 |
| 184 | 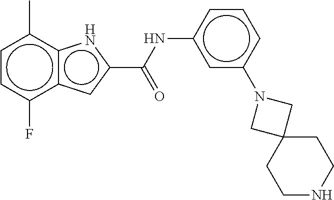 |
| 185 | 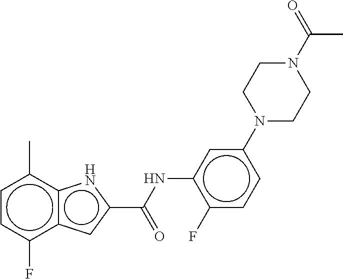 |
| 186 | 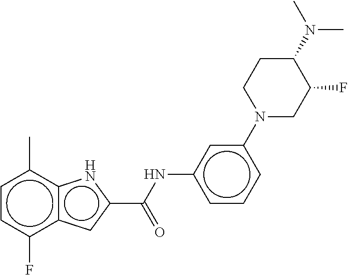 |
| 187 | 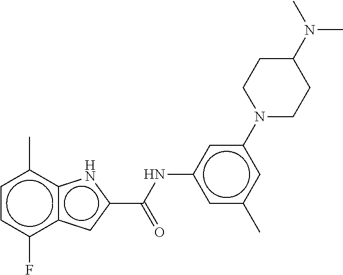 |
| 188 | 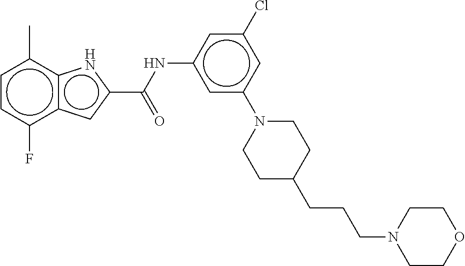 |
| 189 | 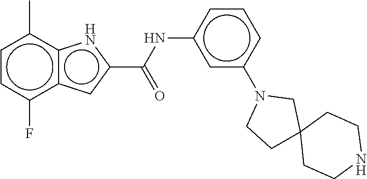 |
| 190 | 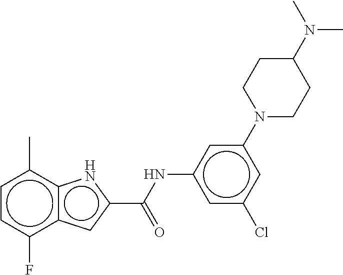 |
| 191 | 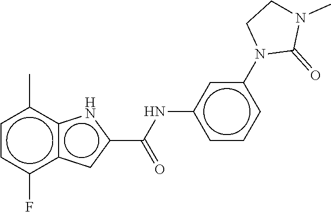 |
| 192 | 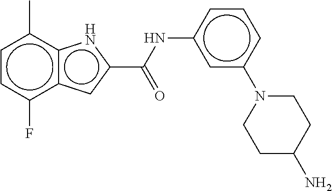 |
| 193 | 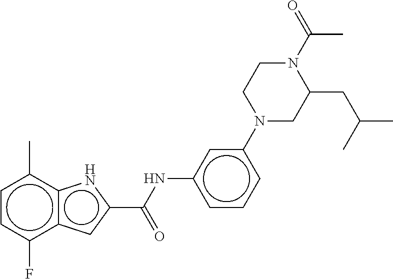 |
| 194 | 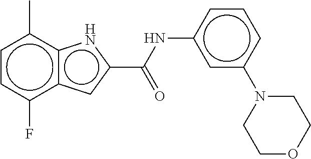 |
| 195 | 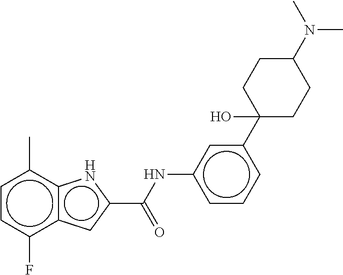 |
| 196 | 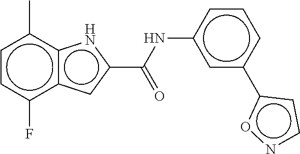 |
| 197 | 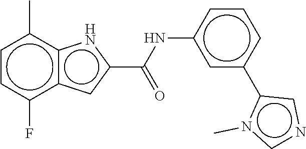 |
| 198 | 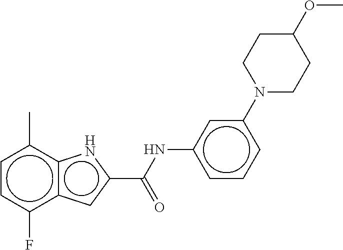 |
| 199 | 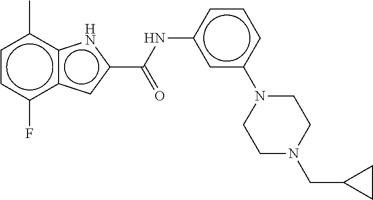 |
| 200 | 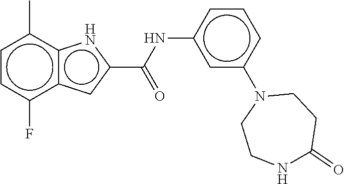 |
| 201 | 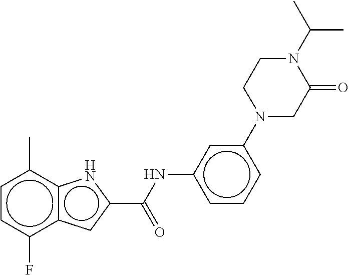 |
| 202 | 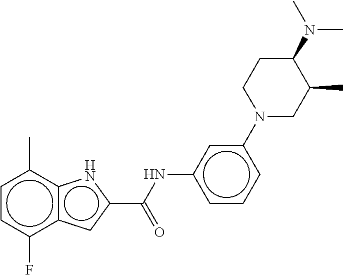 |
| 203 | 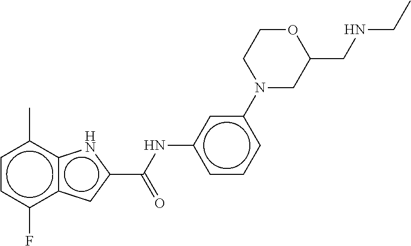 |
| 204 | 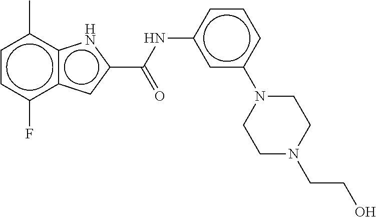 |
| 205 | 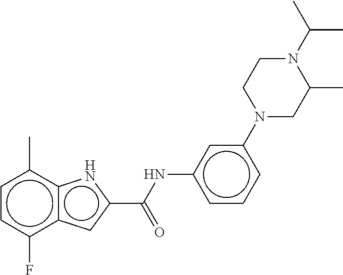 |
| 206 | 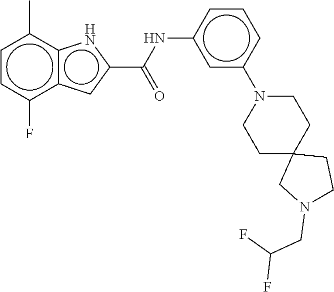 |
| 207 | 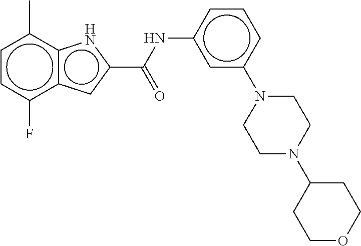 |
| 208 | 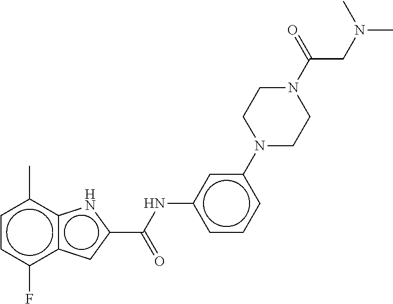 |
| 209 | 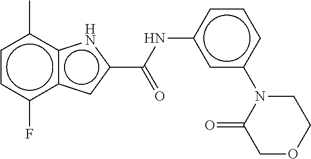 |
| 210 | 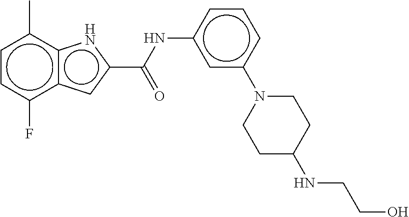 |
| 211 | 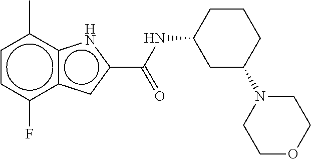 |
| 212 | 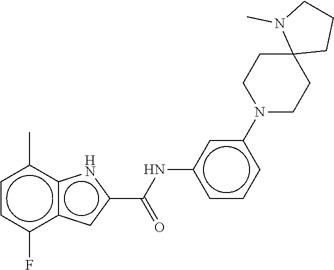 |
| 213 | 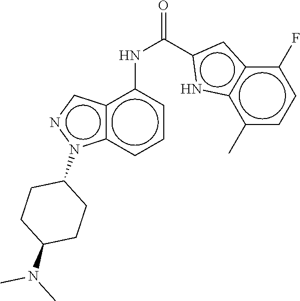 |
| 214 | 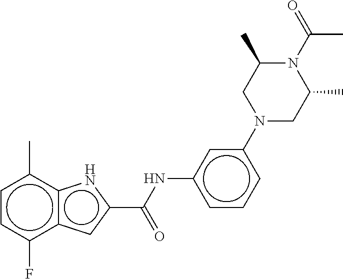 |
| 215 | 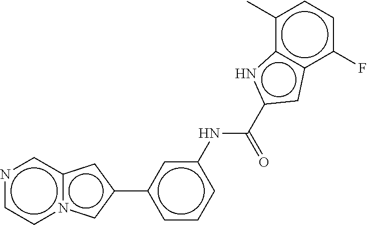 |
| 216 | 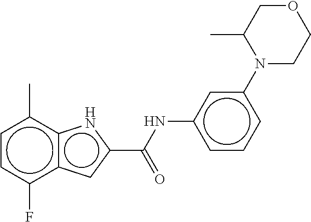 |
| 217 | 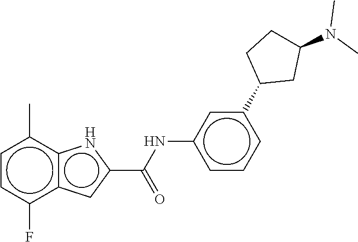 |
| 218 | 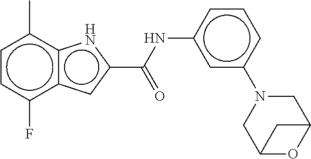 |
| 219 | 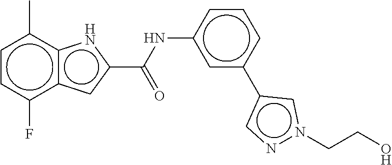 |
| 220 | 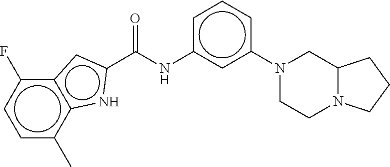 |
| 221 | 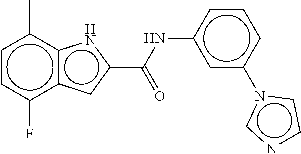 |
| 222 | 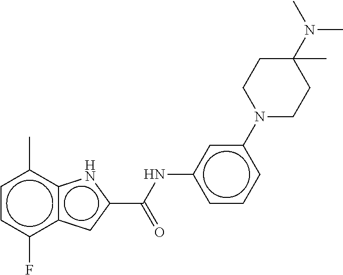 |
| 223 | 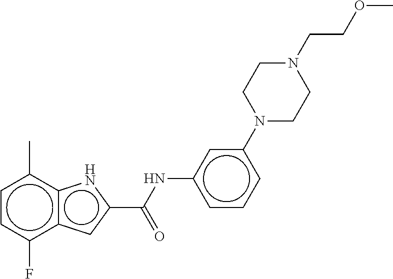 |
| 224 | 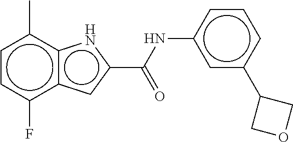 |
| 225 | 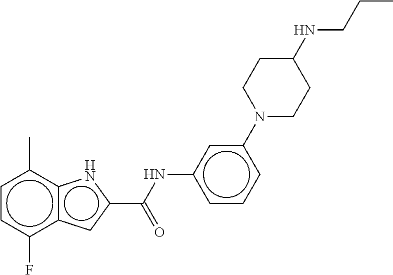 |
| 226 | 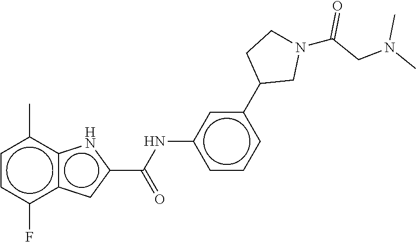 |
| 227 | 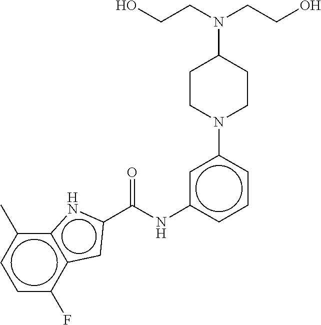 |
| 228 | 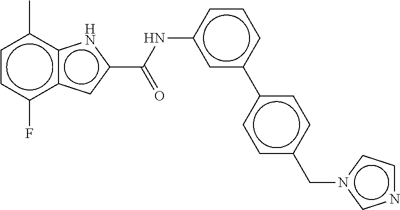 |
| 229 | 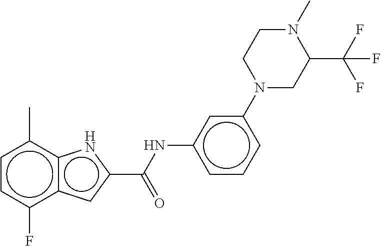 |
| 230 | 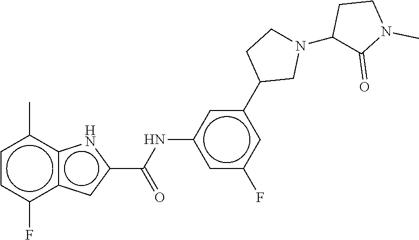 |
| 231 | 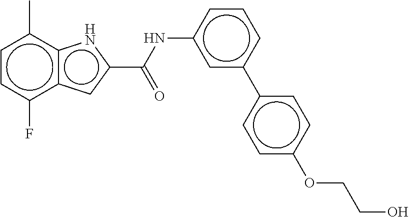 |
| 232 | 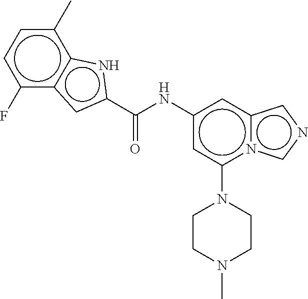 |
| 233 | 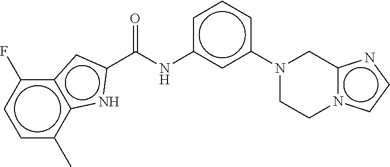 |
| 234 | 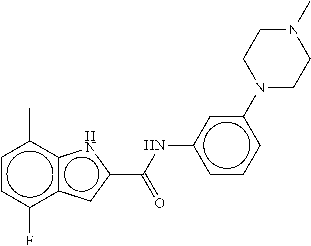 |
| 235 | 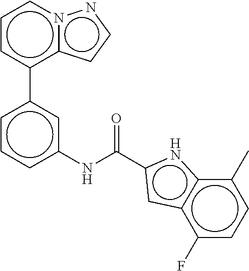 |
| 236 | 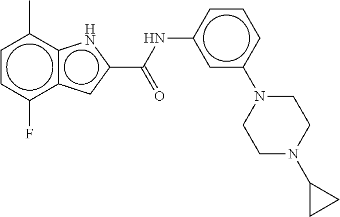 |
| 237 | 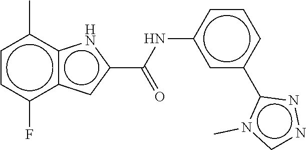 |
| 238 | 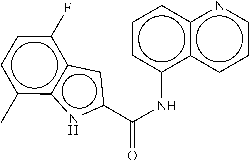 |
| 239 | 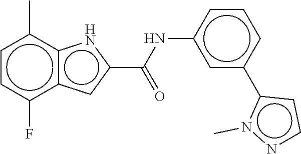 |
| 240 | 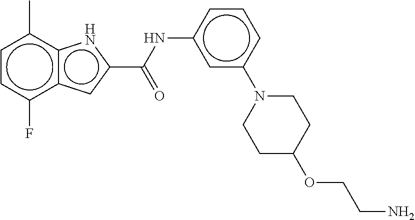 |
| 241 | 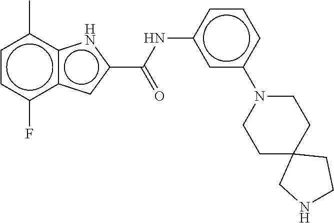 |
| 242 | 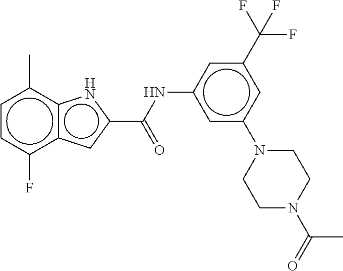 |
| 243 | 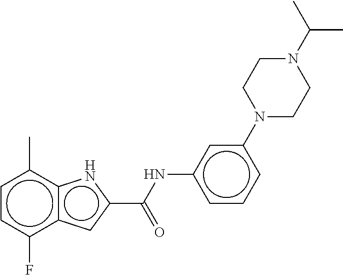 |
| 244 | 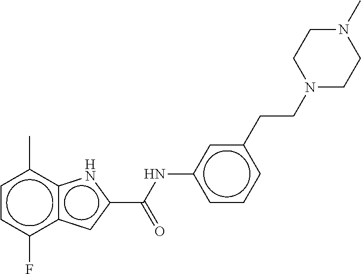 |
| 245 | 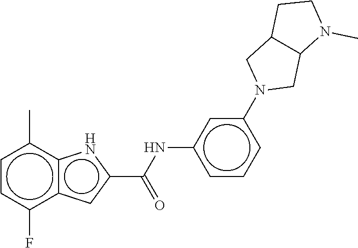 |
| 246 | 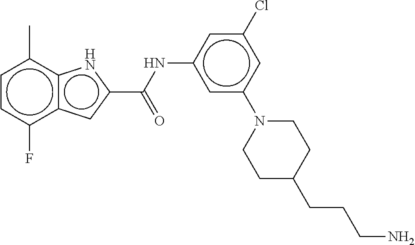 |
| 247 | 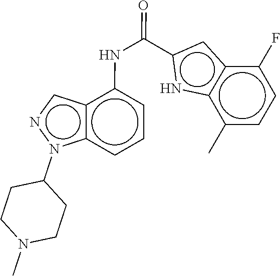 |
| 248 | 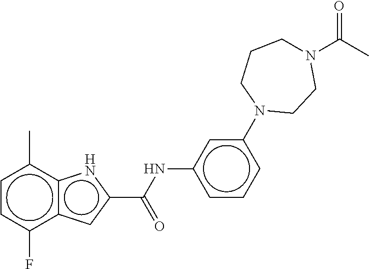 |
| 249 | 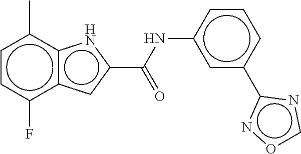 |
| 250 | 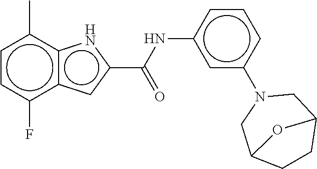 |
| 251 | 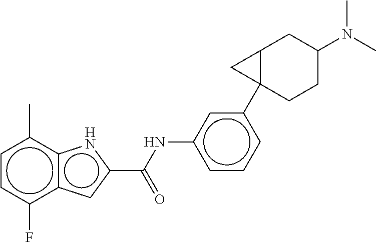 |
| 252 | 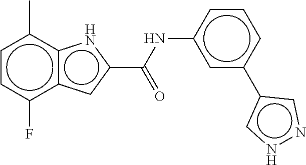 |
| 253 | 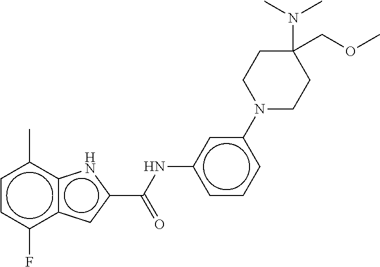 |
| 254 | 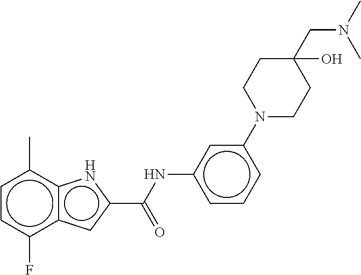 |
| 255 | 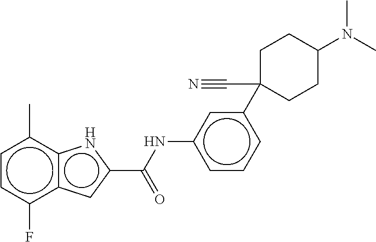 |
| 256 | 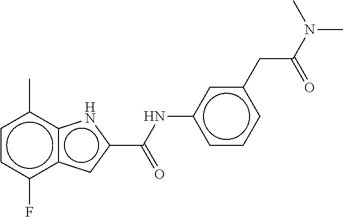 |
| 257 | 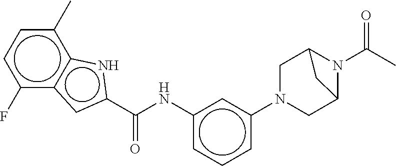 |
| 258 | 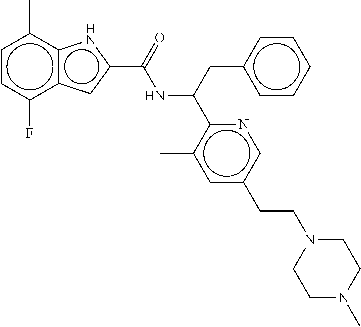 |
| 259 | 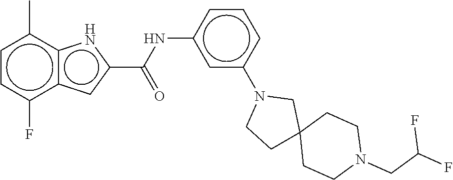 |
| 260 | 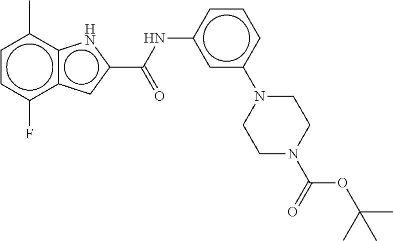 |
| 261 | 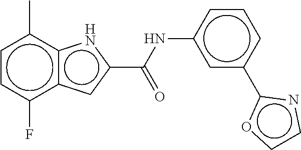 |
| 262 | 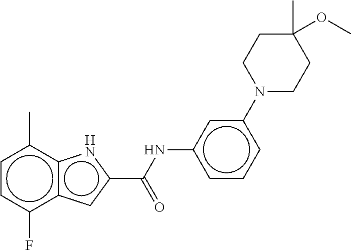 |
| 263 | 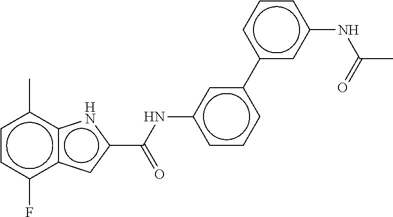 |
| 264 | 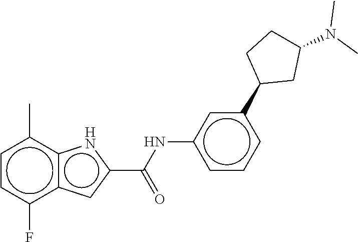 |
| 265 | 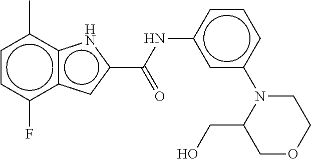 |
| 266 | 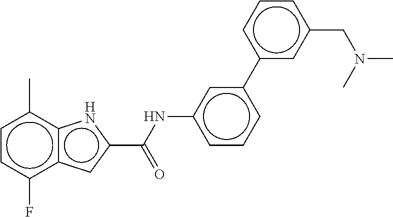 |
| 267 | 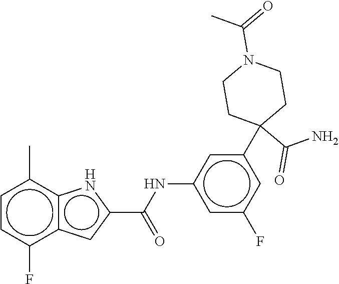 |
| 268 | 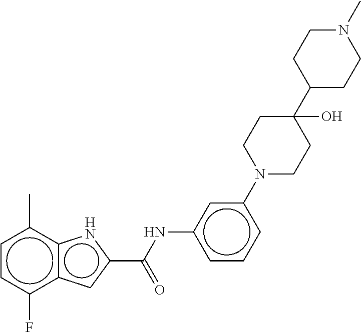 |
| 269 | 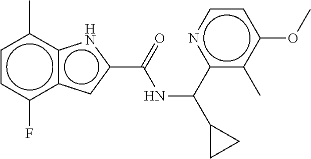 |
| 270 | 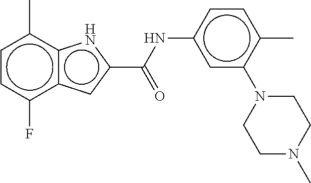 |
| 271 | 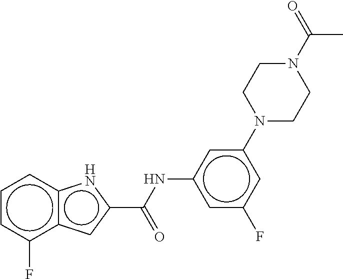 |
| 272 | 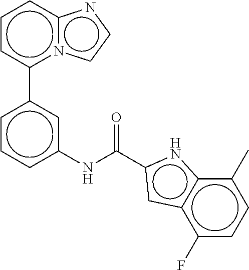 |
| 273 | 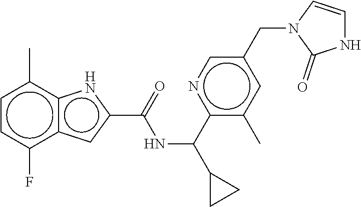 |
| 274 | 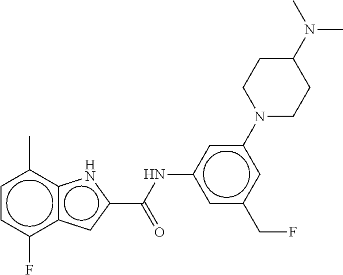 |
| 276 | 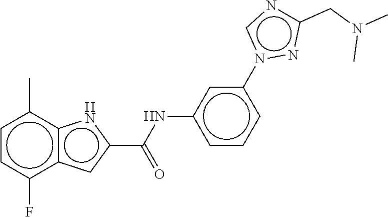 |
| 277 | 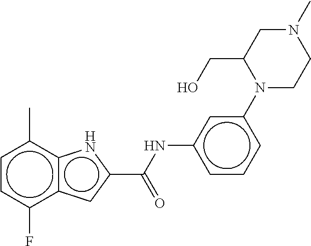 |
| 278 | 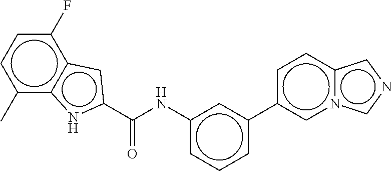 |
| 279 | 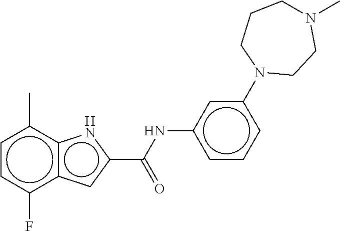 |
| 280 | 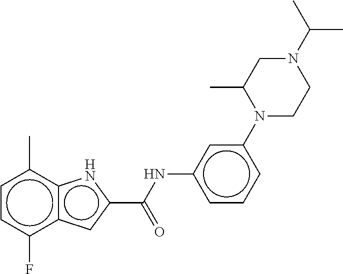 |
| 281 | 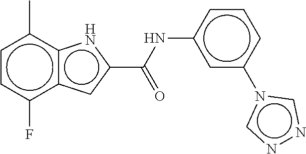 |
| 282 | 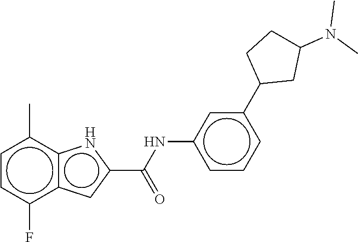 |
| 283 | 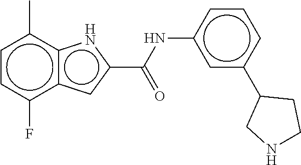 |
| 284 | 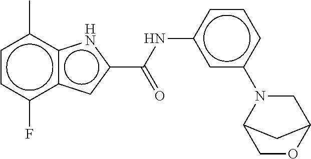 |
| 285 | 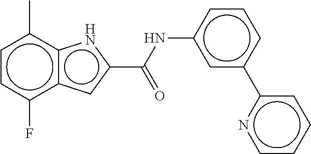 |
| 286 | 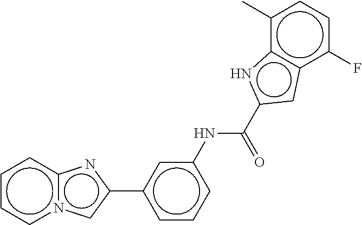 |
| 287 | 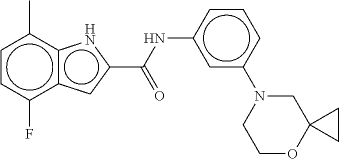 |
| 288 | 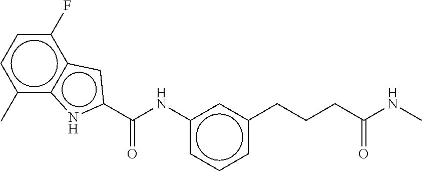 |
| 289 | 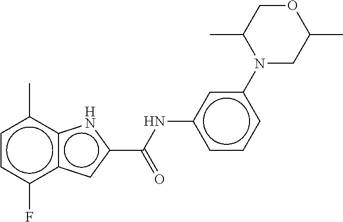 |
| 290 | 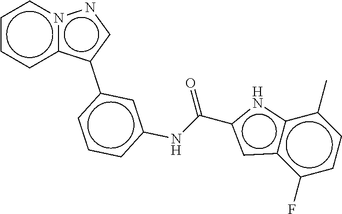 |
| 291 | 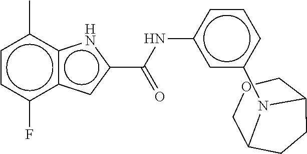 |
| 292 | 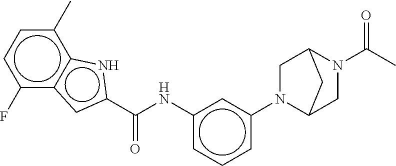 |
| 293 | 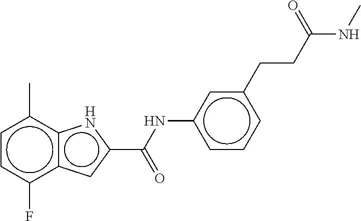 |
| 294 | 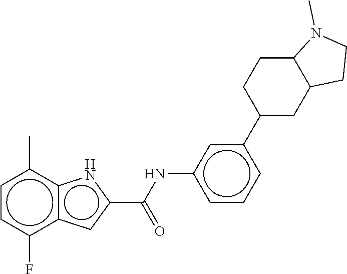 |
| 295 | 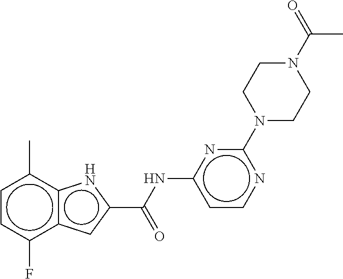 |
| 296 | 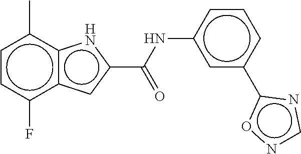 |
| 297 | 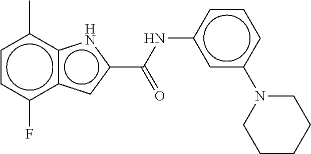 |
| 298 | 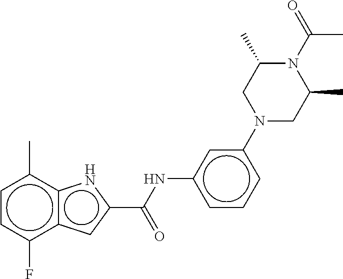 |
| 299 | 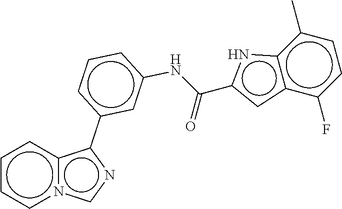 |
| 300 | 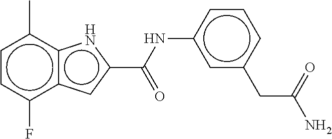 |
| 301 | 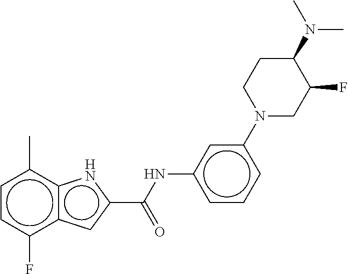 |
| 302 | 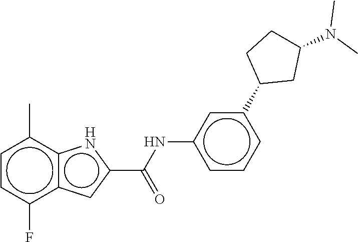 |
| 303 | 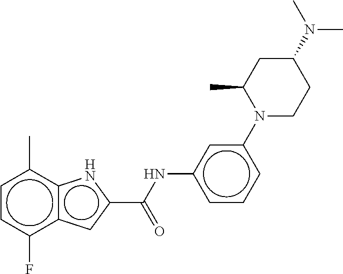 |
| 304 | 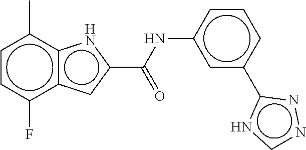 |
| 305 | 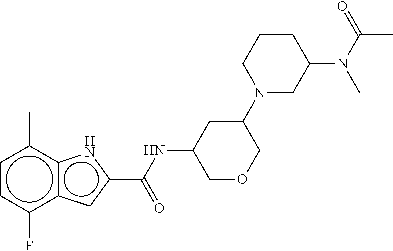 |
| 306 | 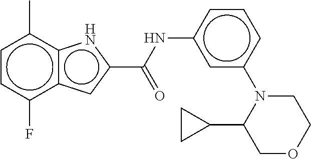 |
| 307 | 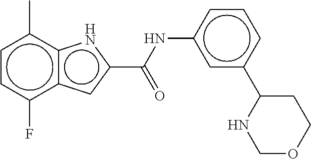 |
| 308 | 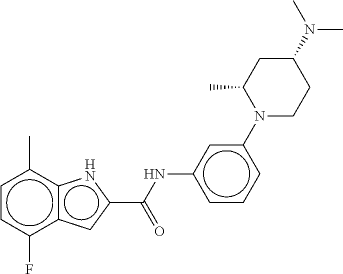 |
| 309 | 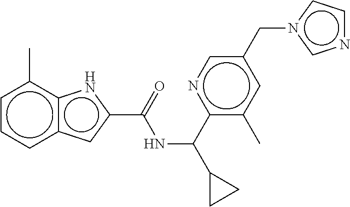 |
| 310 | 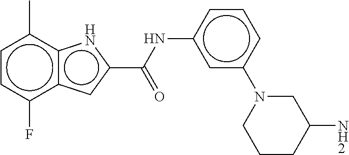 |
| 311 | 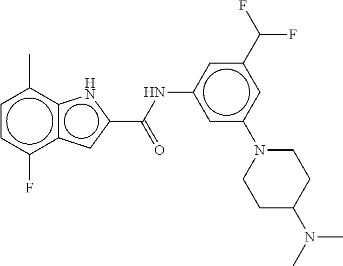 |
| 312 | 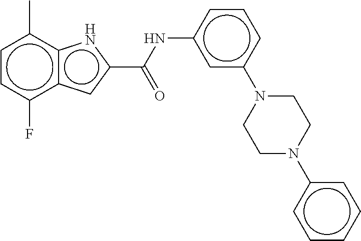 |
| 313 | 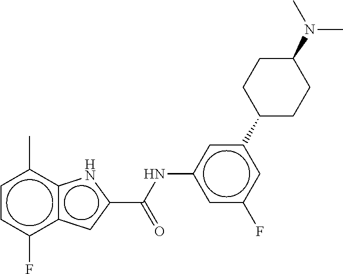 |
| 314 | 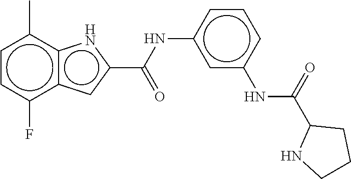 |
| 315 | 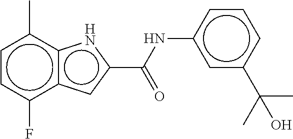 |
| 316 | 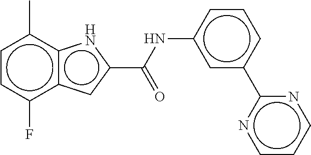 |
| 317 | 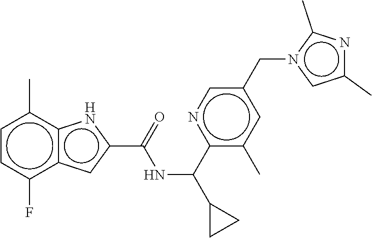 |
| 318 | 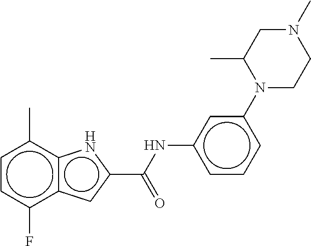 |
| 319 | 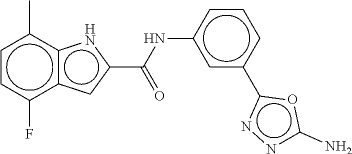 |
| 320 | 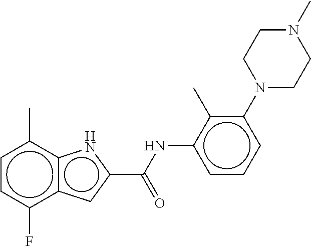 |
| 321 | 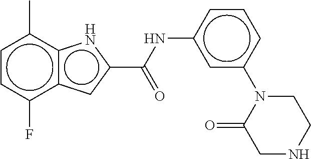 |
| 322 | 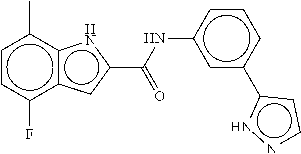 |
| 323 | 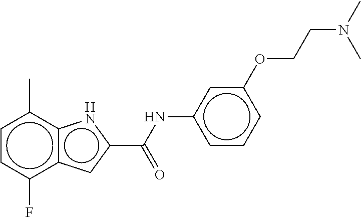 |
| 324 | 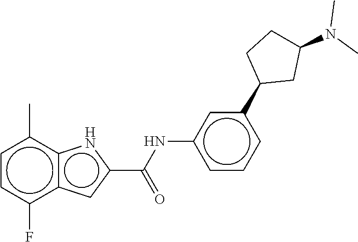 |
| 325 | 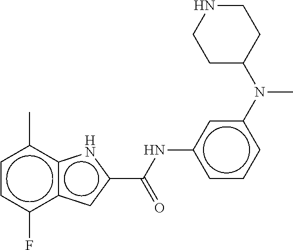 |
| 326 | 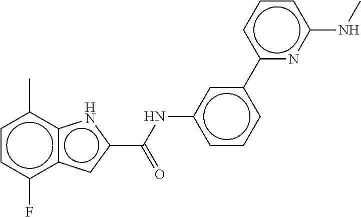 |
| 327 | 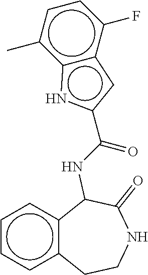 |
| 328 | 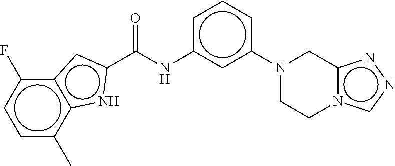 |
| 329 | 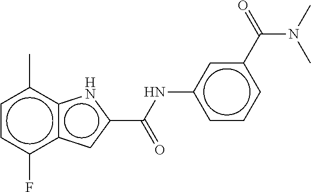 |
| 330 | 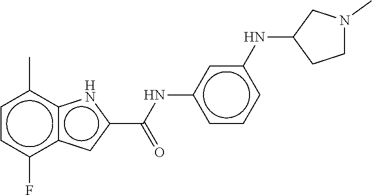 |
| 331 | 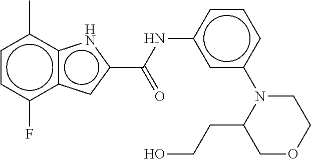 |
| 332 | 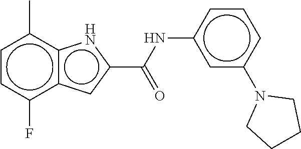 |
| 333 | 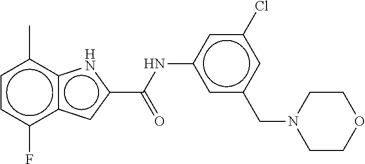 |
| 334 | 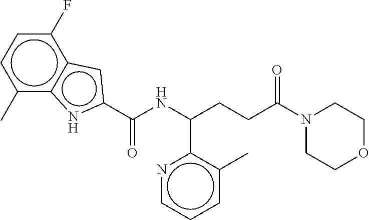 |
| 335 | 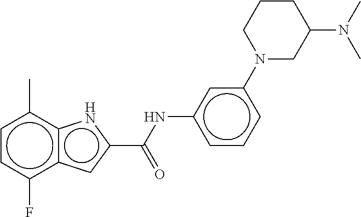 |
| 336 | 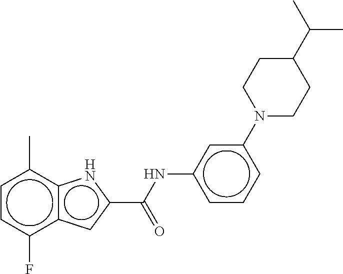 |
| 337 | 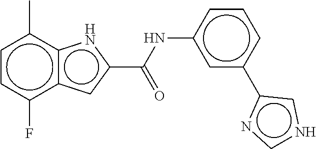 |
| 338 | 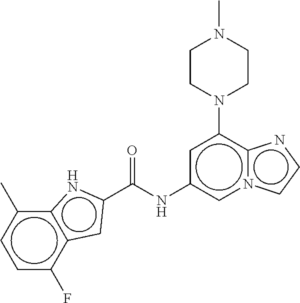 |
| 339 | 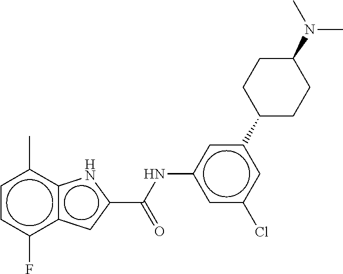 |
| 340 | 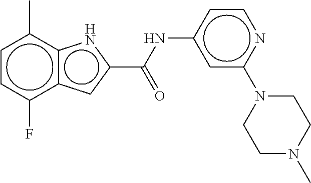 |
| 341 | 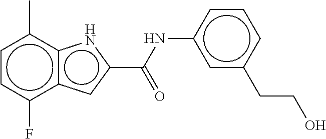 |
| 342 | 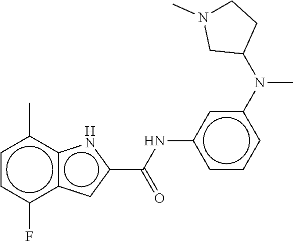 |
| 343 | 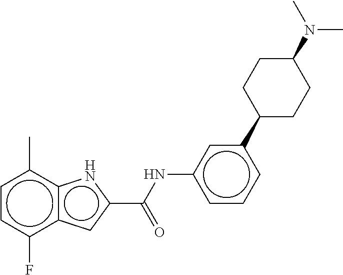 |
| 344 | 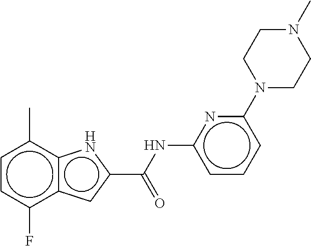 |
| 345 | 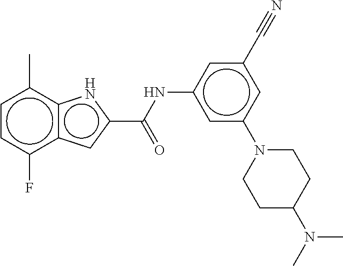 |
| 346 | 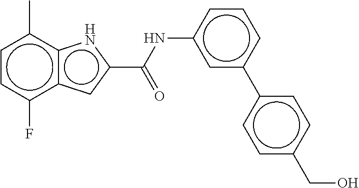 |
| 347 | 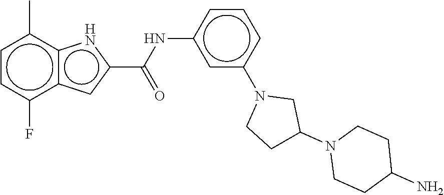 |
| 348 | 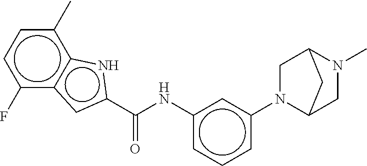 |
| 349 | 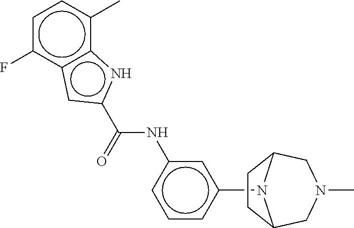 |
| 350 | 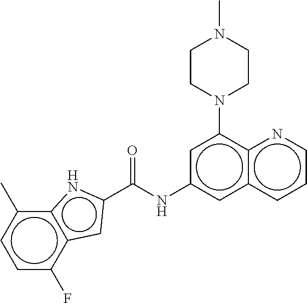 |
| 351 | 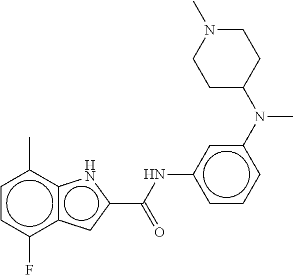 |
| 352 | 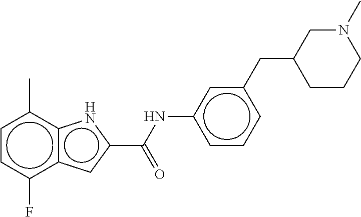 |
| 353 | 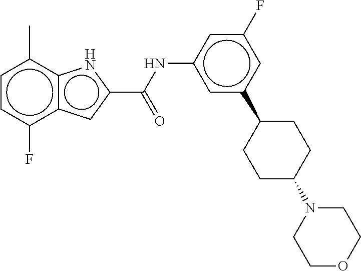 |
| 354 | 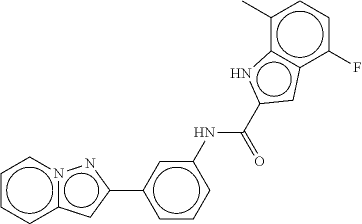 |
| 355 | 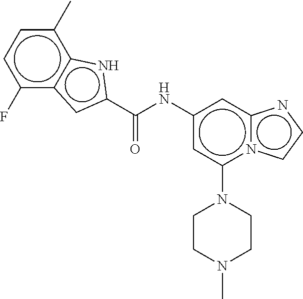 |
| 356 | 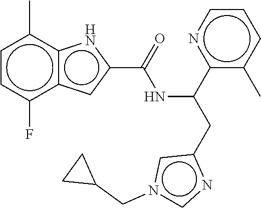 |
| 357 | 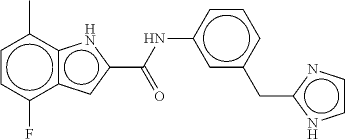 |
| 358 | 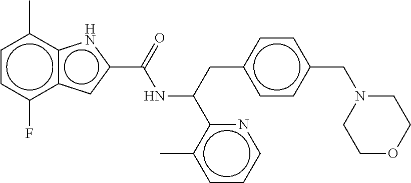 |
| 359 | 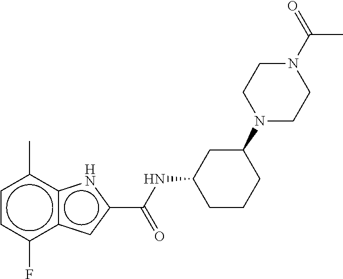 |
| 360 | 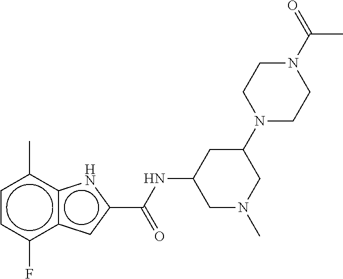 |
| 361 | 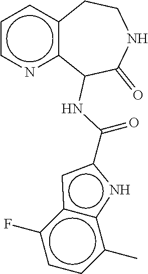 |
| 362 | 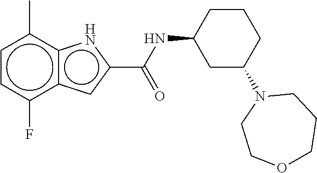 |
| 363 | 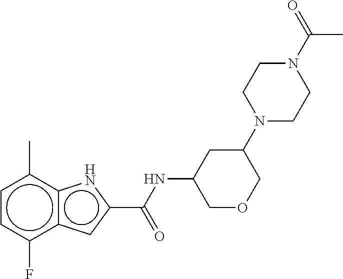 |
| 364 | 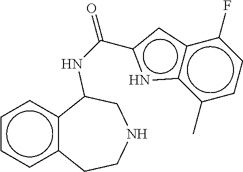 |
| 365 | 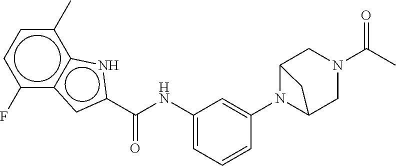 |
| 366 | 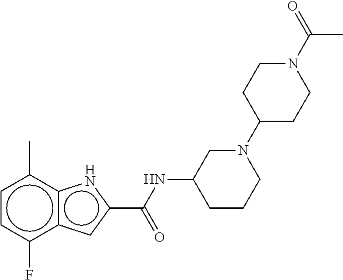 |
| 367 | 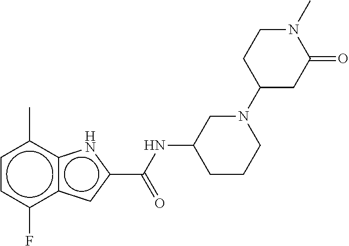 |
| 368 | 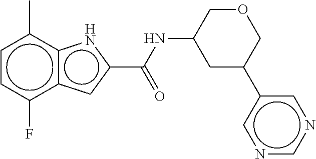 |
| 369 | 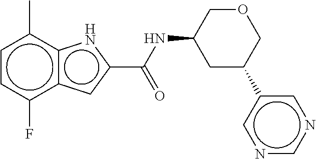 |
| 370 | 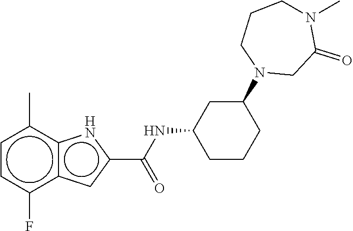 |
| 371 | 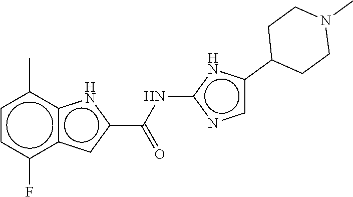 |
| 372 | 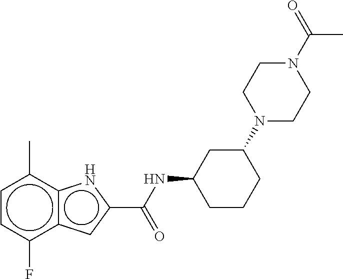 |
| 373 | 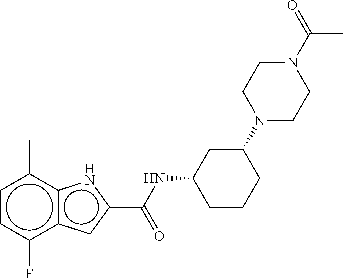 |
| 374 | 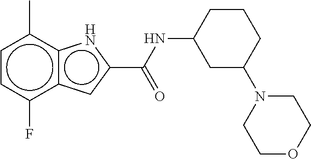 |
| 375 | 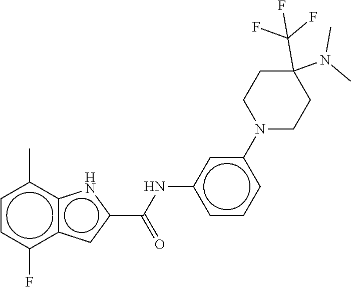 |
| 376 | 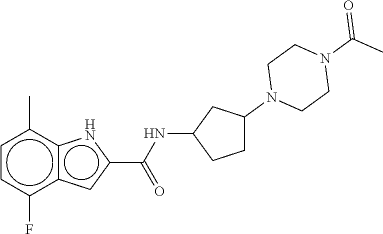 |
| 377 | 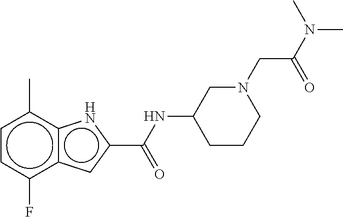 |
| 378 | 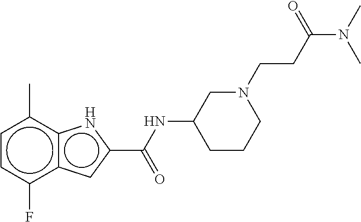 |
| 379 | 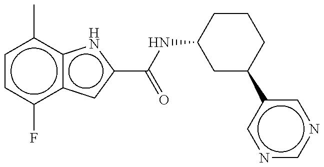 |
| 380 | 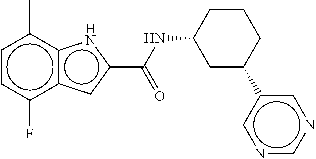 |
| 381 | 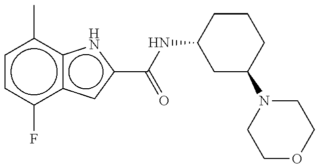 |
| 382 | 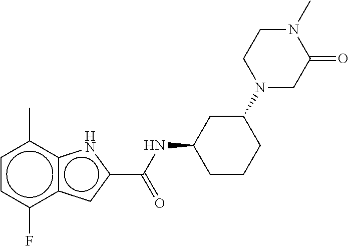 |
| 383 | 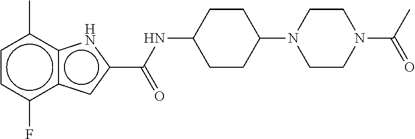 |
| 384 | 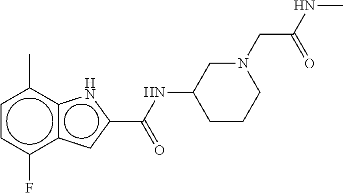 |
| 385 | 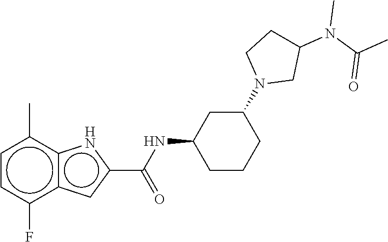 |
| 386 | 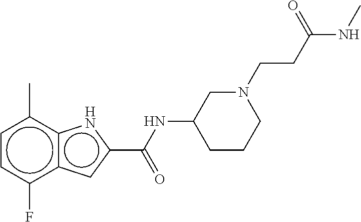 |
| 387 | 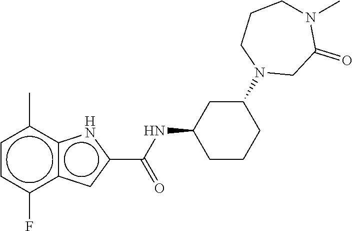 |
| 388 | 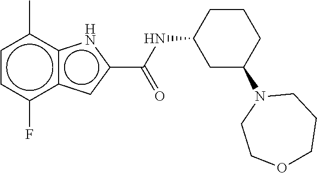 |
| 389 | 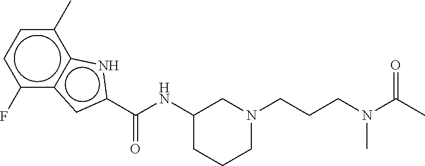 |
| 390 | 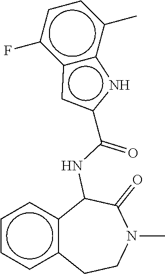 |
| 391 | 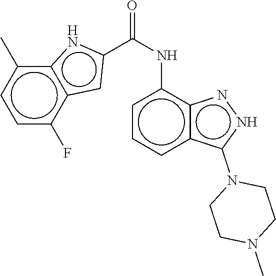 |
| 392 | 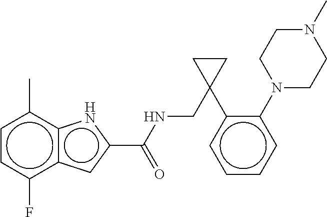 |
| 393 | 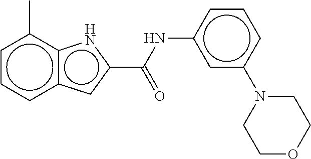 |
| 394 | 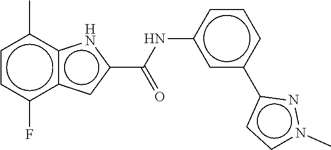 |
| 395 | 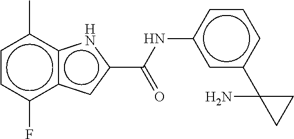 |
| 396 | 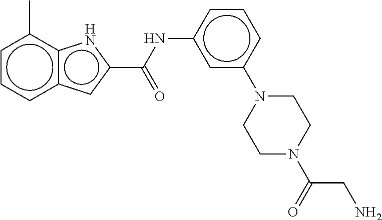 |
| 397 | 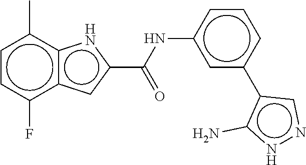 |
| 398 | 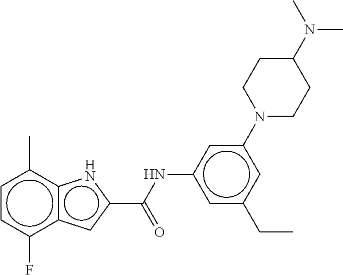 |
| 399 | 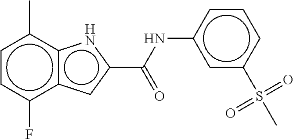 |
| 400 | 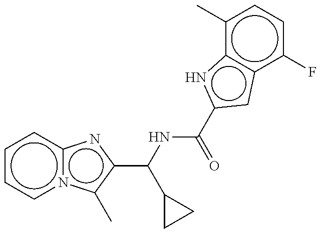 |
| 401 | 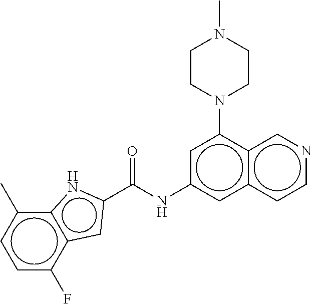 |
| 402 | 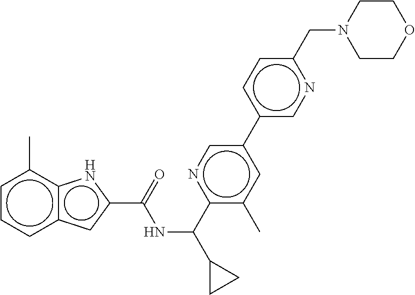 |
| 403 | 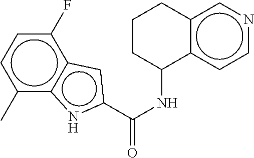 |
| 404 | 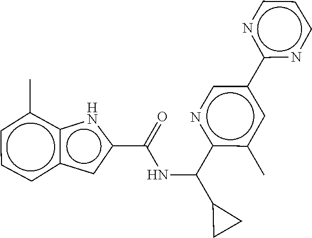 |
| 405 | 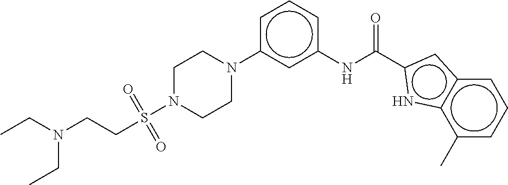 |
| 406 | 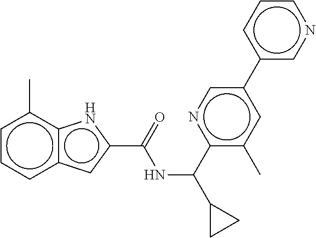 |
| 407 | 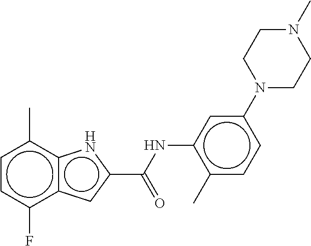 |
| 408 | 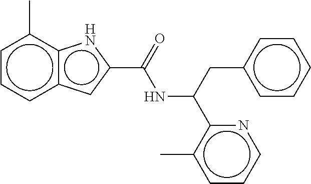 |
| 409 | 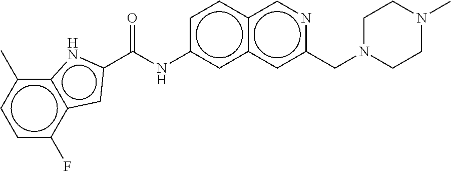 |
| 410 | 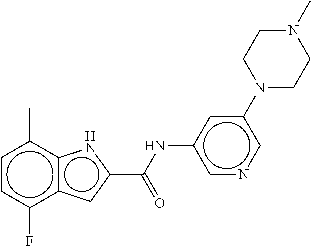 |
| 411 | 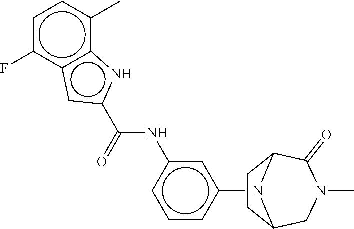 |
| 412 | 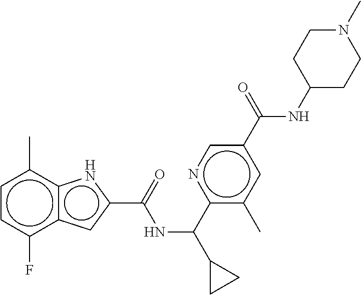 |
| 413 | 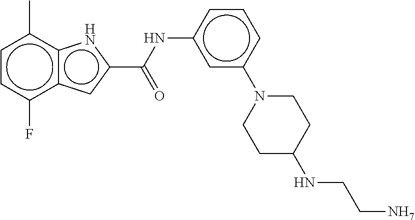 |
| 414 | 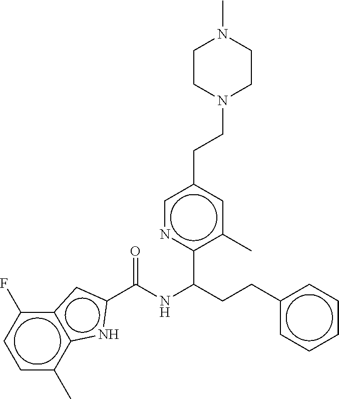 |
| 415 | 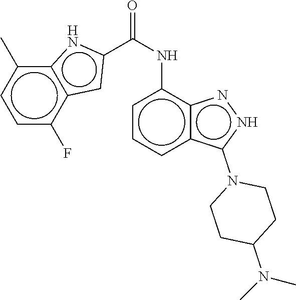 |
| 416 | 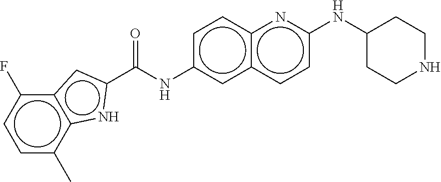 |
| 417 | 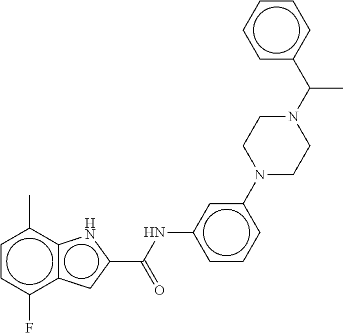 |
| 418 | 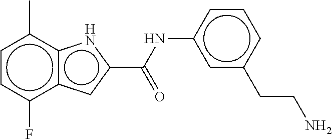 |
| 419 | 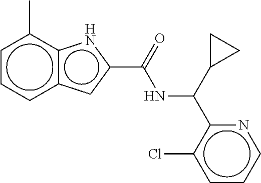 |
| 420 | 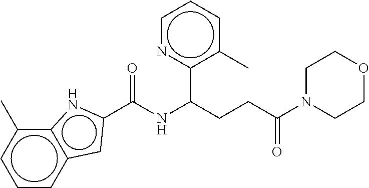 |
| 421 | 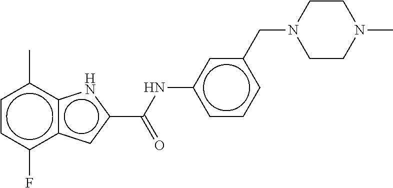 |
| 422 | 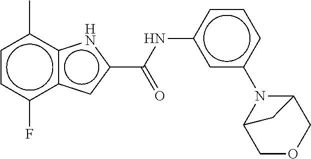 |
| 423 | 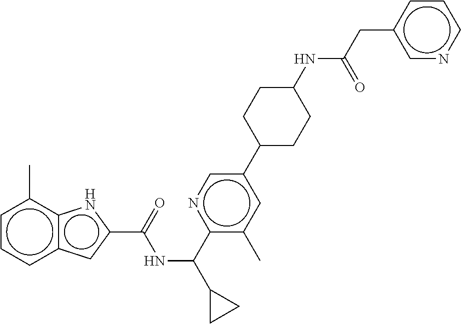 |
| 424 | 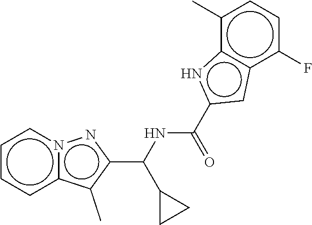 |
| 425 | 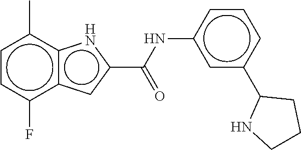 |
| 426 | 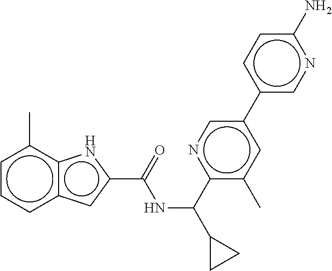 |
| 427 | 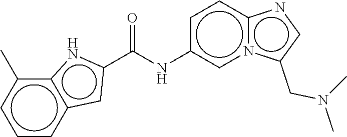 |
| 428 | 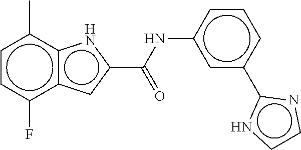 |
| 429 | 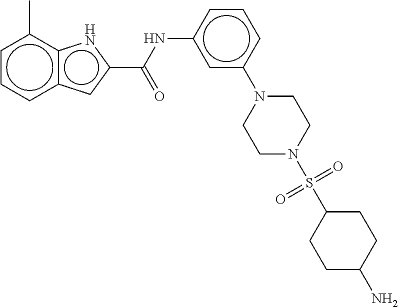 |
| 430 | 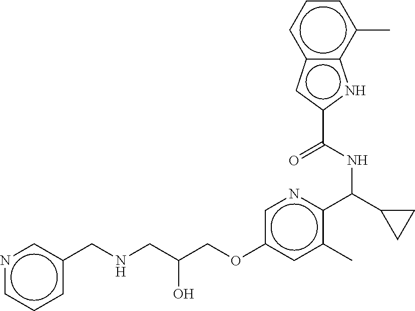 |
| 431 | 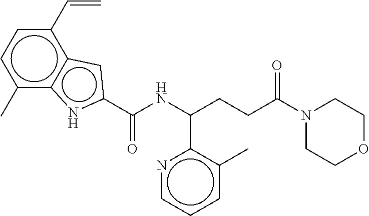 |
| 432 | 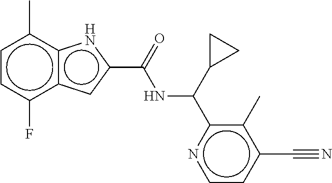 |
| 433 | 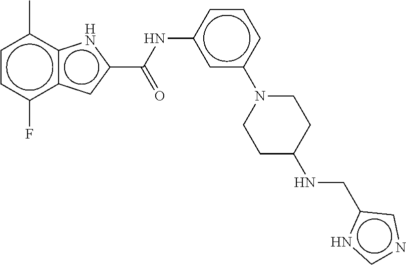 |
| 434 | 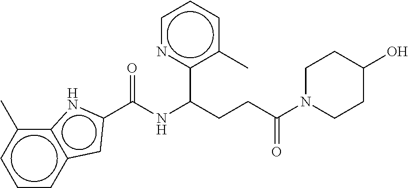 |
| 435 | 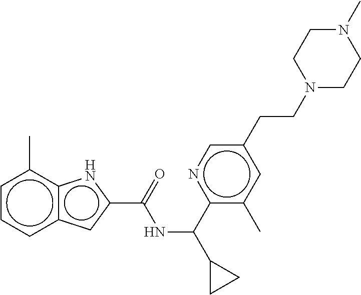 |
| 436 | 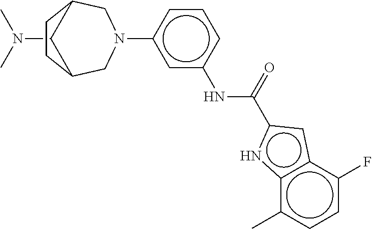 |
| 437 | 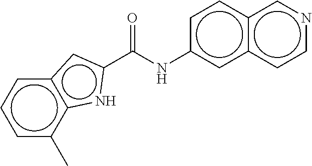 |
| 438 | 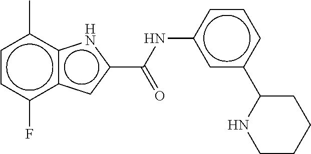 |
| 439 | 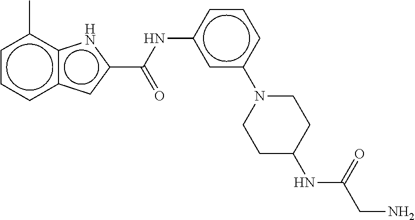 |
| 440 | 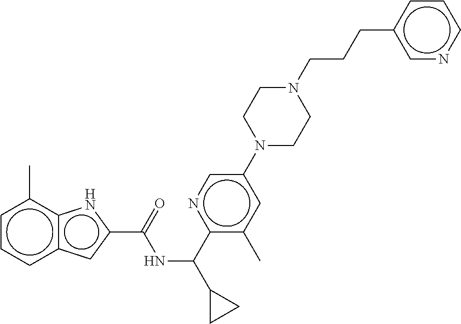 |
| 441 | 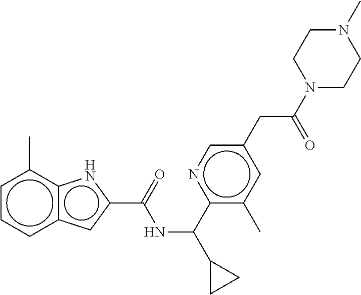 |
| 442 | 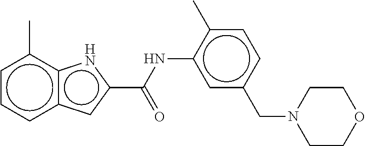 |
| 443 | 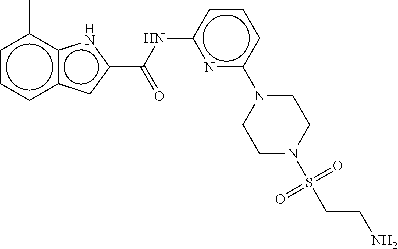 |
| 444 | 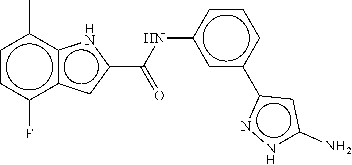 |
| 445 | 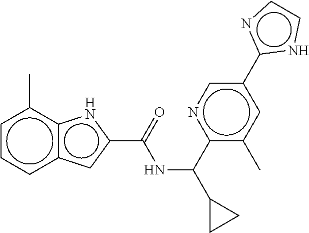 |
| 446 | 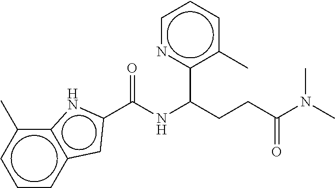 |
| 447 | 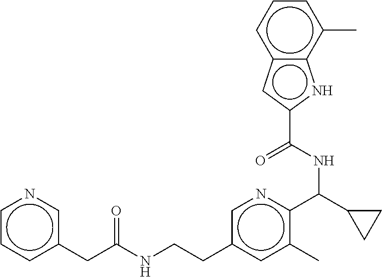 |
| 448 | 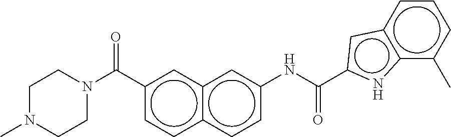 |
| 449 | 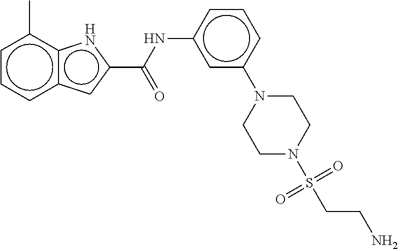 |
| 450 | 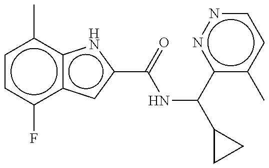 |
| 451 | 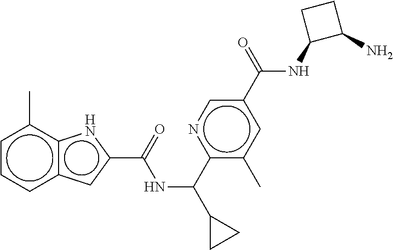 |
| 452 | 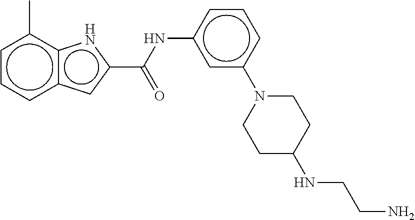 |
| 453 | 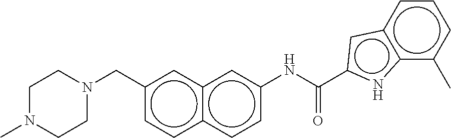 |
| 454 | 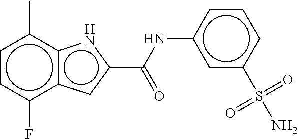 |
| 455 | 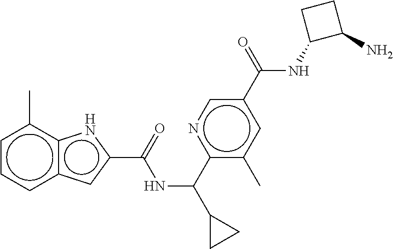 |
| 456 | 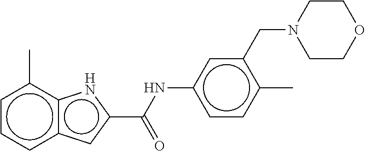 |
| 457 | 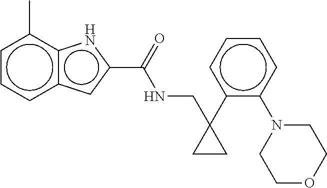 |
| 458 | 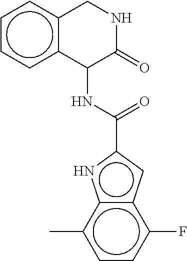 |
| 459 | 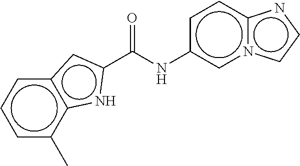 |
| 460 | 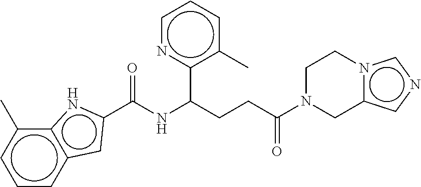 |
| 461 | 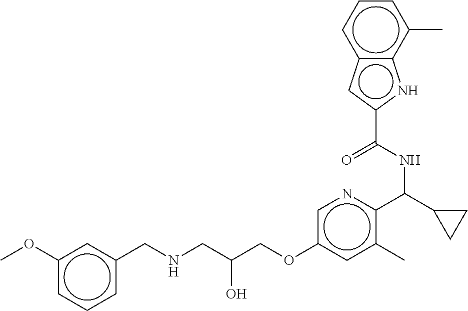 |
| 462 | 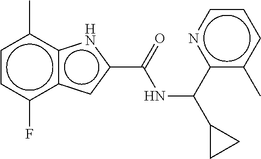 |
| 463 | 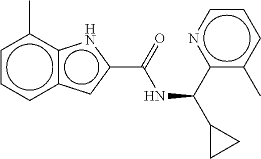 |
| 464 | 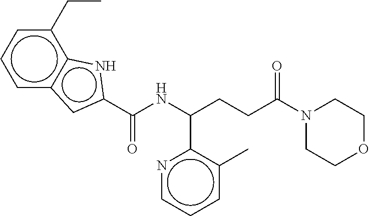 |
| 465 | 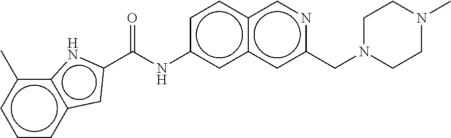 |
| 466 | 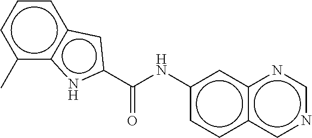 |
| 467 | 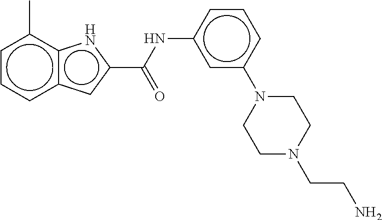 |
| 468 | 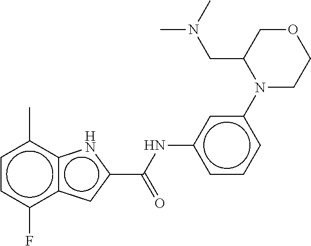 |
| 469 | 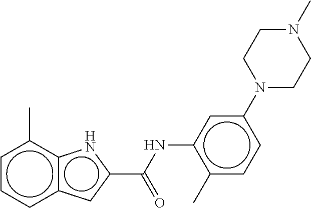 |
| 470 | 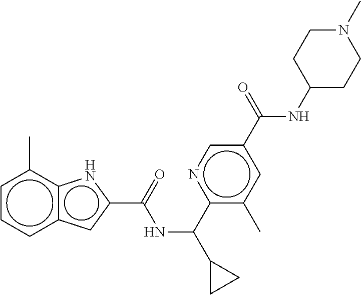 |
| 471 | 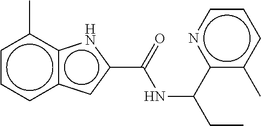 |
| 472 | 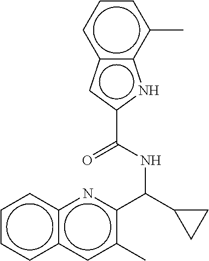 |
| 473 | 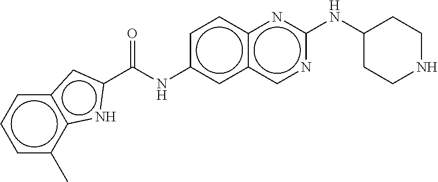 |
| 474 | 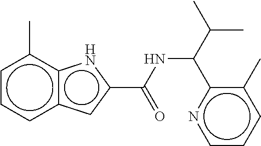 |
| 475 | 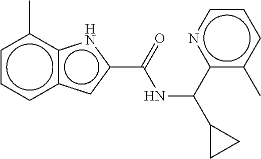 |
| 476 | 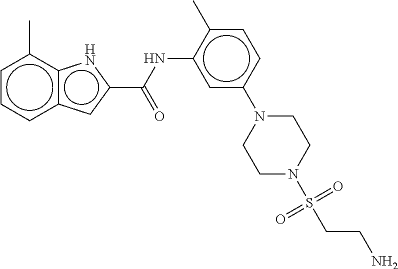 |
| 477 | 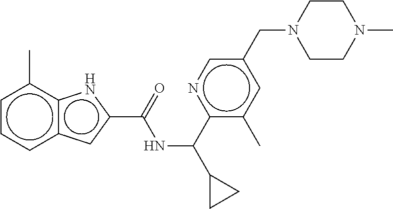 |
| 478 | 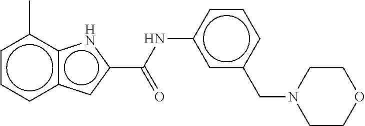 |
| 479 | 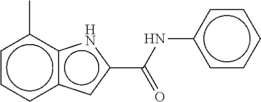 |
| 480 | 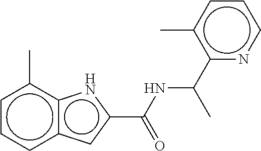 |
| 481 | 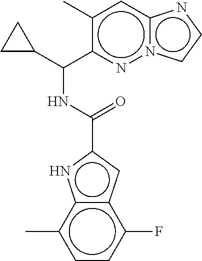 |
| 482 | 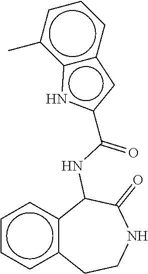 |
| 483 | 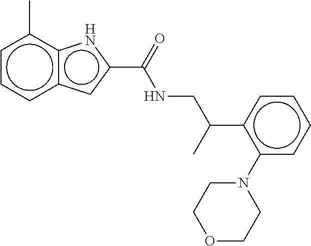 |
| 484 | 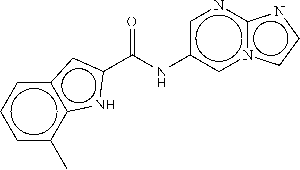 |
| 485 | 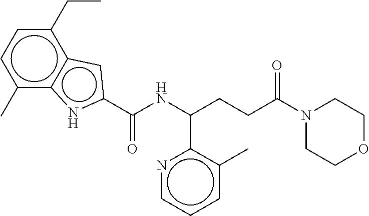 |
| 486 | 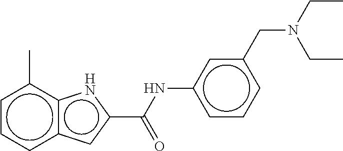 |
| 487 | 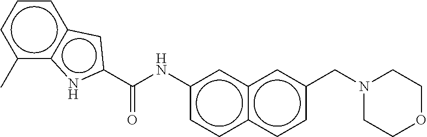 |
| 488 | 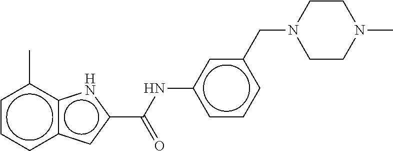 |
| 489 | 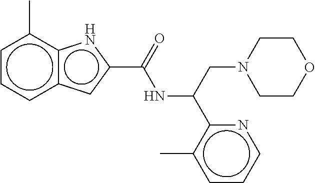 |
| 490 | 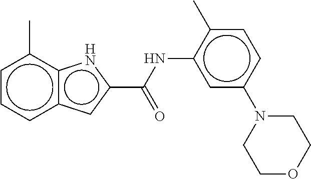 |
| 491 | 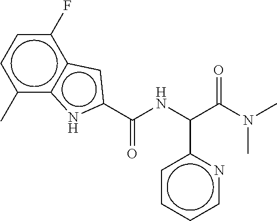 |
| 492 | 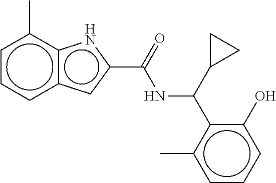 |
| 493 | 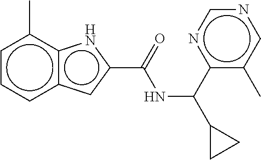 |
| 494 | 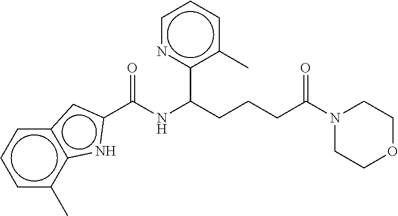 |
| 495 | 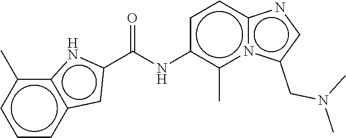 |
| 496 | 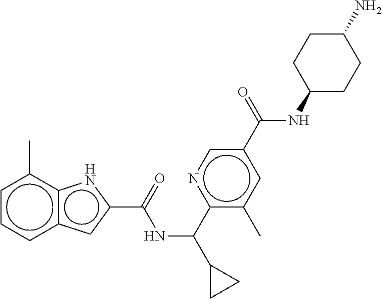 |
| 497 | 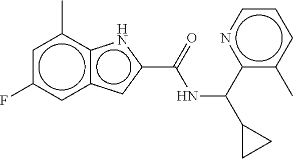 |
| 498 | 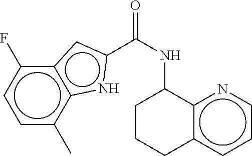 |
| 499 | 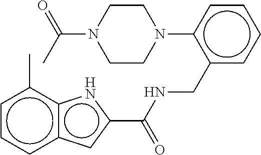 |
| 500 | 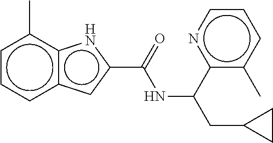 |
| 501 | 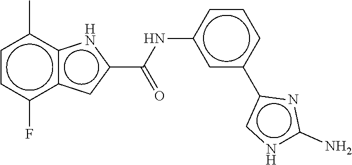 |
| 502 | 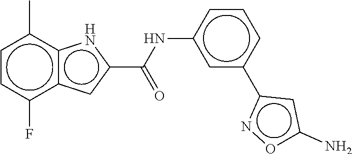 |
| 503 | 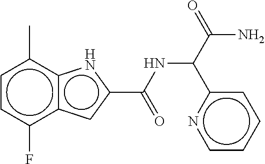 |
| 504 | 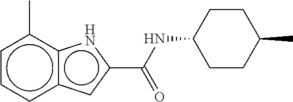 |
| 505 | 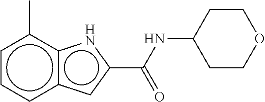 |
| 506 | 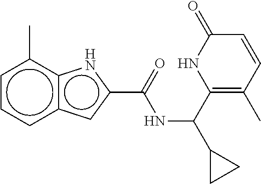 |
| 507 | 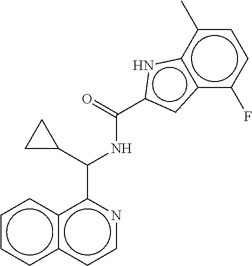 |
| 508 | 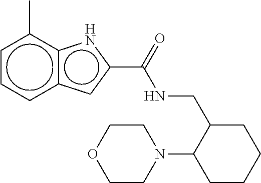 |
| 509 | 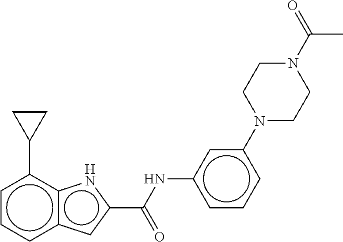 |
| 510 | 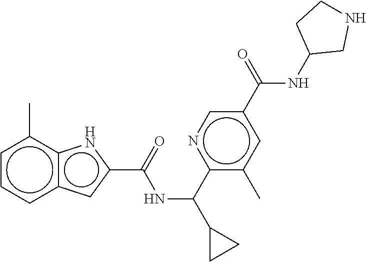 |
| 511 | 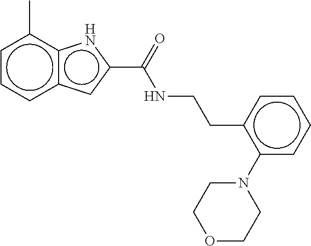 |
| 512 | 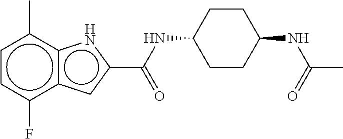 |
| 513 | 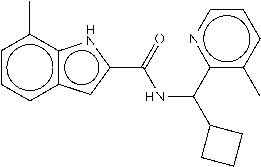 |
| 514 | 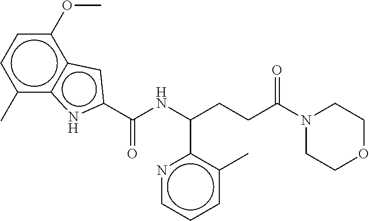 |
| 515 | 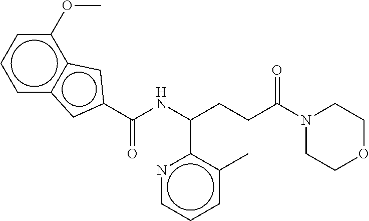 |
| 516 | 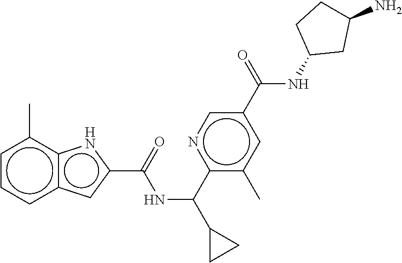 |
| 517 | 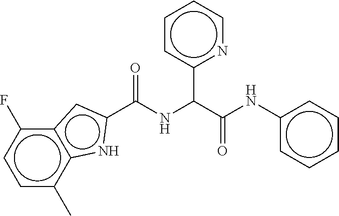 |
| 518 | 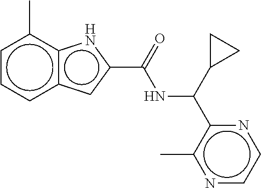 |
| 519 | 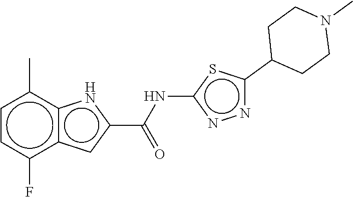 |
| 520 | 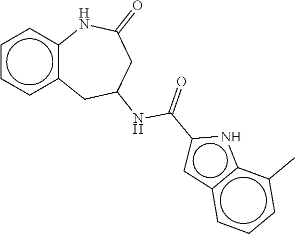 |
| 521 | 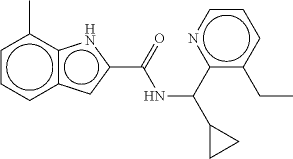 |
| 522 | 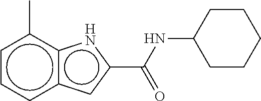 |
| 523 | 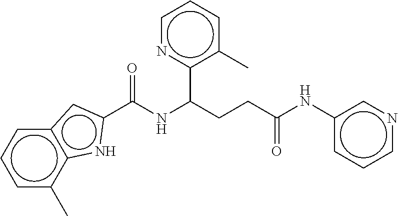 |
| 524 | 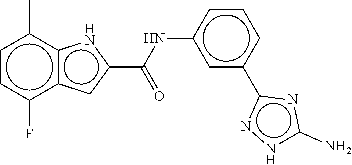 |
| 525 | 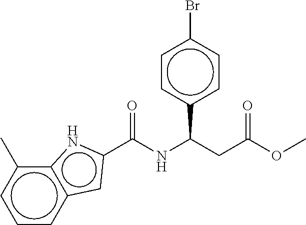 |
| 526 | 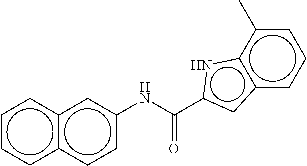 |
| 527 | 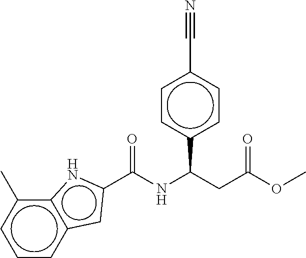 |
| 528 | 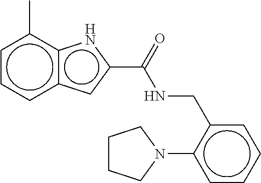 |
| 529 | 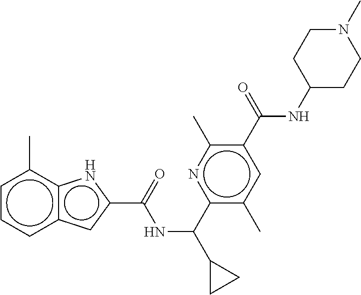 |
| 530 | 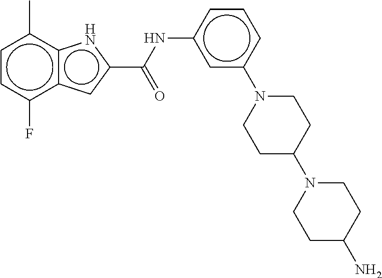 |
| 531 | 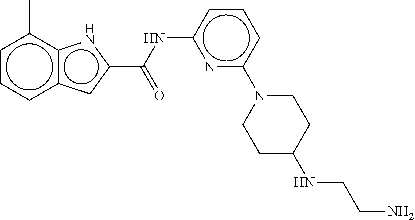 |
| 532 | 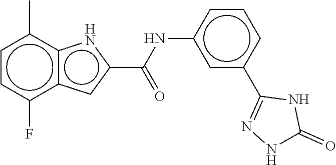 |
| 533 | 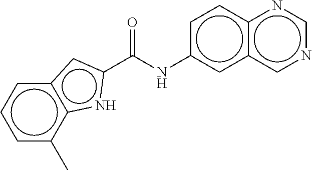 |
| 534 | 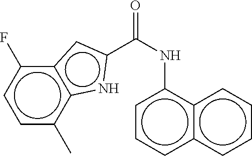 |
| 535 | 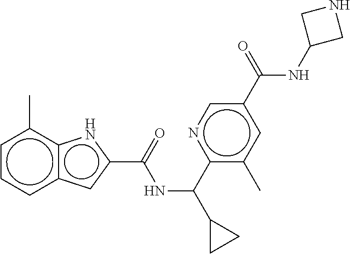 |
| 536 | 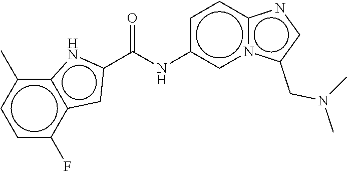 |
| 537 | 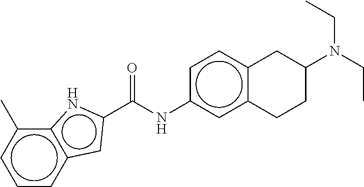 |
| 538 | 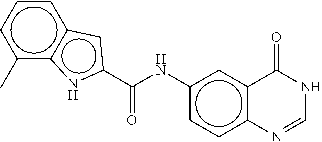 |
| 539 | 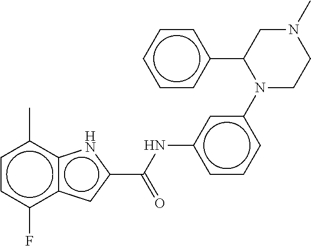 |
| 540 | 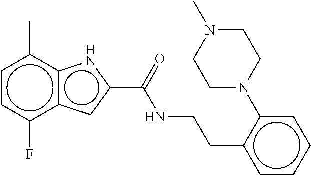 |
| 541 | 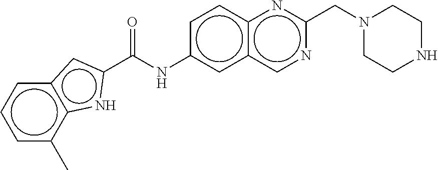 |
| 542 | 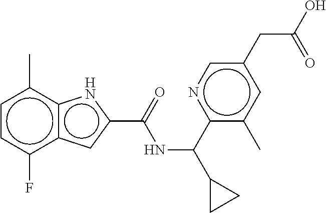 |
| 543 | 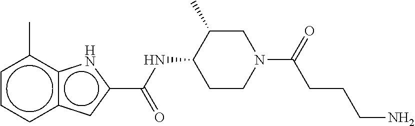 |
| 544 | 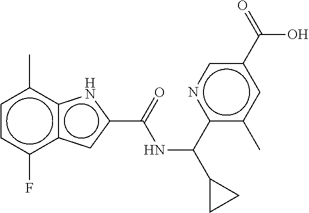 |
| 545 | 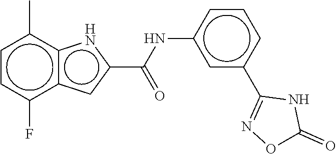 |
| 546 | 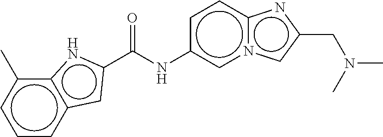 |
| 547 | 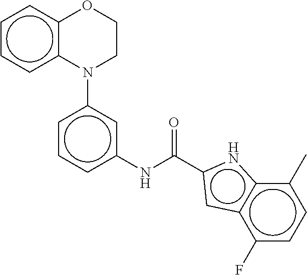 |
| 548 | 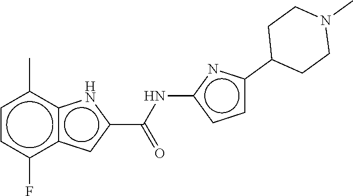 |
| 549 | 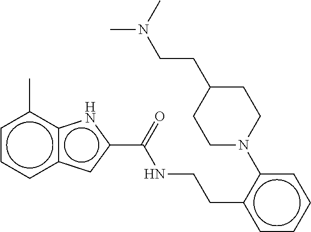 |
| 550 | 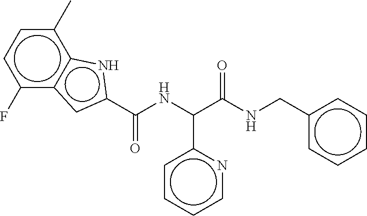 |
| 551 | 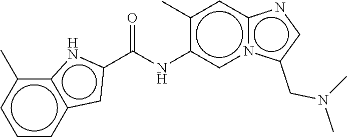 |
| 552 | 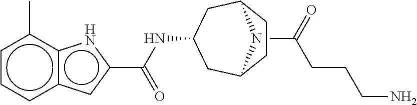 |
| 553 | 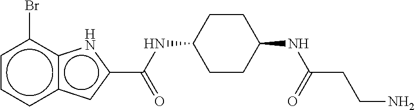 |
| 554 | 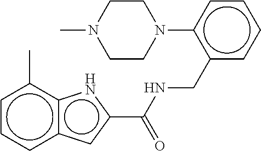 |
| 555 | 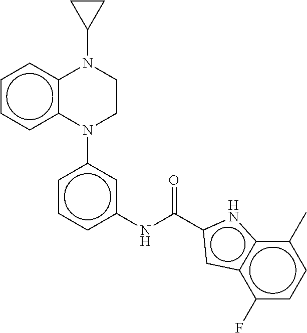 |
| 556 | 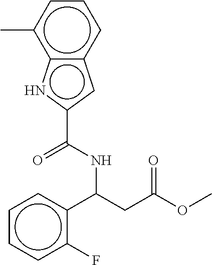 |
| 557 | 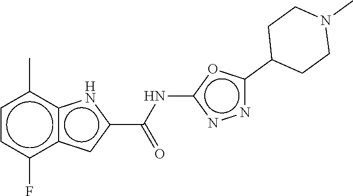 |
| 558 | 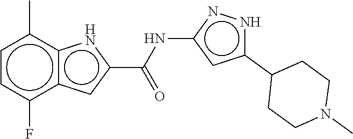 |
| 559 | 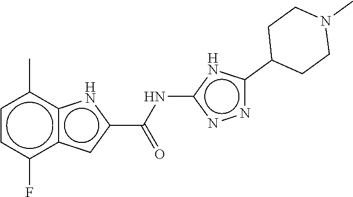 |
| 560 | 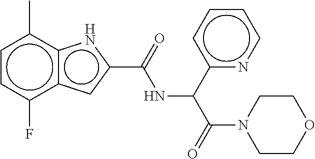 |
| 561 | 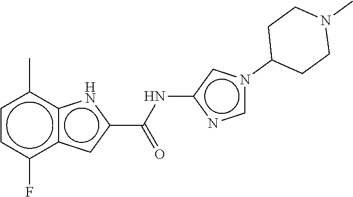 |
| 562 | 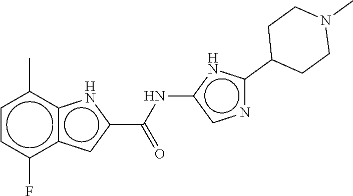 |
| 563 | 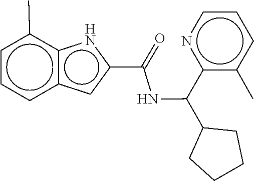 |
| 564 | 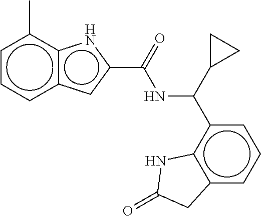 |
| 565 | 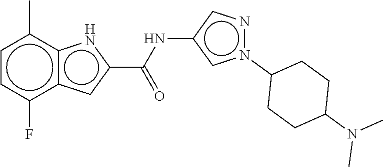 |
| 566 | 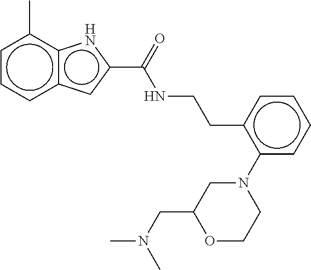 |
| 567 | 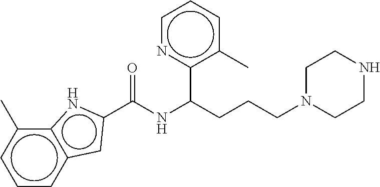 |
| 568 | 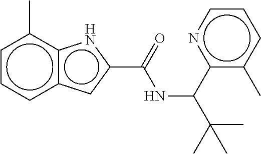 |
| 569 | 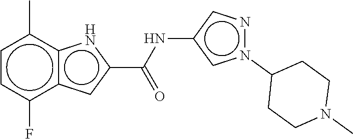 |
| 570 | 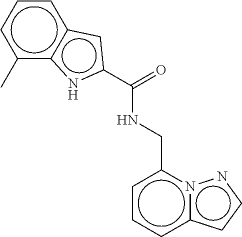 |
| 571 | 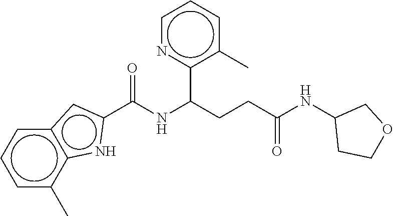 |
| 572 | 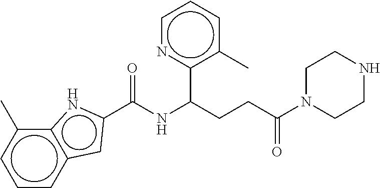 |
| 573 | 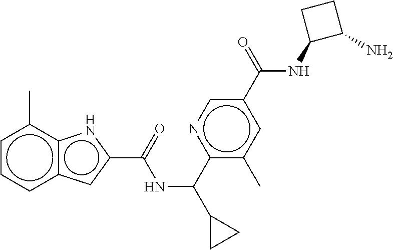 |
| 574 | 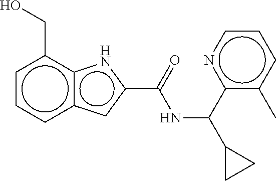 |
| 575 | 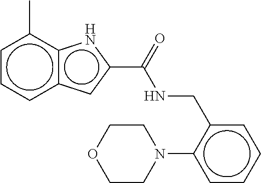 |
| 576 | 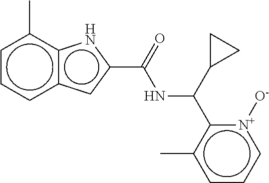 |
| 577 | 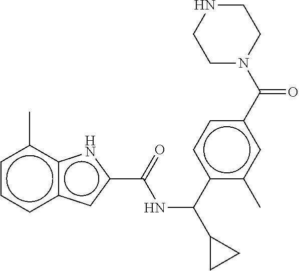 |
| 578 | 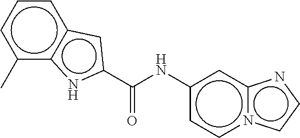 |
| 579 | 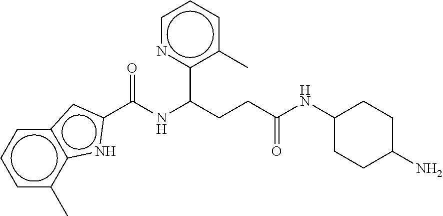 |
| 580 | 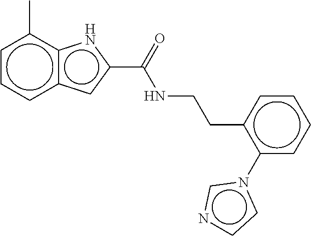 |
| 581 | 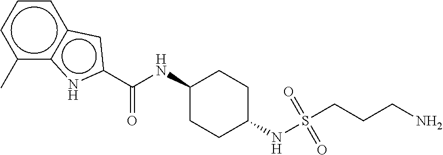 |
| 582 | 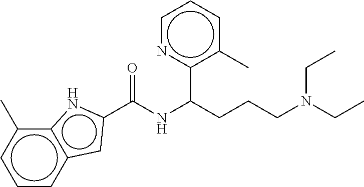 |
| 583 | 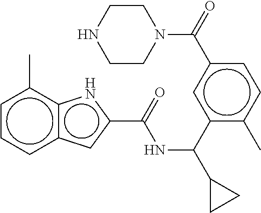 |
| 584 | 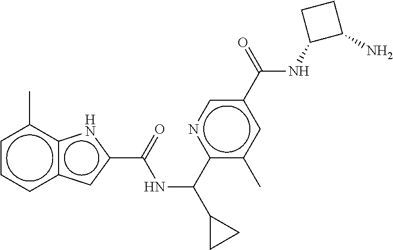 |
| 585 | 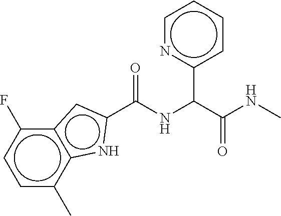 |
| 586 | 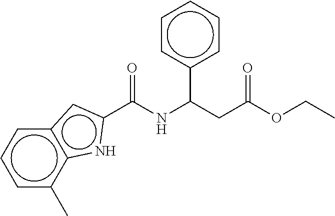 |
| 587 | 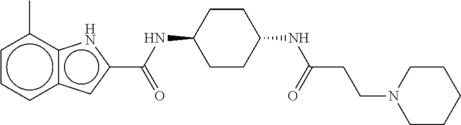 |
| 588 | 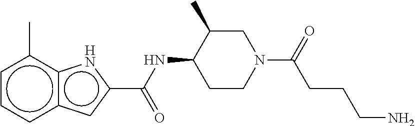 |
| 589 | 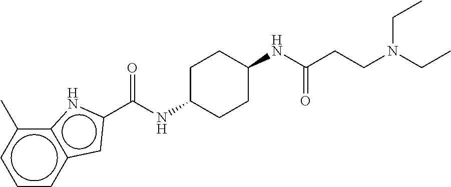 |
| 590 | 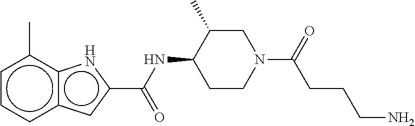 |
| 591 | 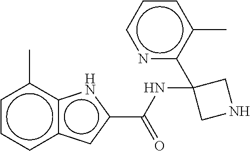 |
| 592 | 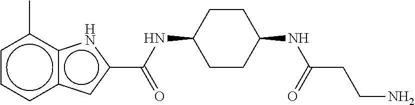 |
| 593 | 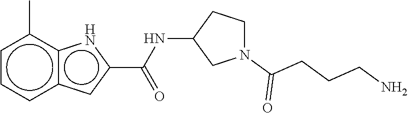 |
| 594 | 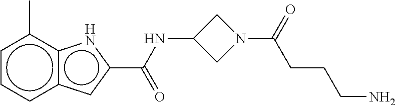 |
| 595 | 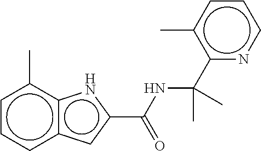 |
| 596 | 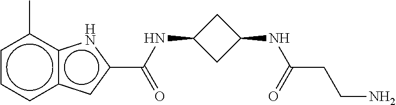 |
| 597 | 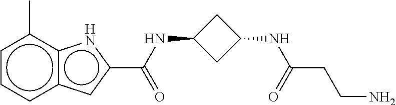 |
| 598 | 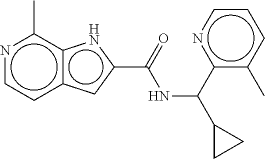 |
| 599 | 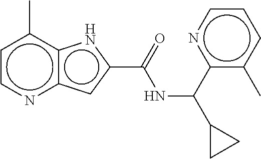 |
| 600 | 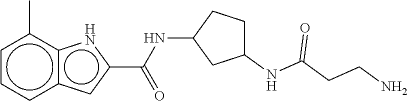 |
| 601 | 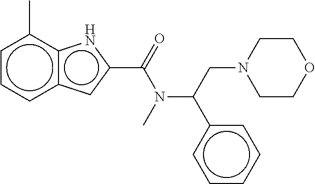 |
| 602 | 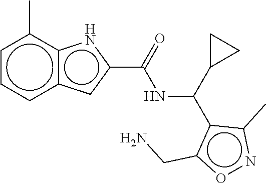 |
| 603 | 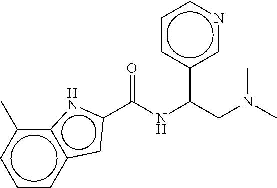 |
| 604 | 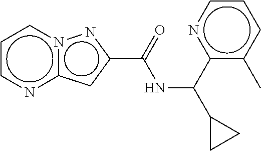 |
| 605 | 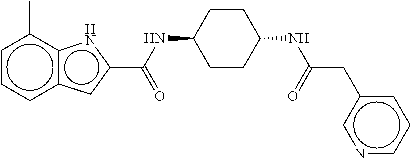 |
| 606 | 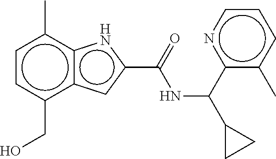 |
| 607 | 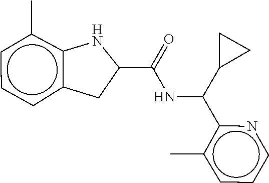 |
| 608 | 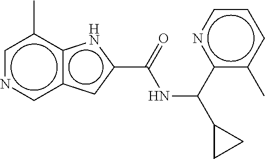 |
| 609 | 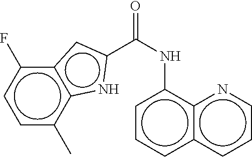 |
| 612 | 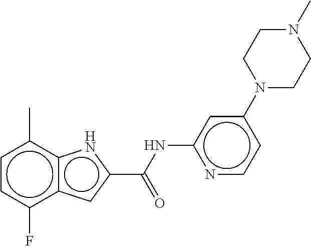 |
| 613 | 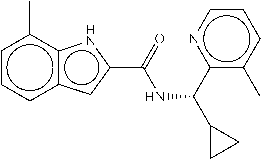 |
| 614 | 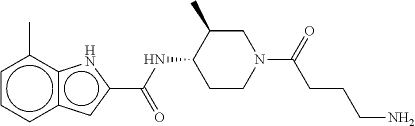 |
| 615 | 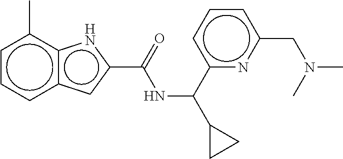 |
| 616 | 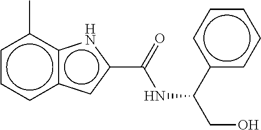 |
| 617 | 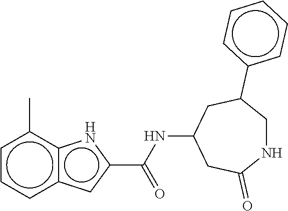 |
| 618 | 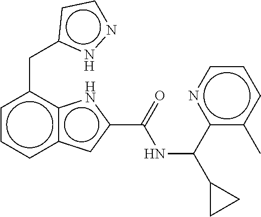 |
| 619 | 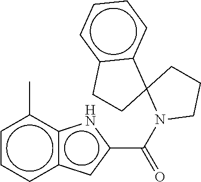 |
| 620 | 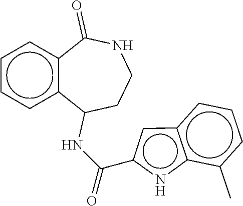 |
| 622 | 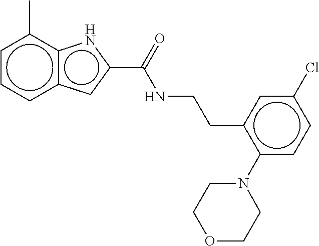 |
| 623 | 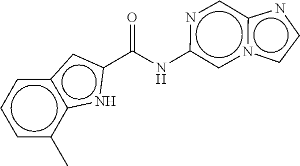 |
| 624 | 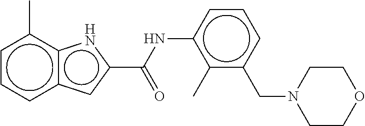 |
| 625 | 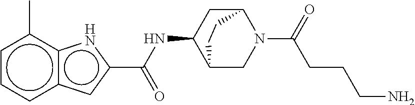 |
| 626 | 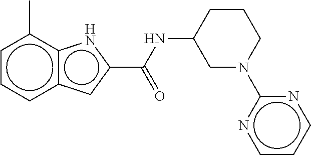 |
| 627 | 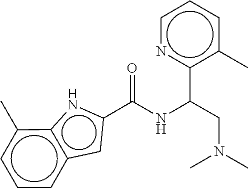 |
| 628 | 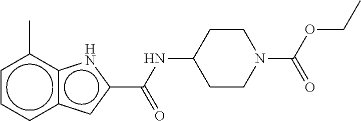 |
| 629 | 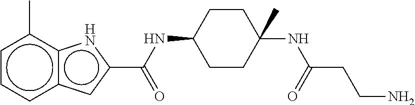 |
| 630 | 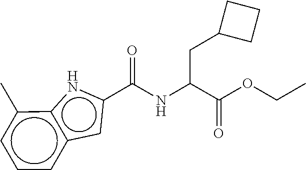 |
| 631 | 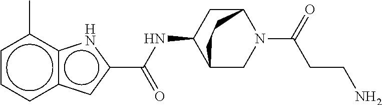 |
| 632 | 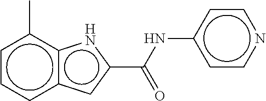 |
| 633 | 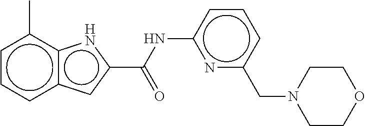 |
| 634 | 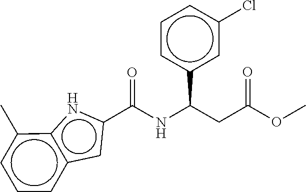 |
| 635 | 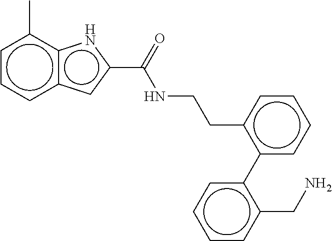 |
| 636 | 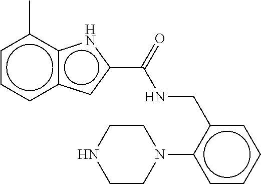 |
| 637 | 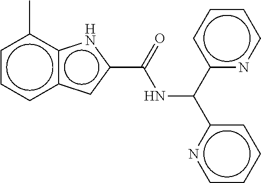 |
| 638 | 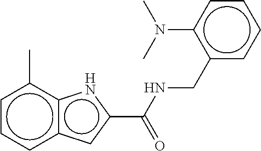 |
| 639 | 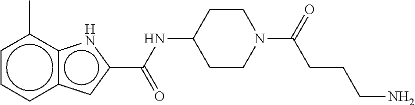 |
| 640 | 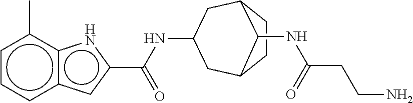 |
| 641 | 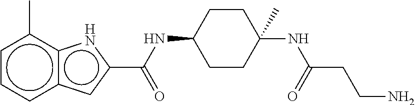 |
| 642 | 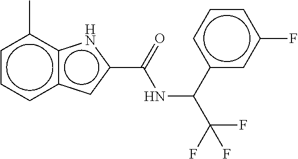 |
| 643 | 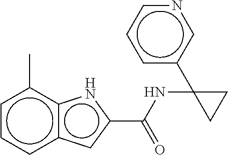 |
| 644 | 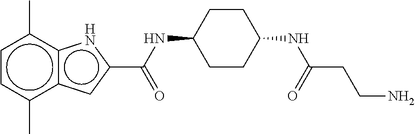 |
| 645 | 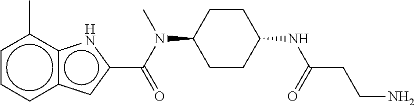 |
| 646 | 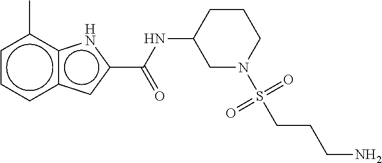 |
| 648 | 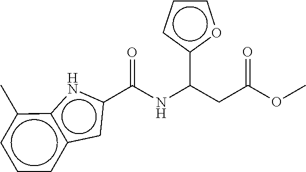 |
| 649 | 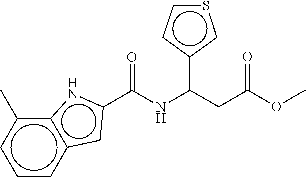 |
| 650 | 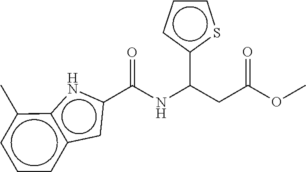 |
| 652 | 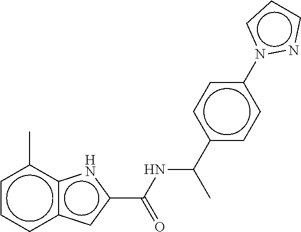 |
| 653 | 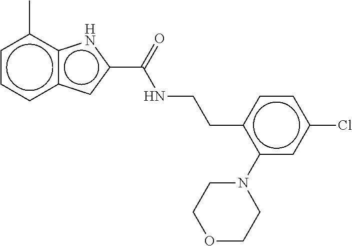 |
| 654 | 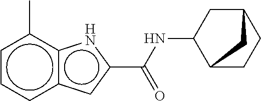 |
| 655 | 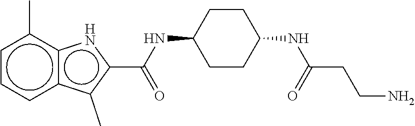 |
| 656 | 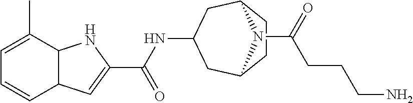 [SMILES: CC1=CC=CC2C=C(C(=O)NC3C[C@H]4CC[C@@H](C3)N4C(=O)CCCN)NC12] |
| 657 | 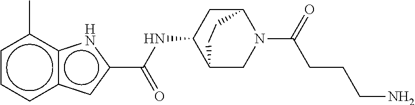 |
| 658 | 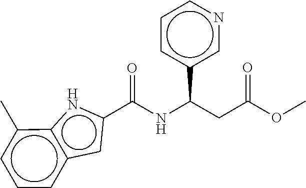 |
| 659 | 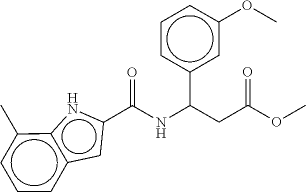 |
| 660 | 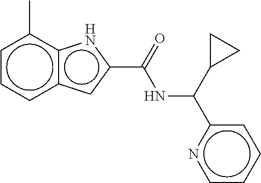 |
| 661 | 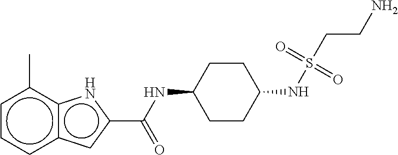 |
| 662 | 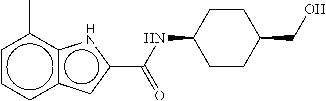 |
| 663 | 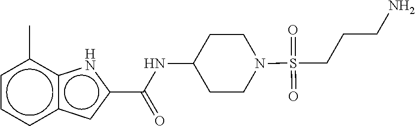 |
| 664 | 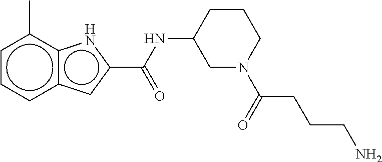 |
| 665 | 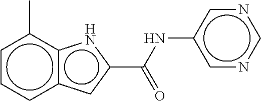 |
| 666 | 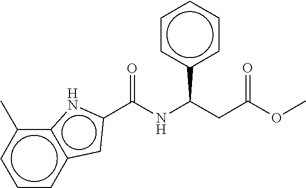 |
| 667 | 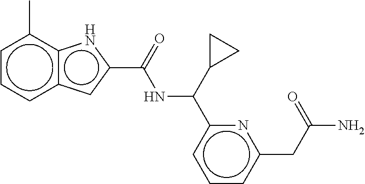 |
| 668 | 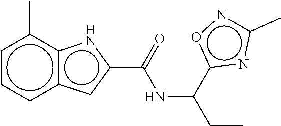 |
| 669 | 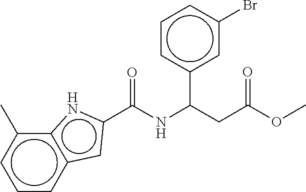 |
| 670 | 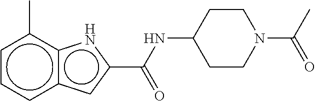 |
| 671 | 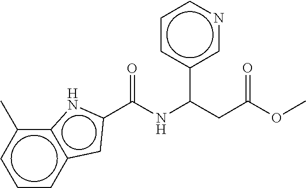 |
| 672 | 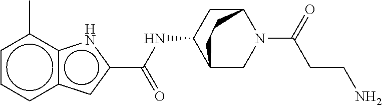 |
| 673 | 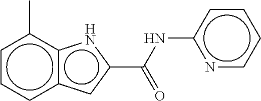 |
| 674 | 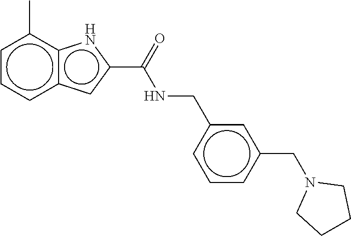 |
| 675 | 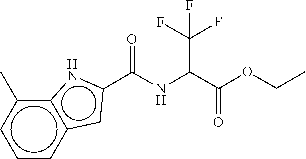 |
| 676 | 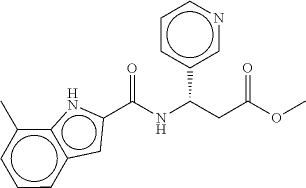 |
| 677 | 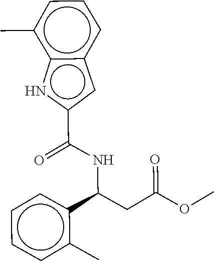 |
| 678 | 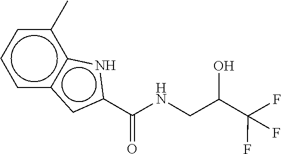 |
| 679 | 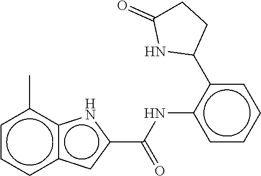 |
| 680 | 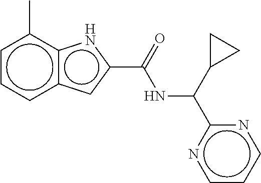 |
| 681 | 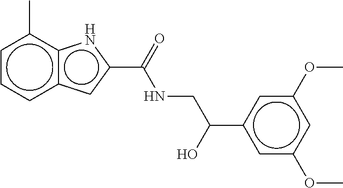 |
| 682 | 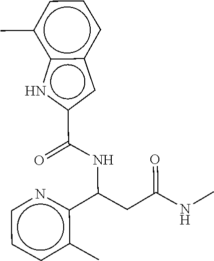 |
| 683 | 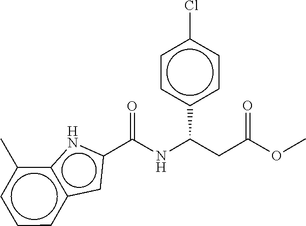 |
| 684 | 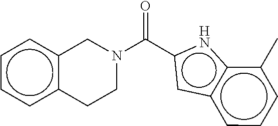 |
| 685 | 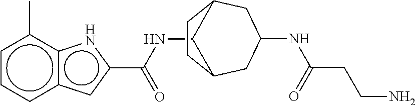 |
| 686 | 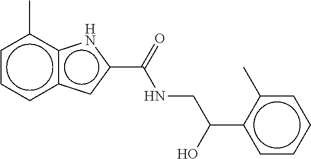 |
| 687 | 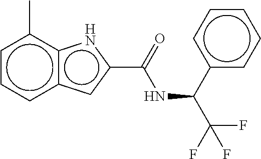 |
| 688 | 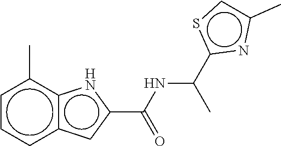 |
| 689 | 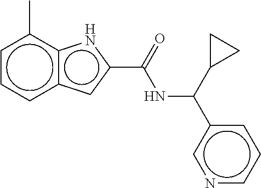 |
| 690 | 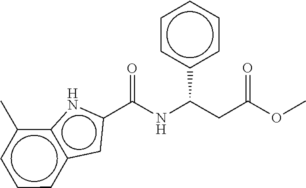 |
| 691 | 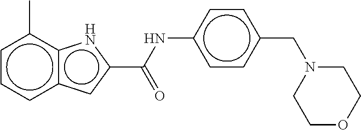 |
| 692 | 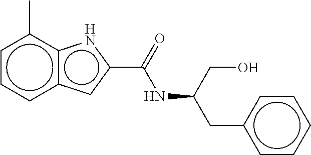 |
| 693 | 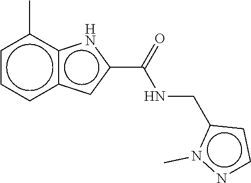 |
| 694 | 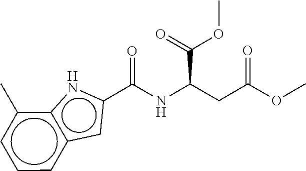 |
| 695 | 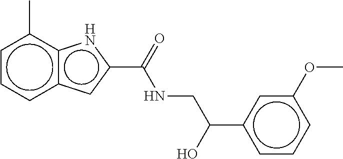 |
| 697 | 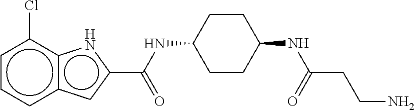 |
| 698 | 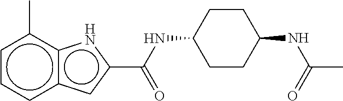 |
| 699 | 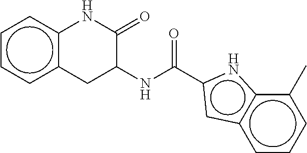 |
| 700 | 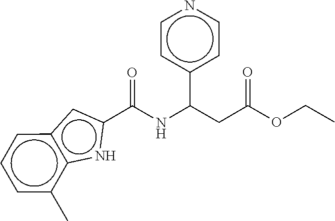 |
| 701 | 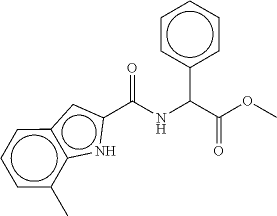 |
| 702 | 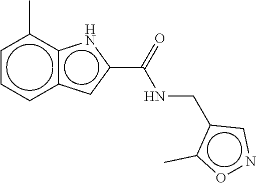 |
| 703 | 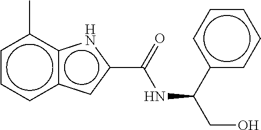 |
| 704 | 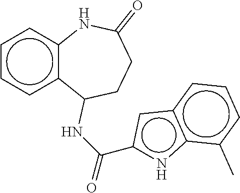 |
| 705 | 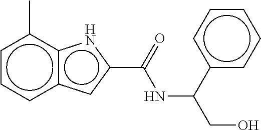 |
| 706 | 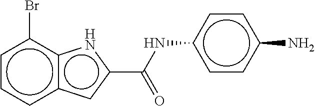 |
| 707 | 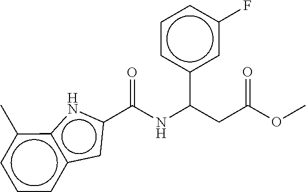 |
| 708 | 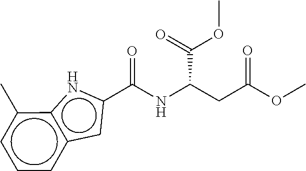 |
| 709 | 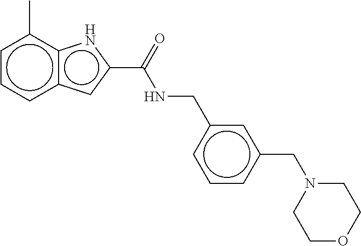 |
| 710 | 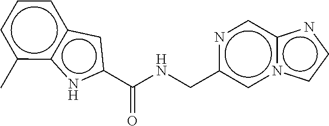 |
| 711 | 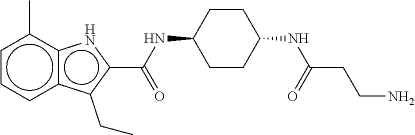 |
| 712 | 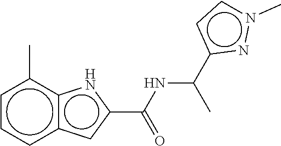 |
| 713 | 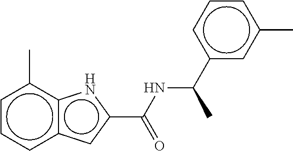 |
| 714 | 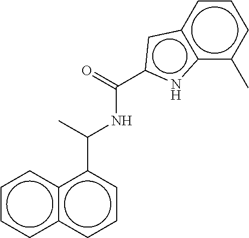 |
| 715 | 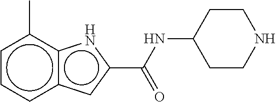 |
| 716 | 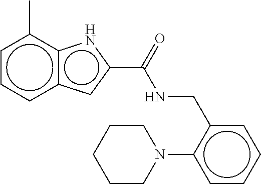 |
| 717 | 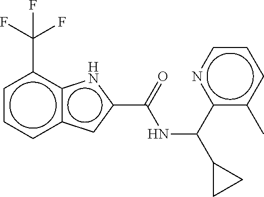 |
| 718 | 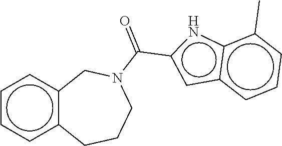 |
| 719 | 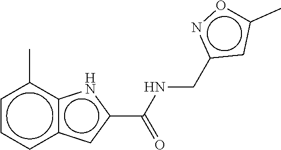 |
| 720 | 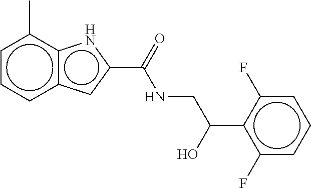 |
| 721 | 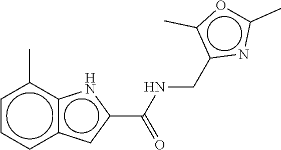 |
| 722 | 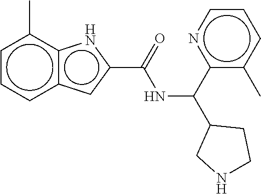 |
| 723 | 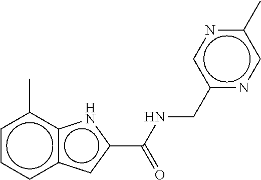 |
| 724 | 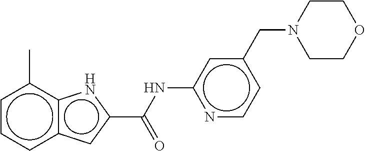 |
| 725 | 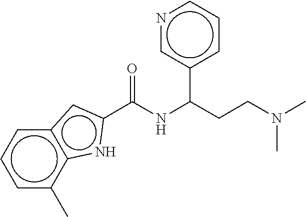 |
| 726 | 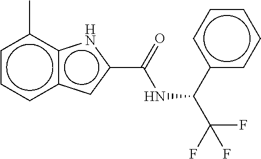 |
| 727 | 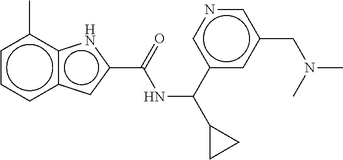 |
| 728 | 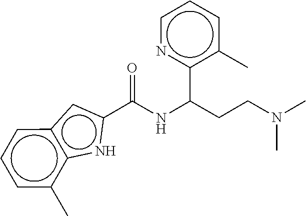 |
| 729 | 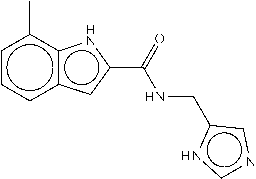 |
| 730 | 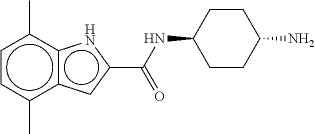 |
| 731 | 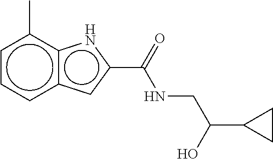 |
| 732 | 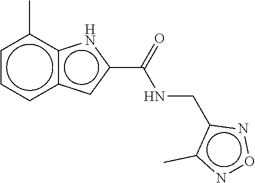 |
| 733 | 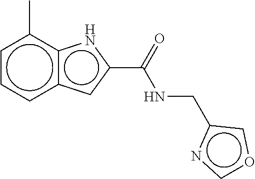 |
| 735 | 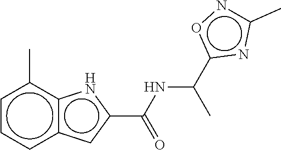 |
| 736 | 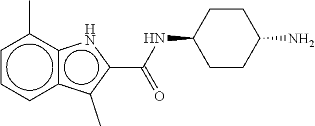 |
| 738 | 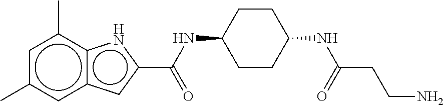 |
| 746 | 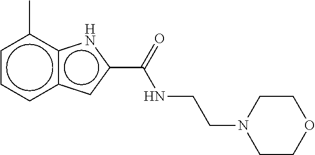 |
| 747 | 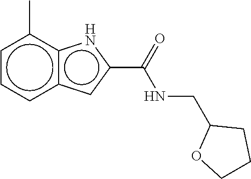 |
| 748 | 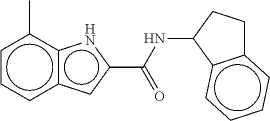 |
| 749 | 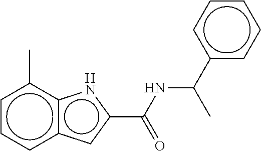 |
| 750 | 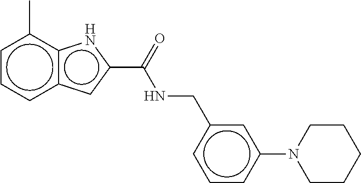 |
| 751 | 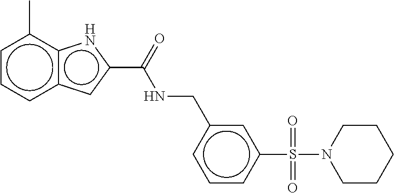 |
| 752 | 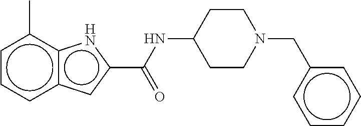 |
| 753 | 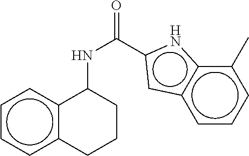 |
| 754 | 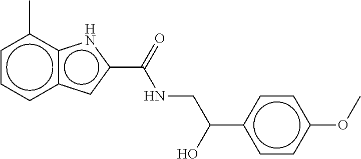 |
| 755 | 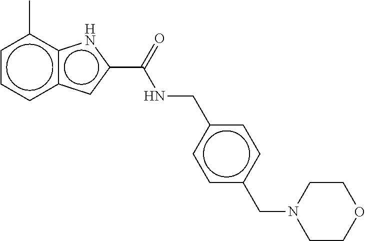 |
| 756 | 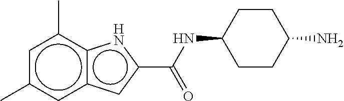 |
| 757 | 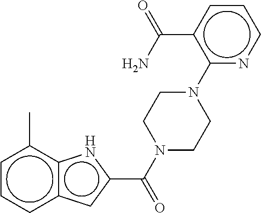 |
| 758 | 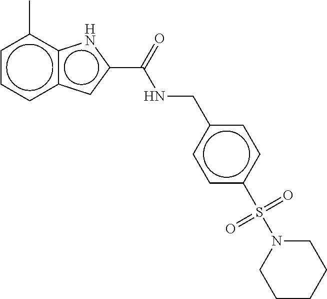 |
| 759 | 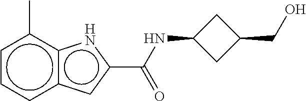 |
| 761 | 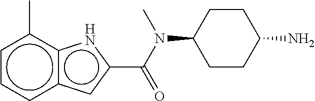 |
| 763 | 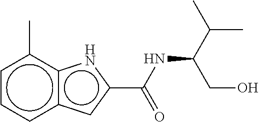 |
| 764 | 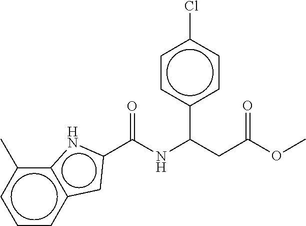 |
| 765 | 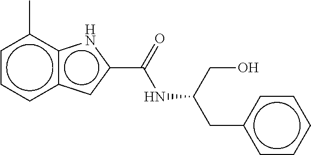 |
| 766 | 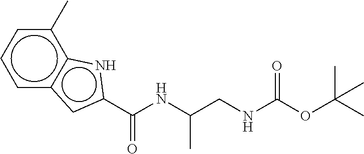 |
| 767 | 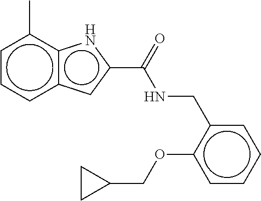 |
| 768 | 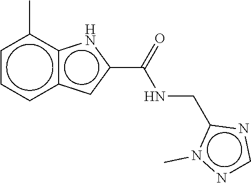 |
| 769 | 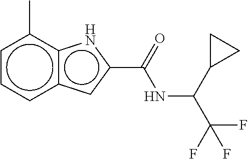 |
| 771 | 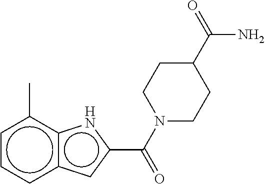 |
| 773 | 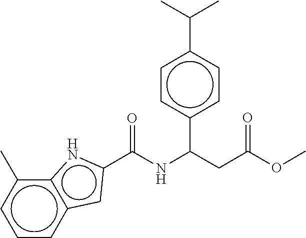 |
| 774 | 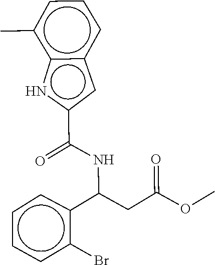 |
| 775 | 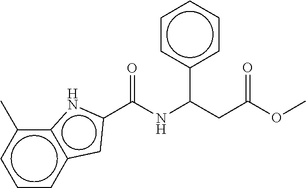 |
| 776 | 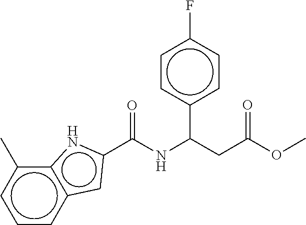 |
| 777 | 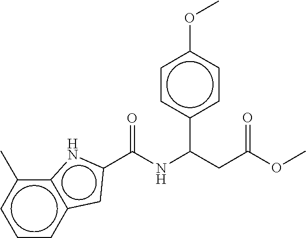 |
| 778 | 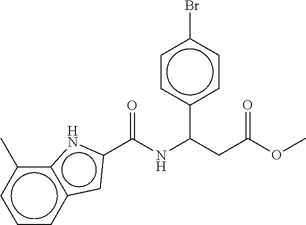 |
| 779 | 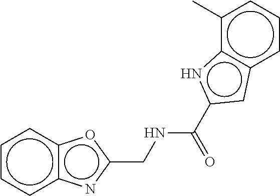 |
| 780 | 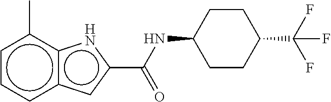 |
| 781 | 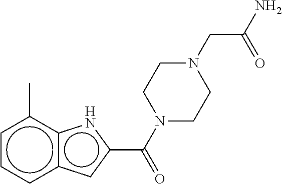 |
| 782 | 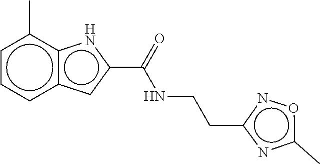 |
| 783 | 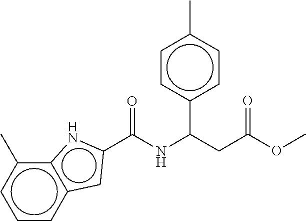 |
| 784 | 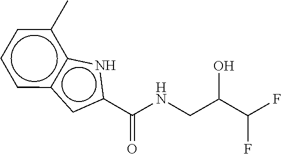 |
| 785 | 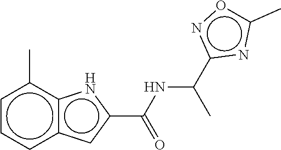 |
| 786 | 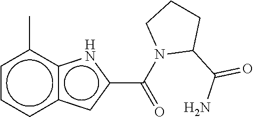 |
| 787 | 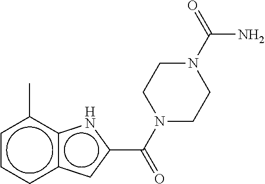 |
| 788 | 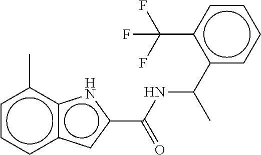 |
| 790 | 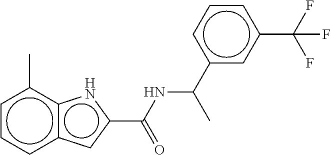 |
| 791 | 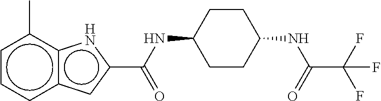 |
| 793 | 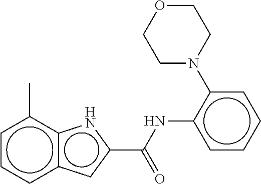 |
| 794 | 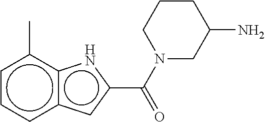 |
| 795 | 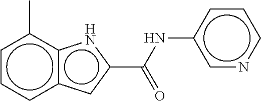 |
| 796 | 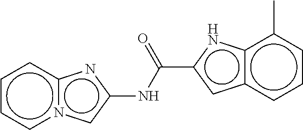 |
| 797 | 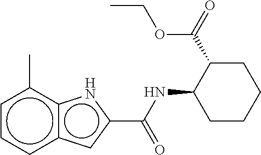 |
| 798 | 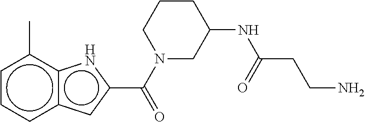 |
| 799 | 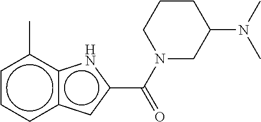 |
| 800 | 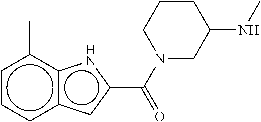 |
| 801 | 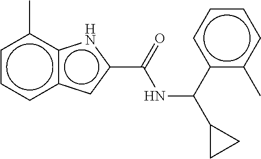 |
| 802 | 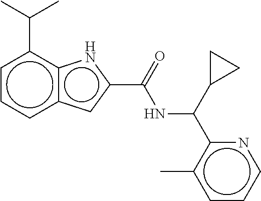 |
| 803 | 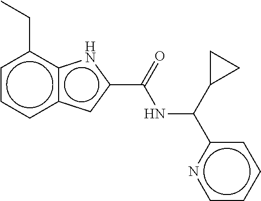 |
| 804 | 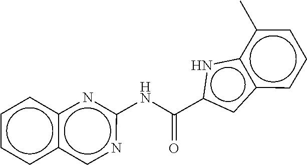 |
| 805 | 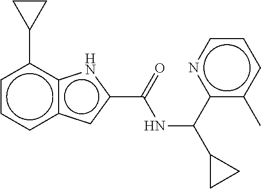 |
| 808 | 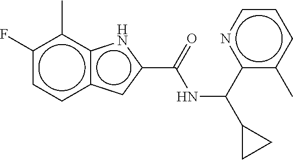 |
| 809 | 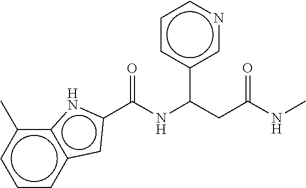 |
| 810 | 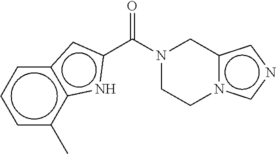 |
| 811 | 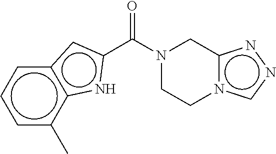 |
| 812 | 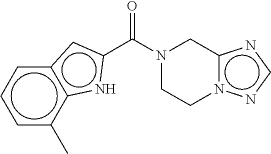 |
| 814 | 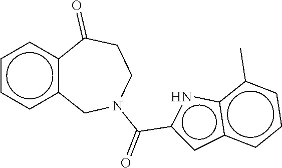 |
| 815 | 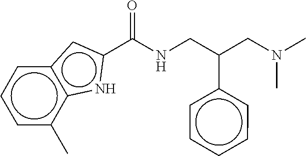 |
| 816 | 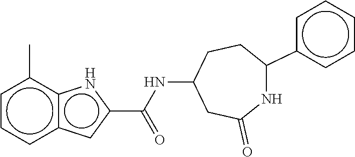 |
| 817 | 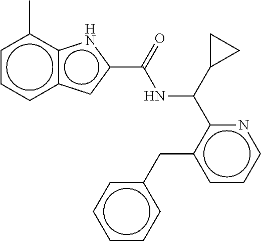 |
| 818 | 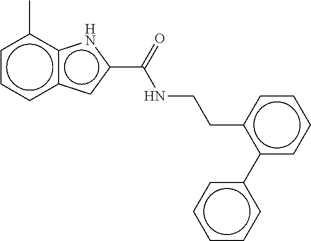 |
| 819 | 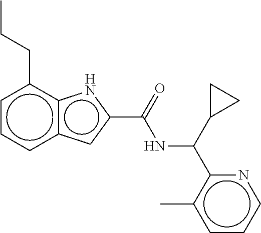 |
| 820 | 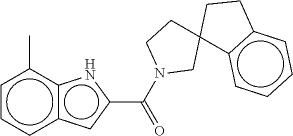 |
| 821 | 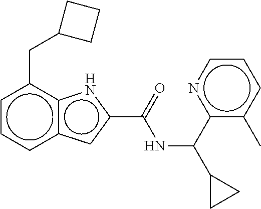 |
| 822 | 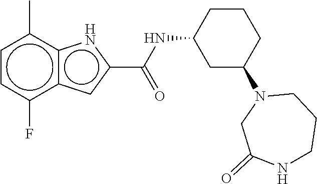 |
| 823 | 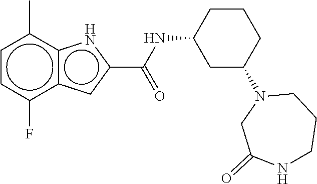 |
| 824 | 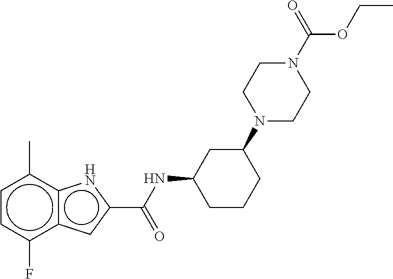 |
| 825 | 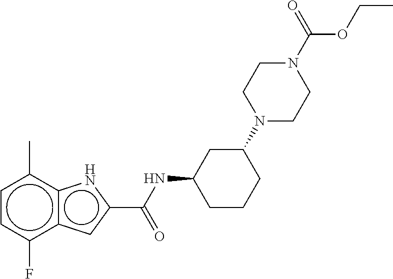 |
| 826 | 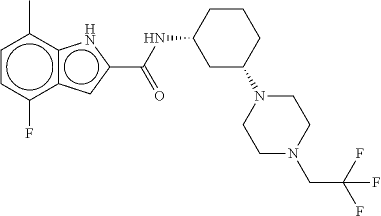 |
| 827 | 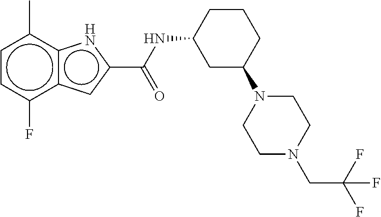 |
| 828 | 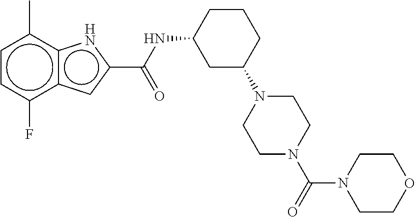 |
| 829 | 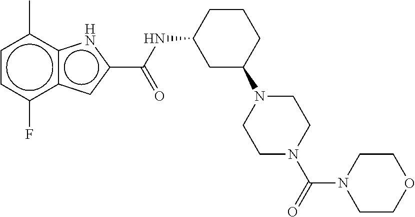 |
| 830 | 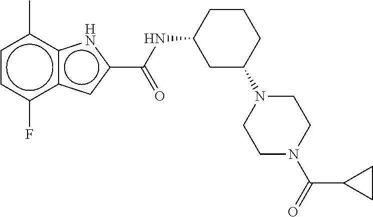 |
| 831 | 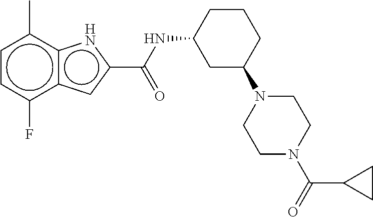 |
| 832 | 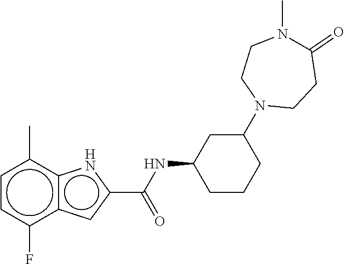 |
| 833 | 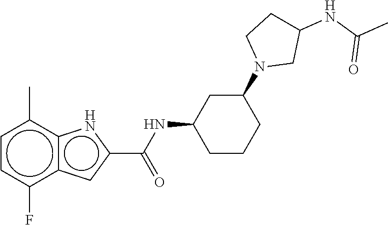 |
| 834 | 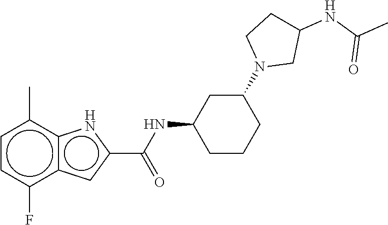 |
| 835 | 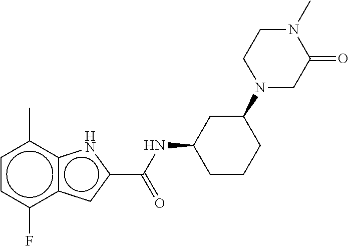 |
| 836 | 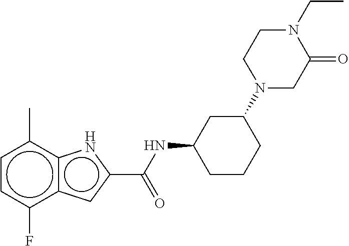 |
| 837 | 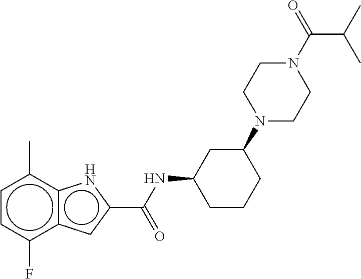 |
| 838 | 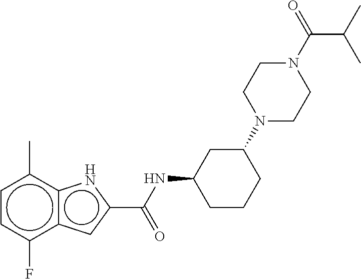 |
| 839 | 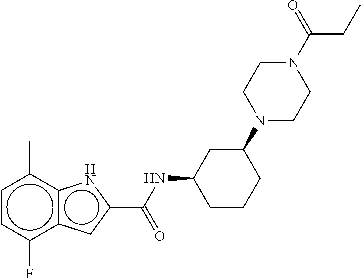 |
| 840 | 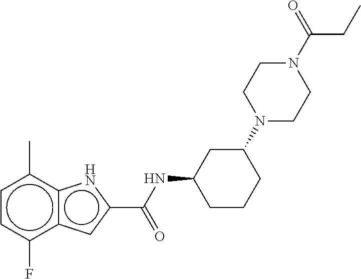 |
| 841 | 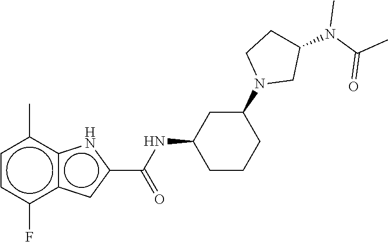 |
| 842 | 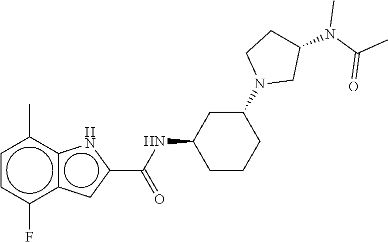 |
| 843 | 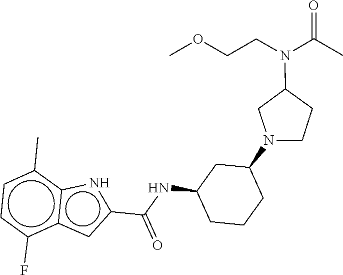 |
| 844 | 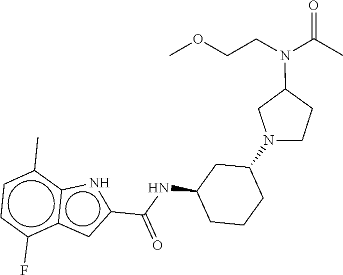 |
| 845 | 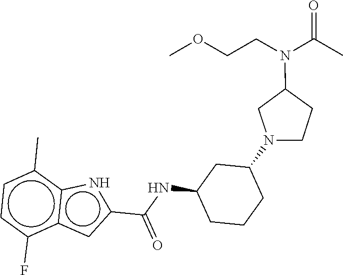 |
| 846 | 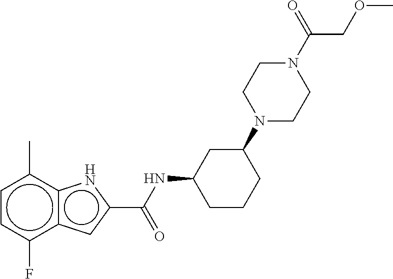 |
| 847 | 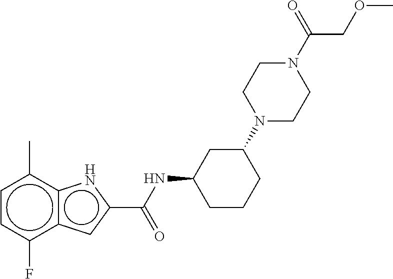 |
| 848 | 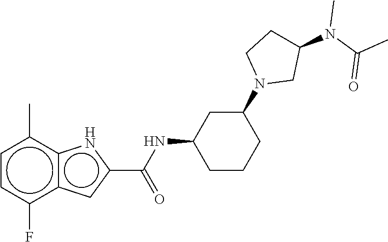 |
| 849 | 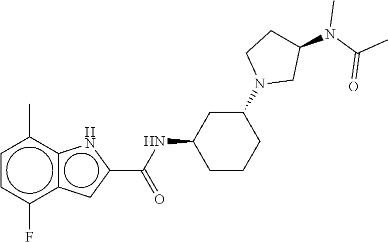 |
| 850 | 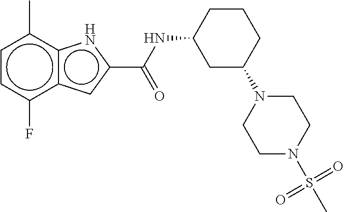 |
| 851 | 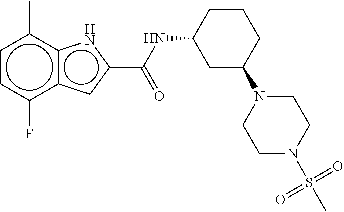 |
| 852 | 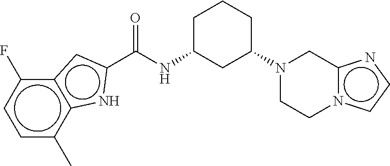 |
| 853 | 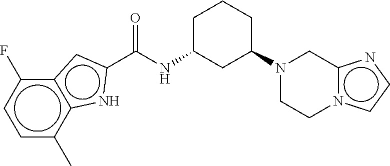 |
| 854 | 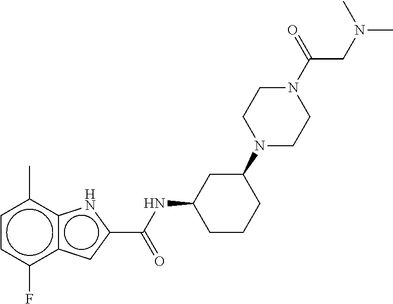 |
| 855 | 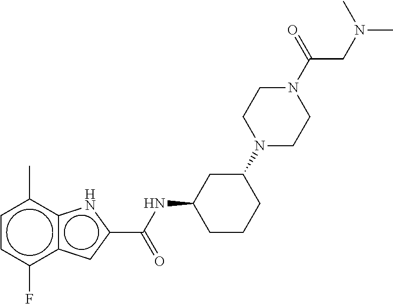 |
| 856 | 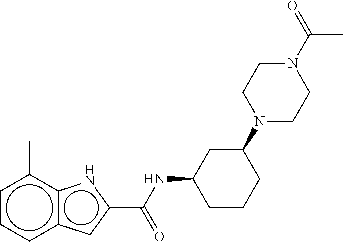 |
| 857 | 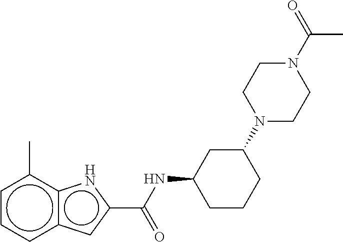 |
| 858 | 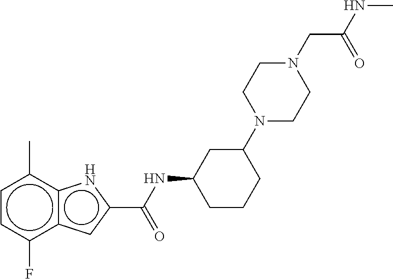 |
| 859 | 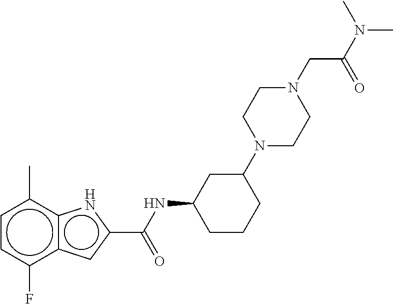 |
| 860 | 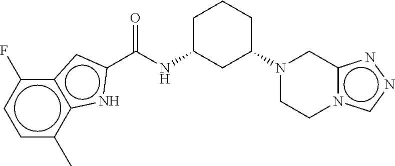 |
| 861 | 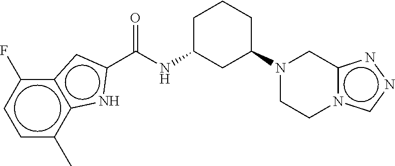 |
| 862 | 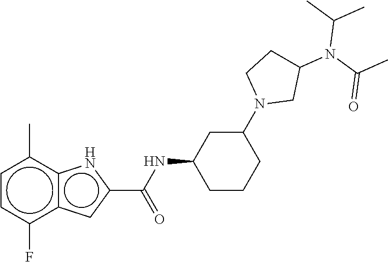 |
| 863 | 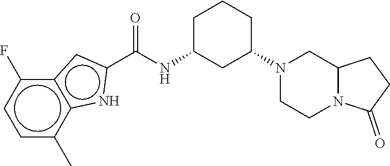 |
| 864 | 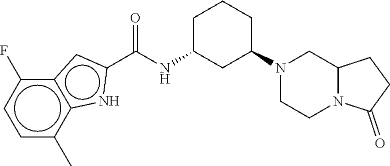 |
| 865 | 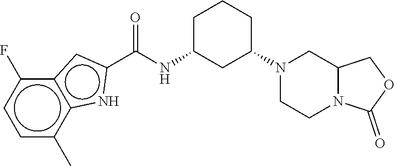 |
| 866 | 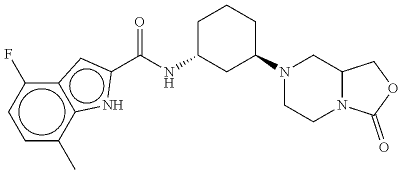 |
| 867 | 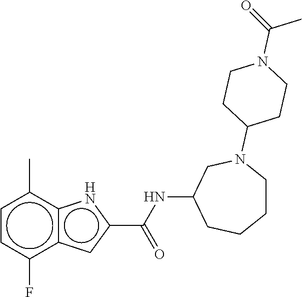 |
| 868 | 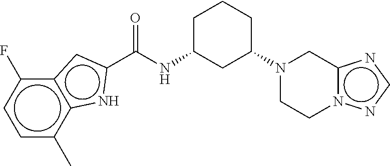 |
| 869 | 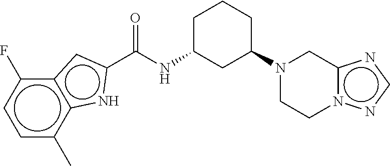 |
| 870 | 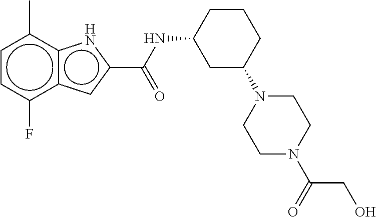 |
| 871 | 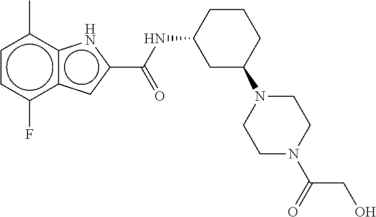 |
| 872 | 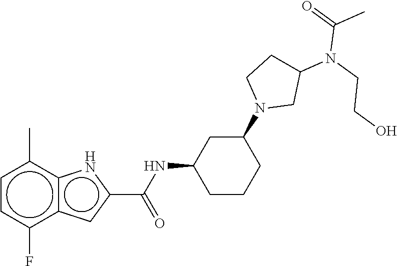 |
| 873 | 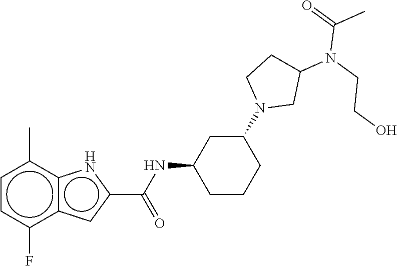 |
| 874 | 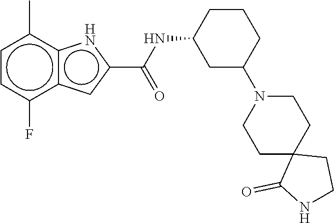 |
| 875 | 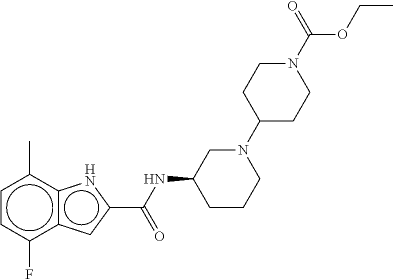 |
| 876 | 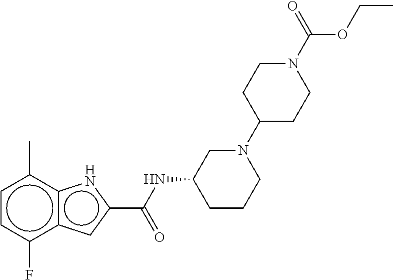 |
| 877 | 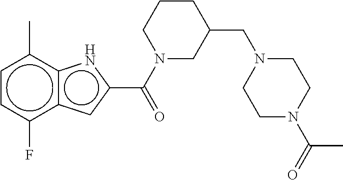 |
| 878 | 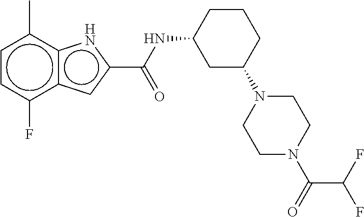 |
| 879 | 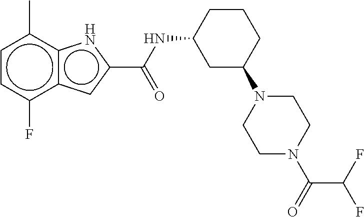 |
| 880 | 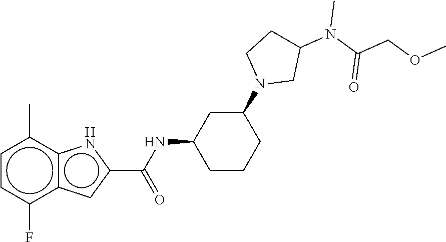 |
| 881 | 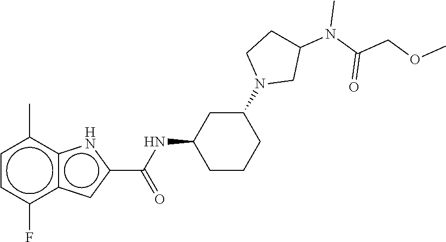 |
| 882 | 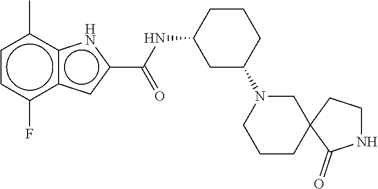 |
| 883 | 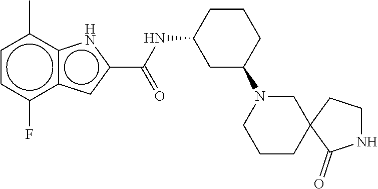 |
| 884 | 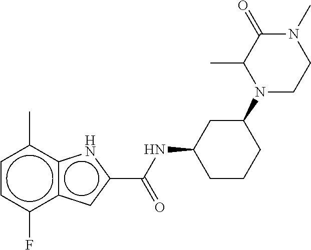 |
| 885 | 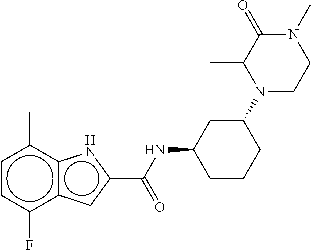 |
| 886 | 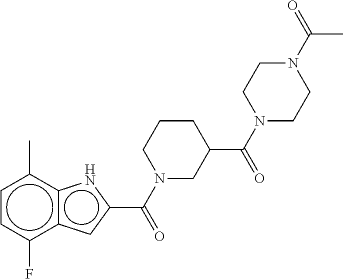 |
| 887 | 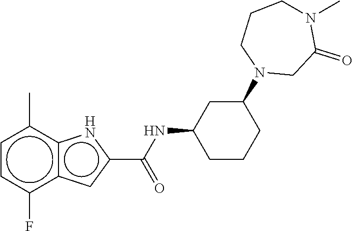 |
| 888 | 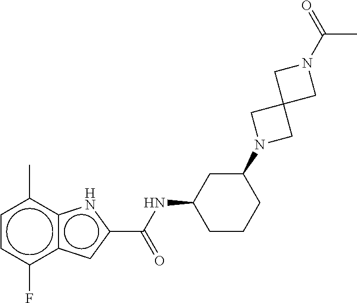 |
| 889 | 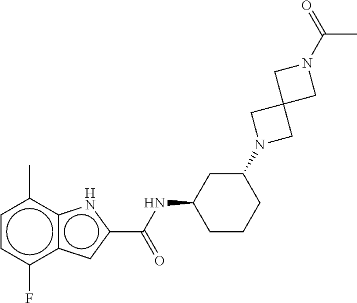 |
| 890 | 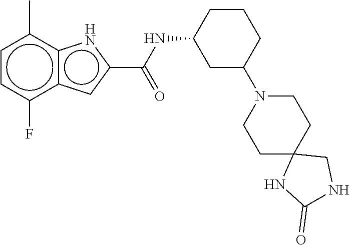 |
| 891 | 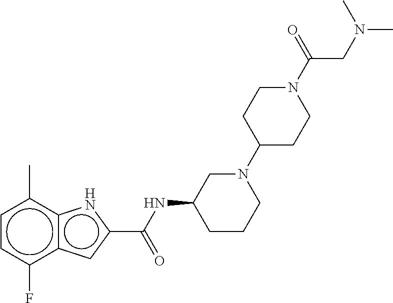 |
| 892 | 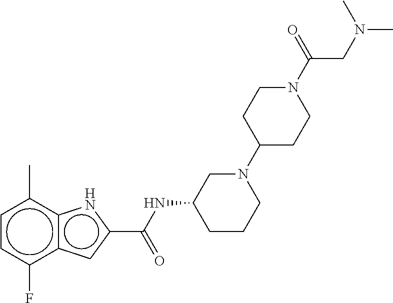 |
| 893 | 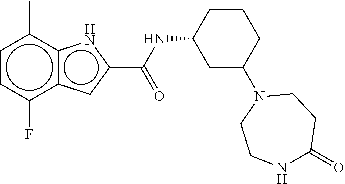 |
| 894 | 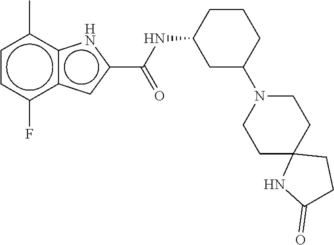 |
| 895 | 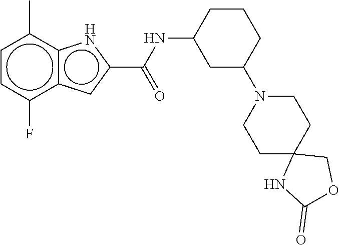 |
| 896 | 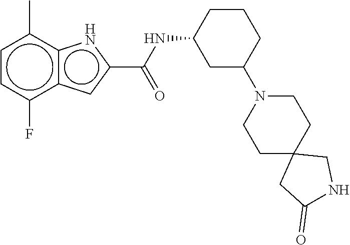 |
| 897 | 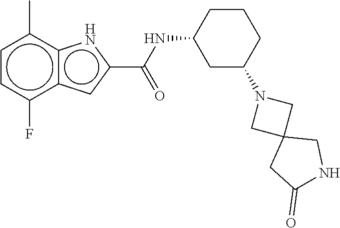 |
| 898 | 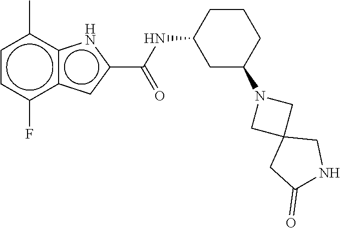 |
| 899 | 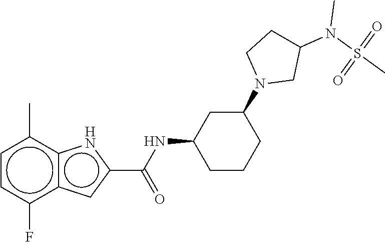 |
| 900 | 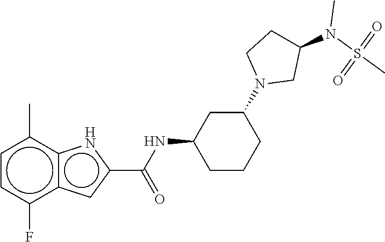 |
| 901 | 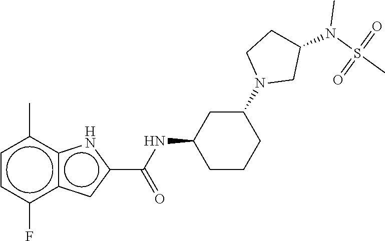 |
| 902 | 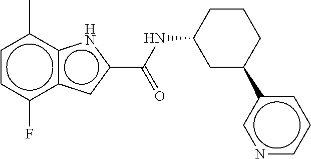 |
| 903 | 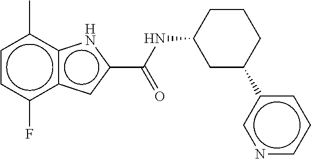 |
| 904 | 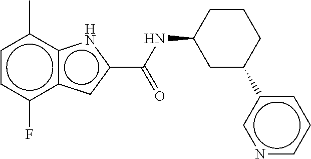 |
| 905 | 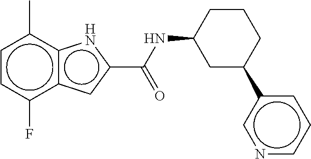 |
| 906 | 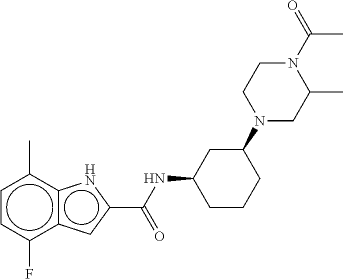 |
| 907 | 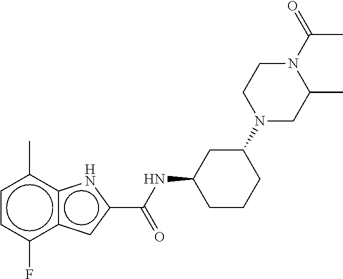 |
| 908 | 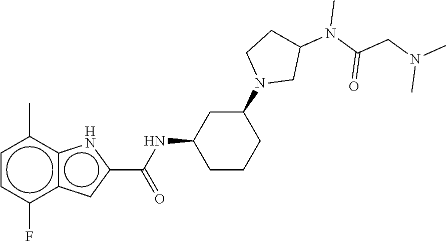 |
| 909 | 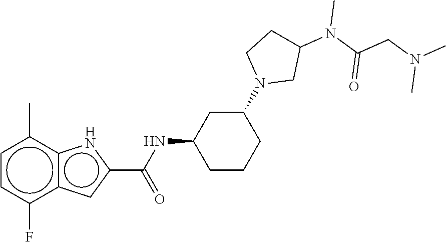 |
| 910 | 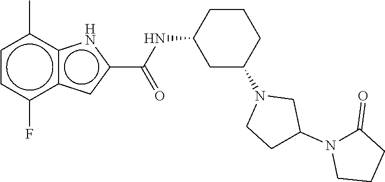 |
| 911 | 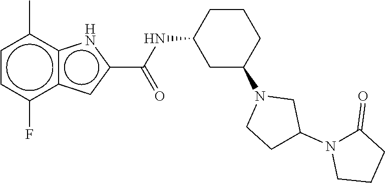 |
| 912 | 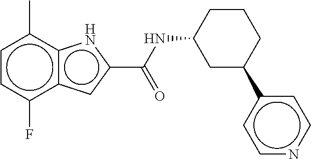 |
| 913 | 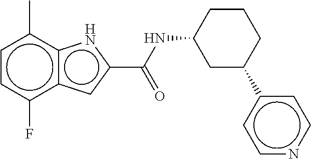 |
| 914 | 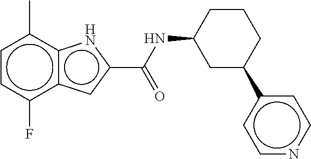 |
| 915 | 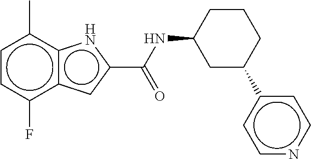 |
| 916 | 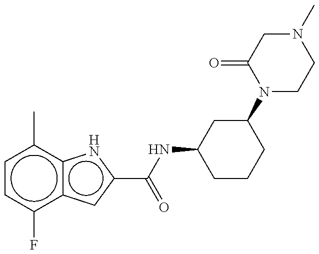 |
| 917 | 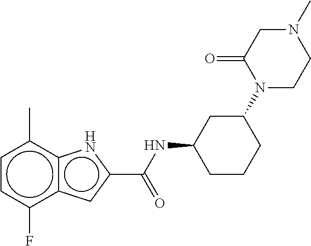 |
| 918 | 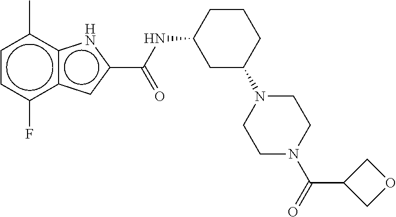 |
| 919 | 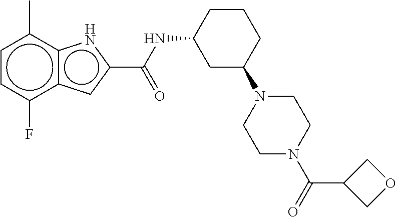 |
| 920 | 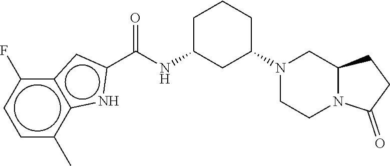 |
| 921 | 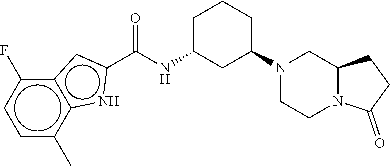 |
| 922 | 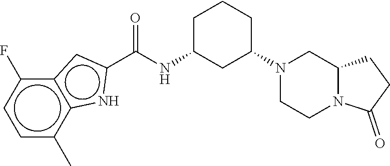 |
| 923 | 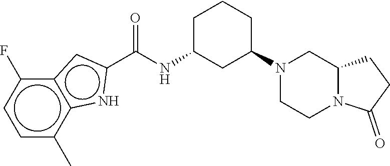 |
| 924 | 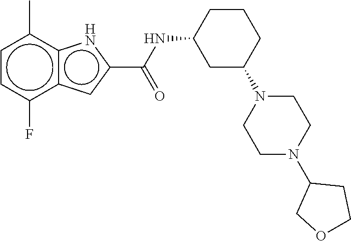 |
| 925 | 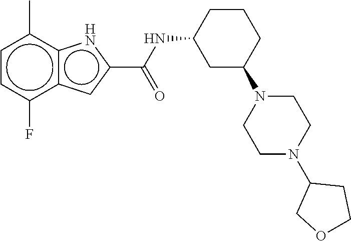 |
| 926 | 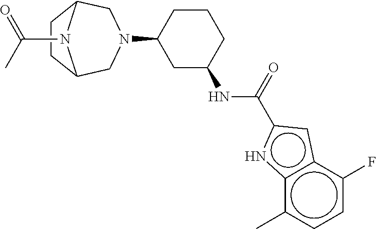 |
| 927 | 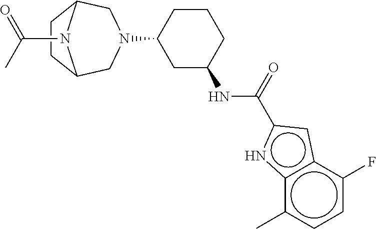 |
| 928 | 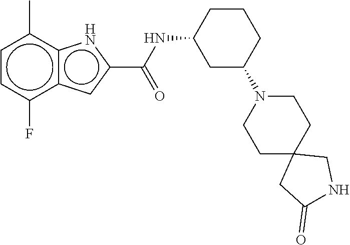 |
| 929 | 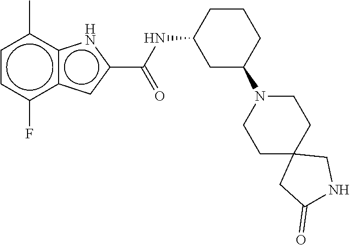 |
| 930 | 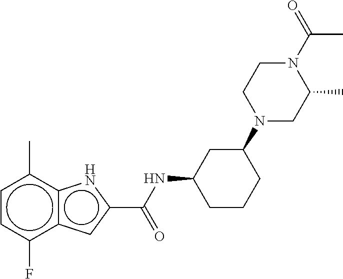 |
| 931 | 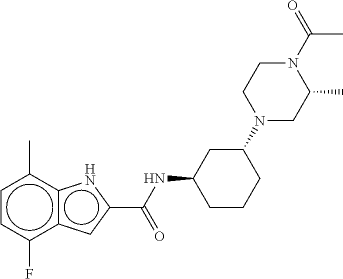 |
| 932 | 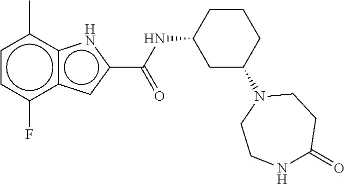 |
| 933 | 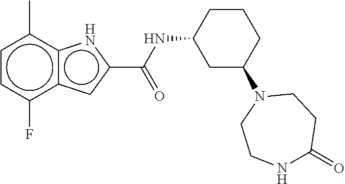 |
| 934 | 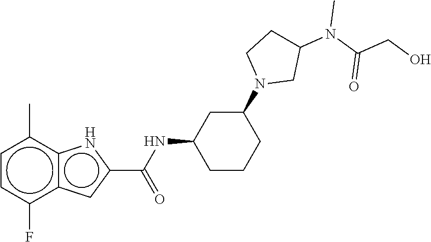 |
| 935 | 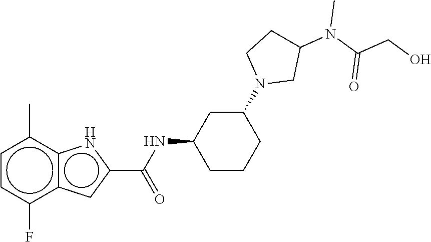 |
| 936 | 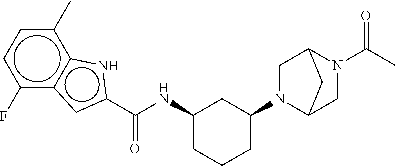 |
| 937 | 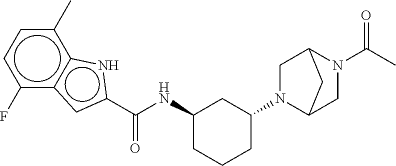 |
| 938 | 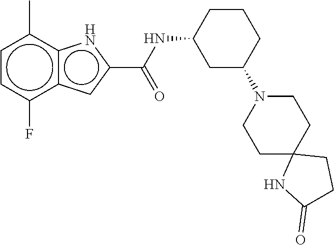 |
| 939 | 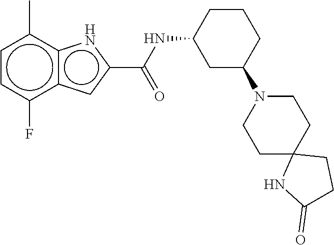 |
| 940 | 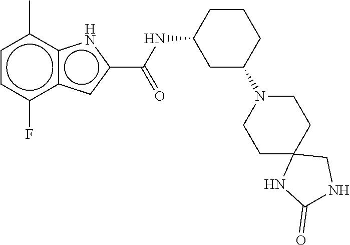 |
| 941 | 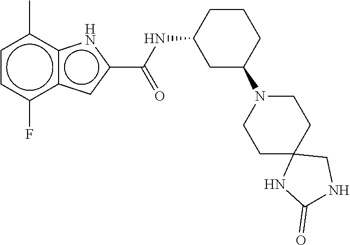 |
| 942 | 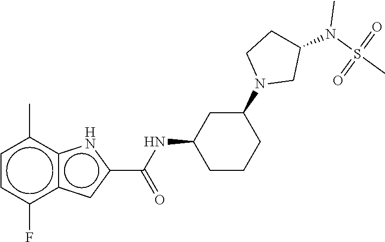 |
| 943 | 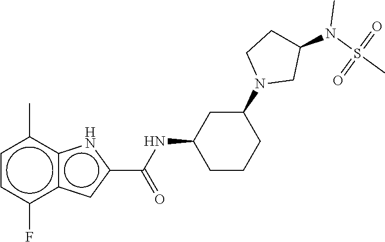 |
| 944 | 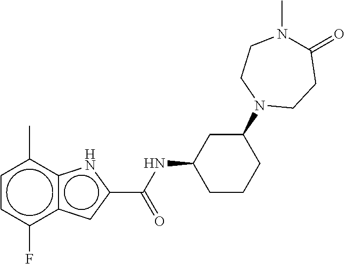 |
| 945 | 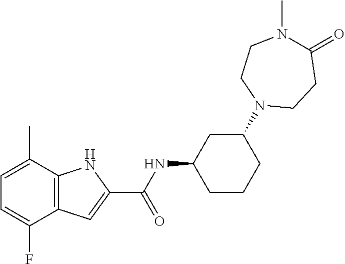 [SMILES: Cc1ccc(F)c2cc(C(=O)N[C@@H]3CCC[C@@H](N4CCC(=O)N(C)CC4)C3)[nH]c12] |
| 946 | 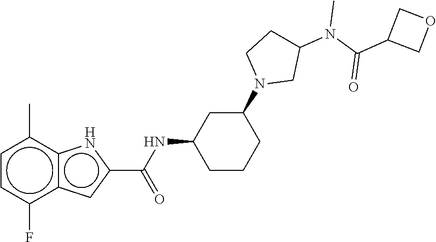 |
| 947 | 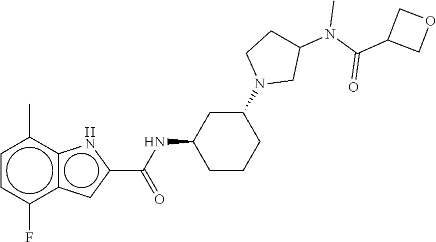 |
| 948 | 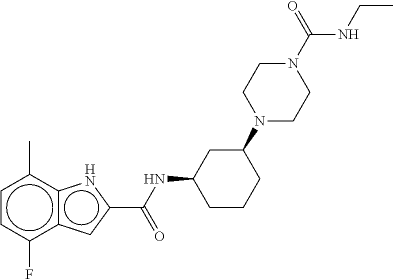 |
| 949 | 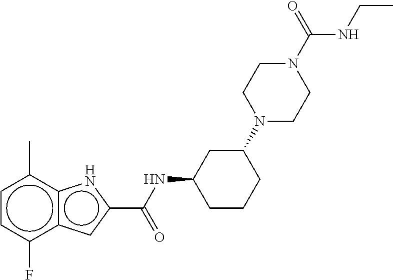 |
| 950 | 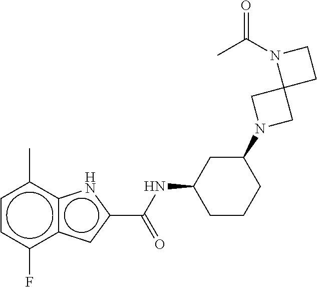 |
| 951 | 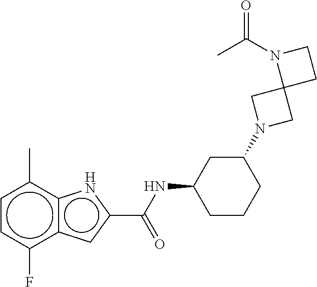 |
| 952 | 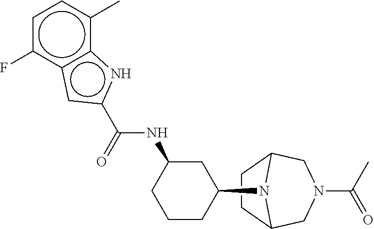 |
| 953 | 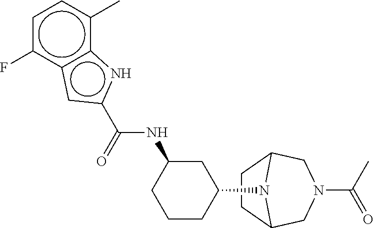 |
| 954 | 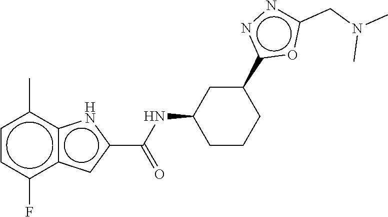 |
| 955 | 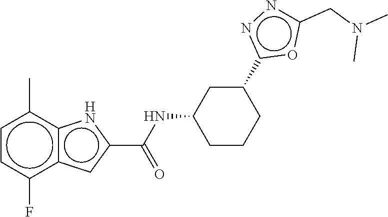 |
| 956 | 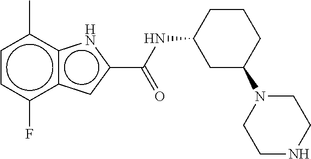 |
| 957 | 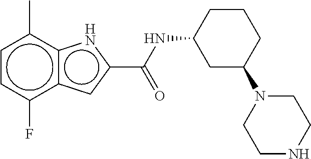 |
| 958 | 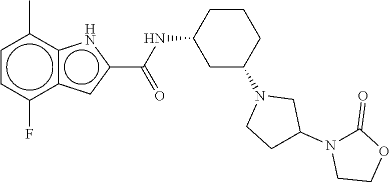 |
| 959 | 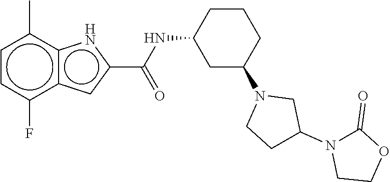 |
| 960 | 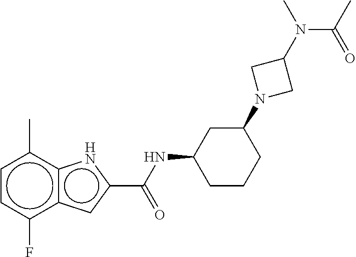 |
| 961 | 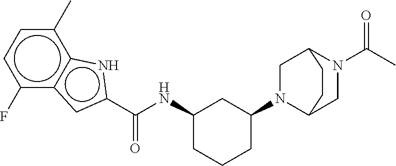 |
| 962 | 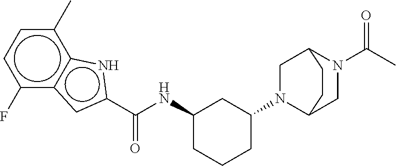 |
| 963 | 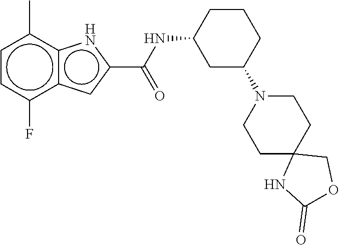 |
| 964 | 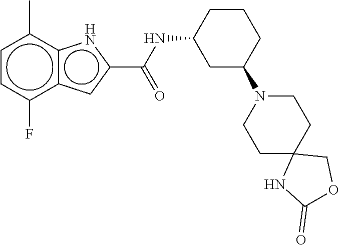 |
| 965 | 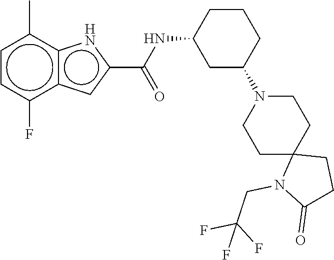 |
| 966 | 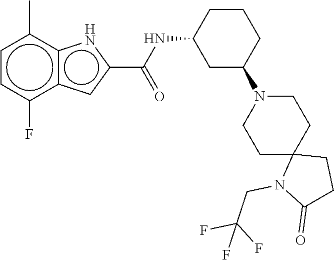 |
| 967 | 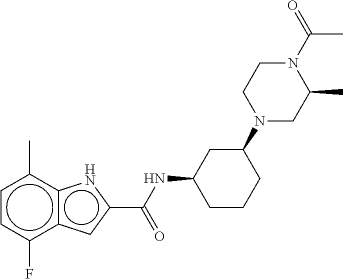 |
| 968 | 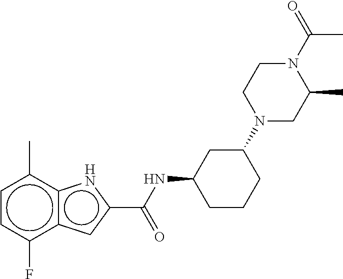 |
| 969 | 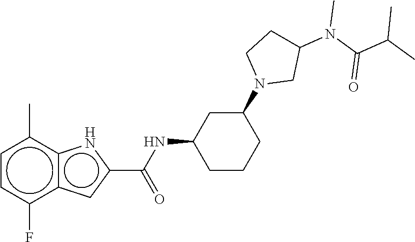 |
| 970 | 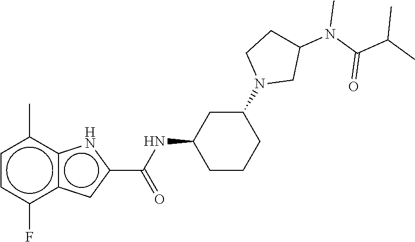 |
| 971 | 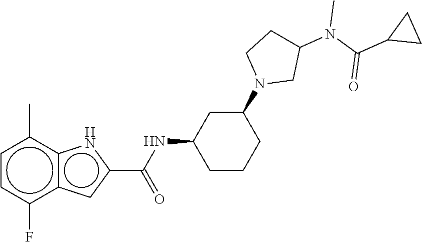 |
| 972 | 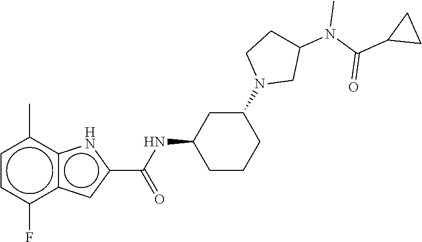 |
| 973 | 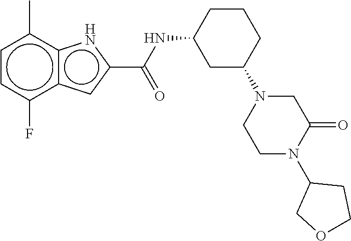 |
| 974 | 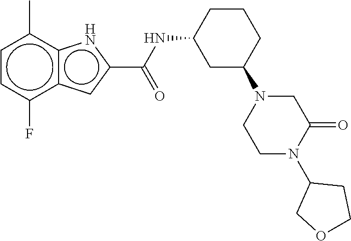 |
| 975 | 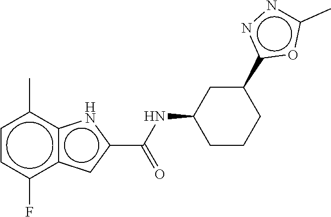 |
| 976 | 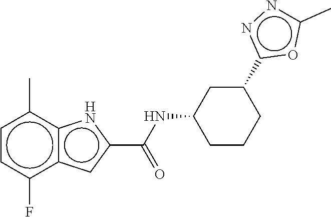 |
| 977 | 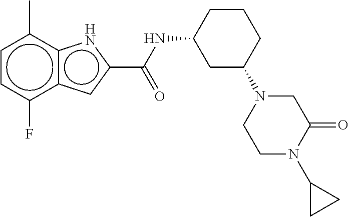 |
| 978 | 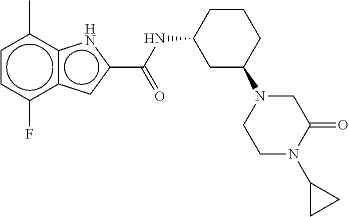 |
| 979 | 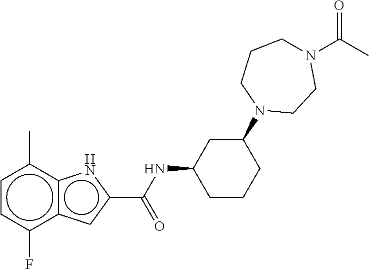 |
| 980 | 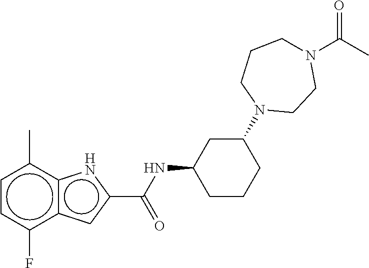 |
| 981 | 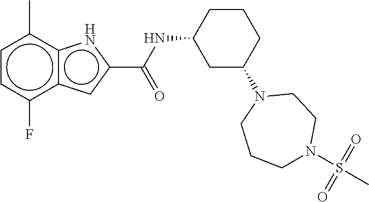 |
| 982 | 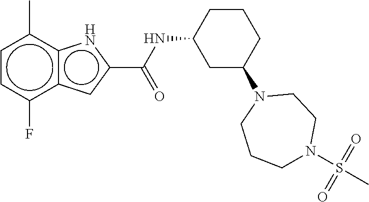 |
| 983 | 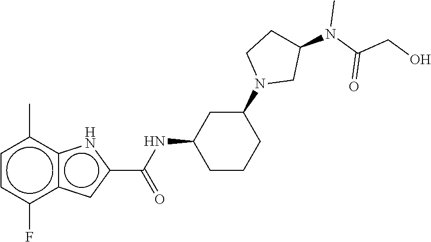 |
| 984 | 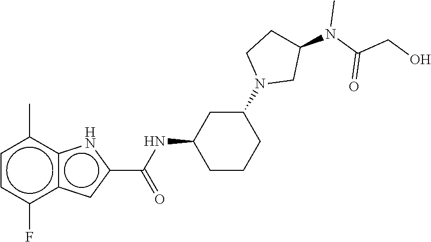 |
| 985 | 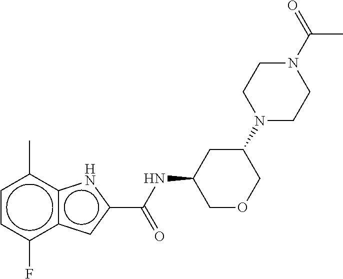 |
| 986 | 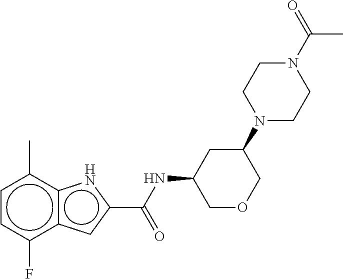 |
| 987 | 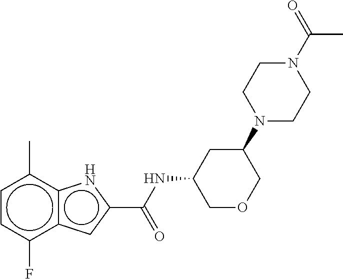 |
| 988 | 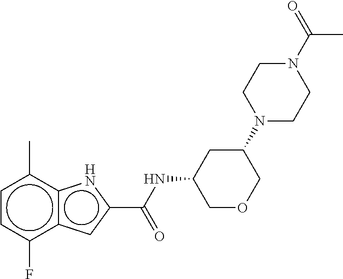 |
| 989 | 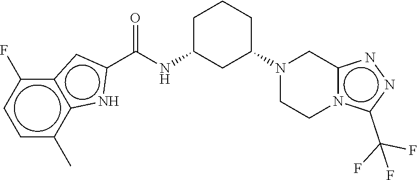 |
| 990 | 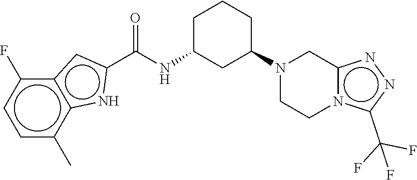 |
| 991 | 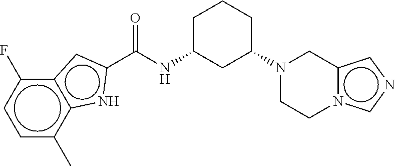 |
| 992 | 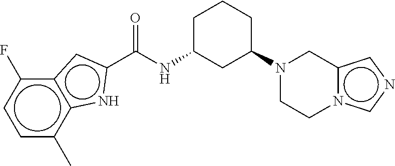 |
| 993 | 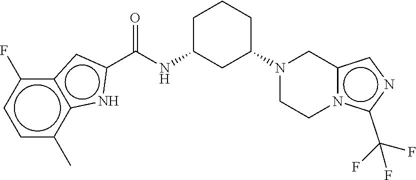 |
| 994 | 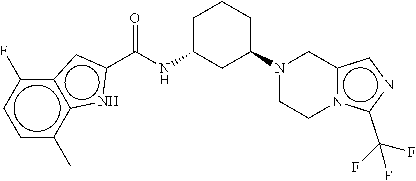 |
| 995 | 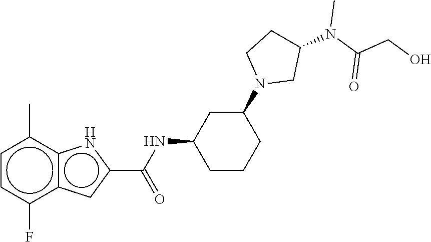 |
| 996 | 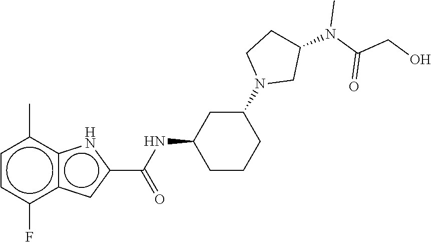 |
| 997 | 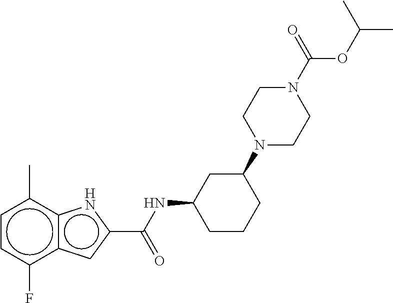 |
| 998 | 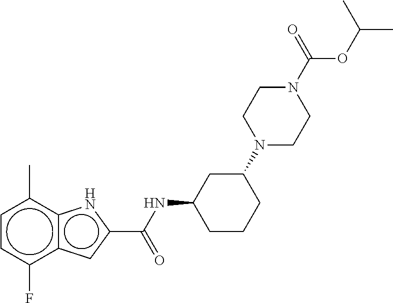 |
| 999 | 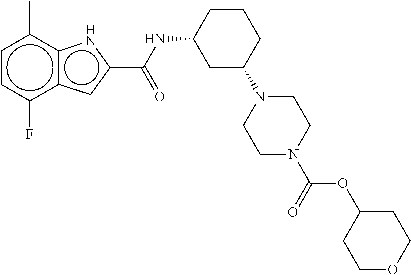 |
| 1000 | 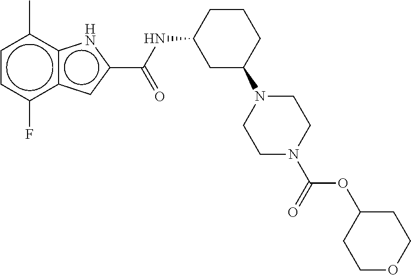 |
| 1002 | 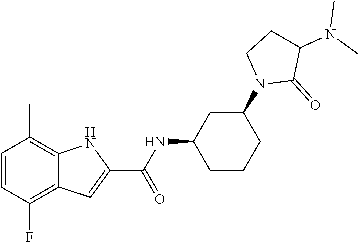 [SMILES: Cc1ccc(F)c2cc(C(=O)N[C@@H]3CCC[C@H](N4CCC(N(C)C)C4=O)C3)[nH]c12] |
| 1003 | 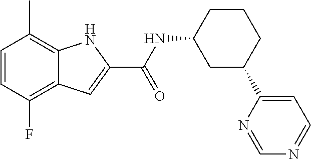 [SMILES: Cc1ccc(F)c2cc(C(=O)N[C@@H]3CCC[C@H](c4ccncn4)C3)[nH]c12] |
| 1004 | 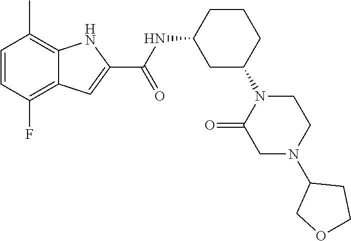 [SMILES: Cc1ccc(F)c2cc(C(=O)N[C@@H]3CCC[C@H](N4CCN(C5CCOC5)CC4=O)C3)[nH]c12] |
| 1005 | 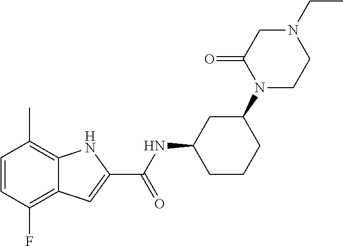 [SMILES: CCN1CCN([C@H]2CCC[C@@H](NC(=O)c3cc4c(F)ccc(C)c4[nH]3)C2)C(=O)C1] |
| 1006 | 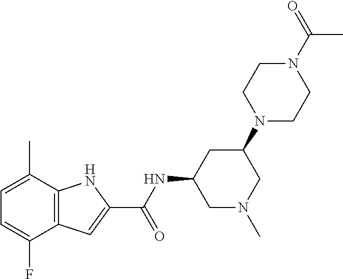 [SMILES: CC(=O)N1CCN([C@@H]2C[C@H](NC(=O)c3cc4c(F)ccc(C)c4[nH]3)CN(C)C2)CC1] |
| 1007 | 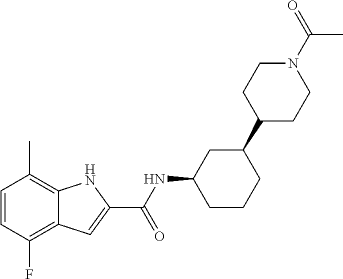 [SMILES: CC(=O)N1CCC([C@H]2CCC[C@@H](NC(=O)c3cc4c(F)ccc(C)c4[nH]3)C2)CC1] |
| 1008 | 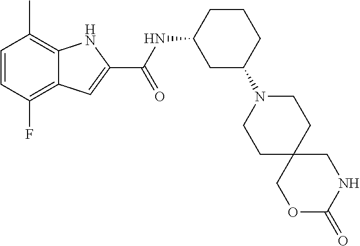 [SMILES: Cc1ccc(F)c2cc(C(=O)N[C@@H]3CCC[C@H](N4CCC5(CC4)CNC(=O)OC5)C3)[nH]c12] |
| 1009 | 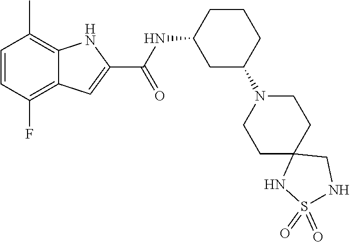 [SMILES: Cc1ccc(F)c2cc(C(=O)N[C@@H]3CCC[C@H](N4CCC5(CC4)CNS(=O)(=O)N5)C3)[nH]c12] |
| 1010 | 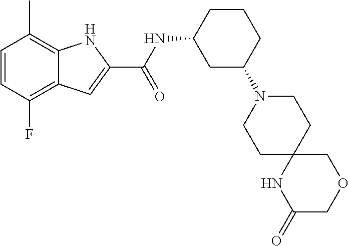 [SMILES: Cc1ccc(F)c2cc(C(=O)N[C@@H]3CCC[C@H](N4CCC5(CC4)COCC(=O)N5)C3)[nH]c12] |
| 1011 | 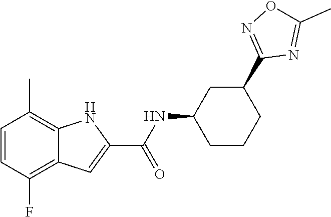 [SMILES: Cc1nc([C@H]2CCC[C@@H](NC(=O)c3cc4c(F)ccc(C)c4[nH]3)C2)no1] |
| 1012 | 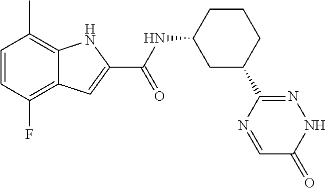 |
| 1013 | 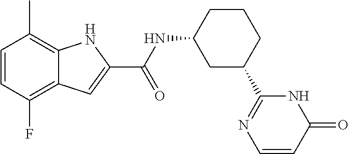 [SMILES: Cc1ccc(F)c2cc(C(=O)N[C@@H]3CCC[C@H](c4nccc(=O)[nH]4)C3)[nH]c12] |
| 1014 | 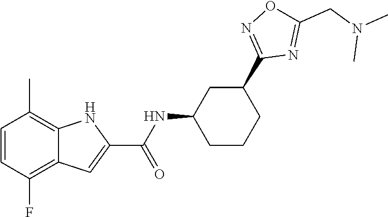 [SMILES: Cc1ccc(F)c2cc(C(=O)N[C@@H]3CCC[C@H](c4noc(CN(C)C)n4)C3)[nH]c12] |
| 1015 | 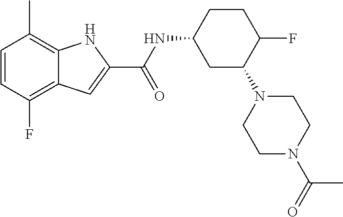 [SMILES: CC(=O)N1CCN([C@@H]2C[C@H](NC(=O)c3cc4c(F)ccc(C)c4[nH]3)CCC2F)CC1] |
| 1016 | 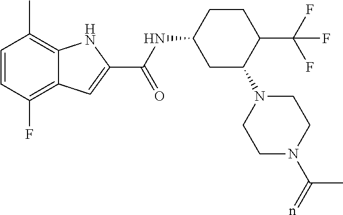 |
| 1017 | 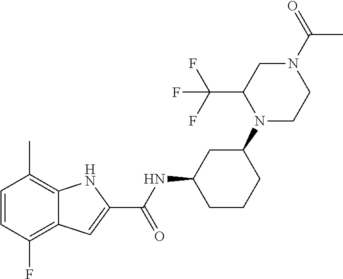 [SMILES: CC(=O)N1CCN([C@H]2CCC[C@@H](NC(=O)c3cc4c(F)ccc(C)c4[nH]3)C2)C(C(F)(F)F)C1] |
| 1018 | 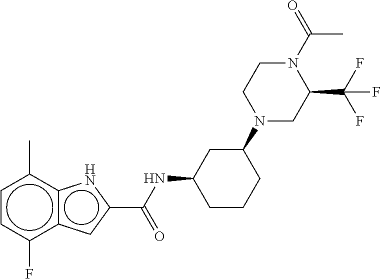 |
| 1020 | 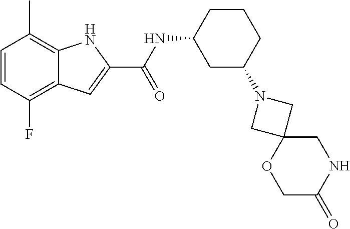 [SMILES: Cc1ccc(F)c2cc(C(=O)N[C@@H]3CCC[C@H](N4CC5(CNC(=O)CO5)C4)C3)[nH]c12] |
| 1021 | 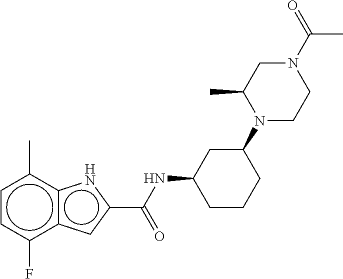 |
| 1022 | 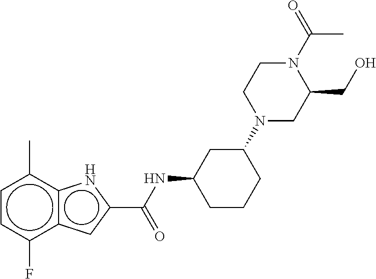 |
| 1023 | 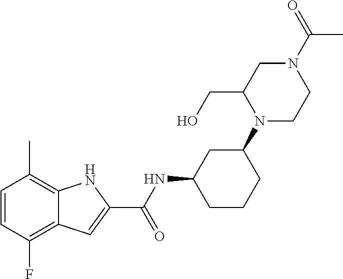 [SMILES: CC(=O)N1CCN([C@H]2CCC[C@@H](NC(=O)c3cc4c(F)ccc(C)c4[nH]3)C2)C(CO)C1] |
| 1024 | 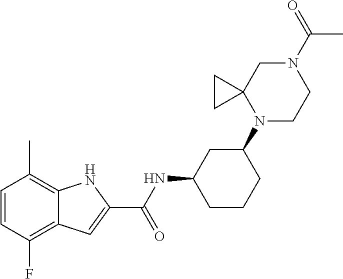 [SMILES: CC(=O)N1CCN([C@H]2CCC[C@@H](NC(=O)c3cc4c(F)ccc(C)c4[nH]3)C2)C2(CC2)C1] |
| 1025 | 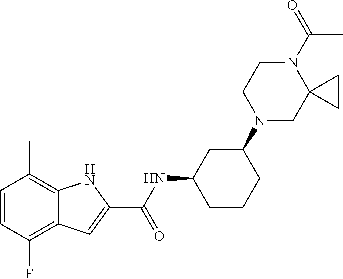 [SMILES: CC(=O)N1CCN([C@H]2CCC[C@@H](NC(=O)c3cc4c(F)ccc(C)c4[nH]3)C2)CC12CC2] |
| 1026 | 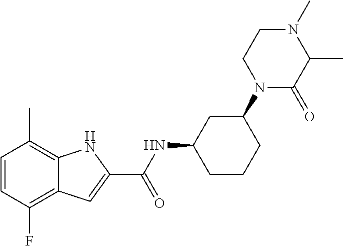 [SMILES: Cc1ccc(F)c2cc(C(=O)N[C@@H]3CCC[C@H](N4CCN(C)C(C)C4=O)C3)[nH]c12] |
| 1027 | 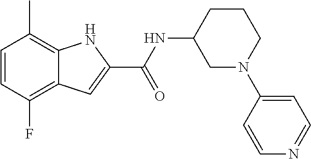 [SMILES: Cc1ccc(F)c2cc(C(=O)NC3CCCN(c4ccncc4)C3)[nH]c12] |
| 1031 | 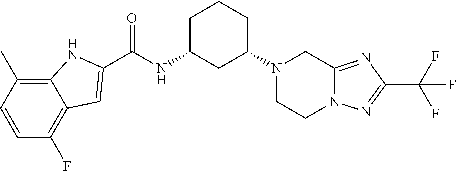 [SMILES: Cc1ccc(F)c2cc(C(=O)N[C@@H]3CCC[C@H](N4CCn5nc(C(F)(F)F)nc5C4)C3)[nH]c12] |
| 1032 | 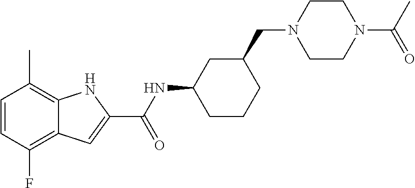 [SMILES: CC(=O)N1CCN(C[C@H]2CCC[C@@H](NC(=O)c3cc4c(F)ccc(C)c4[nH]3)C2)CC1] |
| 1035 | 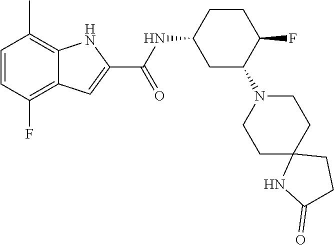 [SMILES: Cc1ccc(F)c2cc(C(=O)N[C@@H]3CC[C@@H](F)[C@H](N4CCC5(CCC(=O)N5)CC4)C3)[nH]c12] |
| 1036 | 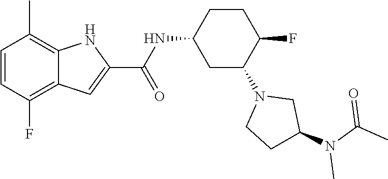 [SMILES: CC(=O)N(C)[C@H]1CCN([C@@H]2C[C@H](NC(=O)c3cc4c(F)ccc(C)c4[nH]3)CC[C@H]2F)C1] |
| 1037 | 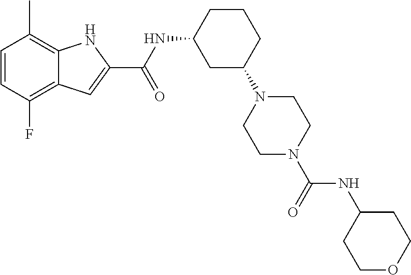 [SMILES: Cc1ccc(F)c2cc(C(=O)N[C@@H]3CCC[C@H](N4CCN(C(=O)NC5CCOCC5)CC4)C3)[nH]c12] |
| 1038 | 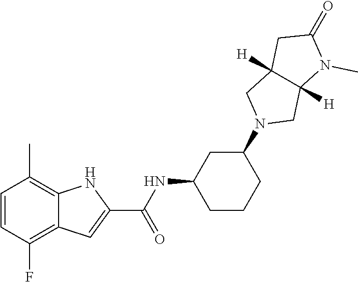 [SMILES: Cc1ccc(F)c2cc(C(=O)N[C@@H]3CCC[C@H](N4C[C@@H]5CC(=O)N(C)[C@@H]5C4)C3)[nH]c12] |
| 1039 | 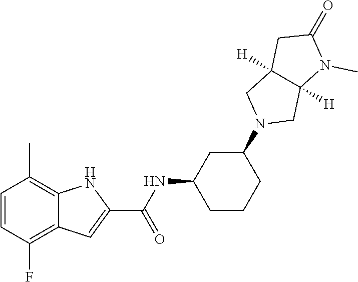 [SMILES: Cc1ccc(F)c2cc(C(=O)N[C@@H]3CCC[C@H](N4C[C@H]5CC(=O)N(C)[C@H]5C4)C3)[nH]c12] |
| 1040 | 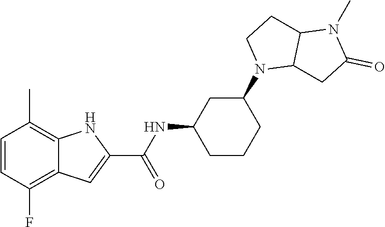 [SMILES: Cc1ccc(F)c2cc(C(=O)N[C@@H]3CCC[C@H](N4CCC5C4CC(=O)N5C)C3)[nH]c12] |
| 1042 | 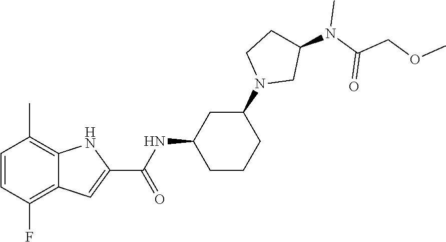 [SMILES: COCC(=O)N(C)[C@@H]1CCN([C@H]2CCC[C@@H](NC(=O)c3cc4c(F)ccc(C)c4[nH]3)C2)C1] |
| 1043 | 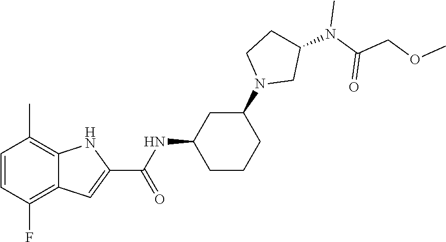 [SMILES: COCC(=O)N(C)[C@H]1CCN([C@H]2CCC[C@@H](NC(=O)c3cc4c(F)ccc(C)c4[nH]3)C2)C1] |
| 1044 | 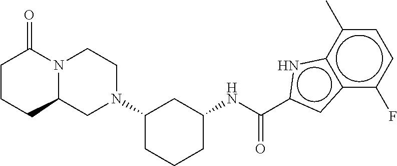 |
| 1045 | 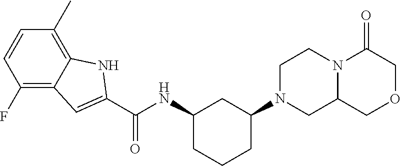 [SMILES: Cc1ccc(F)c2cc(C(=O)N[C@@H]3CCC[C@H](N4CCN5C(=O)COCC5C4)C3)[nH]c12] |
| 1046 | 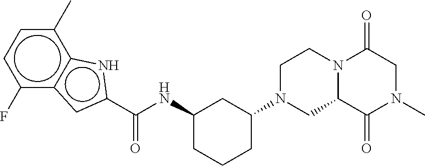 |
| 1047 | 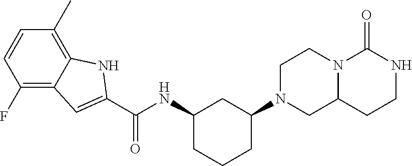 [SMILES: Cc1ccc(F)c2cc(C(=O)N[C@@H]3CCC[C@H](N4CCN5C(=O)NCCC5C4)C3)[nH]c12] |
| 1048 | 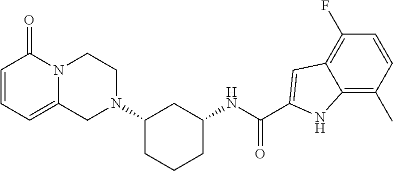 [SMILES: Cc1ccc(F)c2cc(C(=O)N[C@@H]3CCC[C@H](N4CCn5c(cccc5=O)C4)C3)[nH]c12] |
| 1049 | 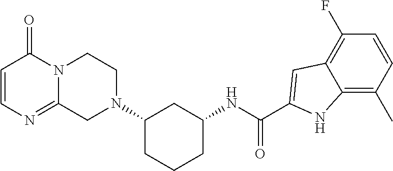 [SMILES: Cc1ccc(F)c2cc(C(=O)N[C@@H]3CCC[C@H](N4CCn5c(nccc5=O)C4)C3)[nH]c12] |
| 1050 | 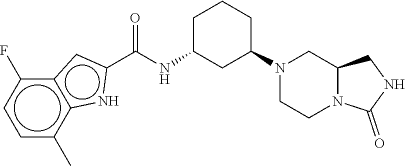 |
| 1051 | 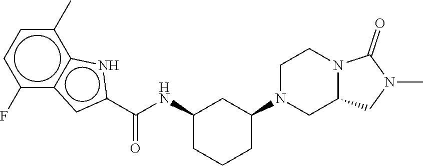 |
| 1052 | 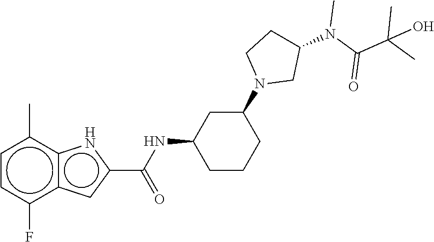 |
| 1053 | 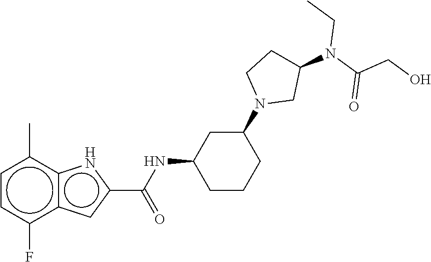 |
| 1054 | 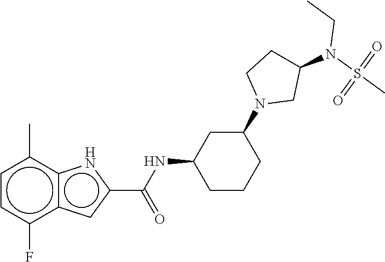 |
| 1055 | 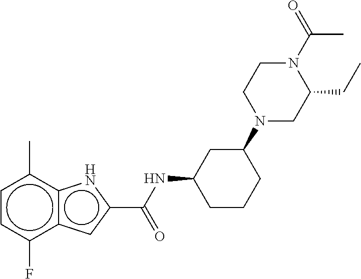 |
| 1056 | 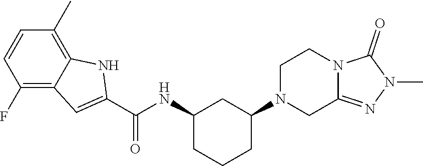 [SMILES: Cc1ccc(F)c2cc(C(=O)N[C@@H]3CCC[C@H](N4CCn5c(nn(C)c5=O)C4)C3)[nH]c12] |
| 1057 | 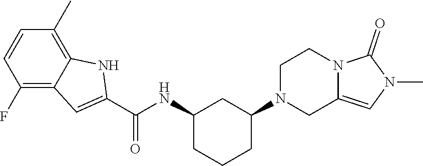 [SMILES: Cc1ccc(F)c2cc(C(=O)N[C@@H]3CCC[C@H](N4CCn5c(cn(C)c5=O)C4)C3)[nH]c12] |
| 1058 | 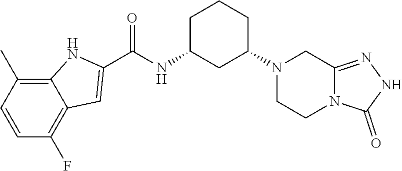 [SMILES: Cc1ccc(F)c2cc(C(=O)N[C@@H]3CCC[C@H](N4CCn5c(n[nH]c5=O)C4)C3)[nH]c12] |
| 1059 | 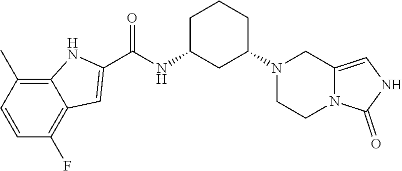 [SMILES: Cc1ccc(F)c2cc(C(=O)N[C@@H]3CCC[C@H](N4CCn5c(c[nH]c5=O)C4)C3)[nH]c12] |
| 1060 | 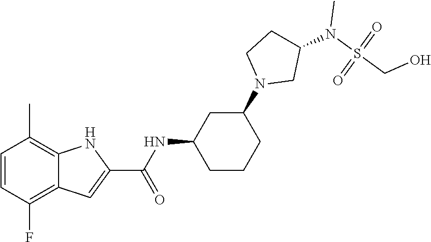 [SMILES: Cc1ccc(F)c2cc(C(=O)N[C@@H]3CCC[C@H](N4CC[C@H](N(C)S(=O)(=O)CO)C4)C3)[nH]c12] |
| 1061 | 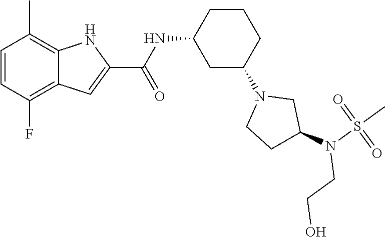 [SMILES: Cc1ccc(F)c2cc(C(=O)N[C@@H]3CCC[C@H](N4CC[C@H](N(CCO)S(C)(=O)=O)C4)C3)[nH]c12] |
| 1062 | 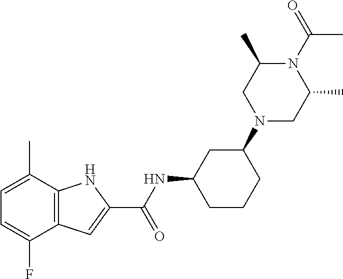 [SMILES: CC(=O)N1[C@H](C)CN([C@H]2CCC[C@@H](NC(=O)c3cc4c(F)ccc(C)c4[nH]3)C2)C[C@H]1C] |
| 1063 | 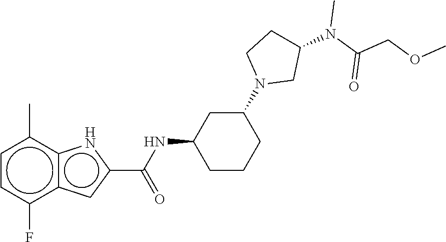 |
| 1064 | 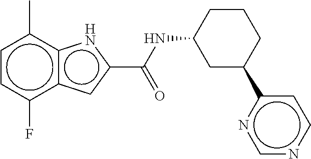 |
| 1065 | 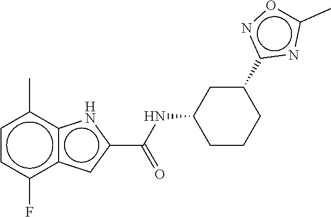 |
| 1066 | 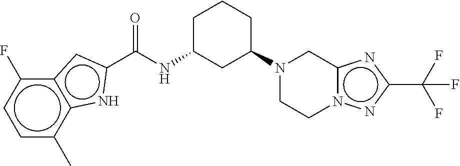 |
| 1067 | 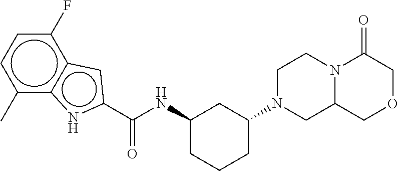 |
| 1068 | 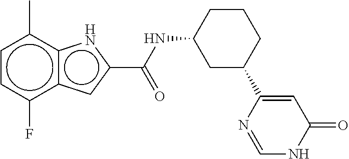 |
| 1070 | 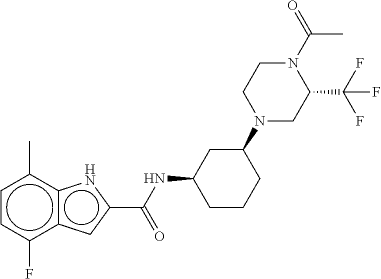 |
| 1071 | 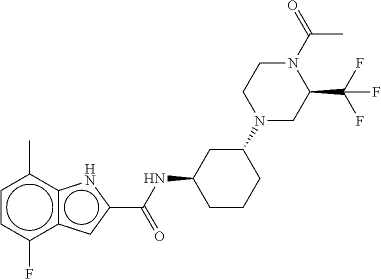 |
| 1072 | 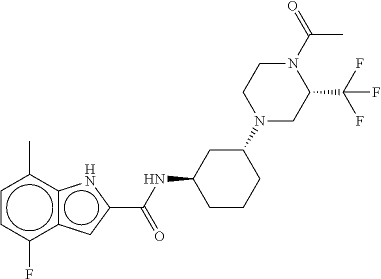 |
| 1074 | 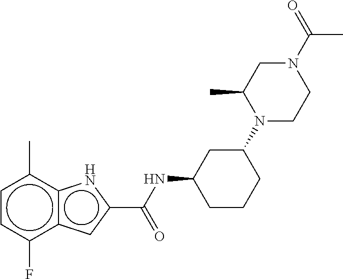 |
| 1076 | 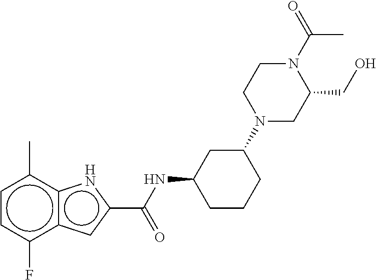 |
| 1077 | 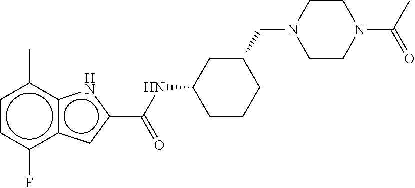 |
| 1078 | 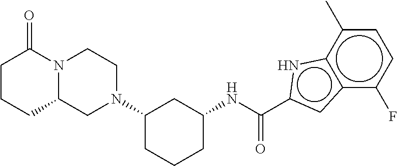 |
| 1079 | 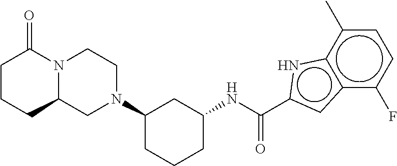 |
| 1080 | 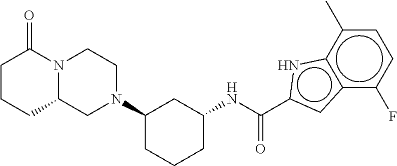 |
| 1082 | 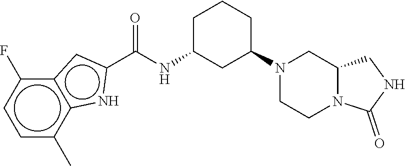 |
| 1083 | 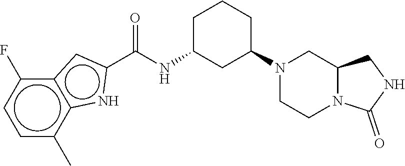 |
| 1084 | 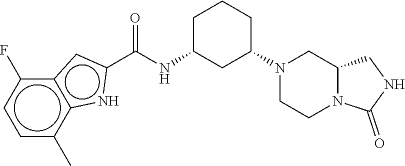 |
| 1085 | 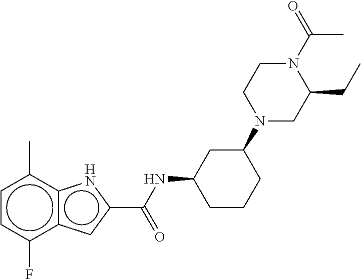 |
| 1086 | 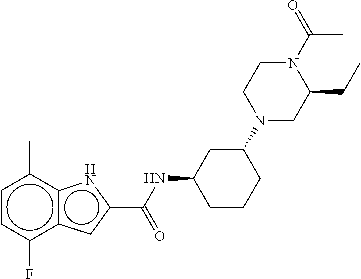 |
| 1087 | 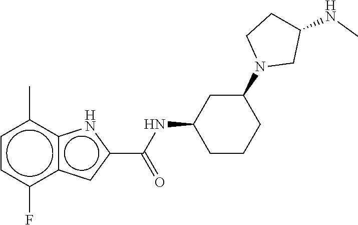 |
| 1088 | 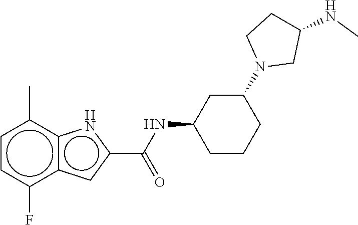 |
| 1090 | 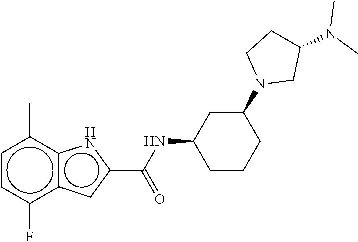 |
| 1091 | 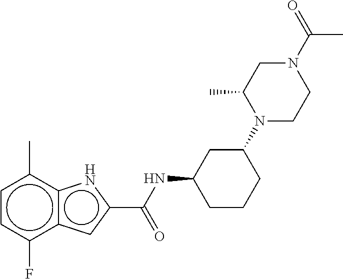 |
| 1092 | 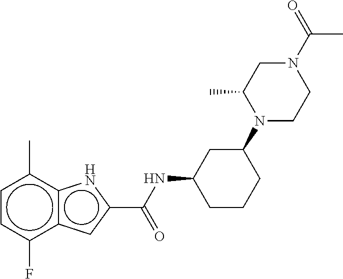 |
| 1095 | 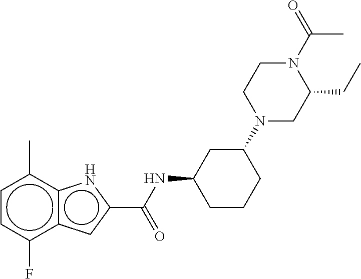 |
| 1096 | 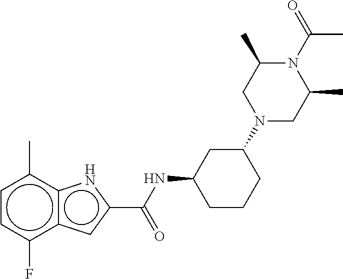 |
| 1097 | 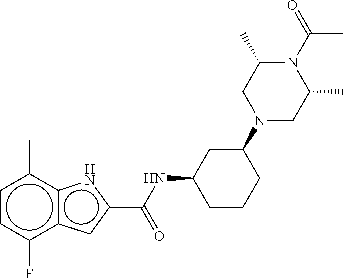 |
| 1098 | 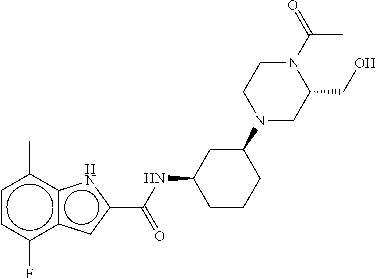 |
| 1099 | 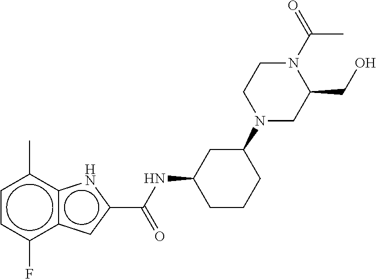 |
| 1100 | 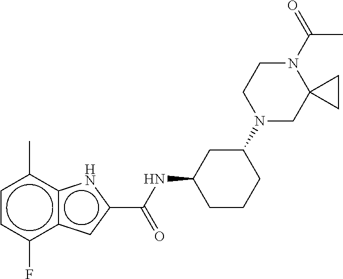 |
| 1102 | 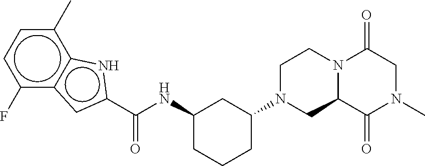 |
| 1103 | 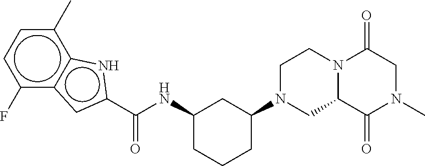 |
| 1104 | 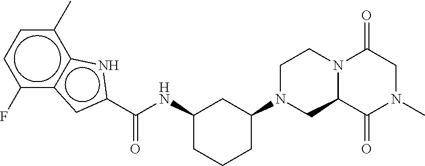 |
| 1106 | 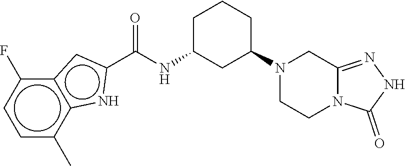 |
| 1107 | 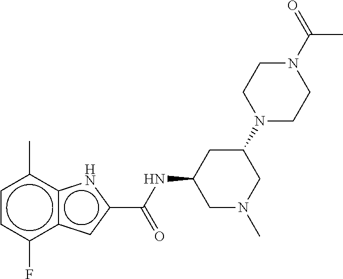 |
| 1108 | 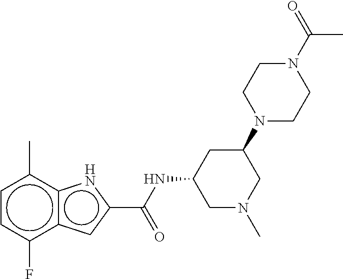 |
| 1109 | 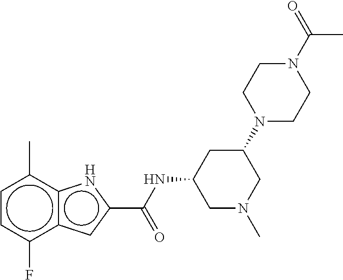 |
| 1110 | 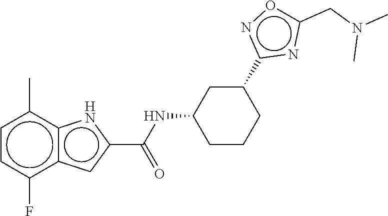 |
| 1111 | 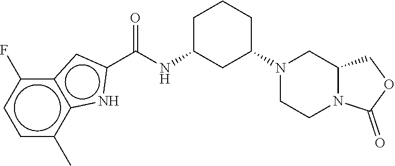 |
| 1112 | 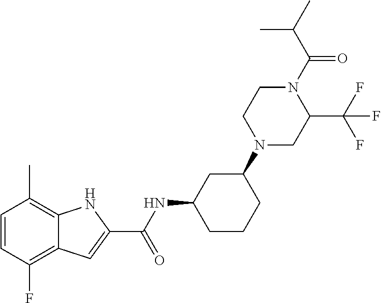 [SMILES: Cc1ccc(F)c2cc(C(=O)N[C@@H]3CCC[C@H](N4CCN(C(=O)C(C)C)C(C(F)(F)F)C4)C3)[nH]c12] |
| 1113 | 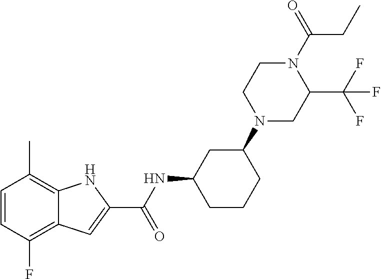 [SMILES: CCC(=O)N1CCN([C@H]2CCC[C@@H](NC(=O)c3cc4c(F)ccc(C)c4[nH]3)C2)CC1C(F)(F)F] |
| 1114 | 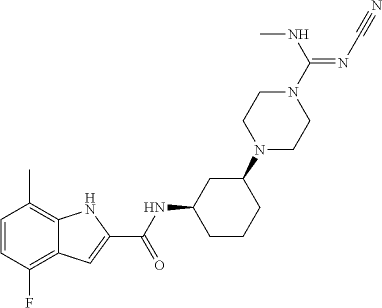 [SMILES: CN/C(=N\C#N)N1CCN([C@H]2CCC[C@@H](NC(=O)c3cc4c(F)ccc(C)c4[nH]3)C2)CC1] |
| 1115 | 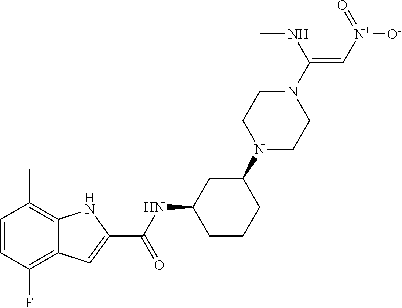 [SMILES: CN/C(=C\[N+](=O)[O-])N1CCN([C@H]2CCC[C@@H](NC(=O)c3cc4c(F)ccc(C)c4[nH]3)C2)CC1] |
| 1116 | 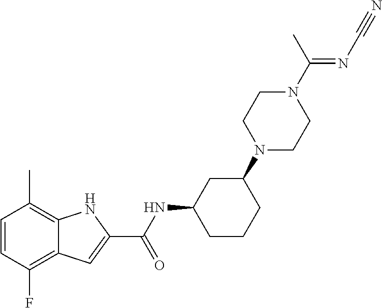 [SMILES: C/C(=N\C#N)N1CCN([C@H]2CCC[C@@H](NC(=O)c3cc4c(F)ccc(C)c4[nH]3)C2)CC1] |
| 1117 | 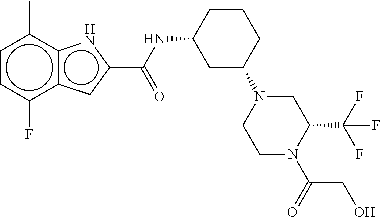 |
| 1118 | 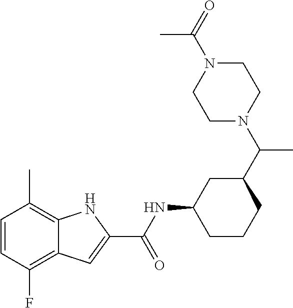 [SMILES: CC(=O)N1CCN(C(C)[C@H]2CCC[C@@H](NC(=O)c3cc4c(F)ccc(C)c4[nH]3)C2)CC1] |
| 1120 | 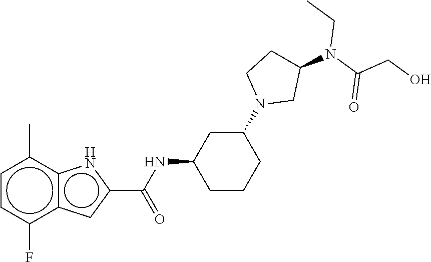 |
| 1122 | 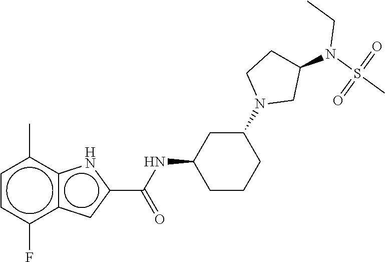 |
| 1123 | 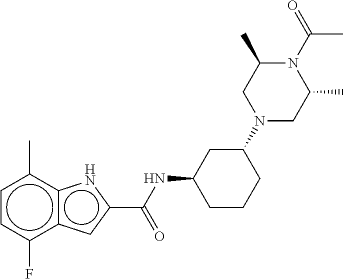 |
| 1124 | 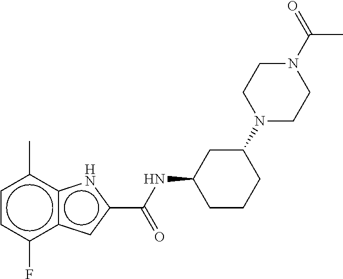 |
| 1126 | 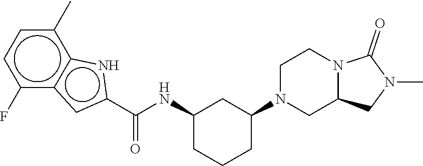 |
| 1127 | 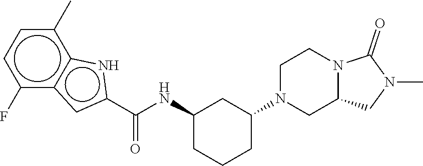 |
| 1128 | 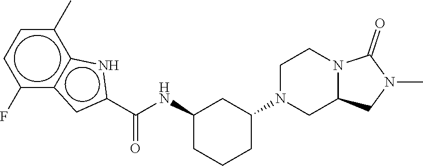 |
| 1129 | 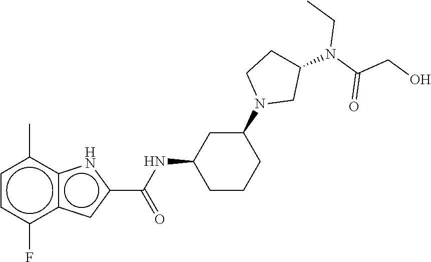 |
| 1130 | 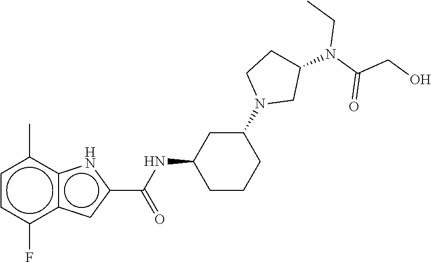 |
| 1131 | 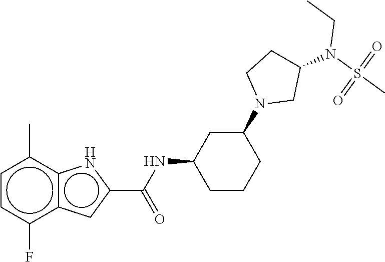 |
| 1132 | 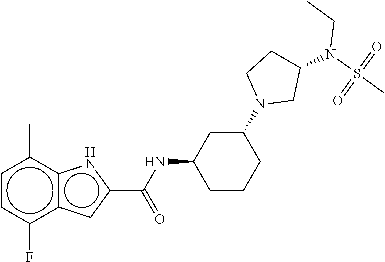 |
| 1134 | 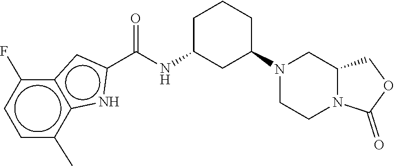 |
| 1135 | 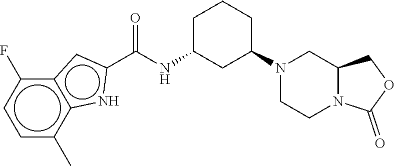 |
| 1136 | 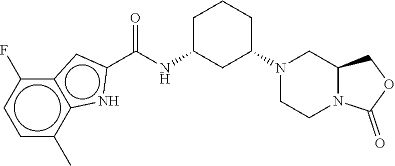 |
| 1137 | 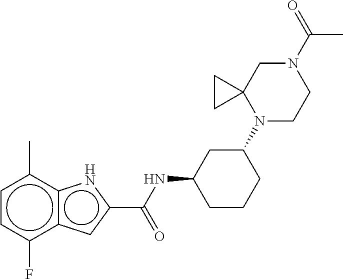 |
| 1138 | 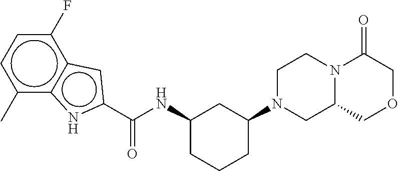 |
| 1139 | 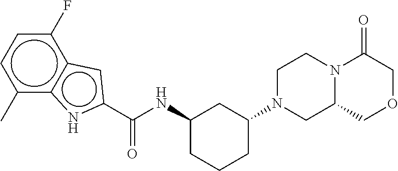 |
| 1140 | 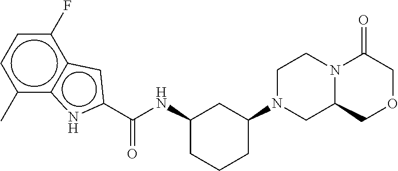 |
| 1141 | 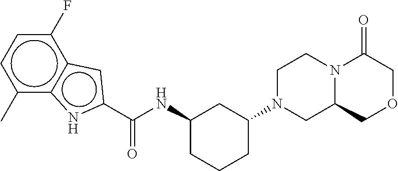 |
| 1142 | 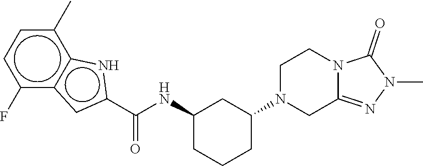 |
| 1143 | 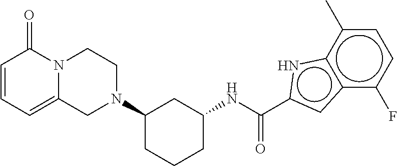 |
| 1145 | 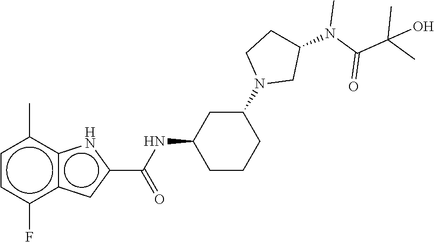 |
| 1146 | 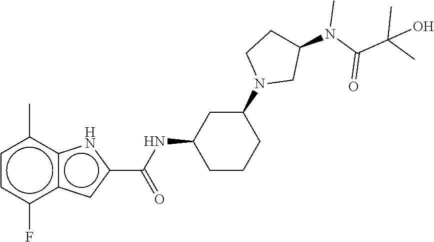 |
| 1147 | 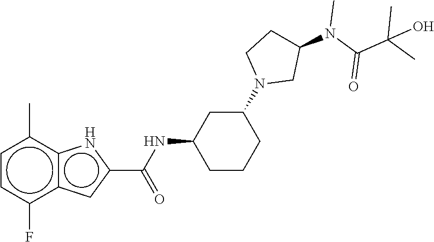 |
| 1148 | 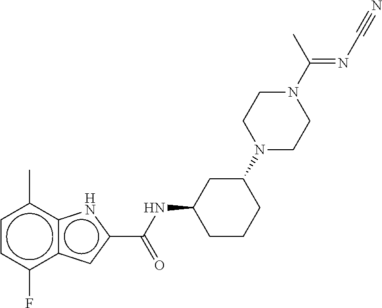 |
| 1150 | 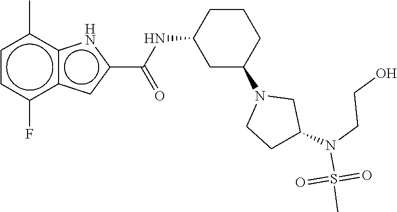 |
| 1151 | 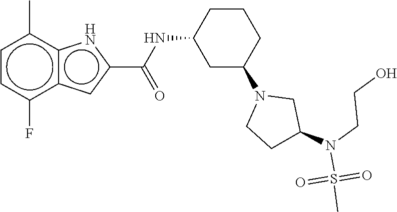 |
| 1152 | 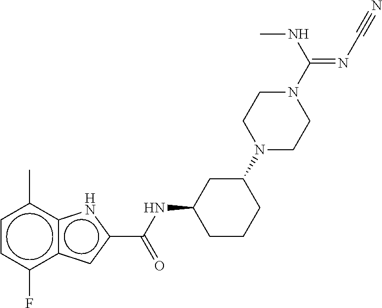 |
| 1153 | 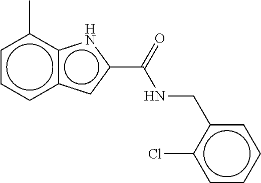 |
| 1154 | 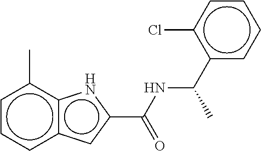 |
| 1155 | 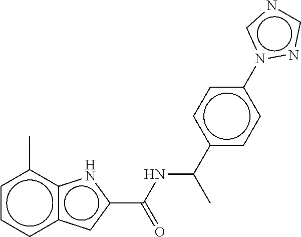 |
| 1156 | 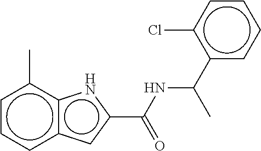 |
| 1157 | 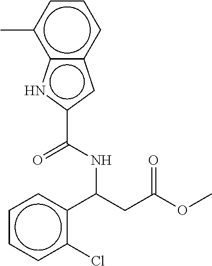 |
| 1158 | 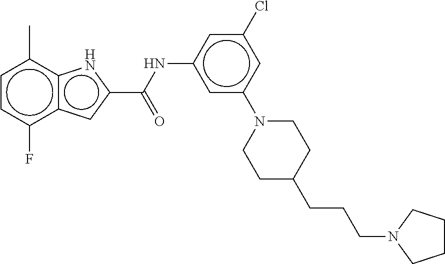 |
| 1159 | 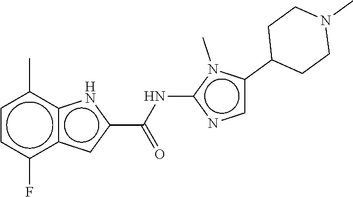 |
| 1160 | 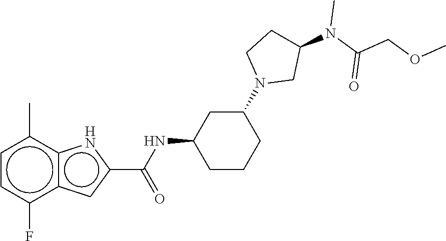 |
| 1161 | 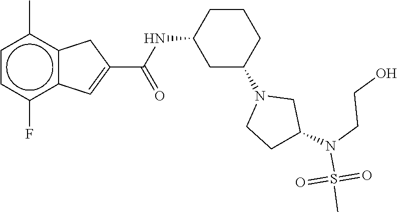 |
| 1162 | 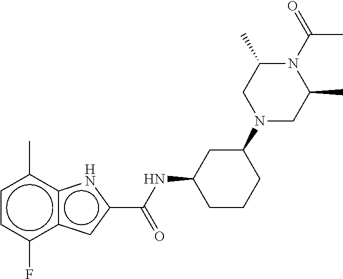 |
| 1163 | 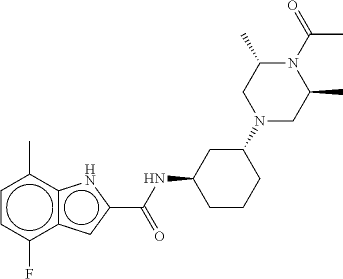 |
| 1164 | 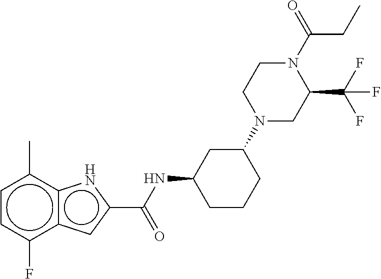 |
| 1165 | 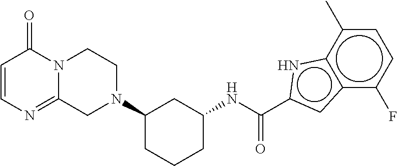 |
| 1166 | 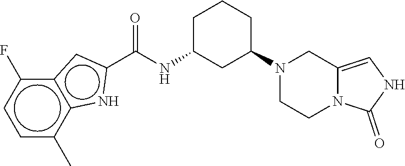 |
| 1167 | 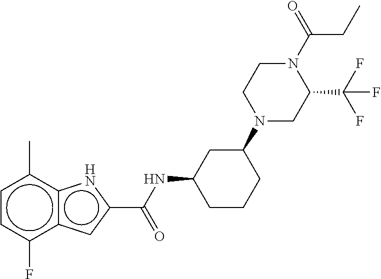 |
| 1168 | 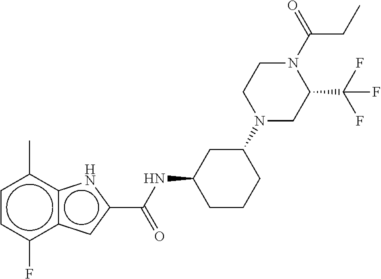 |
| 1169 | 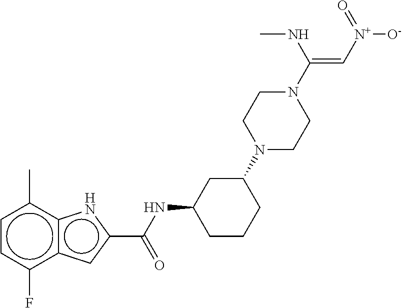 |
| 1170 | 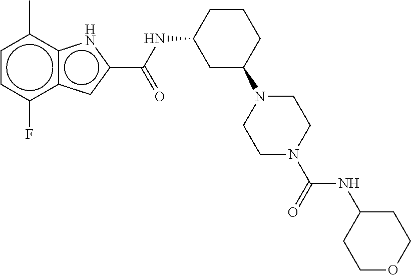 |
| 1171 | 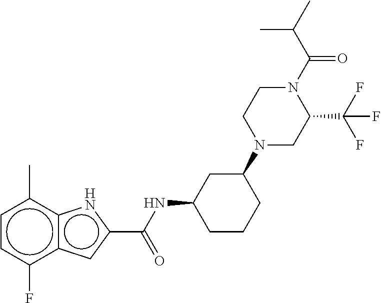 |
| 1172 | 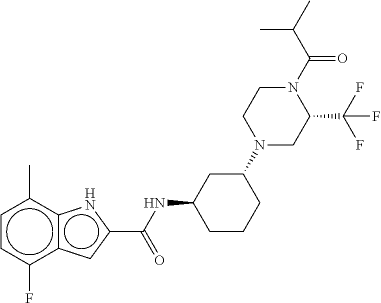 |
| 1173 | 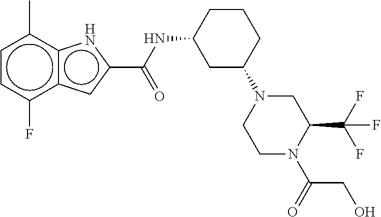 |
| 1174 | 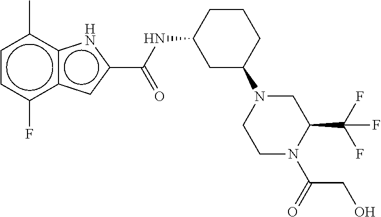 |
| 1175 | 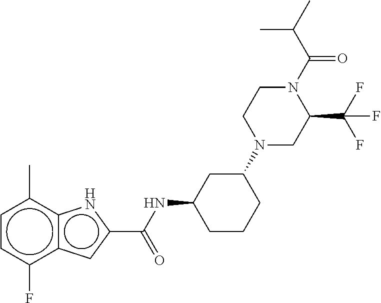 |
| 1176 | 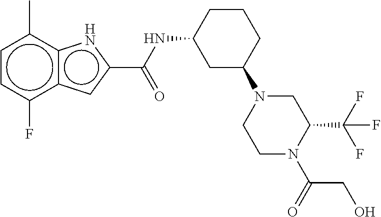 |
| 1177 | 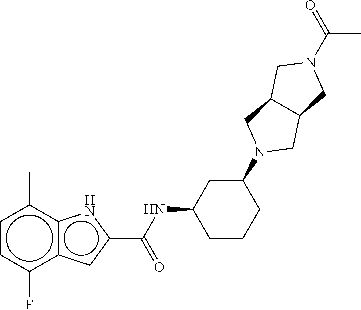 |
| 1178 | 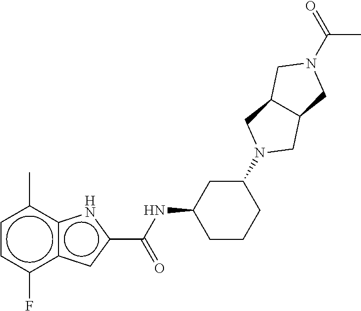 |
| 1179 | 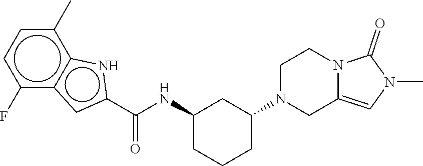 |
| 1180 | 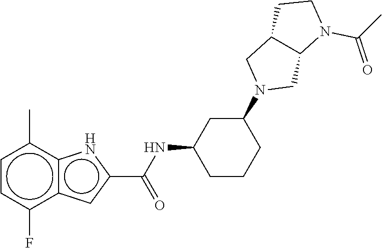 |
| 1181 | 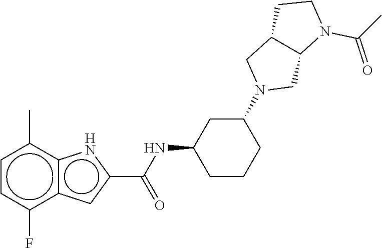 |
| 1182 | 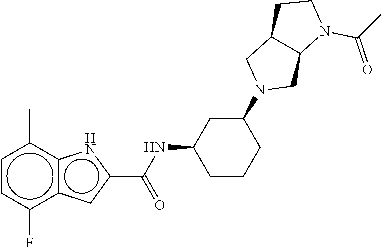 |
| 1183 | 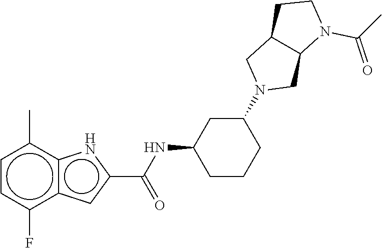 |
| 1184 | 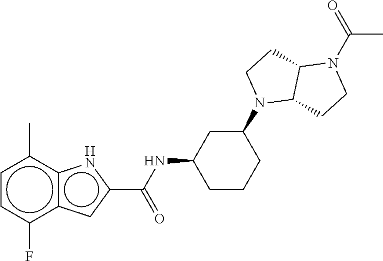 |
| 1185 | 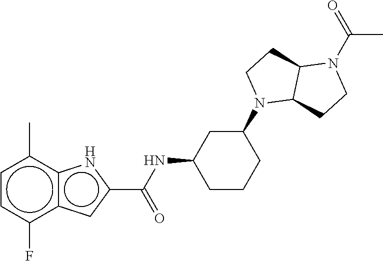 |
| 1186 | 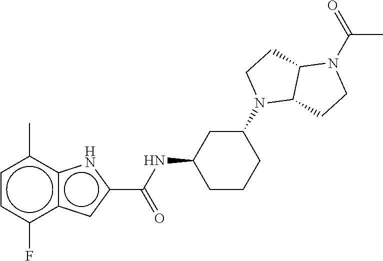 |
| 1187 | 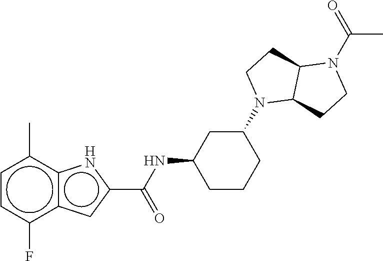 |
| 1188 | 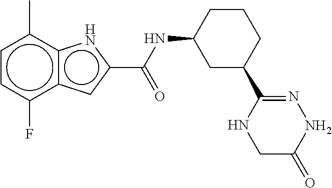 |
| 1189 | 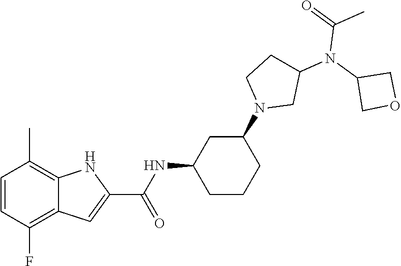 [SMILES: CC(=O)N(C1COC1)C1CCN([C@H]2CCC[C@@H](NC(=O)c3cc4c(F)ccc(C)c4[nH]3)C2)C1] |
| 1190 | 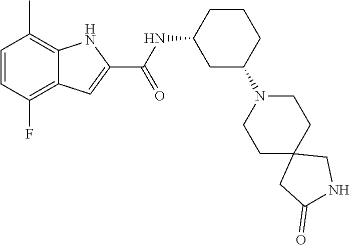 [SMILES: Cc1ccc(F)c2cc(C(=O)N[C@@H]3CCC[C@H](N4CCC5(CC4)CNC(=O)C5)C3)[nH]c12] |
| 1191 | 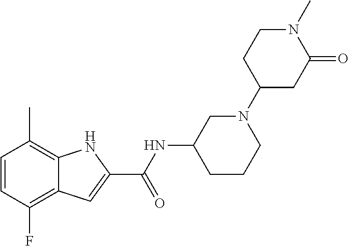 [SMILES: Cc1ccc(F)c2cc(C(=O)NC3CCCN(C4CCN(C)C(=O)C4)C3)[nH]c12] |
| 1192 | 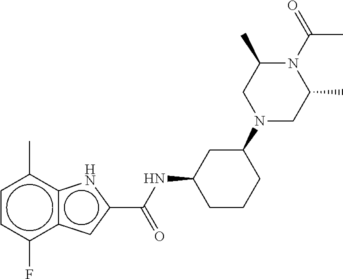 |
| 1193 | 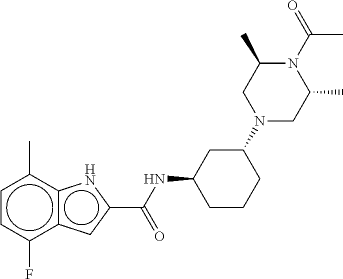 |
| 1194 | 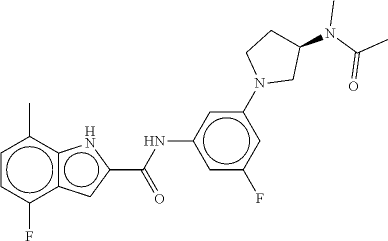 |
| 1195 | 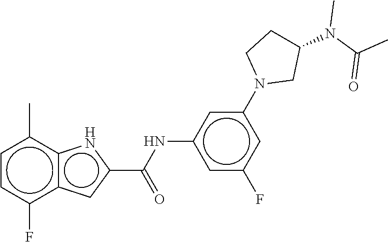 |
| 1196 | 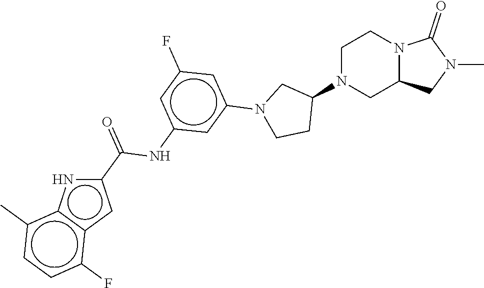 |
| 1197 | 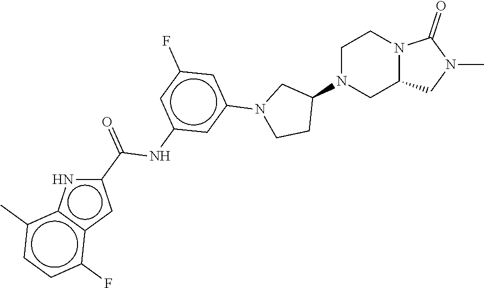 |
| 1198 | 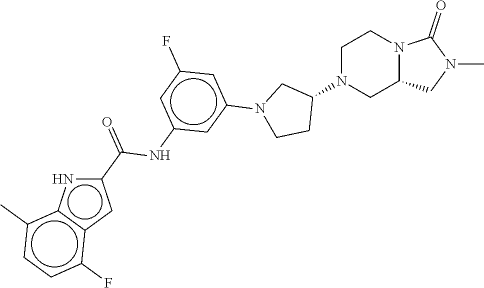 |
| 1199 | 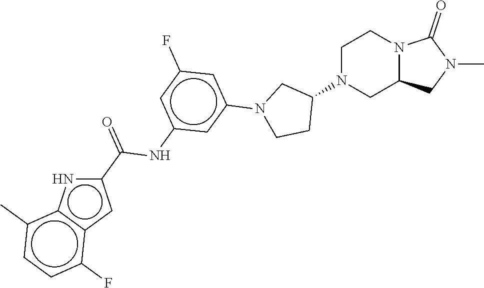 |
| 1200 | 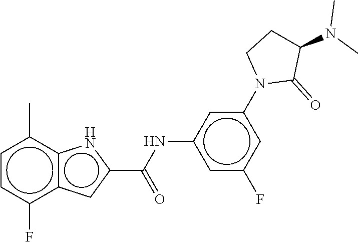 |
| 1201 | 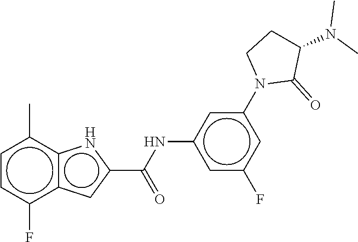 |
| 1202 | 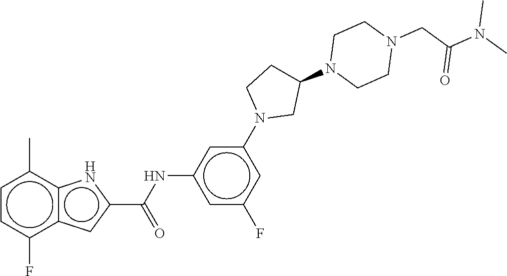 |
| 1203 | 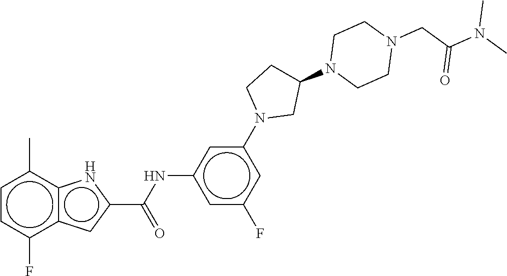 |
| 1204 | 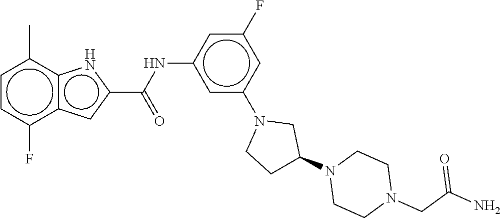 |
| 1205 | 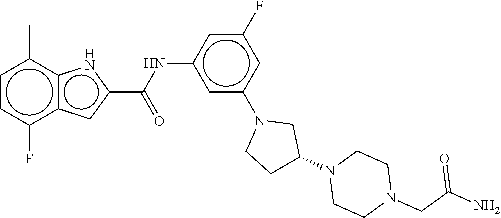 |
| 1206 | 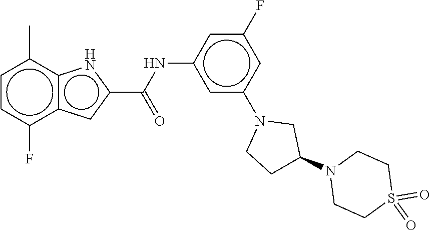 |
| 1207 | 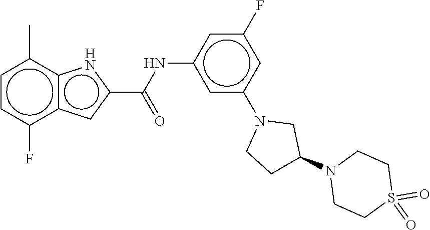 |
| 1208 | 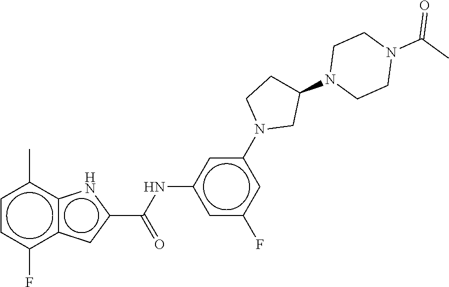 |
| 1209 | 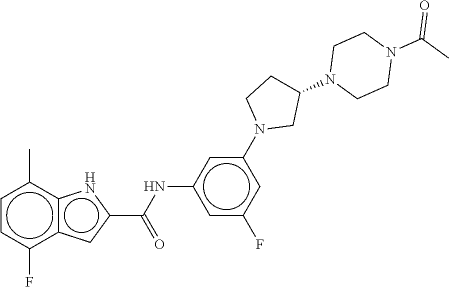 |
| 1210 | 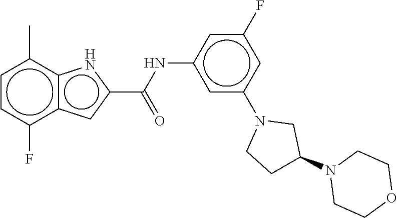 |
| 1211 | 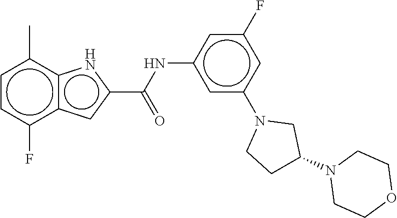 |
| 1212 | 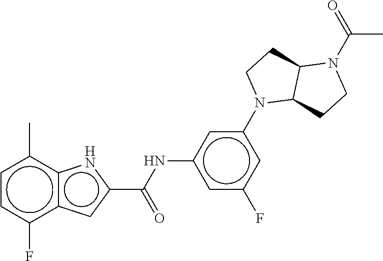 |
| 1213 | 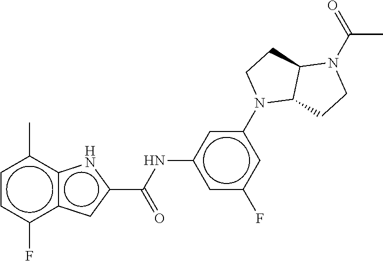 |
| 1214 | 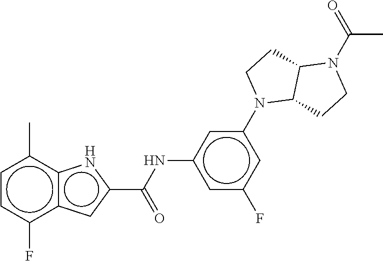 |
| 1215 | 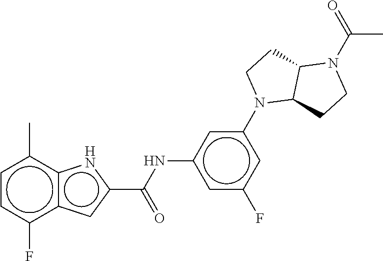 |
| 1216 | 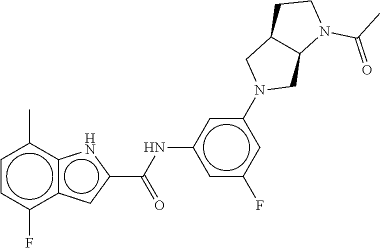 |
| 1217 | 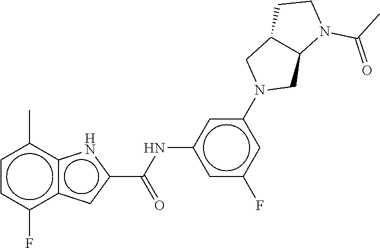 |
| 1218 | 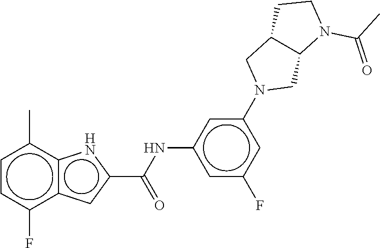 |
| 1219 | 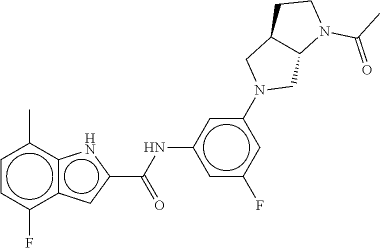 |
| 1220 | 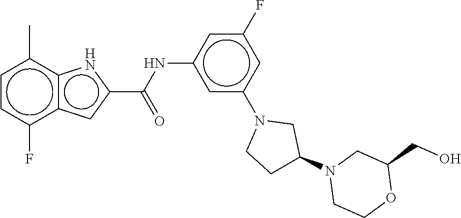 |
| 1221 | 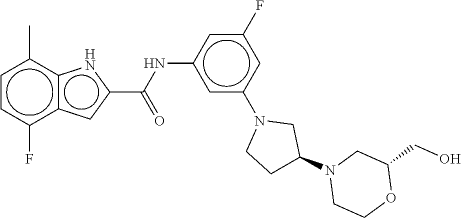 |
| 1222 | 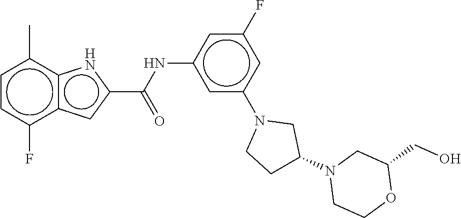 |
| 1223 | 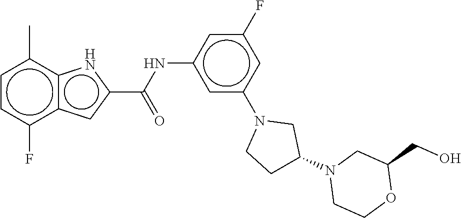 |
| 1224 | 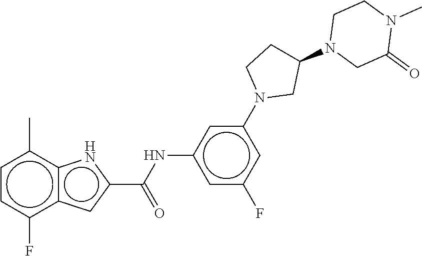 |
| 1225 | 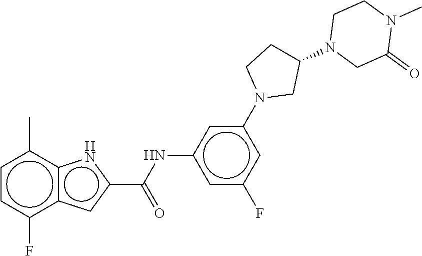 |
| 1226 | 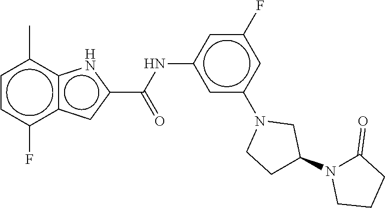 |
| 1227 | 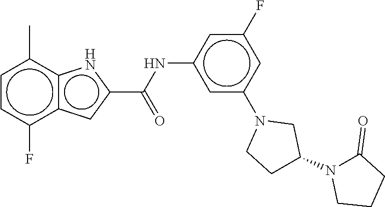 |

or a pharmaceutically acceptable salt or solvate thereof.

Embodiment 72. The compound of Embodiment 71 selected from the group consisting of selected from the group consisting of Cpd. Nos. 1, 2, 3, 4, 5, 6, 7, 8, 9, 10, 11, 12, 13, 14, 15, 16, 17, 18, 824, 828, 839, 870, 922, 930, 942, 995, 1007, 1025, 1043, 1044, 1045, 1048, 1051, 1055, 1070, 1078, 1083, 1097, 1117, 1138, 1180, 1184, and 1192, or a pharmaceutically acceptable salt or solvate thereof.

Embodiment 73. The compound of Embodiment 73 selected from the group consisting of selected from the group consisting of Cpd. Nos. 15, 922, 930, 942, 1055, 1070, 1117, 1180, 1184, and 1192, or a pharmaceutically acceptable salt or solvate thereof.

The term “SETD2” (also known as SET Domain Containing 2, Huntingtin-Interacting Protein B, Lysine A-Methyltransferase 3A, Huntingtin Yeast Partner B, EC 2.1.1.43, P231HBP, HIP-1, HIF-1, KMT3A, HYPB, SET2, Histone-Lysine N-Methyltransferase SETD2, Huntingtin Interacting Protein 1, Huntingtin-Interacting Protein 1, SET Domain-Containing Protein 2, KIAA1732, HSPC069, HBP231, HSET2, HIF1, and LLS) refers to native histone methyltransferase SETD2, unless otherwise indicated. “Human SETD2” refers to native human histone methyltransferase SETD2. “SETD2” encompasses full-length, unprocessed SETD2, as well as any form of SETD2 that results from processing within the cell. The term also encompasses naturally occurring variants of SETD2, e.g., splice variants, allelic variants, and isoforms. SETD2 can be isolated from a variety of sources, e.g., from human tissue types or other animal tissue types, or prepared by recombinant or synthetic methods. Examples of human gene sequences encoding SETD2 or SETD2 polypeptide sequences include, but are not limited to, NCBI Gene ID 29072, HGNC: 18420, and SETD2 transcript variant 1, mRNA-NCBI Reference Sequence: NM 014159.6. The human gene encoding SETD2 is located on the short arm of chromosome 3. While the term “SETD2,” as used herein, generally refers to the gene encoding human SETD2, other mammalian forms of SETD2 are contemplated as well.

As used herein, a “functional domain of SETD2” refers to one of the three conserved functional domains of SETD2 that are believed to define the biological function of SETD2. These functional domains are (1) the triplicate AWS-SET-PostSET domain; (2) a WW domain; and (3) a Set2-Rbp1 interacting (“SRI”) domain (Li, J. et al., Oncotarget 7:50719-50734 (2016)), which can be described as follows:

AWS-SET-PostSETdomain. Without wishing to be bound by any theory, it is believed that the human SET domain is a motif of 130 amino acids that is evolutionarily conserved from yeast to mammals and is also found in some bacteria and viruses. The SET domain is usually present as part of a multi-domain, flanked by an AWS (Associated with SET) and a PostSET domain. Generally, SET-domain-containing proteins transfer one or several methyl groups from k-adenosyl-L-methionine to the amino group of a lysine or an arginine residue of histones or other proteins. It is believed that this transfer is dependent on the flanking AWS and PostSET regions, which contain several conserved cysteine residues. In contrast to other methyltransferases, SET-domain-containing methyltransferases have an α-sheet structure that facilitates multiple rounds of methylation without substrate disassociation.

WW domain. The “WW domain” refers to the presence of two conserved tryptophan (W) residues spaced 20-22 amino acids apart. Binding assays show that the WW domain preferentially binds to proline-rich segments, mediating protein-protein interactions to participate in a variety of molecular processes. Without wishing to be bound by any theory, it is believed that the WW domain recognizes motifs like Proline-Proline-x-Tyrosine (PPxY), phospho-Serine-Proline (p-SP) or phospho-Threonine-Proline (p-ST), and mediates protein binding. Aberrant expression of WW-domain-containing genes has been associated with diseases such as HD, Alzheimer's disease, and multiple cancer subtypes. Without wishing to be bound by any theory, it is believed that the WW domain in the C-terminal region of SETD2 interacts with the Huntingtin protein via its proline-rich segment, regardless of the length of the HD-associated polyglutamine track, and may also interact with TP53. SETD2 contains a proline-rich stretch that precedes the WW domain. This proline-rich stretch functions as an intramolecular WW-interacting domain that can block the WW domain of SETD2 from interacting with the proline-rich stretch of Huntingtin, and most likely of other proteins as well.

SRI domain. Without wishing to be bound by any theory, it is believed that the Set2 Rpb1 Interacting (“SRI”) domain interacts with the hyperphosphorylated C-terminal domain (CTD) of Rpb1, the largest subunit of RNA Pol II. Also without wishing to be bound by any theory, it is believed that in humans, the primary C-terminal domain-docking site of RNA Pol II is located at the first and second helices of SETD2. This domain is believed to direct the activity of SETD2 towards actively transcribed genes.

As used herein, the term “WHSC1” (also known as Wolf-Hirschhorn Syndrome Candidate Gene 1, MMSET, NSD2, REIIBP, TRX5, and WHS) refers to a histone methyltransferase enzyme located at the chromosome 4p16.3 locus. WHSC1 is significantly overexpressed in multiple cancer types compared to their normal counterparts. Furthermore, WHSC1 is associated with tumor aggressiveness or prognosis in many types of these cancers. See, Kassambara, A. et al., Biochem. Biophys. Res. Commun. 379:840-845 (2009). See also, Hudlebusch H. R. et al., Clin Cancer Res. 17:2919-2933 (2011). In a subset of multiple myeloma, a chromosomal translocation occurs where the 4p16.3 locus of WHSC1 is fused to the 14q32 locus, and WHSC1 is significantly overexpressed. This translocation is commonly known as t(4; 14), and is described in further detail below.

As used herein, the term “overexpression” means expression (e.g., of a WHSC1 polynucleotide or polypeptide) at levels exceeding those present in normal cells or cells of a different phenotypic status. In one embodiment, WHSC1 expression is differentially present (e.g., overexpressed) in a subject of one phenotypic status, e.g., a subject having a hematological cancer, as compared with another phenotypic status, e.g., a normal undiseased subject or a patient having cancer without overexpression of WHSC1. Comparison may be carried out by statistical analyses on numeric measurements of the expression, or, it may be done through visual examination of experimental results.

As used herein, the term “subject” refers to any animal (e.g., a mammal), including, but not limited to humans, non-human primates, rodents, and the like, which is to be the recipient of a particular treatment. Typically, the terms “subject” and “patient” are used interchangeably herein in reference to a human subject.

“Tumor” and “neoplasm” refer to any mass of tissue that results from excessive cell growth or proliferation, either benign (noncancerous) or malignant (cancerous) including pre-cancerous and in situ lesions.

The terms “cancer,” “cancerous,” or “malignancy,” are used interchangeably, and refer to the physiological condition in mammals (i.e., humans) in which a population of cells are characterized by uncontrolled or unregulated cell growth or proliferation. Examples of cancer include, e.g., carcinoma, lymphoma, blastoma, sarcoma, myeloma, and leukemia. Non-limiting examples of cancer types that may be treated with the methods and pharmaceutical compositions of the present disclosure include esophageal cancer, kidney cancer, stomach cancer, hepatocellular carcinoma, glioblastoma, central nervous system (CNS) cancer, soft tissue cancer, lung cancer, breast cancer, bladder/urinary tract cancer, head and neck cancer, prostate cancer, testicular cancer, melanoma, hematological cancer, multiple myeloma, pancreatic cancer, colorectal cancer, skin cancer, endometrial cancer, ovarian cancer, colon cancer, and colorectal cancer.

The term “relapsed” cancer in a patient refers to patients who have previously achieved either a complete or partial remission, but after a period of 6 or more months, demonstrate evidence of disease progression. The term “refractory” cancer in a patient refers to patients who have experienced treatment failure or disease progression within 6 months from the last anti-cancer therapy.

A tumor which “does not respond” or “responds poorly” to treatment (with, for example, a particular chemotherapeutic regimen) does not show statistically significant improvement in response to that treatment when compared to no treatment or treatment with a placebo in a recognized animal model or human clinical trial, or which responds to an initial treatment, but grows as treatment continues.

The term “multiple myeloma” or “MM” refers to a heterogeneous plasma cell disorder/hematologic cancer characterized by multiple molecularly-defined subtypes each with varying clinicopathological features and disease outcomes. MM is also known as “plasma cell myeloma” or “PCM.” The American Cancer Society estimates that in 2019 in the U.S, about 32,000 new cases will be diagnosed and about 13,000 deaths are expected to occur. Genetic abnormalities in MM include, e.g., chromosomal translocations, deletions, duplications, copy number variations from gain or loss of DNA (e.g., hyperdiploidy, gain of 1q, loss of 1p, loss of chromosome 13/13q, loss of 17p) and genetic mutations. Prideaux, S. M. et al., Advances in Hematology Volume 2014:1-16 (2014).

Chromosomal translocations are early events in MM pathogenesis that are followed by secondary changes. The term “chromosomal translocation” refers to a genetic abnormality whereby genetic material from one chromosome is transferred to a different position on (most often) a nonhomologous chromosome. Translocations can be classified into two main categories—reciprocal (or balanced) and nonreciprocal. In the more typical “reciprocal translocation,” genetic material is exchanged between the two-non-homologous chromosomes. In a non-reciprocal translocation, there is a one-way transfer of genetic material from one chromosome to another chromosome.

In approximately 40-50% of patients with multiple myeloma, translocations occur between the immunoglobulin heavy chain alleles at chromosome 14q32 and various partner chromosomes. Pawlyn, C. et al., Nat. Rev. Cancer 77:543-556 (2017). Translocation of oncogenes into this region may lead to their increased expression, contributing to disease initiation, disease progression, and therapeutic resistance. Several chromosomal translocations have been identified in patients with multiple myeloma, and include t(4; 14), t(14;16), t(14;20), t(8; 14), t(11; 14), and t(6;14), although t(11; 14), and t(6;14) are reported as neutral translocations. See, Kalff and Spencer, Blood Cancer Journal 2:e89 (2012).

As used here, the term “t(4;14) multiple myeloma” or “t(4;14) MM” refers to a subset of MM whereby a translocation between chromosomes 4 and 14 is present. The t(4; 14) translocation is associated with upregulation of the fibroblast growth factor receptor 3 (FGFR3) and WHSC1. More particularly, in t(4:14) MM, a chromosomal translocation occurs where the 4p16.3 locus of WHSC1 is fused to the 14q32 locus. The result of t(4; 14) in multiple myeloma is that the WHSC1 gene is placed under the transcriptional control of the immunoglobulin heavy chain (IgH) promoter/enhancer region. This leads to a massive up-regulation and overexpression of WHSC1 (Chesi et al., Blood 92:3025-3034 (1998)). The overexpression of WHSC1 leads to a global increase in the dimethylation of histone H3 at lysine 36 (H3K36me2). (Kuo et al., Mol. Cell 44:609-620 (2011)). WHSC1 is now recognized as a driver in t(4; 14) pathogenesis. Like WHSC1, SETD2 also methylates H3K36, by adding a third methyl group (H3K36me3, trimethylation) utilizing the WHSC1-catalyzed H3K36me2 as its substrate. SETD2 is the only known HMT capable of catalyzing H3K36 trimethylation.

Based on the sensitivity of t(4; 14) multiple myeloma cell lines to SETD2 inhibition (as demonstrated in the Example below), the oncogenic function resulting from increased H3K36me2 driven by WHSC1 overexpression in t(4; 14) MM likely also requires the ability of SETD2 to add an additional methyl group. In other words, SETD2 inhibition in t(4; 14) multiple myeloma cells is dependent on the overexpression of WHSC1. Therefore, without wishing to be bound by any theory, it is believed that the aberrant H3K36me2 driven by WHSC1 overexpression in t(4; 14) MM presents additional substrates for trimethylation that leads to an oncogenic dependence on SETD2.

The term “non-t(4;14) multiple myeloma” or “non-t(4;14) MM” refers to a subset of MM whereby there is a chromosomal translocation present other than t(4;14). For example, translocations in “non-t(4;14) multiple myeloma” include, e.g., t(14; 16), t(14;20), t(8; 14), t(11;14), and t(6;14).

The term “pharmaceutical formulation” refers to a preparation that is in such a form as to permit the biological activity of the active ingredient(s) to be effective, and which contains no additional components which are unacceptably toxic to a subject to which the formulation would be administered. Such a formulation can be sterile.

The term “therapeutically effective amount” refers to the amount of a therapeutic agent (e.g., a small molecule inhibitor of SETD2) that is effective to “treat” a disease or disorder in a subject or mammal. In the case of cancer, the therapeutically effective amount of the agent can reduce the number of cancer cells, reduce the proliferation of cancer cells, reduce the tumor size, inhibit (i.e., slow to some extent and in some embodiments, stop) cancer cell infiltration into peripheral organs, inhibit (i.e., slow to some extent and in some embodiments, stop) tumor metastasis, inhibit, to some extent, tumor growth, and/or relieve to some extent one or more of the symptoms associated with the cancer. See the definition herein of “treating.” To the extent the agent can prevent growth and/or kill existing cancer cells, it can be cytostatic and/or cytotoxic.

Terms such as “treating,” “treatment,” “to treat,” “having a therapeutic effect,” “alleviating,” “to alleviate,” or “slowing the progression of” refer to both 1) therapeutic measures that cure, eradicate, slow down, lessen symptoms of, and/or halt progression of a diagnosed pathologic disorder, such as cancer; and 2) prophylactic or preventative measures that prevent and/or slow the development of cancer. Thus, those in need of treatment include those already with the disorder; those prone to have the disorder; and those in whom the disorder is to be prevented. In certain embodiments, a subject is successfully “treated” for cancer, according to the methods of the present disclosure, if the patient shows one or more of the following: reduction in cachexia, increase in survival time, elongation in time to tumor progression, reduction in tumor mass, reduction in tumor burden and/or a prolongation in time to tumor metastasis, time to tumor recurrence or progressive disease, tumor response, complete response (CR), partial response (PR), stable disease, progression free survival (PFS), overall survival (OS), each as measured by standards set by the National Cancer Institute and the U.S. Food and Drug Administration (FDA) for the approval of new drugs. See, Johnson et al, J. Clin. Oncol. 27:1404-1411 (2003). In some embodiments, the “therapeutic effect,” as defined above, also encompasses a reduction in toxicity or adverse side effects, and/or an improvement in tolerability.

“Administering” refers to the physical introduction of a SETD2 inhibitor as described herein to a subject, using any of the various methods and delivery systems known to those skilled in the art. Routes of administration include oral, mucosal, topical, intravenous, intramuscular, subcutaneous, intraperitoneal, spinal, or other parenteral routes of administration, for example, by injection or infusion. As used herein, the phrase “parenteral administration” means modes of administration including, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. Administering can be performed, for example, once, a plurality of times, and/or over one or more extended periods.

All numbers in this disclosure indicating amounts, ratios, physical properties of materials, and/or use are to be understood as modified by the word “about,” except as otherwise indicated. The term “about,” when referring to a number or numerical range, means that the number or range referred to is an approximation, e.g., within experimental variability (or within statistical experimental error), and thus, the number or numerical range can vary from, e.g., between 1% and 15% of the stated number or numerical range. SETD2 Inhibitors

The present disclosure provides a method for treating cancers that overexpress WHSC1 by inhibiting the histone methyltransferase, SETD2. The present disclosure relates to the unexpected discovery that inhibiting SETD2, despite its known functionality as a tumor suppressor, can be used to treat cancers that overexpress another histone methyltransferase, WHSC1. In a particular embodiment, the present disclosure relates to using a SETD2 inhibitor to treat t(4; 14) Multiple Myeloma (MM). The treatment includes, inter alia, administering to a subject in need thereof a therapeutically effective amount of an inhibitor of SETD2, and treating the cancer.

As used herein, “an inhibitor of SETD2” or “a SETD2 inhibitor” refers to any molecule or compound that modulates, e.g., downregulates, an activity of human SETD2. For example, a SETD2 inhibitor may inhibit histone methyltransferase activity of SETD2. For instance, a SETD2 inhibitor can be a compound that exhibits a biochemical 50% inhibitory concentration (IC₅₀) with respect to SETD2 in a purified enzyme assay of between about 1 nM and about 10,000 nM, between about 1 nM and about 1,000 nM, between about 1 nM and about 500 nM, between about 1 nM and about 100 nM, between about 1 nM and about 50 nM, or between about 1 nM and about 10 nM.

In some embodiments, “downregulating (or inhibiting) an activity of human SETD2” refers to inhibiting trimethylation of the lysine 36 of histone 3. In some embodiments, the SETD2 inhibitor used in the methods of the present disclosure is a small molecule (i.e., a molecule of molecular weight less than about 1,500 g/mol, e.g., between about 100 g/mol and about 1,500 g/mol) chemical compound that selectively targets and downregulates one or more activities of SETD2.

In some embodiments, the small molecule inhibitor of SETD2 is a “Substituted Indole Compound” as defined in the “Definitions” section of the DETAILED DESCRIPTION.

In some embodiments, the SETD2 inhibitor is a compound of Table 1, or a pharmaceutically acceptable salt thereof.

In some embodiments, the SETD2 inhibitor is not a Substituted Indole Compound. In some embodiments, the SETD2 inhibitor is a sinefungin derivative. Sinefungin is an analog of k-adenosyl methionine (SAM). In some embodiments, the sinefungin analog is an A-alkyl (methyl, ethyl, propyl, benzyl) sinefungin. In some embodiments, the A-alkyl sinefungin is A-propyl sinefungin (Pr-SNF) or A-benzyl sinefungin (Bn-SNF). The synthesis of sinefungin derivatives and their inhibition profile against the human methyltransferase SETD2 is described in Zheng, W. et al., J. Am. Chem. Soc. 134:18004-18014 (2012), which is incorporated by reference in its entirety.

In some embodiments, the SETD2 inhibitor is a compound from traditional Chinese medicine (TCM), such as, e.g., coniselin, coniferyl ferulate, and 1-O-cisferuloyl-3-O-trans-p-coumaroylglycerol (FOC). See, Chang, Y. L. et al., SAR and QSAR in Environmental Research 27:589-608 (2016), which is incorporated by reference in its entirety. Coniselin is isolated from the alcoholic extract of Comiselinum vaginatum Thell. Coniferyl ferulate can be isolated from Angelica sinensis, Poria cocos (SchwJ Wolf, and Notopterygium forbesii. FOC is isolated from rhizomes of Sparganium stoloniferum. Id.

In some embodiments, the SETD2 inhibitor can be, for example, a polypeptide, DNA, or RNA. The inhibitor of SETD2 can also be, for example, a molecule that specifically binds to a SETD2 polypeptide, a molecule that specifically binds to a ligand of a SETD2 polypeptide, an antisera raised against a SETD2 polypeptide, a soluble SETD2 polypeptide, or a soluble SETD2 polypeptide comprising, consisting essentially of, or consisting of an extracellular domain of a SETD2 polypeptide.

In some embodiments, the SETD2 inhibitor can also be, for example, an antibody that specifically binds to a SETD2 polypeptide or an antigen binding fragment of an antibody that specifically binds to a SETD2 polypeptide. In some embodiments, the antibody is a polyclonal, monoclonal, murine, human, humanized, or chimeric antibody. Monoclonal and polyclonal anti-SETD2 antibodies are commercially available and may be purchased, for example, from Thermo Fisher Scientific and Millipore Sigma. In some embodiments, the antigen binding fragment is a Fab, Fab′, F(ab′)₂, Fv, scFv, sdFv fragment, VH domain, or VL domain.

In some embodiments, the SETD2 inhibitor can also be, for example, an RNAi, miRNA, siRNA, shRNA, antisense RNA, antisense DNA, decoy molecule, decoy DNA, double-stranded DNA, single-stranded DNA, complexed DNA, encapsulated DNA, viral DNA, plasmid DNA, naked RNA, encapsulated RNA, viral RNA, double-stranded RNA, molecule capable of generating RNA interference, or combinations thereof, that hybridizes to a nucleotide sequence encoding a SETD2 polypeptide.

Downregulation of SETD2 can also be achieved by gene editing technologies. In some embodiments, the SETD2 inhibitor can be, for example, a clustered regularly interspaced short palindromic repeat (CRISPR)-Cas9 system. CRISPR-Cas9 systems have been described in the literature with applications in cancer biology, and can include, for example, a Cas9 nuclease and a single guide RNA (sgRNA). See, Sanchez-Rivera, F. J. and Jacks, T., “Applications of the CRISPR-Cas9 System in Cancer Biology,” Nat Rev Cancer 15: 387-395 (2015); Chen, S. et al., “CRISPR-Cas9: from Genome Editing to Cancer Research,” Int. J. Biol. Sci. 12: 1427-1436 (2016). For example, a sgRNA targeting the SETD2 gene together with a Cas9 nuclease can be administered to a subject resulting in ablation of a particular sequence of the SETD2 gene resulting in downregulated SETD2 activity (i.e., inhibited trimethylation of lysine 36 of histone H3). In particular, the SET, AWS, PS, SRI, or WW domains could be targeted with CRISPR-Cas9 for ablation. A non-limiting example of a CRISPR-Cas9 system comprises an sgRNA Target Sequence #1 having the sequence AGCACCAGTAACAGAGCCAG (SEQ ID NO: 7), an sgRNA Target Sequence #2 having the sequence GACTGTGAACGGACAACTGA (SEQ ID NO: 8), and a Cas9 mRNA. In some embodiments, the sgRNAs and Cas9 mRNA may each be contained in separate vectors. In some embodiments, the sgRNAs may both be contained in a first vector and the Cas9 mRNA may be contained in a second vector. In some embodiments, the sgRNAs and the Cas9 mRNA may all be contained in a single vector. Those skilled in the art are aware of reagents and methods for formulating a CRISPR-Cas9 system for administration to a subject in need thereof.

In addition to CRISPR-Cas9-based systems, other alternative CRISPR-based systems can be used to inhibit SETD2, such as, e.g., the CRISPR/Cpf1 system of the bacterium Francisella novicida. See, Zetsche, B. et al., Cell 763:759-771 (2015); Fonfara, I et al., Nature 532: 517-521 (2016).

In addition to CRISPR-based systems, other gene editing techniques, such as, e.g., zinc finger nucleases (ZFNs), transcription activator-like effector nucleases (TALENs), and engineered homing meganucleases can also be used to inhibit SETD2. See, e.g., Maeder, M. L. and Gersbach, C. A., “Genome-editing Technologies for Gene and Cell Therapy,” Mol. Ther. 27: 430-446 (2016); Gaj, T. et al., “ZFN, TALEN, and CRISPR/Cas-based methods for genome engineering,” Trends Biotechnol 37:397-405 (2013); Perez-Pinera, P. et al., “Advances in targeted genome editing,” Curr Opin Chem Biol 76:268-277 (2012).

In one embodiment, the SETD2 inhibitor is an antisense nucleic acid or oligonucleotide that is wholly or partially complementary to, and can hybridize with, a target nucleic acid encoding the SETD2 polypeptide (either DNA or RNA). For example, an antisense nucleic acid or oligonucleotide can be complementary to 5′ or 3′ untranslated regions, or can overlap the translation initiation codon (5′ untranslated and translated regions) of at least one nucleic acid molecule encoding SETD2. As non-limiting examples, antisense oligonucleotides may be targeted to hybridize to the following regions: mRNA cap region, translation initiation site; translational termination site; transcription initiation site; transcription termination site; polyadenylation signal; 3′ untranslated region; 5′ untranslated region; 5′ coding region, mid coding region; 3′ coding region: DNA replication initiation and elongation sites.

In some embodiments, oligonucleotides can be constructed that will bind to duplex nucleic acid (i.e., DNA:DNA or DNA:RNA), to form a stable triple helix or triplex nucleic acid. Such triplex oligonucleotides can inhibit transcription and/or expression of a nucleic acid encoding SETD2. Triplex oligonucleotides are constructed using the base-pairing rules of triple helix formation.

In yet a further embodiment, oligonucleotides can be used in the present method that contain moieties having non-naturally-occurring portions. Thus oligonucleotides may have altered sugar moieties or inter-sugar linkages. Exemplary among these are phosphorothioate and other sulfur containing species which are known in the art. In preferred embodiments, at least one of the phosphodiester bonds of the oligonucleotide has been substituted with a structure that functions to enhance the ability of the compositions to penetrate into the region of cells where the RNA whose activity is to be modulated is located. It is preferred that such substitutions comprise phosphorothioate bonds, methyl phosphonate bonds, or short chain alkyl or cycloalkyl structures.

In other embodiments, the phosphodiester bonds are substituted with structures which are, at once, substantially non-ionic and non-chiral, or with structures which are chiral and enantiomerically specific. Persons of ordinary skill in the art will be able to select other linkages for use in the disclosed methods, including inverted terminal nucleotides.

Oligonucleotides may also include species that include at least some modified base forms. Thus, purines and pyrimidines other than those normally found in nature may be so employed. Similarly, modifications on the furanosyl portions of the nucleotide subunits may also be affected. Examples of such modifications are 2′-O-alkyl- and 2′-halogen-substituted nucleotides. Some non-limiting examples of modifications at the 2′ position of sugar moieties include OH, SH, SCH₃, F, OCH₃, OCN, O(CH₂), NH₂, and O(CH₂)nCH₃, where n is from 1 to about 10. Such oligonucleotides are functionally interchangeable with natural oligonucleotides or synthesized oligonucleotides, which have one or more differences from the natural structure. All such analogs are comprehended herewith so long as they function effectively to hybridize with at least one nucleic acid molecule encoding SETD2 to inhibit the function thereof.

A polypeptide, antibody, polynucleotide, vector, cell, or composition which is “isolated” is a polypeptide, antibody, polynucleotide, vector, cell, or composition that is in a form not found in nature. Isolated polypeptides, antibodies, polynucleotides, vectors, cells or compositions include those which have been purified to a degree that they are no longer in a form found in nature. In some embodiments, an antibody, polynucleotide, vector, cell, or composition which is isolated is substantially pure. As used herein, “substantially pure” refers to material which is at least 50% pure (i.e., free from contaminants), at least 90% pure, at least 95% pure, at least 98% pure, or at least 99% pure.

“Polynucleotide” or “nucleic acid,” as used interchangeably herein, refer to polymers of nucleotides of any length, and include both DNA and RNA. The nucleotides can be deoxyribonucleotides, ribonucleotides, modified nucleotides or bases, and/or their analogs, or any substrate that can be incorporated into a polymer by DNA or RNA polymerase. A polynucleotide can comprise modified nucleotides, such as methylated nucleotides and their analogs. If present, modification to the nucleotide structure can be imparted before or after assembly of the polymer. The sequence of nucleotides can be interrupted by non-nucleotide components.

The terms “polypeptide,” “peptide,” and “protein” are used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides of the present disclosure are based upon antibodies, in certain embodiments, the polypeptides can occur as single chains or associated chains.

The terms “identical” or percent “identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection.

Various algorithms and software are known in the art that can be used to obtain alignments of amino acid or nucleotide sequences. One such non-limiting example of a sequence alignment algorithm is the algorithm described in Karlin et al., Proc. Natl. Acad Sci. 87:2264-2268 (1990), as modified in Karlin et al., Proc. Natl. Acad Sci. 90:5873-5877 (1993), and incorporated into the NBLAST and XBLAST programs (Altschul et al., Nucleic Acids Res. 25:3389-3402 (1991)). In certain embodiments, Gapped BLAST can be used as described in Altschul et al., Nucleic Acids Res. 25:3389-3402 (1997). BLAST-2, WU-BLAST-2 (Altschul et al., Methods in Enzymology 266:460-480 (1996)), ALIGN, ALIGN-2 (Genentech, South San Francisco, Calif.) or Megalign (DNASTAR) are additional publicly available software programs that can be used to align sequences. In certain embodiments, the percent identity between two nucleotide sequences is determined using the GAP program in GCG software (e.g., using a NWSgapdna.CMP matrix and a gap weight of 40, 50, 60, 70, or 90 and a length weight of 1, 2, 3, 4, 5, or 6). In certain alternative embodiments, the GAP program in the GCG software package, which incorporates the algorithm of Needleman and Wunsch (J. Mol. Biol. 45:444-453 (1970)), can be used to determine the percent identity between two amino acid sequences (e.g., using either a Blossum 62 matrix or a PAM250 matrix, and a gap weight of 16, 14, 12, 10, 8, 6, or 4 and a length weight of 1, 2, 3, 4, 5).

Alternatively, in certain embodiments, the percent identity between nucleotide or amino acid sequences is determined using the algorithm of Myers and Miller (CABIOS, 4:11-17 (1989)). For example, the percent identity can be determined using the ALIGN program (version 2.0) and using a PAM120 with residue table, a gap length penalty of 12 and a gap penalty of 4. Appropriate parameters for maximal alignment by particular alignment software can be determined by one skilled in the art. In certain embodiments, the default parameters of the alignment software are used. In certain embodiments, the percentage identity “X” of a first amino acid sequence to a second amino acid sequence is calculated as 100×(Y/Z), where Y is the number of amino acid residues scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be larger than the percent identity of the second sequence to the first sequence.

As a non-limiting example, whether any particular polynucleotide has a certain percentage sequence identity (e.g., is at least 80% identical, at least 85% identical, at least 90% identical, and in some embodiments, at least 95%, 96%, 97%, 98%, or 99% identical) to a reference sequence can, in certain embodiments, be determined using the Bestfit program (Wisconsin Sequence Analysis Package, Version 8 for Unix, Genetics Computer Group, University Research Park, 575 Science Drive, Madison, Wis. 53711). Bestfit uses the local homology algorithm of Smith and Waterman, Advances in Applied Mathematics 2: 482-489 (1981), to find the best segment of homology between two sequences. When using Bestfit or any other sequence alignment program to determine whether a particular sequence is, for instance, 95% identical to a reference sequence according to the present disclosure, the parameters are set such that the percentage of identity is calculated over the full length of the reference nucleotide sequence and that gaps in homology of up to 5% of the total number of nucleotides in the reference sequence are allowed.

In some embodiments, two nucleic acids or polypeptides described herein are substantially identical, meaning they have at least 70%, at least 75%, at least 80%, at least 85%, at least 90%, and in some embodiments at least 95%, 96%, 97%, 98%, 99% nucleotide or amino acid residue identity, when compared and aligned for maximum correspondence, as measured using a sequence comparison algorithm or by visual inspection. In certain embodiments, identity exists over a region of the sequences that is at least about 10, about 20, about 40-60 residues in length or any integral value there between, or over a longer region than 60-80 residues, at least about 90-100 residues, or the sequences are substantially identical over the full length of the sequences being compared, such as the coding region of a nucleotide sequence, for example.

Alternatively, expression vectors derived from retroviruses, adenovirus, herpes or vaccinia viruses or from various bacterial plasmids may be used for delivery of nucleotide sequences to the targeted organ, tissue or cell population. Methods which are well known to those skilled in the art can be used to construct recombinant vectors which will express nucleic acid sequence that is complementary to the nucleic acid sequence encoding a human SETD2 polypeptide.

RNA interference (RNAi) is a post-transcriptional gene silencing process that is induced by a miRNA or a dsRNA (a small interfering RNA; siRNA), and has been used to modulate gene expression. RNAi can be used in the therapeutic method describe herewith to inhibit SETD2. Generally, RNAi is being performed by contacting cells with a double stranded siRNA or a small hairpin RNA (shRNA). However, manipulation of RNA outside of cells is tedious due to the sensitivity of RNA to degradation. It is thus also encompassed herein deoxyribonucleic acid (DNA) compositions encoding small interfering RNA (siRNA) molecules, or intermediate siRNA molecules (such as shRNA), comprising one strand of an siRNA to be used. Accordingly, the present application provides an isolated DNA molecule, which includes an expressible template nucleotide sequence of at least about 16 nucleotides encoding an Intermediate siRNA, which, when a component of an siRNA, mediates RNA interference (RNAi) of a target RNA. The present application further concerns the use of RNA interference (RNAi) to modulate the expression of nucleic acid molecules encoding SETD2 in target cells. While the therapeutic applications are not limited to a particular mode of action, RNAi may involve degradation of messenger RNA (e.g., mRNA of genes of SETD2) by an RNA induced silencing complex (RISC), preventing translation of the transcribed targeted mRNA. Alternatively, it may also involve methylation of genomic DNA, which shuts down transcription of a targeted gene. The suppression of gene expression caused by RNAi may be transient or it may be more stable, even permanent.

“Small interfering RNA” (siRNA) can also be used in the present methods as a SETD2 inhibitor. siRNA refers to any nucleic acid molecule capable of mediating RNA interference (RNAi) or gene silencing. For example, siRNA can be double stranded RNA molecules from about 10 to about 30 nucleotides long that are named for their ability to specifically interfere with protein expression (e.g., SETD2 protein expression). In one embodiment, siRNAs of the present disclosure are 12-28 nucleotides long, more preferably 15-25 nucleotides long, even more preferably 19-23 nucleotides long, and most preferably 21-23 nucleotides long. Therefore, preferred siRNA are 12, 13, 14, 15, 16, 17, 18, 19, 20, 21, 22, 23, 24, 25, 26, 27, 28 nucleotides in length. As used herein, siRNA molecules need not to be limited to those molecules containing only RNA, but further encompass chemically modified nucleotides and non-nucleotides. siRNA can be designed to decrease expression of SETD2 in a target cell by RNA interference. siRNAs can comprise a sense region and an antisense region wherein the antisense region comprises a sequence complementary to an mRNA sequence for a nucleic acid molecule encoding SETD2 and the sense region comprises a sequence complementary to the antisense sequence of the gene's mRNA. An siRNA molecule can be assembled from two nucleic acid fragments wherein one fragment comprises the sense region and the second fragment comprises the antisense region of the siRNA molecule. The sense region and antisense region can also be covalently connected via a linker molecule. The linker molecule can be a polynucleotide linker or a non-polynucleotide linker.

In one embodiment, the SETD2 inhibitor is a human SETD2 siRNA selected from the group consisting of: SEQ ID NO: 1, SEQ ID NO: 2, SEQ ID NO: 3, and SEQ ID NO: 4.

(SEQ ID NO: 1)
UAAAGGAGGUAUAUCGAAU
(SEQ ID NO: 2)
GAGAGGUACUCGAUCAUAA
(SEQ ID NO: 3)
GCUCAGAGUUAACGUUUGA
(SEQ ID NO: 4)
CCAAAGAUUCAGACAUAUA

A ribozyme (from ribonucleic acid enzyme, also called RNA enzyme or catalytic RNA) is an RNA molecule that catalyzes a chemical reaction. Some ribozymes may play an important role as therapeutic agents, as enzymes which target defined RNA sequences, as biosensors, and for applications in functional genomics and gene discovery. Ribozymes can be genetically engineered to specifically cleave a transcript of a gene from a nucleic acid molecule encoding SETD2 whose expression is desired to be downregulated.

The delivery of the gene or genetic material into the cell (encoding partly or wholly the sequence that will lower the expression of SETD2) is the first step in gene therapy treatment of any disorder. A large number of delivery methods are well known to those of skill in the art. Preferably, the nucleic acids are administered for in vivo or ex vivo gene therapy uses. Non-viral vector delivery systems include DNA plasmids, naked nucleic acid, and nucleic acid complexed with a delivery vehicle such as a liposome. Viral vector delivery systems include DNA and RNA viruses, which have either episomal or integrated genomes after delivery to the cell.

The use of RNA or DNA based viral systems for the delivery of nucleic acids take advantage of highly evolved processes for targeting a virus to specific cells in the body and trafficking the viral payload to the nucleus. Viral vectors can be administered directly to patients (in vivo) or they can be used to treat cells in vitro and the modified cells then administered to patients (ex vivo). Conventional viral based systems for the delivery of nucleic acids could include retroviral, lentiviral, adenoviral, adeno-associated and herpes simplex virus vectors for gene transfer. Viral vectors are currently the most efficient and versatile method of gene transfer in target cells and tissues. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

In applications where transient expression of the nucleic acid is preferred, adenoviral based systems are typically used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors are also used to transduce cells with target nucleic acids, e.g., in the in vitro production of nucleic acids and peptides, and for in vivo and ex vivo gene therapy procedures.

Recombinant adeno-associated virus vectors (rAAV) are a promising alternative gene delivery systems based on the defective and nonpathogenic parvovirus adeno-associated type 2 virus. All vectors are derived from a plasmid that retains only the AAV 145 bp inverted terminal repeats flanking the transgene expression cassette. Efficient gene transfer and stable transgene delivery due to integration into the genomes of the transduced cell are key features for this vector system.

Replication-deficient recombinant adenoviral vectors (Ad) are predominantly used in transient expression gene therapy; because they can be produced at high titer and they readily infect a number of different cell types. Most adenovirus vectors are engineered such that a transgene replaces the Ad E1a, E1b, and E3 genes; subsequently the replication defective vector is propagated in human 293 cells that supply the deleted gene function in trans. Ad vectors can transduce multiple types of tissues in vivo, including non-dividing, differentiated cells such as those found in the liver, kidney and muscle tissues. Conventional Ad vectors have a large carrying capacity.

In many gene therapy applications, it is desirable that the gene therapy vector be delivered with a high degree of specificity to a particular tissue type, such as for example, the glial cells. A viral vector is typically modified to have specificity for a given cell type by expressing a ligand as a fusion protein with a viral coat protein on the viruses outer surface. The ligand is chosen to have affinity for a receptor known to be present on the cell type of interest.

Gene therapy vectors can be delivered in vivo by administration to an individual subject, typically by systemic administration (e.g., intravenous, intratumoral, intrapentoneal, intramuscular, subdermal, or intracranial infusion) or topical application. Alternatively, vectors can be delivered to cells ex vivo, such as cells explanted from an individual patient (e.g., lymphocytes, bone marrow aspirates, and tissue biopsy) or universal donor hematopoietic stem cells, followed by re-implantation of the cells into the subject, usually after selection for cells which have incorporated the vector.

In one embodiment, stem cells are used in ex vivo procedures for cell transfection and gene therapy. The advantage to using stem cells is that they can be differentiated into other cell types in vitro, or can be introduced into a mammal (such as the donor of the cells) where they will engraft at an appropriate location (such as in the bone marrow). Methods for differentiating CD34+ cells in vitro into clinically important immune cell types using cytokines such as for example GM-CSF, IFN-γ and TNF-α are known.

Stem cells are isolated for transduction and differentiation using known methods. For example, stem cells can be isolated from bone marrow cells by panning the bone marrow cells with antibodies which bind unwanted cells, such as CD4+ and CD8+(T cells), CD45+(panB cells), GR-1 (granulocytes), and lad (differentiated antigen presenting cells).

### Administration of SETD2 Inhibitors

Suitable methods of administering the SETD2 inhibitors described herein will be based on the nature of the inhibitor (i.e., small molecule, DNA, RNA, protein, antibody) and are well known to those skilled in the art. The SETD2 inhibitors described herein may be administered by the oral, parenteral, subcutaneous, intravenous, intramuscular, intraperitoneal, transdermal, intrathecal, intranasal, transmucosal, intratumoral, rectal, intravaginal, or buccal route, or by inhalation. For example, intravenous injection such as drip infusion, intramuscular injection, intraperitoneal injection, subcutaneous injection, suppositories, intestinal lavage, oral enteric-coated tablets, and the like can be selected, and the method of administration may be chosen, as appropriate, depending on the age and condition of the patient. The SETD2 inhibitors described herein may be administered systemically (e.g., by intravenous injection) or locally (e.g., intrathecally, intratumorally, or into a lymph node).

The appropriate dosage of a SETD2 inhibitor of the present disclosure depends on several factors, such as, e.g., the type of cancer to be treated, the severity, course, and stage of the cancer, the responsiveness of the cancer, previous therapy, the patient's clinical history, and so on, all at the discretion of the treating physician. In some embodiments, the dosage of the SETD2 inhibitor is from about 0.01 mg/kg to about 1000 mg/kg of body weight. In some embodiments, the dosage of the SETD2 inhibitor is about 1 mg/kg to about 500 mg/kg of body weight. In some embodiments, the dosage of the SETD2 inhibitor is about 0.1 mg/day to about 50 g/day; about 0.1 mg/day to about 25 g/day; about 0.1 mg/day to about 10 g/day; about 0.1 mg/day to about 3 g/day; or about 0.1 mg/day to about 1 g/day. Alternatively, the dosage of the SETD2 inhibitor is in the range of 1 to 2000 mg, and preferably 100 to 1000 mg per patient.

In some embodiments, the SETD2 inhibitor can be administered one time or over a series of treatments lasting from several days to several months, or until a cure is effected or a diminution of the disease state is achieved (e.g., reduction in tumor size).

In some embodiments, the SETD2 inhibitors described herein can be given once or more daily, weekly, monthly, or yearly. In certain embodiments, the SETD2 inhibitor is given once every day, once every two days, once every three days, or once every four days. In certain embodiments, the SETD2 inhibitor is given twice per day, three times per day, or four times per day. In certain embodiments, the SETD2 inhibitor is given once a week. In certain embodiments, the SETD2 inhibitor is given once every two weeks. In certain embodiments, the SETD2 inhibitor is given once every three weeks. In some embodiments, the SETD2 inhibitor is given once every four weeks. In some embodiments, the SETD2 inhibitor is given once a month.

In some embodiments, the SETD2 inhibitors described herein may be administered at an initial higher “loading” dose, followed by one or more lower doses. In some embodiments, the frequency of administration may also change. In some embodiments, a dosing regimen may comprise administering an initial dose, followed by additional doses (or “maintenance” doses) twice a day, once per day, once every two days, once every three days, or once every week. For example, a dosing regimen may comprise administering an initial loading dose, followed by a daily maintenance dose of, for example, one-half of the initial dose. Or a dosing regimen may comprise administering an initial loading dose, followed by maintenance doses of, for example one-half of the initial dose every other day. Or a dosing regimen may comprise administering three initial doses for 3 days, followed by maintenance doses of, for example, the same amount every other day.

One of ordinary skill in the art will appreciate that the dosage administered, route of administration, and frequency of administration will vary, depending upon the circumstances of the particular subject being treated, and taking into account such factors as age, gender, health, and weight of the recipient, condition or disorder to be treated, severity of the disorder, type of concurrent treatment(s), if any, and the nature of the effect desired.

One skilled in the art will also appreciate that the dosage of SETD2 inhibitor and/or frequency of administration may change during the course of therapy (lowered or increased) depending upon the patient's clinical response, side effects, etc., or during different phases of therapy (i.e., treatment or maintenance).

### Pharmaceutical Compositions

The SETD2 inhibitors used in the methods described herein can be formulated into pharmaceutical compositions suitable for administration to subjects in need thereof (i.e., subjects afflicted with cancers that overexpress WHSC1). As used herein, a “pharmaceutical composition” refers to a preparation of one or more agents as described herein (e.g., a SETD2 inhibitor), or physiologically acceptable salts or prodrugs thereof, with other chemical components, including, but not limited to, pharmaceutically acceptable carriers, excipients, lubricants, buffering agents, antibacterial agents, bulking agents (e.g., mannitol), antioxidants (e.g., ascorbic acid or sodium bisulfite), and the like. The purpose of the pharmaceutical composition is to facilitate administration of the agent(s) to a subject.

The terms “pharmaceutically acceptable carrier,” “excipients,” and “adjuvant” and “physiologically acceptable vehicle” and the like are to be understood as referring to an acceptable carrier or adjuvant that may be administered to a patient, together with the SETD2 inhibitor described herein, and which does not destroy or abrogate the pharmacological activity thereof. A pharmaceutically acceptable excipient, as used herein, includes, but are not limited to, any and all solvents, dispersion media, or other liquid vehicles, dispersion or suspension aids, diluents, granulating and/or dispersing agents, surface active agents, isotonic agents, thickening or emulsifying agents, preservatives, binders, lubricants or oil, coloring, sweetening or flavoring agents, stabilizers, antioxidants, antimicrobial or antifungal agents, osmolality adjusting agents, pH adjusting agents, buffers, chelants, cryoprotectants, and/or bulking agents, as suited to the particular dosage form desired. Various excipients for formulating pharmaceutical compositions and techniques for preparing the composition are well known in the art (see, Remington: The Science and Practice of Pharmacy, 21^{st} Edition, A. R. Gennaro (Lippincott, Williams & Wilkins, Baltimore, Md., 2006; incorporated herein by reference in its entirety).

Exemplary diluents include, but are not limited to, calcium or sodium carbonate, calcium phosphate, calcium hydrogen phosphate, sodium phosphate, lactose, sucrose, cellulose, microcrystalline cellulose, kaolin, mannitol, sorbitol, etc., and/or combinations thereof.

Exemplary granulating and/or dispersing agents include, but are not limited to, starches, pregelatinized starches, or microcrystalline starch, alginic acid, guar gum, agar, poly(vinyl-pyrrolidone), (providone), cross-linked poly(vinyl-pyrrolidone) (crospovidone), cellulose, methylcellulose, carboxymethyl cellulose, cross-linked sodium carboxymethyl cellulose (croscarmellose), magnesium aluminum silicate (VEEGUM®), sodium lauryl sulfate, etc., and/or combinations thereof.

Exemplary surface active agents and/or emulsifiers include, but are not limited to, natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), sorbitan fatty acid esters (e.g., polyoxyethylene sorbitan monooleate [TWEEN®80], sorbitan monopalmitate [SPAN®40], glyceryl monooleate, polyoxyethylene esters, polyethylene glycol fatty acid esters (e.g., CREMOPHOR®), polyoxyethylene ethers (e.g., polyoxyethylene lauryl ether [BRIJ®30]), PLUORINC® F 68, POLOXAMER®188, etc.) and/or combinations thereof.

Exemplary binding agents include, but are not limited to, starch, gelatin, sugars (e.g., sucrose, glucose, dextrose, dextrin, molasses, lactose, lactitol, mannitol), amino acids (e.g., glycine), natural and synthetic gums (e.g., acacia, sodium alginate), ethylcellulose, hydroxyethylcellulose, hydroxypropyl methylcellulose, etc., and combinations thereof.

Exemplary antioxidants include, but are not limited to, alpha tocopherol, ascorbic acid, ascorbyl palmitate, benzyl alcohol, butylated hydroxyanisole, m-cresol, methionine, butylated hydroxytoluene, monothioglycerol, sodium or potassium metabisulfite, propionic acid, propyl gallate, sodium ascorbate, etc., and combinations thereof.

Exemplary chelating agents include, but are not limited to, ethylenediaminetetraacetic acid (EDTA), citric acid monohydrate, disodium edetate, fumaric acid, malic acid, phosphoric acid, sodium edetate, tartaric acid, trisodium edetate, etc., and combinations thereof.

Exemplary antimicrobial or antifungal agents include, but are not limited to, benzalkonium chloride, benzethonium chloride, methyl paraben, ethyl paraben, propyl paraben, butyl paraben, benzoic acid, hydroxybenzoic acid, potassium or sodium benzoate, potassium or sodium sorbate, sodium propionate, sorbic acid, etc., and combinations thereof.

Exemplary preservatives include, but are not limited to, vitamin A, vitamin C, vitamin E, beta-carotene, citric acid, ascorbic acid, butylated hydroxyanisol, ethylenediamine, sodium lauryl sulfate (SLS), sodium lauryl ether sulfate (SLES), etc., and combinations thereof.

Exemplary buffers to control pH can include, but are not limited to sodium phosphate, sodium citrate, sodium succinate, histidine (or histidine-HCl), sodium malate, sodium carbonate, etc., and/or combinations thereof.

Exemplary lubricating agents include, but are not limited to, magnesium stearate, calcium stearate, stearic acid, silica, talc, malt, hydrogenated vegetable oils, polyethylene glycol, sodium benzoate, sodium or magnesium lauryl sulfate, etc., and combinations thereof.

The pharmaceutical composition or formulation described here may contain a cryoprotectant to stabilize a polynucleotide described herein during freezing. Exemplary cryoprotectants include, but are not limited to mannitol, sucrose, trehalose, lactose, glycerol, dextrose, etc., and combinations thereof.

Administration of the SETD2 inhibitor of the present disclosure is by any of the routes normally used for introducing a molecule into ultimate contact with tumor cells. The pharmaceutical compositions comprising a SETD2 inhibitor can be administered by any suitable method, e.g., parenterally, intraventricularly, orally, topically, rectally, vaginally, nasally, buccally, or via an implanted reservoir. As used herein, the term “parenteral” includes subcutaneous, intravenous, intramuscular, intra-articular, intra-synovial, intrasternal, intrathecal, intrahepatic, intralesional and intracranial injection or infusion techniques.

Parenteral formulations can be a single bolus dose, an infusion, or a loading bolus dose followed with a maintenance dose. These compositions can be administered at specific fixed or variable intervals, e.g., twice a week or once a week. In some embodiments, the SETD2 inhibitor is administered intravenously.

In certain embodiments, the pharmaceutical compositions can be orally administered in an acceptable dosage form including, e.g., capsules, tablets, aqueous suspensions or solutions. In certain embodiments, the pharmaceutical compositions also can be administered by nasal aerosol or inhalation. Such compositions can be prepared as solutions in saline, employing benzyl alcohol or other suitable preservatives, absorption promoters to enhance bioavailability, and/or other conventional solubilizing or dispersing agents.

Those skilled in the art will appreciate that specific dosage and treatment regimen for any particular patient will depend upon a variety of factors, including the particular therapeutic agents used, the patient's age, body weight, general health, sex, and diet, and the time of administration, rate of excretion, drug combination, and the severity of the particular disease being treated. Judgment of such factors by medical caregivers is within the ordinary skill in the art. The amount will also depend on the individual patient to be treated, the route of administration, the type of formulation, the characteristics of the compound used, the severity of the disease, and the desired effect. The amount used can be determined by pharmacological and pharmacokinetic principles well-known in the art.

Methods of Treating Cancers that Overexpress WHSC1

In one aspect, the present disclosure provides a method of treating a cancer that over-expresses WHSC1 in a subject, by administering to the subject in need thereof a therapeutically effective amount of a SETD2 inhibitor, such as any of those described herein.

In some embodiments, the SETD2 inhibitor is a Substituted Indole Compound as defined in the “Definitions” section of the DETAILED DESCRIPTION.

In some embodiments, the SETD2 inhibitor is a compound of Table 1, or a pharmaceutically acceptable salt thereof.

In some embodiments, the SETD2 inhibitor is not a Substituted Indole Compound. For example, the SETD2 inhibitor can be a sinefungin derivative selected from the group consisting of N-propyl sinefungin and N-benzyl sinefungin, or any other SETD2 described herein.

In some embodiments, overexpression of WHSC1 by said cancer is determined prior to administering said SETD2 inhibitor. Those skilled in the art would be able to determine expression and overexpression of WHSC1 using any of the many methods known and commonly used in the art. Examples include, but are not limited to, PCR (polymerase chain reaction), or RT-PCR, flow cytometry, Northern blot, Western blot, ELISA (enzyme linked immunosorbent assay), RIA (radioimmunoassay), gene chip analysis of RNA expression, immunohistochemistry or immunofluorescence. See, e.g., Slagle et al., Cancer 83:1401 (1998). Certain embodiments include methods wherein WHSC1 RNA expression (transcription) is determined. Other embodiments of the disclosure include methods wherein WHSC1 protein expression in the biological sample (i.e., tumor tissue) is determined. See, e.g., Harlow et al., Antibodies: A Laboratory Manual, Cold Spring Harbor Laboratory, Cold Spring Harbor, N.Y., (1988); Ausubel et al., Current Protocols in Molecular Biology, John Wiley & Sons, New York 3rd Edition, (1995); Kamel and Al-Amodi, Genomics Proteomics Bioinformatics 75:220-235 (2017). For northern blot or RT-PCR analysis, RNA is isolated from the tumor tissue sample using RNAse free techniques. WHSC1 protein expression in tumor samples can be measured using standard immunohistochemical and immunostaining techniques. See, e.g., Hudlebusch, H. R. et al., Clinical Cancer Research 77:2919-2933 (2011).

Expression level measured between different phenotypic statuses can be considered different, for example, if the mean or median expression level of WHSC1 is calculated to be statistically significant. Common tests for statistical significance include, among others, t-test, ANOVA, Kruskal-Wallis, Wilcoxon, Mann-Whitney, Significance Analysis of Microarrays, odds ratio, etc. Biomarkers (e.g., WHSC1), alone or in combination, provide measures of relative likelihood that a subject belongs to one phenotypic status or another. Therefore, they are useful, inter alia, as markers for disease and as indicators that particular therapeutic treatment regimens will likely result in beneficial patient outcomes.

In one embodiment of the disclosure, a biological sample is obtained from the patient and the biological sample is assayed for determination of WHSC1 expression or mutation status.

In another embodiment of the disclosure, Northern blot analysis of WHSC1 transcription in a tumor cell sample is performed. Northern analysis is a standard method for detection and/or quantitation of mRNA levels in a sample. Initially, RNA is isolated from a sample to be assayed using Northern blot analysis. In the analysis, the RNA samples are first separated by size via electrophoresis in an agarose gel under denaturing conditions. The RNA is then transferred to a membrane, crosslinked and hybridized with a labeled probe. Typically, Northern hybridization involves polymerizing radiolabeled or nonisotopically labeled DNA, in vitro, or generation of oligonucleotides as hybridization probes. Typically, the membrane holding the RNA sample is prehybridized or blocked prior to probe hybridization to prevent the probe from coating the membrane and, thus, to reduce non-specific background signal. After hybridization, typically, unhybridized probe is removed by washing in several changes of buffer. Stringency of the wash and hybridization conditions can be designed, selected and implemented by any practitioner of ordinary skill in the art. Detection is accomplished using detectably labeled probes and a suitable detection method. Radiolabeled and non-radiolabeled probes and their use are well known in the art. The presence and or relative levels of expression of WHSC1 can be quantified using, for example, densitometry.

In another embodiment, WHSC1 expression and/or mutation status is determined using RT-PCR. RT-PCR allows detection of the progress of a PCR amplification of a target gene in real time. Design of the primers and probes required to detect expression and/or mutation status of WHSC1 is within the skill of a practitioner of ordinary skill in the art. RT-PCR can be used to determine the level of RNA encoding WHSC1 in a tumor tissue sample. In an embodiment of the disclosure, RNA from the biological sample is isolated, under RNAse free conditions, than converted to DNA by treatment with reverse transcriptase. Methods for reverse transcriptase conversion of RNA to DNA are well known in the art. A description of PCR is provided in the following references: Mullis et al., Cold Spring Harbor Symp. Quant. Biol. 57:263 (1986); EP 50,424; EP 84,796; EP 258,017; EP 237,362; EP 201,184; U.S. Pat. Nos. 4,683,202; 4,582,788; 4,683,194.

RT-PCR probes depend on the 5′-3′ nuclease activity of the DNA polymerase used for PCR to hydrolyze an oligonucleotide that is hybridized to the target amplicon (WHSC1 gene). RT-PCR probes are oligonucleotides that have a fluorescent reporter dye attached to the 5′ end and a quencher moiety coupled to the 3′ end (or vice versa). These probes are designed to hybridize to an internal region of a PCR product. In the unhybridized state, the proximity of the fluor and the quench molecules prevents the detection of fluorescent signal from the probe. During PCR amplification, when the polymerase replicates a template on which an RT-PCR probe is bound, the 5′-3′ nuclease activity of the polymerase cleaves the probe. This decouples the fluorescent and quenching dyes and FRET no longer occurs. Thus, fluorescence increases in each cycle, in a manner proportional to the amount of probe cleavage. Fluorescence signal emitted from the reaction can be measured or followed over time using equipment which is commercially available using routine and conventional techniques.

In another embodiment of the disclosure, expression of proteins encoded by WHSC1 is detected by western blot analysis. A western blot (also known as an immunoblot) is a method for protein detection in a given sample of tissue homogenate or extract. It uses gel electrophoresis to separate denatured proteins by mass. The proteins are then transferred out of the gel and onto a membrane (e.g., nitrocellulose or polyvinylidene fluoride (PVDF)), where they are detected using a primary antibody that specifically bind to the protein. The bound antibody can then detected by a secondary antibody that is conjugated with a detectable label (e.g., biotin, horseradish peroxidase, or alkaline phosphatase). Detection of the secondary label signal indicates the presence of the protein.

In another embodiment of the disclosure, the expression of a protein encoded by WHSC1 is detected by enzyme-linked immunosorbent assay (ELISA). In one embodiment of the disclosure, “sandwich ELISA” comprises coating a plate with a capture antibody; adding sample wherein any antigen present binds to the capture antibody; adding a detecting antibody which also binds the antigen; adding an enzyme-linked secondary antibody which binds to detecting antibody; and adding substrate which is converted by an enzyme on the secondary antibody to a detectable form. Detection of the signal from the secondary antibody indicates presence of the WHSC1 antigen protein.

Numerous types of cancers that overexpress WHSC1 can be treated by the disclosed methods and pharmaceutical compositions. In some embodiments, the cancer is selected from the group consisting of adrenal cancer, acinic cell carcinoma, acoustic neuroma, acral lentigious melanoma, acrospiroma, adenocarcinoma, adenoid cystic carcinoma, adenoma, adenomatoid odontogenic tumor, adenosquamous carcinoma, adipose tissue neoplasm, adrenocortical carcinoma, AIDS-related lymphoma, alveolar rhabdomyosarcoma, alveolar soft part sarcoma, ameloblastic fibroma, anaplastic large cell lymphoma, anaplastic thyroid cancer, angiomyolipoma, angiosarcoma, astrocytoma, atypical teratoid rhabdoid tumor, basal cell carcinoma, biliary tract cancer, bladder cancer, blastoma, bone cancer, breast cancer, brain cancer, carcinoma, carcinoma in situ, carcinosarcoma, cartilage tumor, cementoma, myeloid sarcoma, chondroma, chordoma, choriocarcinoma, choroid plexus papilloma, clear-cell sarcoma of the kidney, craniopharyngioma, cutaneous T-cell lymphoma, cervical cancer, colorectal cancer, Degos disease, desmoplastic small round cell tumor, dysembryoplastic neuroepithelial tumor, dysgerminoma, embryonal carcinoma, endocrine gland neoplasm, endodermal sinus tumor, esophageal cancer, fibrosarcoma, follicular lymphoma, follicular thyroid cancer, ganglioneuroma, gastrointestinal cancer, germ cell tumor, gestational choriocarcinoma, giant cell fibroblastoma, giant cell tumor of the bone, glial tumor, glioblastoma, glioma, gliomatosis cerebri, glucagonoma, gonadoblastoma, granulosa cell tumor, gynandroblastoma, gallbladder cancer, gastric cancer, hemangioblastoma, head and neck cancer, hemangiopericytoma, hepatoblastoma, hepatocellular carcinoma, hepatosplenic T-cell lymphoma, invasive lobular carcinoma, intestinal cancer, kidney cancer, laryngeal cancer, lentigo maligna, lethal midline carcinoma, leukemia, leydig cell tumor, liposarcoma, lung cancer, lymphangioma, lymphangiosarcoma, lymphoepithelioma, liver cancer, small cell lung cancer, non-small cell lung cancer, malignant fibrous histiocytoma, malignant peripheral nerve sheath tumor, malignant triton tumor, mediastinal germ cell tumor, medullary carcinoma of the breast, medullary thyroid cancer, medulloblastoma, melanoma, meningioma, merkel cell cancer, mesothelioma, metastatic urothelial carcinoma, mixed Mullerian tumor, mucinous tumor, muscle tissue neoplasm, mycosis fungoides, myxoid liposarcoma, myxoma, myxosarcoma, nasopharyngeal carcinoma, neurinoma, neuroblastoma, neurofibroma, neuroma, nodular melanoma, ocular cancer, oligoastrocytoma, oligodendroglioma, oncocytoma, optic nerve sheath meningioma, optic nerve tumor, oral cancer, osteosarcoma, ovarian cancer, papillary thyroid cancer, paraganglioma, pinealoblastoma, pineocytoma, pituicytoma, pituitary adenoma, pituitary tumor, plasmacytoma, polyembryoma, primary central nervous system lymphoma, primary effusion lymphoma, preimary peritoneal cancer, prostate cancer, pancreatic cancer, pharyngeal cancer, pseudomyxoma periotonei, renal cell carcinoma, renal medullary carcinoma, retinoblastoma, rhabdomyoma, rhabdomyosarcoma, rectal cancer, sarcoma, Schwannomatosis, seminoma, Sertoli cell tumor, sex cord-gonadal stromal tumor, skin cancer, small cell carcinoma, soft tissue sarcoma, somatostatinoma, spinal tumor, squamous cell carcinoma, synovial sarcoma, small intestine cancer, squamous carcinoma, stomach cancer, testicular cancer, thyroid cancer, transitional cell carcinoma, throat cancer, urachal cancer, urogenital cancer, urothelial carcinoma, uveal melanoma, uterine cancer, verrucous carcinoma, visual pathway glioma, vulvar cancer, vaginal cancer, Warthin's tumor, Wilms' tumor, squamous cell carcinoma of the head and neck, adenocarcinoma squamous cell carcinoma of the esophagus, adenocarcinoma of the stomach, adenocarcinoma of the colon, hepatocellular carcinoma, cholangiocarcinoma of the biliary system, adenocarcinoma of gall bladder, adenocarcinoma of the pancreas, ductal carcinoma in situ of the breast, adenocarcinoma of the breast, adenocarcinoma of the lungs, squamous cell carcinoma of the lungs, transitional cell carcinoma of the bladder, squamous cell carcinoma of the bladder, squamous cell carcinoma of the cervix, adenocarcinoma of the cervix, endometrial carcinoma, penile squamous cell carcinoma, and squamous cell carcinoma of the skin.

In some embodiments, the cancer is esophageal cancer, kidney cancer, stomach cancer, hepatocellular carcinoma, glioblastoma, central nervous system (CNS) cancer, soft tissue cancer, lung cancer, breast cancer, bladder/urinary tract cancer, head and neck cancer, melanoma, prostate cancer, testicular cancer, hematological cancer, pancreatic cancer, skin cancer, endometrial cancer, ovarian cancer, colon cancer, or colorectal cancer.

In some embodiments, the cancer is a hematologic cancer that overexpresses WHSC1, selected from the group consisting of acute lymphocytic leukemia (ALL), acute myeloid leukemia (AML), chronic lymphocytic leukemia (CLL), small lymphocytic lymphoma (SLL), multiple myeloma (MM), Hodgkin's lymphoma (HL), non-Hodgkin's lymphoma (NHL), mantle cell lymphoma (MCL), marginal zone B-cell lymphoma, splenic marginal zone lymphoma, follicular lymphoma (FL), Waldenstrom's macroglobulinemia (WM), diffuse large B-cell lymphoma (DLBCL), marginal zone lymphoma (MZL), hairy cell leukemia (HCL), Burkitt's lymphoma (BL), Richter's transformation, acute eosinophilic leukemia, acute erythroid leukemia, acute lymphoblastic leukemia, acute megakaryoblastic leukemia, acute monocytic leukemia, acute promyelocytic leukemia, acute myelogenous leukemia, B-cell prolymphocytic leukemia, B-cell lymphoma, MALT lymphoma, precursor T-lymphoblastic lymphoma, T-cell lymphoma, mast cell leukemia, adult T cell leukemia/lymphoma, aggressive NK-cell leukemia, and angioimmunoblastic T-cell lymphoma.

In some embodiments, the hematological cancer that overexpresses WHSC1 is selected from the group consisting of acute lymphocytic leukemia (ALL), acute myeloid leukemia (AML), chronic lymphocytic leukemia (CLL), small lymphocytic lymphoma (SLL), multiple myeloma (MM), non-Hodgkin's lymphoma (NHL), mantle cell lymphoma (MCL), follicular lymphoma (FL), Waldenstrom's macroglobulinemia (WM), diffuse large B-cell lymphoma (DLBCL), marginal zone lymphoma (MZL), including extranodal and nodal MZL, hairy cell leukemia (HCL), Burkitt's lymphoma (BL), and Richter's transformation.

In some embodiments, the hematologic cancer that overexpresses WHSC1 is multiple myeloma.

In some embodiments, the hematologic cancer that overexpresses WHSC1 is t(4; 14) multiple myeloma, as described herein.

In some embodiments, the hematologic cancer that overexpresses WHSC1 is a non-t(4;14) multiple myeloma, such as t(14; 16); t(11; 14); t(14;20), t(8; 14), and t(6;14), as described herein. In some embodiments, the non-t(4;14) multiple myeloma cells do not overexpress WHSC1, but are still responsive to SETD2 inhibition. This is shown in the Example below.

In some embodiments, the cancer is refractory to traditional chemotherapy.

In some embodiments, the cancer has relapsed.

In some embodiments, treatment by the methods described herein can last indefinitely (i.e., as a maintenance therapy). In some embodiments, treatment by the methods described herein can last up to about 18 weeks, up to about 17 weeks, up to about 16 weeks, up to about 15 weeks, up to about 14 weeks, up to about 13 weeks, or up to about 12 weeks. In some embodiments, treatment lasts about 12 weeks. In some embodiments, treatment by the methods described herein can last between about 1 week and about 52 weeks, between about 1 week and about 26 weeks, between about 1 week and about 12 weeks, between about 1 week and about 6 weeks, between about 6 weeks and about 52 weeks, between about 6 weeks and about 26 weeks, or between about 12 weeks and about 52 weeks. In some embodiments, treatment by the methods described herein can last more than 52 weeks.

### Methods of Inhibiting Trimethylation of Lysine 36 on Histone H3 (H3K36me3) in a Cell

In one aspect, the present disclosure provides a method of inhibiting the trimethylation of lysine 36 on histone H3 (H3K36me3) in a cell that overexpresses WHSC1, the method comprising contacting said cell with a SETD2 inhibitor, as described herein.

In some embodiments, overexpression of WHSC1 by said cancer is determined prior to administering said SETD2 inhibitor.

In some embodiments, the SETD2 inhibitor is a Substituted Indole Compound as defined in the “Definitions” section of the DETAILED DESCRIPTION.

In some embodiments, the SETD2 inhibitor is a compound of Table 1, or a pharmaceutically acceptable salt thereof.

In some embodiments, the SETD2 inhibitor is not a Substituted Indole Compound. For example, the SETD2 inhibitor can be a sinefungin derivative selected from the group consisting of N-propyl sinefungin and N-benzyl sinefungin, or any other SETD2 inhibitor described herein.

In some embodiments, inhibiting trimethylation of lysine 36 on histone H3 in a cell occurs in vitro. In some embodiments, inhibiting trimethylation of lysine 36 on histone H3 in a cell occurs in vivo. In some embodiments, the in vivo cell is in a mammal. In some embodiments, the in vivo cell is in a human.

In some embodiments, the cell is derived from a hematologic cancer. In some embodiments, the hematologic cancer is selected from the group consisting of acute lymphocytic leukemia (ALL), acute myeloid leukemia (AML), chronic lymphocytic leukemia (CLL), small lymphocytic lymphoma (SLL), multiple myeloma (MM), Hodgkin's lymphoma (HL), non-Hodgkin's lymphoma (NHL), mantle cell lymphoma (MCL), marginal zone B-cell lymphoma, splenic marginal zone lymphoma, follicular lymphoma (FL), Waldenstrom's macroglobulinemia (WM), diffuse large B-cell lymphoma (DLBCL), marginal zone lymphoma (MZL), hairy cell leukemia (HCL), Burkitt's lymphoma (BL), Richter's transformation, acute eosinophilic leukemia, acute erythroid leukemia, acute lymphoblastic leukemia, acute megakaryoblastic leukemia, acute monocytic leukemia, acute promyelocytic leukemia, acute myelogenous leukemia, B-cell prolymphocytic leukemia, B-cell lymphoma, MALT lymphoma, precursor T-lymphoblastic lymphoma, T-cell lymphoma, mast cell leukemia, adult T cell leukemia/lymphoma, aggressive NK-cell leukemia, and angioimmunoblastic T-cell lymphoma.

In some embodiments, the hematologic cancer is multiple myeloma.

In some embodiments, the multiple myeloma contains a chromosomal translocation or a chromosomal deletion.

In some embodiments, the multiple myeloma contains a chromosomal translocation.

In some embodiments, the chromosomal translocation involves chromosome 14.

In some embodiments, the chromosomal translocation is a t(4;14) translocation. In t(4; 14) translocations, the MM cells overexpress WHSC1.

In some embodiments, the chromosomal translocation is a non-t(4;14) translocation. In some embodiments, the non-t(4;14) translocation is selected from the group consisting of a t(14;16); t(11; 14); t(14;20), t(8; 14), and t(6; 14) translocation. In some embodiments, the non-t(4;14) MM cells do not overexpress WHSC1, but the SETD2 inhibitor can still inhibit trimethylation of Lysine 36 on histone H3 (H3K36me3) in a cell.

In some embodiments, the multiple myeloma contains a deletion. In some embodiments, the deletion is selected from the group consisting of del(17p) and del(13).

In some embodiments, the cell is derived from a solid tumor. In some embodiments, the solid tumor is selected from the group consisting of esophageal cancer, kidney cancer, stomach cancer, hepatocellular carcinoma, glioblastoma, central nervous system (CNS) cancer, soft tissue cancer, lung cancer, breast cancer, bladder/urinary tract cancer, head and neck cancer, melanoma, prostate cancer, testicular cancer, pancreatic cancer, skin cancer, endometrial cancer, ovarian cancer, colon cancer, and colorectal cancer.

The present invention is further illustrated by the following example which should not be construed as further limiting. The contents of all patent and non-patent references cited throughout this application are expressly incorporated herein by reference in their entireties.

### Example 1

### Small Molecule SETD2 Inhibitor Shows Potent Anti-Proliferative Activity in t(4;14) Multiple Myeloma

t(4; 14) chromosomal translocations are found in 15% of newly diagnosed multiple myeloma (MM) patients and are associated with high risk and poor prognosis. t(4; 14) MM cells are known to overexpress the histone methyltransferase (HMT) WHSC1, which leads to deregulation of gene expression due to increased dimethylation of histone H3 at lysine 36 (H3K36me2). Another HMT, SETD2, is the only enzyme capable of trimethylation of H3K36 (H3K36me3). Since t(4; 14) MM overexpresses WHSC1 resulting in ubiquitous H3K36me2 (dimethylation), this Example evaluates if t(4; 14) MM is dependent on H3K36me3 catalyzed by SETD2. Also, in this Example, a panel of MM cell lines (with and without a t(4; 14) translocation) were tested against small molecule inhibitors of SETD2. t(4; 14) multiple myeloma cell lines showed substantial loss in proliferation/viability in response to SETD2 inhibitor Cpd. No. 15 compared to non t(4; 14). Finally, this Example shows robust tumor growth regression in a KMS11 t(4; 14) xenograft model by Cpd. No. 15, indicating that SETD2 is a viable therapeutic target in t(4; 14) MM.

### Materials and Methods

Tissue Culture and Cell Lines

Cell lines used in this Example were obtained from the following sources and were cultured according to conditions specified by their respective cell banks. A549 (CCL-185), MM.1R (CRL-2975), MM.1S (CRL-2974), NCI-H929 (CRL-9068), U266B1 (TIB-196), and RPMI-8226 (CCL-155) were obtained from ATCC (Manassas, Va., USA). KMS-12-BM (ACC-551), LP-1 (ACC-41), OPM-2 (ACC-50), EJM (ACC-560), MOLP-2 (ACC-607), MOLP-8 (ACC-569), AMO-1 (ACC-538), L363 (ACC-49), SK-MM-2 (ACC-430), SK-MM-1 (ACC-758) were obtained from DSMZ (Braunschweig, Germany). KMS-28-BM (JCRB1192), KMS-26 (JCRB1187), KMS-34 (JCRB1195), KMS-11 (JCRB1179), delta-47 (JCRB1344), KMM-1 (JCRB1180) were obtained from JCRB (Osaka, Japan) and PCM6 (RCB1460) was obtained from RIKEN (Tsukuba, Japan). All cells were maintained in a humidified incubator set to 37° C., 5% CO₂.

Western Blot Analysis

Whole cell lysates were prepared using 1× NP40 buffer (ThermoFisher Scientific, FNN0021) supplemented with 1 mM PMSF and Halt™ Protease Inhibitor Cocktail (ThermoFisher Scientific, 78440). Cells were pelleted, washed with ice-cold 1×PBS, resuspended in ice-cold NP40 buffer, and incubated on ice for 30 minutes before sonication (Amplitude 30%/5 sec×1). Lysates were centrifuged at 13,200 rpm for 10 minutes at 4° C. and normalized for protein concentration by Pierce BCA Protein Assay Kit (ThermoFisher Scientific, 23225). Twenty-five micrograms of lysate was resolved on a 4-12% Bis-Tris Protein gel (ThermoFisher Scientific, WG1402BOX) and transferred using the iBlot (Program 3-7 minutes, nitrocellulose transfer stacks). The blots were probed overnight (O/N) with the following primary antibodies in Odyssey Blocking Buffer (LI-COR Biosciences, 927-40000) with 0.1% Tween 20 (v/v): rabbit anti-Tri-Methyl Histone H3 (Lys36) (D5A7) Antibody (Cell Signaling Technology, 4909S, 1:1,000 dilution), rabbit anti-Di-Methyl Histone H3 (Lys36) (C75H12) Antibody (Cell Signaling Technology, 2901S, 1:1,000 dilution), and mouse anti-Histone H3 (Cell Signaling Technology, 3638S, 1:20,000 dilution). Membranes were probed for 1 hour with IRDye 800CW Donkey anti-Rabbit IgG (LI-COR Biosciences, 926-32213, 1:20,000 dilution) and IRDye 680RD Donkey anti-Mouse IgG (LI-COR Biosciences, 926-68072, 1:20,000 dilution) secondary antibodies. Blots were imaged using the Odyssey Imaging System (LICOR Biosciences).

Individual sgRNA CRISPR Infections

Single expression system lentivirus containing Cas9 and sgRNA for all targets were purchased from Cellecta, Inc. The sequences for the sgRNAs are as follows: WHSC1-1 sgRNA-CCCATTCACTGTCCACTTGA (SEQ ID NO: 5) and WHSC1-2 sgRNA-CCCTCAAGTGGACAGTGAAT (SEQ ID NO: 6). On day 0, cells were plated at a density of 17,500 cells/cm² in a 100 mm culture dish containing 10 mL complete medium and incubated for 24 hours at 37° C., with 5% CO₂. After 24 hours, the cells were infected with sgRNAs at MOI 3 in the presence of 5 ug/mL Polybrene (Millipore, #TR-1003-G). Viral media was removed 24 hours post infection and selection by puromycin (1 ug/mL) was initiated 48 hours post infection. Infected cells were cultured under puromycin selection for 30 days.

In Cell Western Assay

A549 cells were maintained in growth medium (F12K supplemented with 10% v/v heat inactivated fetal bovine serum and 100 units/mL penicillin-streptomycin) and cultured at 37° C. under 5% CO₂. Compound was added directly to Poly-D-Lysine coated 384 well culture plates. Cells were seeded in assay medium at a density of 80,000 cells per mL (4,000 cells/well) and added to plates in a volume of 50 μL per well. Plates were left on the bench top for 20 minutes to allow cells to settle on the bottom of the well. Plates were incubated at 37° C., 5% CO₂ for 3 days. After three days of incubation, media was removed from plates and cells were permeabilized with ice cold 100% methanol. Plates were incubated for 30 minutes then washed with 1×PBS-Tween 20 (0.5%). Next, plates were blocked with Odyssey blocking buffer (LI-COR Biosciences, 927-40000) for 1 hour at room temperature. Blocking buffer was removed and 20 μL per well of primary antibody (rabbit anti-Tri-Methyl Histone H3 (Lys36) (D5A7) Antibody, Cell Signaling Technology, 4909S, 1:1,000 dilution) in Odyssey buffer with 0.1% Tween 20 (v/v) was added and plates were incubated overnight (16 hours) at 4° C. Plates were washed with 1×PBS-Tween 20 (0.5%) then 20 μL per well of secondary antibody (IRDye 800CW goat anti-rabbit IgG (H+L), LI-COR Biosciences, 926-32211, 1:500 dilution), (DRAQ5 antibody, Cell Signaling Technology, 4048L, 1:1000 dilution) was added in Odyssey buffer with 0.1% Tween 20 (v/v) and incubated for 1 hour at room temperature. The plates were washed first with 1×PBS-Tween 20 (0.5%) then with water. Plates were imaged on the Odyssey Imaging System (LI-COR Biosciences) using both 700 nm and 800 nm channels. Ratios for each well were calculated by dividing the 800 nm (H3K36me3) value by the 700 nm (DRAQ5) value. Percent inhibition values were then calculated using the test sample ratio and the average of positive and negative control ratios.

In Vitro Long-Term Proliferation (LTP) Assay

Long-term proliferation assay plating densities for each suspension cell line were determined based on growth curves (measured by Calcein AM cell viability) and density over a 4 day time course. On day 0, cells were plated in 96-well plates in triplicate and were either untreated, DMSO-treated, or treated with Cpd. No. 15 starting at 10 μM and decreasing in three-fold dilutions. Plates were read on the Acumen on days 0, 4, 7, 11, and 14 using Calcein AM (Invitrogen, C3099). Cells from each treatment were counted and replated at the original seeding density in 96-well plates in triplicate on days 4, 7, and 11. The replated cells were retreated with compound using the same dilution scheme described above. Average of triplicates was used to plot proliferation over the time course, and calculate IC₅₀ values.

Dose Range Finding Studies

All the procedures related to animal handling, care, and treatment in this study were performed according to the guidelines approved by the Institutional Animal Care and Use Committee of Pharmaron, Beijing, China and following the guidance of the Association for Assessment and Accreditation of Laboratory Animal Care.

Six to eight-week-old NOD SCID mice were purchased from Beijing HFK Bioscience Co., Ltd. Mice were housed in polycarbonate cages maintained at a temperature of (22+/−3° C.) and relative humidity of 40-70%. Animals had free access to sterile drinking water and irradiation sterilized dry granule food throughout the study. Mice were randomly assigned to dose groups based on body weight such that each treatment group had the same mean body weight. Compound or vehicle was administered at the indicated doses (n=4, 0.5% NaCMC+0.1% Tween-80 in water vehicle; n=7 for Cpd. No. 15 dose groups) twice a day (every 12 hours) or once a day, by oral gavage. Each dose was delivered in a volume of 10 mL/kg. Body weights were measured daily over the course of 7 days and mice were assessed for abnormal clinical signs. For pharmacokinetic analysis, plasma samples were collected from dosed groups at specified time points through the 7 day study and bioanalytical analyses performed.

Xenograft Studies

KMS11 Xenograft

KMS11 cells were harvested during exponential growth phase, and mixed in a 1:1 ratio of RPMI-1640: Matrigel. NOD SCID mice received 1×10⁷ cells (0.1 mL cell suspension) subcutaneously in the right flank. After 7 days, mice bearing 85 to 150 mm³ tumors were sorted into treatment groups with mean tumor volumes of 117 to 119 mm³ (n=10 mice per group). Cpd. No. 15 or vehicle (0.5% NaCMC+0.1% Tween-80 (v/v) in water) was administered at the indicated doses twice a day (every 12 hours) or once a day, by oral gavage. Each dose was delivered in a volume of 10 mL/kg, and adjusted for the last recorded weight of individual animals. The maximal treatment length was 28 days. Tumor volumes (twice weekly) and body weight (daily) were recorded throughout the experiment. On day 7 or day 28 during the studies, mice were sampled in a pre-specified fashion. Sampling included nonterminal retro-orbital bleeds and full volume blood collection via terminal cardiac puncture under CO₂ anesthesia. Blood samples were processed for plasma, with K2-EDTA as anticoagulant, and PBMCs isolated. The samples were frozen at −80° C. and stored before analysis. Tumors were harvested from specified mice under RNase-free conditions and snap frozen in liquid nitrogen. Bone marrow was flushed from the femur using 1×PBS, strained over a 40 um nylon strainer, and flash frozen. Histones were extracted from tumors, bone marrow, and PBMCs as previously described. See, Daigle et al., Cancer Cell 20:53-65 (2011).

MM.1S Xenograft

CB17 SCID mice were inoculated subcutaneously in the right flank with MM.1S tumor cells (5×10⁶) in 0.2 mL of PBS mixed with Matrigel (50:50). After 18 days, mice bearing 100 to 150 mm³ tumors were sorted into treatment groups with mean tumor volumes of 103 mm³ (n=10 mice per group). Cpd. No. 15 or vehicle (0.5% NaCMC+0.1% Tween-80 (v/v) in water) was administered at the indicated doses twice a day (every 12 hours), by oral gavage. Each dose was delivered in a volume of 10 mL/kg, and adjusted for the last recorded weight of individual animals. The maximal treatment length was 23 days. Tumor volumes and body weight were recorded twice weekly throughout the experiment. On day 18 or day 41 during the studies, mice were sampled in a pre-specified fashion. Sampling included nonterminal retro-orbital bleeds and full volume blood collection via terminal cardiac puncture under CO₂ anesthesia. Blood samples were processed for plasma, with K2-EDTA as anticoagulant, and PBMCs isolated. The samples were frozen at −80° C. and stored before analysis. Tumors were harvested from specified mice under RNase-free conditions and snap frozen in liquid nitrogen. Bone marrow was flushed from the femur using 1×PBS, strained over a 40 um nylon strainer, and flash frozen. Histones were extracted from tumors, bone marrow, and PBMCs as previously described Daigle et al., Cancer Cell 20:53-65 (2011).

Fluorescent-Based ELISA Assay (Fluroimmunoassay (FIA) Detection of Anti-Histone H3 Tri-Methyl K36)

Histone concentration was determined with Pierce BCA Protein Assay Kit (ThermoFisher Scientific, 23225). Histones were prepared in coating buffer and added directly to high-binding 96 well plates. Plates were left at 4° C. overnight to allow histones to adhere. The next morning, coating buffer with histones was discarded and 100 μL per well of primary antibody solution, (anti-histone H3 tri-methyl K36 (Epigentek, 4042-050, 1:100 dilution) and total histone H3 (Cell Signaling Technology, 14269, 1:500 dilution) in Odyssey buffer with 0.1% Tween 20 (v/v), was added to the plate and incubated for 1 hour. Plates were washed 3 times with 1×PBS-Tween 20 (0.5%) wash buffer. Next, 100 μL per well of secondary solution antibody was added, IRDye 800CW donkey anti-rabbit IgG (H+L) antibody (LI-COR Biosciences, 926-32213, 1:200 dilution) and donkey anti-mouse IgG (H+L) Alexa Fluor 680 conjugate (Life Technologies, A10038, 1:1000 dilution) in Odyssey buffer with 0.1% Tween 20 (v/v), and incubated for 1 hour in the dark at room temperature. The plates were washed 3 times with 1×PBS-Tween 20 (0.5%) wash buffer then filled with 100 μL per well of 1×PBS-Tween 20 (0.5%) and kept out of direct exposure to light as much as possible. Plates were imaged on the Odyssey Imaging System (LI-COR Biosciences) using both 700 nm and 800 nm channels. The average of the ratio values for each test sample was calculated by dividing the 800 nm (H3K36me3) value by the 700 nm value (total H3) and used to determine the percent of H3K36me3 from vehicle.

### Results

Small Molecule Inhibition of SETD2 is Enriched in t(4; 14) Cell Lines

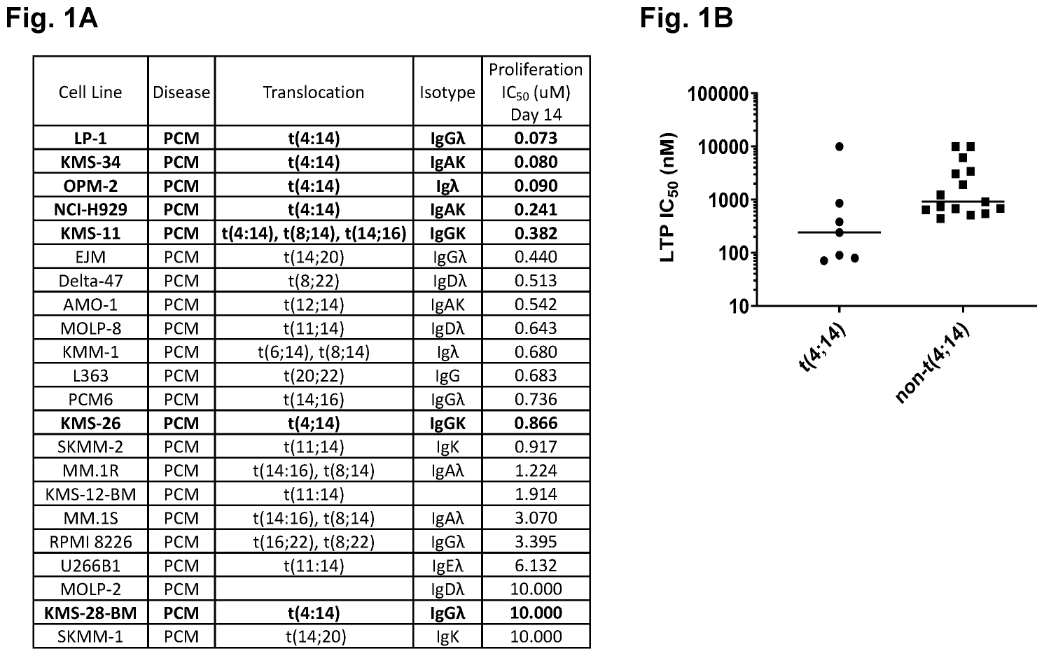

It was hypothesized that the dependency of t(4; 14) on the catalytic activity of WHSC1 was actually reflective of a dependency on subsequent SETD2 activity. To test this, a small molecule inhibitor of SETD2, Cpd. No. 15, was utilized to investigate the effect of SETD2 inhibition in the context of multiple myeloma. A panel of 22 human myeloma cell lines were interrogated for phenotypic effects upon treatment with this compound. These cell lines, representing a broad range of MM translocations, were subjected to long-term proliferation assays over a 14-day period to assess anti-proliferative effects (FIG. 1A). Cpd. No. 15 showed higher activity in the t(4; 14) subset of MM.

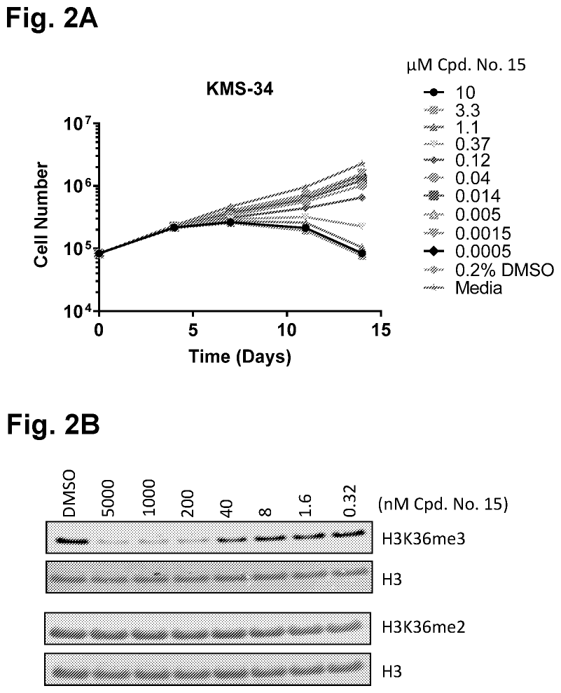

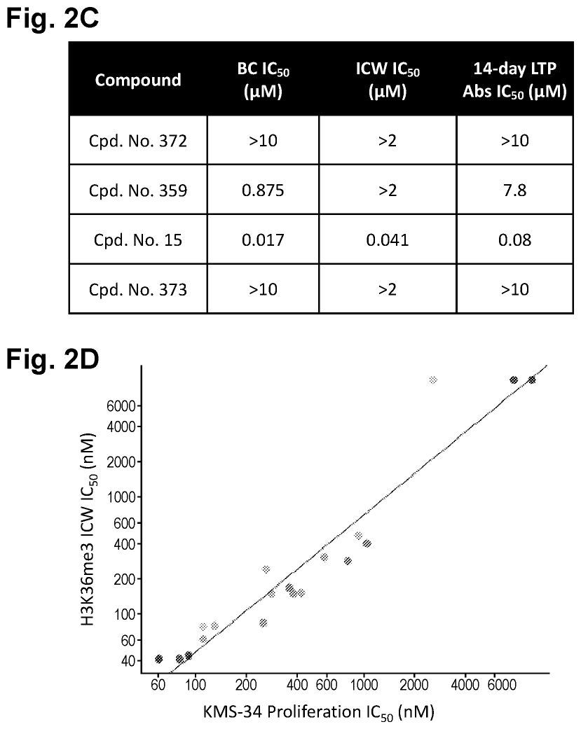

To further interrogate the effect of SETD2 inhibition in a t(4; 14) setting the MM cell line, KMS34, was selected for follow-up assays. Cpd. No. 15 inhibited growth of the KMS-34 line in vitro, in a dose dependent manner, with an IC₅₀ of 80 nM (FIG. 2A). Additionally, H3K36me3 was reduced by Cpd. No. 15 treatment, while H3K36me2 remained unaltered (FIG. 2B). To verify that the activity of Cpd. No. 15 was on-target, we compared it to three less active enantiomers from the same chemical series. While Cpd. No. 15 exhibited biochemical activity against SETD2 protein, as well as a reduction of methyl mark and proliferation defects, none of the less active enantiomers showed activity by these measures (FIG. 2C). The structure activity relationship (SAR) was further tested with a panel of 17 compounds with a range of biochemical potencies, and showed a direct correlation between H3K36me3 inhibition and in vitro proliferation effects (FIG. 2D). Taken together, it was shown that small molecule inhibition of SETD2 dose dependently inhibits proliferation in a t(4; 14) MM line, and that this effect was on target.

In Some Embodiments, the Response to SETD2 Inhibition in t(4; 14) MM is Dependent on WHSC1 Over Expression

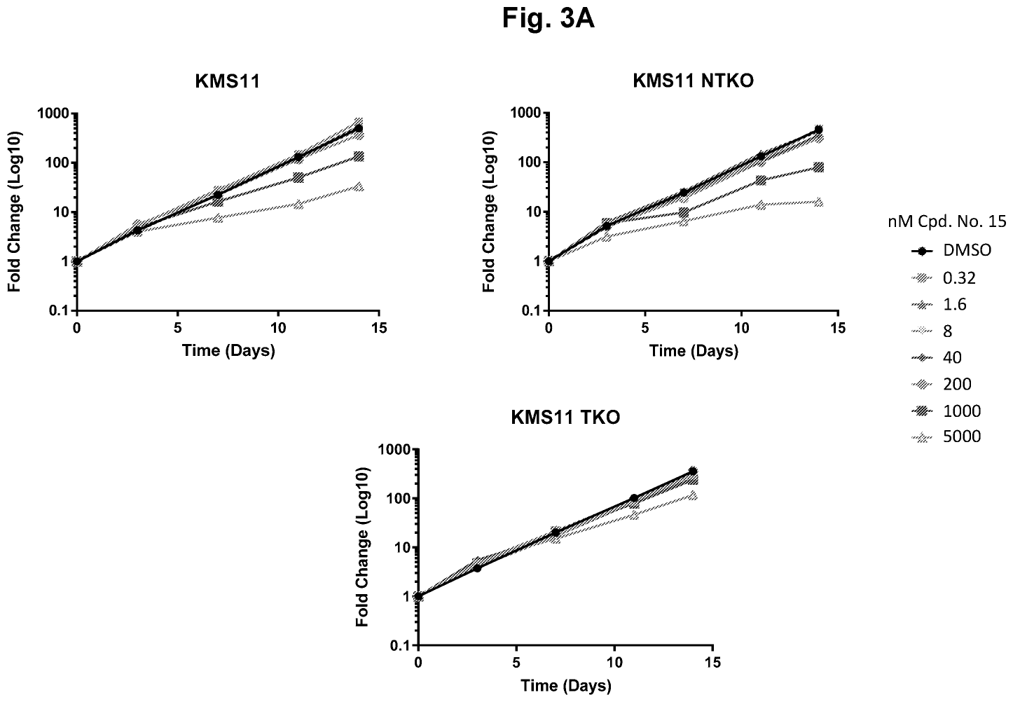

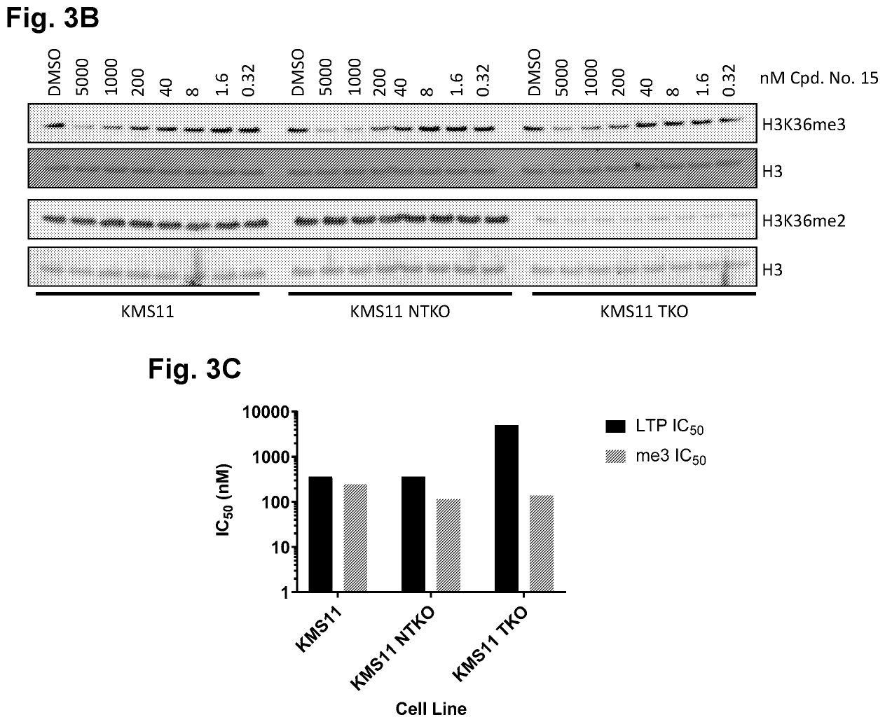

Given the enrichment of SETD2 inhibition in cell lines containing the t(4; 14) translocation, the relationship between WHSC1 overexpression and SETD2 inhibition was sought to be understood. Two isogenic variants of KMS11 cells, the TKO and NTKOs, which have been characterized previously by Kuo, A. J. et al., Mol. Cell. 44:609-620 (2011), were subjected to long-term proliferation assays. NTKO cells express only the translocated WHSC1 allele; whereas, TKO cells express only the non-translocated allele of WHSC1. As a result, NTKO cells overexpress WHSC1, while TKO cells lack WHSC1 overexpression driven by the t(4; 14) translocation. After 14 days of treatment with Cpd. No. 15, both the parental and NTKO lines expressing the translocated allele exhibit inhibited growth with IC₅₀'s of 360 nM and 361 nM, respectively. Conversely, the TKO line, expressing only the non-translocated allele, showed no proliferation effects in response to Cpd. No. 15 (FIG. 3A). Assessment of the methyl mark revealed that H3K36me3 was reduced in a dose responsive manner with an IC₅₀ between of 150-250 nM for all variants. Additionally, assessment of the H3K36me2 methyl mark confirms that only the parental and NTKO lines have elevated levels of H3K36me2 indicative of WHSC1 overexpression. (FIGS. 3B, 3C). These results suggest that SETD2 sensitivity in t(4; 14) MM is dependent on WHSC1 overexpression.

Additionally, CRISPR knockout of WHSC1 was used to further examine a dependence on WHSC1 overexpression for sensitivity to SETD2 inhibition in t(4; 14) MM cell lines. Two t(4; 14) MM cell lines were selected based on their sensitivity to SETD2 inhibition. KMS-34 cells were sensitive to SETD2 inhibition with an IC₅₀ of 80 nM; whereas, KMS-28-BM cells were insensitive to SETD2 inhibition with an IC₅₀ of 10 uM (FIG. 1A). It was hypothesized that KMS-28-BM cells were not sensitive to SETD2 inhibition because they were not dependent on t(4;14)-driven WHSC1 overexpression for survival, potentially due to other drivers such as low Cyclin D2 expression and KRAS mutations. If true, knockout of WHSC1 would not affect the proliferation or viability of these cells. Conversely, the KMS-34 cells were expected to be sensitive to loss of WHSC1, given their sensitivity to SETD2 inhibition.

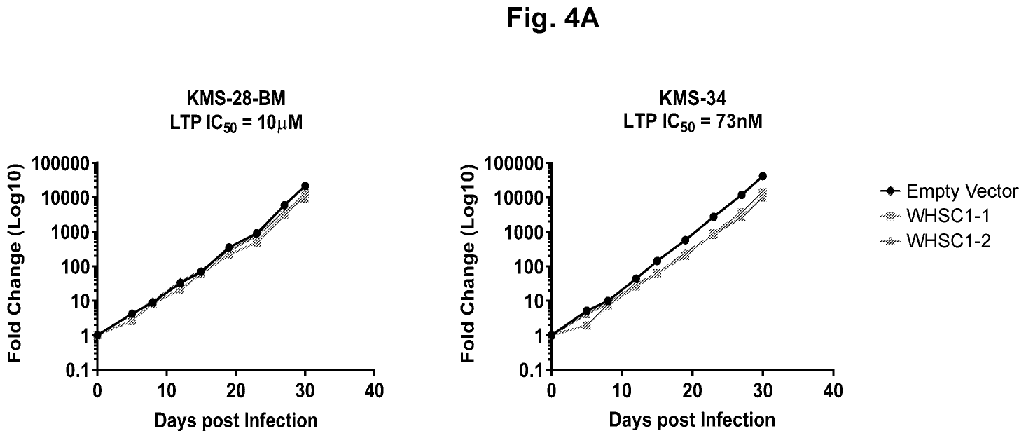

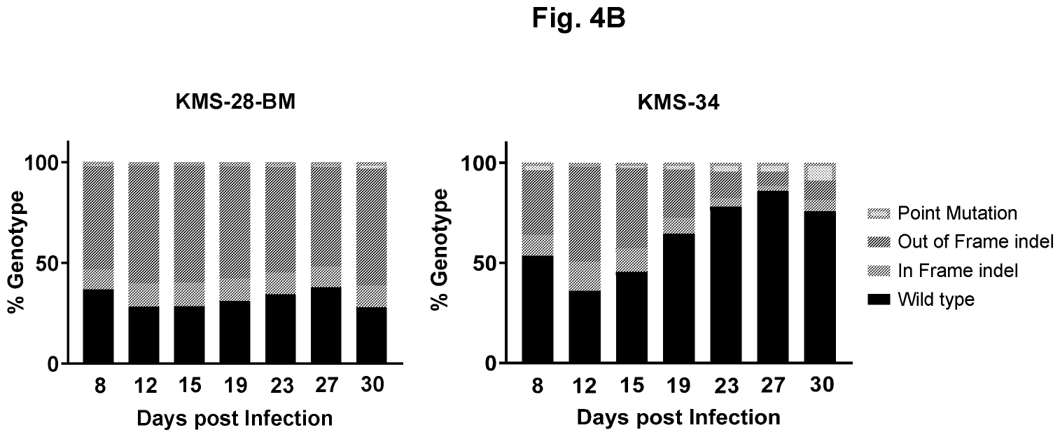

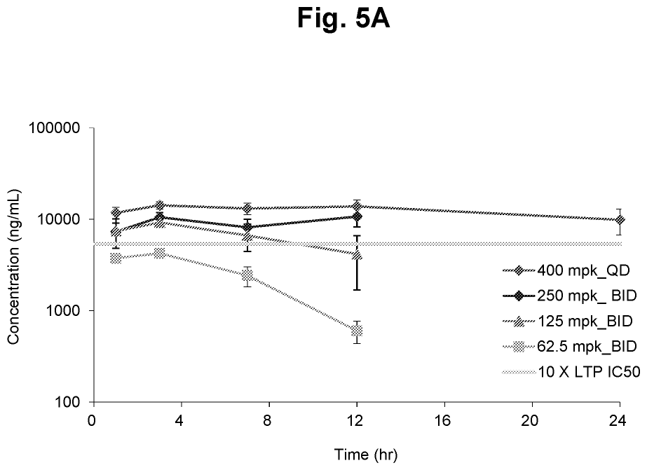

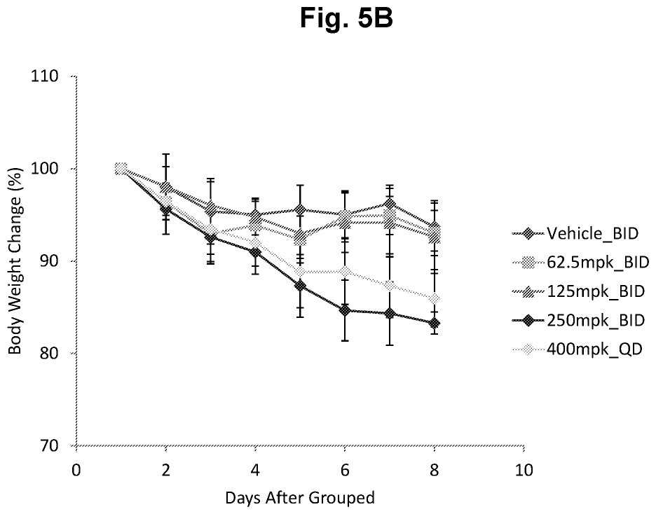

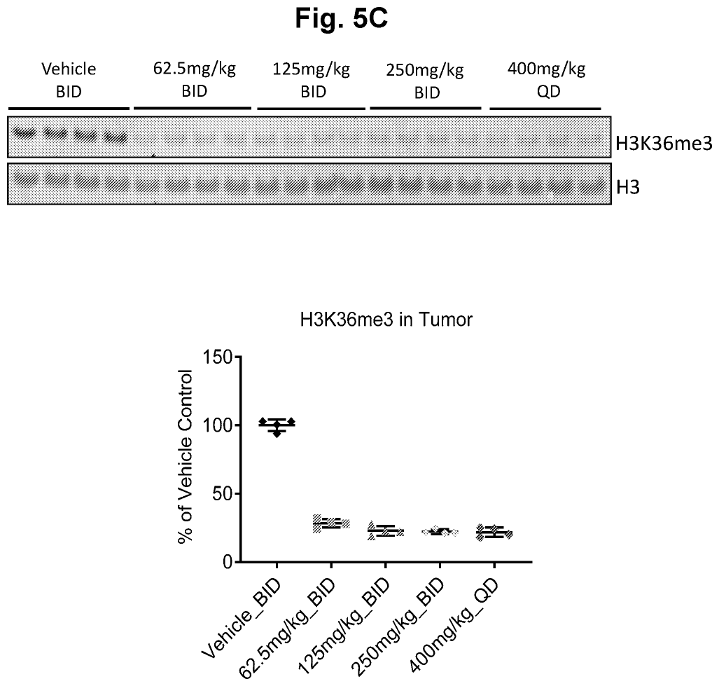

To test these theories, both cell lines were subjected to CRISPR knockout of WHSC1 and growth and genotype were monitored over a period of 4 weeks. It was observed that knockout of WHSC1 in KMS-28-BM cells had no effect on proliferation; whereas, in the KMS-34 cell line, there was a marked decrease in proliferation in the WHSC1 targeted lines (FIG. 4A).

Genotype analysis of the knockout lines over time showed that more than 50% of the KMS-28-BM resulted in out of frame mutations suggesting that the cells survived with the loss of WHSC1. Conversely, KMS-34 genotyping showed a maximal out of frame population at 12-days post infection after which the positive selection for the wild-type population occurred (FIG. 4B), indicating a loss of cells with WHSC1 knockout, and an outgrowth of cells that maintained WHSC1 overexpression. Taken together, these results suggest that sensitivity to SETD2 inhibition in t(4; 14) MM is contingent upon a dependency on overexpression of WHSC1.

Cpd No. 15 is Tolerated In Vivo at Doses 10× In Vitro IC₅₀

In order to determine the doses for in vivo efficacy studies, a dose range finding (DRF) study was performed in the KMS11 xenograft model. The in vitro Cpd. No. 15 IC₅₀ for KMS11 cells was used as the benchmark for selecting the doses used in the DRF. The pharmacokinetics in NOD SCID mice following oral administration of Cpd. No. 15 indicated that drug levels can be maintained that exceed ten times the KMS11 proliferation IC₅₀ in mice with either BID or QD dosing for up to 12 hours (FIG. 3A). No body weight loss is observed at 62.5 and 125 mg/kg BID when compared with vehicle control (FIG. 3B). Furthermore, modulation of the H3K36me3 methyl mark in naïve bone marrow shows a substantial reduction in H3K36me3 levels in response to Cpd. No. 15 treatment (FIG. 3C). Taken together, these results show that SETD2 inhibitor Cpd. No. 15 is very well tolerated at effective concentrations in NOD SCID mice, and shows target engagement of in vivo pharmacodynamic mark.

SETD2 Inhibition Leads to Robust Tumor Regression in a t(4; 14) MM Xenograft Model

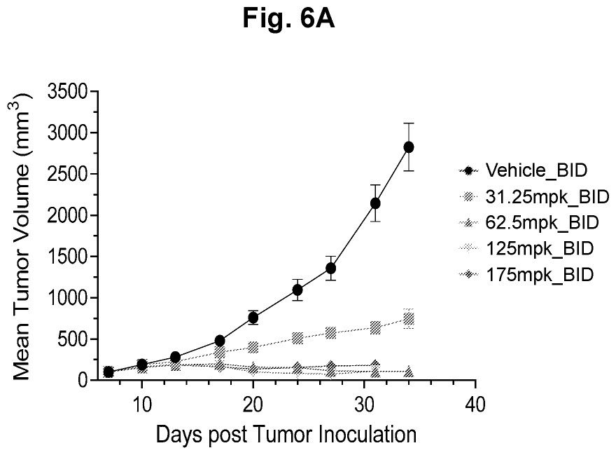

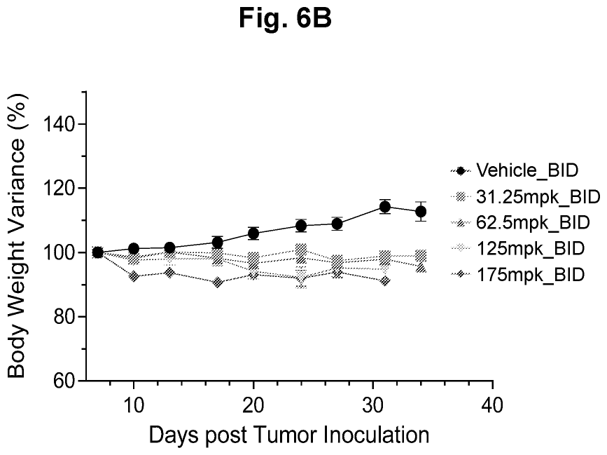

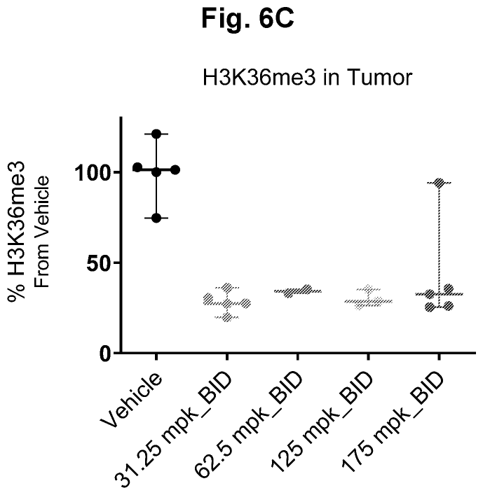

To determine whether the growth inhibitory effects of SETD2 inhibition observed in cell culture translated to in vivo studies, the effect of treatment of Cpd. No. 15 on the KMS11 xenograft model was examined. Studies were performed in NOD SCID mice bearing subcutaneous KMS11 tumors. Mice were dosed orally for 28 days with Cpd. No. 15 or vehicle control. Cpd. No. 15 showed robust tumor regression in a dose-dependent manner with maximum tumor inhibition of 99% at the top three doses (FIG. 6A). Cpd. No. 15 was well tolerated with minimal effects on body weight (BW) at 31.25 mg/kg (28 days BID) and 62.5 mg/kg (28 days BID); while 125 mg/kg (18 days BID, 7 days BID (3D-4D+)) and 175 mg/kg (11 days BID, 14 days BID (3D-4D+) were less tolerated and required dosing holidays after 18 and 11 days of continuous dosing, respectively (FIG. 6B). Fluorescent-based ELISA analysis of H3K36me3 in histones isolated from tumor collected on day 28 showed complete reduction of the methyl mark at all doses (FIG. 6C). Cpd. No. 15 is extremely well tolerated and demonstrates strong antitumor activity in KMS11 t(4; 14) multiple myeloma xenograft model with complete ablation of the H3K36me3 methyl mark at all observed doses.

SETD2 Inhibition Leads to Tumor Regression in a Non-t(4; 14) MM Xenograft Model

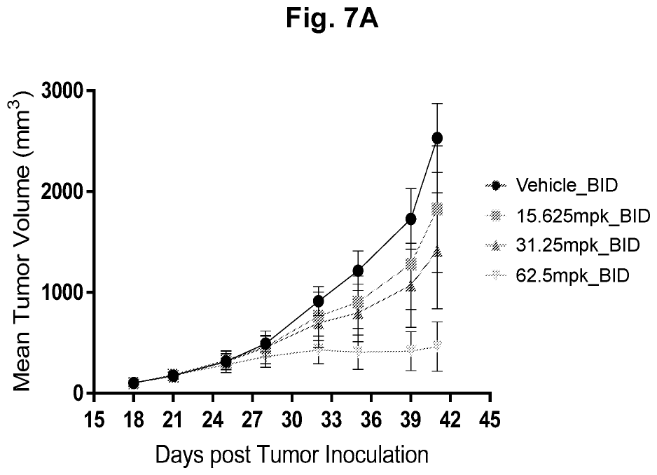

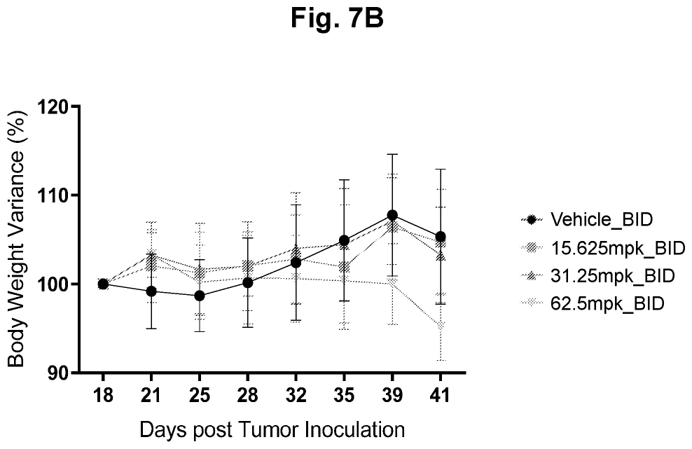

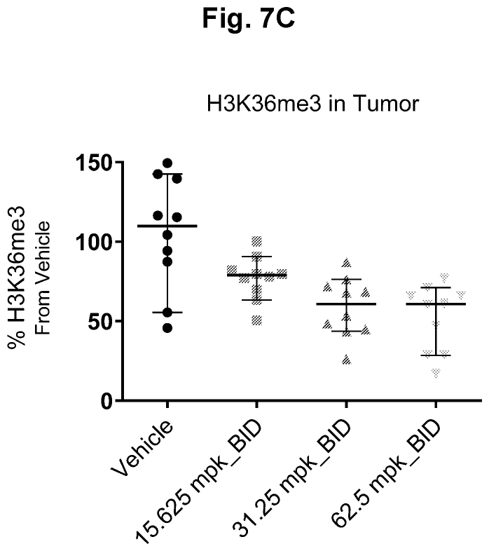

While SETD2 inhibition clearly resulted in growth inhibition of t(4; 14) MM cell lines, we also observed a response in non-t(4;14) cell lines. Given these results, Cpd. No. 15 was tested in the non-t(4;14) MM xenograft model MM.1S. This cell line was ˜10-fold less sensitive in the cellular long-term proliferation assay with an IC₅₀ of 3 uM compared to that of 382 nM for the KMS11 cell line (FIG. 1A). Studies were performed in CB17 SCID mice bearing subcutaneous MM.1S tumors. Cpd. No. 15 or vehicle control were dosed twice daily by oral gavage for 23 days. Cpd. No. 15 showed tumor inhibition in a dose-dependent manner with maximal inhibition seen at the top dose group (62.5 mg/kg—85% TGI) (FIG. 7A). The compound was well tolerated at all doses with minimal changes in body weight after 23 continuous days of dosing (FIG. 7B). A dose dependent decrease in the H3K36me3 methyl mark is seen in the MM. 1S tumor samples (FIG. 7C). As would be expected based on the cellular LTP assay, the MM. 1S xenograft model responded to treatment with Cpd. No. 15 but to a smaller degree than the KMS11 xenograft model. Tumor growth inhibition was seen after treatment, up to 85%, whereas in the KMS11 model, almost complete tumor regression was seen. This suggests that SETD2 inhibition is likely most effective in the t(4; 14) subset of MM, but other MM subtypes are effected as well, albeit to a lesser extent.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance. All patents and publications mentioned in the specification are indicative of the levels of those of ordinary skill in the art to which the disclosure pertains. All patents and publications are herein incorporated by reference to the same extent as if each individual publication was specifically and individually indicated to be incorporated by reference.

1. A method of treating cancer in a subject in need thereof, the method comprising administering to the subject a therapeutically effective amount of a SETD2 inhibitor, wherein the cancer overexpresses WHSC1.

2. The method of claim 1, wherein overexpression of WHSC1 by said cancer is determined prior to administering said SETD2 inhibitor.

3. The method of claims 1-2, wherein the SETD2 inhibitor is a Substituted Indole Compound.

4. The method of claim 3, wherein the SETD2 inhibitor is a compound of Table 1, or a pharmaceutically acceptable salt thereof.

5. The method of claims 1-2, wherein the SETD2 inhibitor is not a Substituted Indole Compound.

6. The method of claims 1-5, wherein the cancer that overexpresses WHSC1 is a hematologic cancer.

7. The method of claim 6, wherein the hematologic cancer is selected from the group consisting of acute lymphocytic leukemia (ALL), acute myeloid leukemia (AML), chronic lymphocytic leukemia (CLL), small lymphocytic lymphoma (SLL), multiple myeloma (MM), Hodgkin's lymphoma (HL), non-Hodgkin's lymphoma (NHL), mantle cell lymphoma (MCL), marginal zone B-cell lymphoma, splenic marginal zone lymphoma, follicular lymphoma (FL), Waldenstrom's macroglobulinemia (WM), diffuse large B-cell lymphoma (DLBCL), marginal zone lymphoma (MZL), hairy cell leukemia (HCL), Burkitt's lymphoma (BL), Richter's transformation, acute eosinophilic leukemia, acute erythroid leukemia, acute lymphoblastic leukemia, acute megakaryoblastic leukemia, acute monocytic leukemia, acute promyelocytic leukemia, acute myelogenous leukemia, B-cell prolymphocytic leukemia, B-cell lymphoma, MALT lymphoma, precursor T-lymphoblastic lymphoma, T-cell lymphoma, mast cell leukemia, adult T cell leukemia/lymphoma, aggressive NK-cell leukemia, and angioimmunoblastic T-cell lymphoma.

8. The method of claim 7, wherein the hematologic cancer is multiple myeloma.

9. The method of claim 8, wherein the multiple myeloma contains a chromosomal translocation or a chromosomal deletion.

10. The method of claim 9, wherein the multiple myeloma contains a chromosomal translocation.

11. The method of claim 10, wherein the chromosomal translocation involves chromosome 14.

12. The method of claim 11, wherein the chromosomal translocation is a t (4;14) translocation.

13. The method of claim 12, wherein the chromosomal translocation is a non-t(4;14) translocation.

14. The method of claim 13, wherein the non-t(4;14) translocation is selected from the group consisting of a t(14; 16); t(11; 14); t(14;20), t(8; 14), and t(6; 14) translocation.

15. The method of claim 9, wherein the multiple myeloma contains a chromosomal deletion.

16. The method of claim 15, wherein the deletion is selected from the group consisting of del(17p) and del(13).

17. The method of any one of claims 1-5, wherein the cancer that overexpresses WHSC1 is a solid tumor.

18. The method of claim 17, wherein the solid tumor is selected from the group consisting of esophageal cancer, kidney cancer, stomach cancer, hepatocellular carcinoma, glioblastoma, central nervous system (CNS) cancer, soft tissue cancer, lung cancer, breast cancer, bladder/urinary tract cancer, head and neck cancer, melanoma, prostate cancer, testicular cancer, pancreatic cancer, skin cancer, endometrial cancer, ovarian cancer, colon cancer, and colorectal cancer.

19. The method of any one of claims 1-18, wherein the subject is a mammal.

20. The method of any one of claims 1-18, wherein the subject is a human.

21. The method of any one of claims 1-20, wherein the compound is formulated for systemic or local administration.

22. The method of any one of claims 1-20, wherein the compound is formulated for oral, nasal, intra-peritoneal, or intra-tumoral administration.

23. The method of any one of claims 1-20, wherein the compound is formulated for intravenous administration, intramuscular administration, or subcutaneous administration.

24. A method of inhibiting the trimethylation of lysine 36 on histone H3 (H3K36me3) in a cell, the method comprising contacting said cell with a SETD2 inhibitor, wherein the cell overexpresses WHSC1.

25. The method of claim 24, wherein the SETD2 inhibitor is a Substituted Indole Compound.

26. The method of claim 25, wherein the SETD2 inhibitor is a compound of Table 1, or a pharmaceutically acceptable salt thereof.

27. The method of claim 24, wherein the SETD2 inhibitor is not a Substituted Indole Compound.

28. The method of any one of claims 24-27, wherein inhibiting trimethylation of lysine 36 on histone H3 in a cell occurs in vitro.

29. The method of any one of claims 24-27, wherein inhibiting trimethylation of lysine 36 on histone H3 in a cell occurs in vivo.

30. The method of any one of claims 24-29, wherein the cell is derived from a hematologic cancer.

31. The method of claim 30, wherein the hematologic cancer is selected from the group consisting of acute lymphocytic leukemia (ALL), acute myeloid leukemia (AML), chronic lymphocytic leukemia (CLL), small lymphocytic lymphoma (SLL), multiple myeloma (MM), Hodgkin's lymphoma (HL), non-Hodgkin's lymphoma (NHL), mantle cell lymphoma (MCL), marginal zone B-cell lymphoma, splenic marginal zone lymphoma, follicular lymphoma (FL), Waldenstrom's macroglobulinemia (WM), diffuse large B-cell lymphoma (DLBCL), marginal zone lymphoma (MZL), hairy cell leukemia (HCL), Burkitt's lymphoma (BL), Richter's transformation, acute eosinophilic leukemia, acute erythroid leukemia, acute lymphoblastic leukemia, acute megakaryoblastic leukemia, acute monocytic leukemia, acute promyelocytic leukemia, acute myelogenous leukemia, B-cell prolymphocytic leukemia, B-cell lymphoma, MALT lymphoma, precursor T-lymphoblastic lymphoma, T-cell lymphoma, mast cell leukemia, adult T cell leukemia/lymphoma, aggressive NK-cell leukemia, and angioimmunoblastic T-cell lymphoma.

32. The method of claim 31, wherein the hematologic cancer is multiple myeloma.

33. The method of claim 32, wherein the multiple myeloma contains a chromosomal translocation or a chromosomal deletion.

34. The method of claim 33, wherein the multiple myeloma contains a chromosomal translocation.

35. The method of claim 34, wherein the chromosomal translocation involves chromosome 14.

36. The method of claim 34, wherein the chromosomal translocation is a t(4; 14) translocation.

37. The method of claim 34, wherein the chromosomal translocation is a non-t(4;14) translocation.

38. The method of claim 37, wherein the non-t(4; 14) translocation is selected from the group consisting of a t(14; 16); t(11; 14); t(14;20), t(8; 14), and t(6; 14) translocation.

39. The method of claim 33, wherein the multiple myeloma contains a deletion.

40. The method of claim 39, wherein the deletion is selected from the group consisting of del(17p) and del(13).

41. The method of claim 24, wherein the cell is derived from a solid tumor.

42. The method of claim 41, wherein the solid tumor is selected from the group consisting of esophageal cancer, kidney cancer, stomach cancer, hepatocellular carcinoma, glioblastoma, central nervous system (CNS) cancer, soft tissue cancer, lung cancer, breast cancer, bladder/urinary tract cancer, head and neck cancer, melanoma, prostate cancer, testicular cancer, pancreatic cancer, skin cancer, endometrial cancer, ovarian cancer, colon cancer, and colorectal cancer.

43. The method of any one of claims 29-42, wherein the in vivo cell is in a mammal.

44. The method of any one of claims 29-42, wherein the in vivo cell is in a human.
